(19) United States
(12) Patent Application Publication
(10) Pub. No.: US 2010/0029643 A1
(43) Pub. Date: Feb. 4, 2010

(54) HETEROCYCLYC SULFONAMIDES HAVING EDG-1 ANTAGONISTIC ACTIVITY

(75) Inventors: Gurmit Grewal, Waltham, MA (US); Edward Hennessy, Waltham, MA (US); Victor Kamhi, Waltham, MA (US); Danyang Li, Waltham, MA (US); Paul Lyne, Waltham, MA (US); Vibha Oza, Waltham, MA (US); Jamal Carlos Saeh, Waltham, MA (US); Qibin Su, Waltham, MA (US); Bin Yang, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Södertälje (SE)

(21) Appl. No.: 12/514,247

(22) PCT Filed: Nov. 8, 2007
(86) PCT No.: PCT/GB07/04267

§ 371 Date: May 8, 2009

Related U.S. Application Data
(60) Provisional application No. 60/865,364, filed on Nov. 10, 2006.
(60) Provisional application No. 60/895,699, filed on Mar. 19, 2007.
(60) Provisional application No. 60/947,795, filed on Jul. 3, 2007.
(60) Provisional application No. 60/953,838, filed on Aug. 3, 2007.

(51) Int. Cl. A61K 31/4184; C07D 235/14; C07D 401/06; C07D 471/04; C07D 403/12; C07D 413/12; C07D 401/12; A61K 31/4439; A61K 31/437; A61K 31/538; A61K 31/4709; A61K 31/5377; A61K 31/506; A61P 35/00

(52) U.S. Cl. 514/234.5; 548/309.7; 546/273.4; 546/118; 548/306.1; 544/105; 546/177; 544/139; 544/298; 514/394; 514/338; 514/303; 514/230.5; 514/314; 514/269

(57) ABSTRACT

The invention relates to chemical compounds of formula (I), (Ia) and (Ib) or pharmaceutically acceptable salts thereof, which possess Edg-1 antagonistic activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments for use in the production of an anti-cancer effect in a warm-blooded animal, such as man.

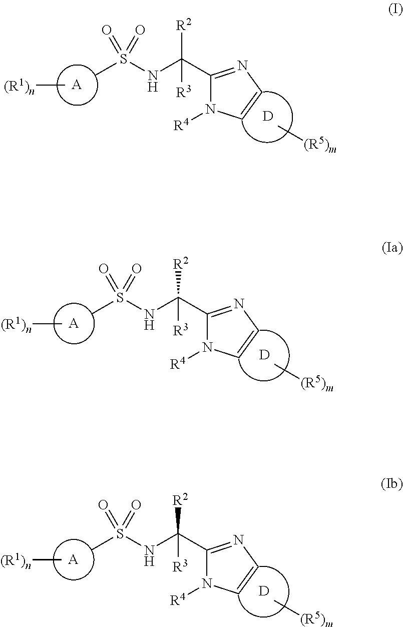

Edg (endothelial differentiation gene) receptors belong to a family of closely related, lipid activated G-protein coupled receptors. Edg-1, Edg-3, Edg-5, Edg-6, and Edg-8 (also known as S1P1, S1P3, S1P2, S1P4, and S1P5) are identified as receptors specific for sphingosine-1-phosphate (SIP). Edg-2, Edg-4, and Edg-7 (known also as LPA1, LPA2, and LPA3, respectively) are receptors specific for lysophosphatidic (LPA). Among the SIP receptor isotypes, Edg-1, Edg-3 and Edg-5 are widely expressed in various tissues, whereas the expression of Edg-6 is confined largely to lymphoid tissues and platelets, and that of Edg-8 to the central nervous system.

Edg receptors are responsible for signal transduction and are thought to play an important role in cell processes involving cell development, proliferation, maintenance, migration, differentiation, plasticity and apoptosis. Certain Edg receptors are associated with diseases mediated by the de novo or deregulated formation of vessels—for example, for diseases caused by ocular neovascularisation, especially retinopathies (diabetic retinopathy, age-related macular degeneration); psoriasis; and haemangioblastomas such as “strawberry-marks”. Edg receptors are also associated with various inflammatory diseases, such as arthritis, especially rheumatoid arthritis, arterial atherosclerosis and atherosclerosis occurring after transplants, endometriosis or chronic asthma; and, especially, tumor diseases or by lymphocyte interactions, for example, in transplantation rejection, autoimmune diseases, inflammatory diseases, infectious diseases and cancer. An alteration in Edg receptor activity contributes to the pathology and/or symptomology of these diseases. Accordingly, molecules that themselves alter the activity of Edg receptors are useful as therapeutic agents in the treatment of such diseases.

Accordingly, the present invention provides a compound of formula (I):

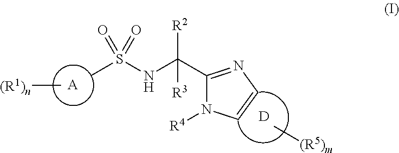

Ring A is carbocyclyl or heterocyclyl; wherein if said heterocyclyl contains an —NH— moiety that nitrogen may be optionally substituted by a group selected from R⁶;

R¹ is independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, hydrazinyl, ureido, N,N-di(C₁₋₃alkyl)ureido, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl; wherein R¹ may be optionally substituted on carbon by one or more R⁷; and wherein if said heterocyclyl contains an —NH— moiety that nitrogen may be optionally substituted by a group selected from R⁸;

n is 0-5; wherein the values of R¹ may be the same or different;

R² is selected from C₁₋₆alkyl, C₂₋₆alkenyl or C₂₋₆alkynyl, carbocyclyl, and heterocyclyl; wherein R² may be optionally substituted on carbon by one or more R⁹; wherein if said heterocyclyl contains an NH moiety that nitrogen may be optionally substituted by a group selected from R¹⁹;

R³ is selected from hydrogen, C₁₋₆alkyl, C₂₋₆alkenyl or C₂₋₆alkynyl, carbocyclyl, heterocyclyl; wherein R³ may be optionally substituted on carbon by one or more R¹¹; wherein if said heterocyclyl contains an NH moiety that nitrogen may be optionally substituted by a group selected from R²⁰;

or, alternatively, R² and R³ may, together with the carbon to which they are attached, form a C₃₋₆-carbocyclic ring;

R⁴ is selected from C₁₋₆alkyl or carbocyclyl; wherein R⁴ may be optionally substituted on carbon by one or more R¹⁰;

Ring D is fused to the imidazole of formula (I) and is a 5-7 membered ring; wherein if said ring contains an —NH— moiety that nitrogen may be optionally substituted by a group selected from R¹⁴;

R⁵ is a substituent on carbon and is independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, heterocyclylcarbonyl, N—(C₁₋₆alkyl)sulphamoyl, N,N—(C₁₋₆alkyl)₂sulphamoyl, C₁₋₆alkylsulphonylamino, carbocyclyl or heterocyclyl, or two R⁵ may together with the carbon atoms of ring D to which they are attached form a 5 to 8-membered carbocyclyl or heterocyclyl ring; wherein R⁵ may be optionally substituted on carbon by one or more R¹⁵; and wherein if said heterocyclyl or heterocyclyl ring contains an —NH— moiety that nitrogen may be optionally substituted by a group selected from R¹⁶;

m is 0-5; wherein the values of R⁵ may be the same or different;

R⁷, R⁹, R¹¹ and R¹⁵ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁷, R⁹, R¹¹ and R¹⁵ may be independently optionally substituted on carbon by one or more R¹⁷; and wherein if said heterocyclyl contains an —NH— moiety that nitrogen may be optionally substituted by a group selected from R¹⁸;

R⁶, R⁸, R¹³, R¹⁴, R¹⁶, R¹⁸, R¹⁹ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁰ is selected from halo, nitro, hydroxy, amino, carboxy, mercapto, sulphamoyl, C₁₋₆alkyl, C₂₋₆alkenyl, C₂₋₆alkynyl, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R¹⁰ may be optionally substituted on carbon by one or more R¹²; and wherein if said heterocyclyl contains an —NH— moiety that nitrogen may be optionally substituted by a group selected from R¹³;

R¹² and R¹⁷ are selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof; provided the compound is not 4-methyl-N-[(1-methyl-1H-benzimidazol-2-yl)phenylmethyl]benzenesulfonamide.

In another embodiment the compounds of the invention are directed to compounds of formula (I) wherein A, D, R¹, R², R³, R⁴, R⁵, m and n are as defined in formula (I), and pharmaceutically acceptable salts thereof, provided R⁴ is not difluoromethyl.

In another embodiment the compounds of the invention are directed to compounds of formula (Ia)

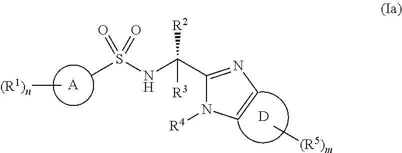

wherein R³ is hydrogen and A, D, R¹, R², R⁴, R⁵, m and n are as defined in formula (I), and pharmaceutically acceptable salts thereof.

In another embodiment the compounds of the invention are directed to compounds of formula (Ia) wherein R³ is hydrogen and A, D, R¹, R², R⁴, R⁵, m and n are as defined in formula (I), and pharmaceutically acceptable salts thereof, provided R⁴ is not difluoromethyl.

In another embodiment the compounds of the invention are directed to compounds of formula (Ib)

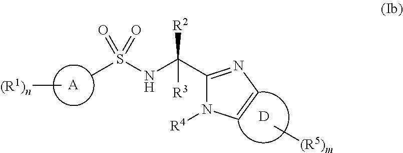

wherein R³ is hydrogen and A, D, R¹, R², R⁴, R⁵, m and n are as defined in formula (I), and pharmaceutically acceptable salts thereof.

In another embodiment the compounds of the invention are directed to compounds of formula (Ib) wherein R³ is hydrogen and A, D, R¹, R², R⁴, R⁵, m and n are as defined in formula (I), and pharmaceutically acceptable salts thereof, provided R⁴ is not difluoromethyl.

In another embodiment, the compounds of the instant invention are directed to compounds of any one of formula (I), (Ia) and (Ib) wherein

Ring A is carbocyclyl or heterocyclyl; wherein if said heterocyclyl contains an —NH— moiety that nitrogen may be optionally substituted by a group selected from R⁶;

R¹ is independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl; wherein R¹ may be optionally substituted on carbon by one or more R⁷; and wherein if said heterocyclyl contains an —NH— moiety that nitrogen may be optionally substituted by a group selected from R⁸;

n is 0-5; wherein the values of R¹ may be the same or different;

R² is selected from C₁₋₆alkyl, C₂₋₆alkenyl or C₂₋₆alkynyl, carbocyclyl, and heterocyclyl; wherein R² may be optionally substituted on carbon by one or more R⁹; wherein if said heterocyclyl contains an NH moiety that nitrogen may be optionally substituted by a group selected from R¹⁹;

R³ is selected from hydrogen, C₁₋₆alkyl, C₂₋₆alkenyl or C₂₋₆alkynyl, carbocyclyl, heterocyclyl; wherein R³ may be optionally substituted on carbon by one or more R¹¹; wherein if said heterocyclyl contains an NH moiety that nitrogen may be optionally substituted by a group selected from R²⁰;

R⁴ is selected from C₁₋₆alkyl or carbocyclyl; wherein R⁴ may be optionally substituted on carbon by one or more R¹⁰;

Ring D is fused to the imidazole of formula (I) and is a 5-7 membered ring; wherein if said ring contains an —NH— moiety that nitrogen may be optionally substituted by a group selected from R¹⁴;

R⁵ is a substituent on carbon and is independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, heterocyclylcarbonyl, N—(C₁₋₆alkyl)sulphamoyl, N,N—(C₁₋₆alkyl)₂sulphamoyl, C₁₋₆alkylsulphonylamino, carbocyclyl or heterocyclyl, or two R⁵ may together with the carbon atoms of ring D to which they are attached form a 5 to 8-membered carbocyclyl or heterocyclyl ring; wherein R⁵ may be optionally substituted on carbon by one or more R¹⁵; and wherein if said heterocyclyl or heterocyclyl ring contains an —NH— moiety that nitrogen may be optionally substituted by a group selected from R¹⁶;

m is 0-5; wherein the values of R⁵ may be the same or different;

R⁷, R⁹, R¹¹ and R¹⁵ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁷, R⁹, R¹¹ and R¹⁵ may be independently optionally substituted on carbon by one or more R¹⁷; and wherein if said heterocyclyl contains an —NH— moiety that nitrogen may be optionally substituted by a group selected from R¹⁸;

R⁶, R⁸, R¹³, R¹⁴, R¹⁶, R¹⁸, R¹⁹ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁰ is selected from halo, nitro, hydroxy, amino, carboxy, mercapto, sulphamoyl, C₁₋₆alkyl, C₂₋₆alkenyl, C₂₋₆alkynyl, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R¹⁰ may be optionally substituted on carbon by one or more R¹²; and wherein if said heterocyclyl contains an —NH— moiety that nitrogen may be optionally substituted by a group selected from R¹³;

R¹² and R¹⁷ are selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof; provided the compound is not 4-methyl-N-[(1-methyl-1H-benzimidazol-2-yl)phenylmethyl]benzenesulfonamide.

In this specification the term “alkyl” includes both straight and branched chain alkyl groups but references to individual alkyl groups such as “propyl” are specific for the straight chain version only. For example, “C₁₋₆alkyl” and “C₁₋₄alkyl” include methyl, ethyl, propyl, isopropyl and t-butyl. However, references to individual alkyl groups such as ‘propyl’ are specific for the straight-chained version only and references to individual branched chain alkyl groups such as ‘isopropyl’ are specific for the branched chain version only. A similar convention applies to other radicals.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, a ring nitrogen atom may optionally bear a C₁₋₆alkyl group and form a quaternary compound or a ring nitrogen and/or sulphur atom may be optionally oxidised to form the N-oxide and or the S-oxides. Examples and suitable values of the term “heterocyclyl” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. Additional suitable values for “heterocyclyl” include 3,4-dihydro-1,4-oxazinyl; 2,3-dihydro-1,4-benzodioxinyl; 2,1,3-benzothiadiazolyl; pyrazolyl. In one aspect of the invention a “heterocyclyl” is a saturated, partially saturated or unsaturated, mono or bicyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

“C₃₋₆-carbocyclic ring” is a saturated monocyclic carbon ring that contains 3-6 carbon atoms wherein a —CH₂— group can optionally be replaced by a —C(O)—. Suitable values for “C₃₋₆-carbocyclic ring” include cyclopropyl, cyclobutyl, cyclopentyl and cyclohexyl. “Ring D is fused to the imidazole of formula (I), formula (Ia) or formula (Ib) and is a 5-7 membered ring” said ring includes the carbon-carbon double bond of the imidazole and, apart from said double bond, comprises 3-5 additional ring atoms selected from C, N, O or S joined by single or double bonds. Suitable examples of Ring D fused to the imidazole of formula (I) include 1H-benzimidazolyl, 1H-imidazo[4,5-b]pyridinyl, 1H-imidazo[4,5-c]pyridinyl, 3H-imidazo[4,5-c]pyridinyl, 3H-imidazo[4,5-b]pyridinyl, 5H-imidazo[4,5-c]pyridazinyl and 7H-purinyl.

Two R⁵ (m=2) may together with the carbons of ring D to which they are attached form a 5 to 8-membered carbocyclyl or heterocyclyl ring. Examples of such rings include a dioxanyl or dioxolanyl ring.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂-amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N,N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are methylaminocarbonyl and ethylaminocarbonyl. Examples of “N,N—(C₁₋₆alkyl)₂-carbamoyl” are dimethylaminocarbonyl and methylethylaminocarbonyl. Examples of “C₁₋₆alkylsulphonylamino” include methylsulphonylamino, isopropylsulphonylamino and t-butylsulphonylamino. Examples of “C₁₋₆alkylsulphonyl” include methylsulphonyl, isopropylsulphonyl and t-butylsulphonyl.

Some compounds of the formula (I), formula (Ia) or formula (Ib) may have chiral centres and/or geometric isomeric centres (E- and Z-isomers), and it is to be understood that the invention encompasses all such optical, diastereoisomers and geometric isomers that possess Edg-1 antagonistic activity.

The invention relates to any and all tautomeric forms of the compounds of the formula (I), formula (Ia) or formula (Ib) that possess Edg-1 antagonistic activity.

It is also to be understood that certain compounds of the formula (I), formula (Ia) or formula (Ib) can exist in solvated as well as unsolvated forms such as, for example, hydrated forms. It is to be understood that the invention encompasses all such solvated forms which possess Edg-1 antagonistic activity.

Particular values of variable groups are as follows. Such values may be used where appropriate with any of the definitions, claims or embodiments defined hereinbefore or hereinafter.

Ring A is carbocyclyl.

Ring A is heterocyclyl; wherein if said heterocyclyl contains an —NH— moiety that nitrogen may be optionally substituted by a group selected from R⁶.

Ring A is pyrazolyl, imidazolyl, 3,4-dihydro-2H-1,4-benzoxazinyl, pyrrolyl, furanyl, pyridinyl, thiazolyl, isoxazolyl, 3,4-dihydro-2H-1,4-benzoxazinyl, 1,3-benzodioxolyl, 2,1,3-benzothiadiazole, quinolinyl or thienyl wherein said pyrazolyl, imidazolyl, 3,4-dihydro-2H-1,4-benzoxazinyl or pyrrolyl may be optionally substituted on N by a group selected from R⁶.

Ring A is pyrazolyl, imidazolyl, 3,4-dihydro-2H-1,4-benzoxazinyl, pyrrolyl, furanyl, pyridinyl, thiazolyl, isoxazolyl, 3,4-dihydro-2H-1,4-benzoxazine, 1,3-benzodioxolyl, 2,1,3-benzothiadiazole, quinolinyl, thienyl, cyclopropyl, cyclopentyl or cyclohexyl wherein said pyrazolyl, imidazolyl, 3,4-dihydro-2H-1,4-benzoxazinyl or pyrrolyl may be optionally substituted on N by a group selected from R⁶.

R⁶ is C₁₋₃alkyl.

Ring A is aryl.

Ring A is phenyl.

Ring A is phenyl, pyridinyl or pyrimidinyl.

Ring A is phenyl, pyridinyl, pyrimidinyl or pyrrolyl.

Ring A is phenyl, pyridinyl, pyrimidinyl or N-methylpyrrolyl.

Ring A is C₃₋₆cycloalkyl.

Ring A is cyclopropyl, cyclopentyl, cyclohexyl.

R¹ is halo, cyano, C₁₋₃alkanoylamino, C₁₋₃alkyl or C₁₋₃alkoxycarbonyl.

R¹ is halo, cyano, carbamoyl, or C₁₋₃alkyl.

R¹ is halo, cyano, carbamoyl, or methyl.

R¹ is halo, cyano, carbamoyl, C₁₋₃alkoxy or C₂₋₆alkenyl.

R¹ is halo or cyano.

R¹ is halo.

R¹ is bromo, chloro or fluoro.

R¹ is chloro.

n is 0-3.

n is 1.

n is 2.

Ring A is phenyl, R¹ is selected from halo or cyano and n is 1 or 2.

R² is C₁₋₆alkyl.

R² is C₁₋₆alkyl; wherein R² may be independently optionally substituted on carbon by one or more R⁹; wherein:

R⁹ is carbocyclyl.

R² is C₁₋₆alkyl; wherein R² may be independently optionally substituted on carbon by one or more R⁹; wherein:

R⁹ is carbocyclyl wherein R⁹ may be independently optionally substituted on carbon by one or more R¹⁷; wherein:

R¹⁷ is halo.

R² is C₁₋₆alkyl; wherein R² may be independently optionally substituted on carbon by one or more R⁹; wherein:

R⁹ is carbocyclyl; wherein R⁹ may be independently optionally substituted on carbon by one or more R¹⁷; wherein:

R¹⁷ is fluoro.

R² is C₁₋₆alkyl; wherein R² may be independently optionally substituted on carbon by one or more R⁹; wherein:

R⁹ is heterocyclyl.

R¹ is C₁₋₆alkyl; wherein R² may be independently optionally substituted on carbon by one or more R⁹; wherein:

R⁹ is pyridyl.

R² is C₁₋₆alkyl; wherein R² may be independently optionally substituted on carbon by one or more R⁹; wherein:

R⁹ is heterocyclyl, wherein R⁹ may be independently optionally substituted on carbon by one or more R¹⁷; wherein if said heterocyclyl contains an NH moiety that nitrogen may be optionally substituted by a group selected from R¹⁹.

R² is methyl; wherein R² may be independently optionally substituted on carbon by one or more R⁹; wherein:

R⁹ is phenyl.

R² is methyl; wherein R² may be independently optionally substituted on carbon by one or more R⁹; wherein:

R⁹ is phenyl optionally substituted on carbon by one or more R¹⁷.

R² is methyl; wherein R² may be independently optionally substituted on carbon by one or more R⁹; wherein:

R⁹ is phenyl optionally substituted on carbon by one or more halo.

R² is methyl, ethyl, isopropyl, or isobutyl; wherein R² may be independently optionally substituted on carbon by one or more R⁹.

R² is methyl, ethyl, isopropyl, isobutyl or benzyl.

R² is methyl.

R³ is hydrogen.

R³ is C₁₋₆alkyl.

R³ is methyl.

R² and R³ taken together with the carbon to which they are attached form a C₃₋₆-carbocyclic ring.

R² and R³ taken together with the carbon to which they are attached form a cyclopropyl or cyclobutyl ring.

R⁴ is selected from C₁₋₆alkyl; wherein R⁴ may be optionally substituted on carbon by one or more R¹⁰.

R⁴ is selected from methyl, ethyl, propyl or iso-butyl; wherein R⁴ may be optionally substituted on carbon by one or more R¹⁰; wherein:

R¹⁰ is cyclopropyl.

R⁴ is selected from methyl, cyclopropylmethyl, ethyl, propyl, iso-butyl or C₃₋₆cycloalkyl.

R⁴ is C₃₋₆cycloalkyl; wherein R⁴ may be optionally substituted on carbon by one or more R¹⁰.

R⁴ is cyclopropyl.

R⁴ is ethyl.

R⁴ is not difluoromethyl.

Ring D fused to the imidazole of formula (I), formula (Ia) or formula (Ib) forms 1-H-benzimidazole or 3H-imidazo[4,5-b]pyridine.

Ring D fused to the imidazole of formula (I), formula (Ia) or formula (Ib) forms 1-H-benzimidazole or 3H-imidazo[4,5-c]pyridine.

Ring D fused to the imidazole of formula (I), formula (Ia) or formula (Ib) forms 1H-benzimidazole, 1H-imidazo[4,5-b]pyridinyl, 1H-imidazo[4,5-c]pyridinyl, 3H-imidazo[4,5-c]pyridinyl, 3H-imidazo[4,5-b]pyridinyl, 5H-imidazo[4,5-c]pyridazinyl and 7H-purinyl.

Ring D fused to the imidazole of formula (I), formula (Ia) or formula (Ib) forms 1H-imidazo[4,5-c]pyridine or hydrazine-3H-imidazo[4,5-c]pyridine (2:1).

Ring D fused to the imidazole of formula (I), formula (Ia) or formula (Ib) forms 1-H-benzimazole, 3H-imidazo[4,5-b]pyridine, 1H-imidazo[4,5-c]pyridine or hydrazine-3H-imidazo[4,5-c]pyridine (2:1).

R⁵ is a substituent on carbon and is independently selected from halo, carboxy, carbamoyl, C₁₋₆alkyl, C₁₋₆alkoxy, C₁₋₆alkanoyl, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, heterocyclylcarbonyl, carbocyclyl or heterocyclyl, or two R⁵ may together with the carbon atoms of ring D to which they are attached form a 5 to 8-membered carbocyclyl or heterocyclyl ring; wherein R⁵ may be optionally substituted on carbon by one or more R¹⁵; and wherein if said heterocyclyl or heterocyclyl ring contains an —NH— moiety that nitrogen may be optionally substituted by a group selected from R¹⁶;

R⁵ is halo, methyl, trifluoromethyl, N-methylmorpholino, methoxy, trifluoromethoxy, ethoxycarbonyl, hydroxymethyl, difluoromethyl, carboxy, carbamoyl, N,N—(C₁₋₆alkyl)₂-carbamoyl, N-morpholinocarbonyl, N,N-dimethylaminomethyl, N-morpholinomethyl, methoxycarbonyl, methylmercapto, methylsulfonyl, pyridinyl and cyclopropyl.

R⁵ is halo, C₁₋₆alkyl, C₁₋₆alkoxy or C₃₋₆cycloalkyl wherein R⁵ may be optionally substituted on carbon by one or more R¹⁵.

R⁵ is halo, C₁₋₆alkyl, C₁₋₆alkoxy or C₃₋₆cycloalkyl wherein R⁵ may be optionally substituted on carbon by one or more halo.

R⁵ is C₁₋₆alkyl, C₁₋₆alkoxy or C₃₋₆cycloalkyl wherein R⁵ may be optionally substituted on carbon by one or more halo.

R⁵ is halo, C₁₋₆alkyl or C₁₋₆alkoxy.

R⁵ is trifluoromethyl, methoxy or cyclopropyl.

R⁵ is halo.

R⁵ is chloro or fluoro.

R⁵ is halo and m is 1.

R⁵ is halo and m is 2.

R⁵ is C₁₋₆alkyl.

R⁵ is C₁₋₆alkyl wherein R⁵ may be optionally substituted on carbon by halo.

R⁵ is trifluoromethyl.

R⁵ is C₁₋₆alkoxy.

R⁵ is methoxy.

m is 0, 1 or 2.

m is 0, 1, 2 or 3.

m is 0.

m is 1.

m is 2.

m is 3.

In a further aspect of the invention there is provided a compound of formula (I), formula (Ia) or formula (Ib) (as depicted above) wherein:

Ring A is carbocyclyl or heterocyclyl, wherein if said heterocyclyl contains an —NH— moiety that nitrogen may be optionally substituted by a group selected from R⁶;

R¹ is halo, cyano, carbamoyl, C₁₋₆alkoxy, C₁₋₆alky or C₂₋₆alkynyl;

n is 0, 1 or 2;

R² is C₁₋₆alkyl; wherein R² may be independently optionally substituted on carbon by one or more R⁹;

R³ is hydrogen or C₁₋₆alkyl;

or R² and R³ taken together with the carbon to which they are attached form a cyclopropyl or cyclobutyl ring;

R⁴ is selected from C₁₋₆alkyl or C₃₋₆cycloalkyl; wherein R⁴ may be optionally substituted on carbon by one or more R¹⁰;

Ring D fused to the imidazole of formula (I), formula (Ia) or formula (Ib) forms 1-H-benzimazole, 3H-imidazo[4,5-b]pyridine or 1H-imidazo[4,5-c]pyridine;

R⁵ is halo, C₁₋₆alkyl, C₃₋₆cycloalkyl or C₁₋₆alkoxy wherein C₁₋₆alkyl is optionally substituted on carbon with halo; and

m is 0, 1 or 2;

or a pharmaceutically acceptable salt thereof.

In a further aspect of the invention there is provided a compound of formula (I), formula (Ia) or formula (Ib) (as depicted above) wherein:

Ring A is carbocyclyl or heterocyclyl, wherein if said heterocyclyl contains an —NH— moiety that nitrogen may be optionally substituted by a group selected from R⁶;

R⁶ is C₁₋₃alkyl;

R¹ is halo, cyano, carbamoyl, C₁₋₆alkoxy or C₁₋₆alkyl;

n is 1 or 2;

R² is C₁₋₆alkyl; wherein R² may be independently optionally substituted on carbon by one or more R⁹;

R³ is hydrogen;

R⁴ is selected from C₁₋₆alkyl wherein R⁴ may be optionally substituted on carbon by one or more R¹⁰;

Ring D fused to the imidazole of formula (I), formula (Ia) or formula (Ib) forms 1-H-benzimazole, 3H-imidazo[4,5-b]pyridine or 1H-imidazo[4,5-c]pyridine;

R⁵ is halo, C₁₋₆alkyl, C₃₋₆cycloalkyl or C₁₋₆alkoxy wherein C₁₋₆alkyl is optionally substituted on carbon with halo; and

m is 0, 1 or 2;

R⁹ and R¹⁰ are as defined in formula (I);

or a pharmaceutically acceptable salt thereof.

In a further aspect of the invention there is provided a compound of formula (I), formula (Ia) or formula (Ib) (as depicted above) wherein:

Ring A is carbocyclyl or heterocyclyl;

R¹ is halo, cyano, carbamoyl or C₁₋₆alkoxy;

n is 1 or 2;

R² is C₁₋₆alkyl; wherein R² may be independently optionally substituted on carbon by one or more R⁹;

R³ is hydrogen or C₁₋₆alkyl;

or R² and R³ taken together with the carbon to which they are attached form a cyclopropyl or cyclobutyl ring;

R⁴ is selected from C₁₋₆alkyl or C₃₋₆cycloalkyl; wherein R⁴ may be optionally substituted on carbon by one or more R¹⁰;

Ring D fused to the imidazole of formula (I), formula (Ia) or formula (Ib) forms 1-H-benzimazole, 3H-imidazo[4,5-b]pyridine, 1H-imidazo[4,5-c]pyridine or hydrazine-3H-imidazo[4,5-c]pyridine (2:1);

R⁵ is halo, C₁₋₆alkyl or C₁₋₆alkoxy wherein C₁₋₆alkyl is optionally substituted on carbon with halo; and

m is 0, 1 or 2;

or a pharmaceutically acceptable salt thereof.

In a further aspect of the invention there is provided a compound of formula (I), formula (Ia) or formula (Ib) (as depicted above) wherein:

Ring A is carbocyclyl or heterocyclyl; wherein if said heterocyclyl contains an —NH— moiety that nitrogen may be optionally substituted by a group selected from R⁶;

R¹ is halo, cyano, carbamoyl, C₁₋₆alkyl, C₂₋₆alkynyl or C₁₋₆alkoxy;

n is 1 or 2;

R² is C₁₋₆alkyl; wherein R² may be independently optionally substituted on carbon by one or more R⁹;

R³ is hydrogen or C₁₋₆alkyl;

or R² and R³ taken together with the carbon to which they are attached form a cyclopropyl or cyclobutyl ring;

R⁴ is selected from C₁₋₆alkyl or C₃₋₆cycloalkyl; wherein R⁴ may be optionally substituted on carbon by one or more R¹⁰;

Ring D fused to the imidazole of formula (I), formula (Ia) or formula (Ib) forms 1-H-benzimazole, 3H-imidazo[4,5-b]pyridine or 1H-imidazo[4,5-c]pyridine;

R⁵ is halo, C₁₋₆alkyl or C₁₋₆alkoxy wherein C₁₋₆alkyl is optionally substituted on carbon with halo; and

m is 0, 1 or 2;

or a pharmaceutically acceptable salt thereof.

In a further aspect of the invention there is provided a compound of formula (I), formula (Ia) or formula (Ib) (as depicted above) wherein:

Ring A is selected from phenyl, cyclopropyl, cyclopentyl, cyclohexyl, pyrazolyl, imidazolyl, furanyl, pyridinyl, 1,3-thiazolyl, isoxazolyl, thienyl, pyrrolyl, 3,4-dihydro-2H-1,4-benzoxazinyl, 2,3-dihydro-1,4-benzodioxinyl, 2,1,3-benzothiadiazolyl, quinolinyl, dihydronaphthyl, pyrimidinyl, pyridinyl-N-oxide, or 6-oxo-1,6-dihydropyridinyl wherein said pyrazolyl, imidazolyl, pyrrolyl and 3,4-dihydro-2H-1,4-benzoxazinyl may be optionally substituted on nitrogen by a group selected from R⁶;

R¹ selected from halo, nitro, cyano, amino, carbamoyl, C₁₋₆alkyl, C₂₋₆alkynyl C₁₋₆alkoxy, hydrazinyl, ureido, N,N-di(C₁₋₃alkyl)ureido, C₁₋₆alkanoylamino, C₁₋₆alkylS(O)ₐ wherein a is 0 to 2, carbocyclyl, heterocyclyl; wherein R¹ may be optionally substituted on carbon by one or more R⁷; and wherein if said heterocyclyl contains an —NH— moiety that nitrogen may be optionally substituted by a group selected from R⁸;

n is 0, 1, 2 or 3;

R² is selected from methyl, ethyl, isopropyl, isobutyl, phenymethyl, 4-fluorophenylmethyl and pyrdinylmethyl;

R³ is hydrogen or methyl;

or R² and R³ taken together with the carbon to which they are attached form a cyclopropyl or cyclobutyl ring;

R⁴ is selected from methyl, ethyl, propyl, cyclopropyl, cyclopropylmethyl, isobutyl, and 2,2,2-trifluoroethyl;

Ring D fused to the imidazole of formula (I), formula (Ia) or formula (Ib) forms 1-H-benzimidazole, 3H-imidazo[4,5-b]pyridine, 1H-imidazo[4,5-c]pyridine, 1H-imidazo[4,5-b]pyridine, 3H-imidazo[4,5-c]pyridine or 5H-imidazo[4,5-c]pyridazinyl;

R⁵ is selected from chloro, bromo, fluoro, methyl, isobutyl, hydroxymethyl, difluoromethyl, trifluoromethyl, morpholinyl-4-methyl, N,N-dimethylaminomethyl, cyclopropyl, methoxy, trifluoromethoxy, carboxy, ethylcarboxy, methylcarboxy, carbamoyl, N,N-dimethylcarbamoyl, morpholinylcarbonyl, 3-pyridinyl, 4-pyridinyl, methylthio, methylsulfonyl, optionally substituted on carbon with halo; and

m is 0, or 2;

or when m=2, the two R5 together form a dioxinyl or dioxolyl ring;

or a pharmaceutically acceptable salt thereof.

Therefore in a further aspect of the invention there is provided a compound of formula (I), formula (Ia) or formula (Ib) (as depicted above) wherein:

Ring A is carbocyclyl;

R¹ is halo;

n is 1;

R² is C₁₋₆alkyl; wherein R² may be independently optionally substituted on carbon by one or more R⁹;

R³ is hydrogen or C₁₋₆alkyl;

R⁴ is selected from C₁₋₆alkyl or C₃₋₆cycloalkyl; wherein R⁴ may be optionally substituted on carbon by one or more R¹⁰;

Ring D fused to the imidazole of formula (I), formula (Ia) or formula (Ib) forms 1-H-benzimazole, 3H-imidazo[4,5-b]pyridine, 1H-imidazo[4,5-c]pyridine or hydrazine-3H-imidazo[4,5-c]pyridine (2:1);

R⁵ is halo, C₁₋₆alkyl or C₁₋₆alkoxy wherein C₁₋₆alkyl is optionally substituted on carbon with halo;

m is 0, 1 or 2;

R⁹ is carbocyclyl or heterocyclyl; and

R¹⁰ is carbocyclyl;

or a pharmaceutically acceptable salt thereof.

In a further aspect of the invention there is provided a compound of formula (I), formula (Ia) or formula (Ib) (as depicted above) wherein:

Ring A is carbocyclyl;

R¹ is halo;

n is 1;

R² is C₁₋₆alkyl; wherein R² may be independently optionally substituted on carbon by one or more R⁹;

R³ is hydrogen or C₁₋₆alkyl;

R⁴ is selected from C₁₋₆alkyl or C₃₋₆cycloalkyl; wherein R⁴ may be optionally substituted on carbon by one or more R¹⁰;

Ring D fused to the imidazole of formula (I), formula (Ia) or formula (Ib) forms 1-H-benzimazole, 3H-imidazo[4,5-b]pyridinyl, 1H-imidazo[4,5-c]pyridinyl, hydrazine-3H-imidazo[4,5-c]pyridinyl (2:1), 5H-imidazo[4,5-c]pyridazinyl or 7H-purinyl;

R⁵ is halo, C₁₋₆alkyl or C₁₋₆alkoxy wherein C₁₋₆alkyl is optionally substituted on carbon with halo or two R⁵ may together with the carbon atoms of ring D to which they are attached form a 5 to 8-membered carbocyclyl or heterocyclyl ring; wherein the 5 to 8-membered ring may be optionally substituted on carbon by one or more R¹⁵; and wherein if said heterocyclyl ring contains an —NH— moiety that nitrogen may be optionally substituted by a group selected from R¹⁶;

m is 0, 1 or 2;

R⁹ is carbocyclyl or heterocyclyl; and

R¹⁰ is carbocyclyl;

or a pharmaceutically acceptable salt thereof.

Therefore in a further aspect of the invention there is provided a compound of formula (I), formula (Ia) or formula (Ib) (as depicted above) wherein:

Ring A is carbocyclyl;

R¹ is halo;

n is 1;

R² is C₁₋₆alkyl; wherein R² may be independently optionally substituted on carbon by one or more R⁹;

R³ is hydrogen;

R⁴ is selected from C₁₋₆alkyl; wherein R⁴ may be optionally substituted on carbon by one or more R¹⁰;

Ring D fused to the imidazole of formula (I), formula (Ia) or formula (Ib) forms 1-H-benzimazolyl, 3H-imidazo[4,5-b]pyridinyl, 1H-imidazo[4,5-c]pyridinyl, hydrazine-3H-imidazo[4,5-c]pyridinyl (2:1), 5H-imidazo[4,5-c]pyridazinyl and 7H-purinyl;

R⁵ is halo or C₁₋₆alkyl wherein C₁₋₆alkyl is optionally substituted on carbon with halo.

m is 0, 1 or 2;

R⁹ is carbocyclyl or heterocyclyl; and

R¹⁰ is carbocyclyl;

or a pharmaceutically acceptable salt thereof.

In a still further aspect of the invention there is provided a compound of formula (I), formula (Ia) or formula (Ib) (as depicted above) wherein:

Ring A is carbocyclyl;

R¹ is halo or cyano;

n is 1;

R² is C₁₋₆alkyl; wherein R² may be independently optionally substituted on carbon by one or more R⁹;

R³ is hydrogen;

R⁴ is selected from C₁₋₆alkyl; wherein R⁴ may be optionally substituted on carbon by one or more R¹⁰;

Ring D fused to the imidazole of formula (I), formula (Ia) or formula (Ib) forms 1-H-benzimazolyl, 3H-imidazo[4,5-b]pyridinyl, 1H-imidazo[4,5-c]pyridinyl, hydrazine-3H-imidazo[4,5-c]pyridinyl (2:1), 5H-imidazo[4,5-c]pyridazinyl and 7H-purinyl;

R⁵ is halo or C₁₋₆alkyl wherein C₁₋₆alkyl is optionally substituted on carbon with halo;

m is 0, 1 or 2;

R⁹ is carbocyclyl or heterocyclyl; and

R¹⁰ is carbocyclyl;

or a pharmaceutically acceptable salt thereof.

Therefore in a further aspect of the invention there is provided a compound of formula (I), formula (Ia) or formula (Ib) (as depicted above) wherein:

Ring A is phenyl;

R¹ is chloro;

n is 1;

R² is methyl, ethyl, isopropyl, or isobutyl; wherein R² may be independently optionally substituted on carbon by one or more R⁹;

R³ is hydrogen;

R⁴ is selected from methyl, cyclopropylmethyl, ethyl, propyl or iso-butyl;

Ring D fused to the imidazole of formula (I), formula (Ia) or formula (Ib) forms 1-H-benzimazole or 3H-imidazo[4,5-b]pyridine, 1H-imidazo[4,5-c]pyridine or hydrazine-3H-imidazo[4,5-c]pyridine (2:1);

R⁹ is carbocyclyl; and

m is 0;

or a pharmaceutically acceptable salt thereof.

Therefore in a further aspect of the invention there is provided a compound of formula (I), formula (Ia) or formula (Ib) (as depicted above) wherein:

Ring A is phenyl;

R¹ is chloro, cyano or fluoro;

n is 0 or 1;

R² is methyl, ethyl, isopropyl, or isobutyl;

R³ is hydrogen;

R⁴ is selected from methyl, cyclopropylmethyl, ethyl, propyl or iso-butyl;

Ring D fused to the imidazole of formula (I), formula (Ia) or formula (Ib) forms 1-H-benzimazole or 3H-imidazo[4,5-b]pyridine, 1H-imidazo[4,5-c]pyridine or hydrazine-3H-imidazo[4,5-c]pyridine (2:1); and

m is 0;

or a pharmaceutically acceptable salt thereof.

Therefore in a further aspect of the invention there is provided a compound of formula (I), formula (Ia) or formula (Ib) (as depicted above) wherein:

Ring A is phenyl, pyrimidinyl or pyridinyl;

R¹ is chloro, fluoro, cyano, carbamoyl, or;

n is 0 or 1;

R² is methyl;

R³ is hydrogen or methyl;

R⁴ is selected from methyl, cyclopropylmethyl, ethyl, propyl or iso-butyl;

Ring D fused to the imidazole of formula (I), formula (Ia) or formula (Ib) forms 1-H-benzimazole or 3H-imidazo[4,5-b]pyridine, 1H-imidazo[4,5-c]pyridine or hydrazine-3H-imidazo[4,5-c]pyridine (2:1); and

m is 0;

or a pharmaceutically acceptable salt thereof.

In a still further aspect of the invention there is provided a compound of formula (I), formula (Ia) or formula (Ib) (as depicted above) wherein:

Ring A is phenyl or pyridinyl;

R¹ is halo, cyano, carbamoyl or C₁₋₆alkyl;

n is 1 or 2;

R² is methyl;

R³ is hydrogen;

R⁴ is ethyl;

Ring D fused to the imidazole of formula (I), formula (Ia) or formula (Ib) forms 1-H-benzimazole, 3H-imidazo[4,5-b]pyridine, 1H-imidazo[4,5-c]pyridine or 1H-imidazo[4,5-b]pyridine;

R⁵ is trifluoromethyl, methoxy or cyclopropyl; and

m is 1;

or a pharmaceutically acceptable salt thereof.

In another aspect of the invention, preferred compounds of the invention are any one of the Examples or a pharmaceutically acceptable salt thereof.

A further embodiment of the invention is directed to the compounds of Examples 145, 148, 149, 150, 151, 152, 158, 160, 161, 173, 174, 180 and 183 or pharmaceutically acceptable salts thereof.

Another aspect of the present invention provides a process for preparing a compound of formula (I) or a pharmaceutically acceptable salt or an in vivo hydrolysable ester thereof which process (wherein variable groups are, unless otherwise specified, as defined in formula (I)) comprises:

Process a) reacting of a compound of formula (II):

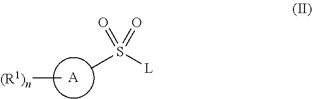

with an amine of formula (III):

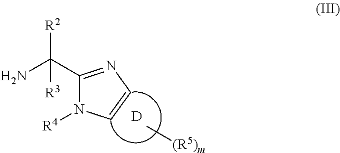

and thereafter if necessary:
i) converting a compound of the formula (I) into another compound of the formula (I);
ii) removing any protecting groups;
iii) forming a pharmaceutically acceptable salt.

L is a displaceable group, suitable values for L are for example, a halo for example a chloro or bromo.

Specific reaction conditions for the above reactions are as follows.

Process a) Compounds of formula (II) and (III) can be reacted together in the presence of a suitable solvent such as DCM and a base such as triethylamine. The reaction may require thermal conditions.

Compounds of formula (II) are commercially available, or they are known in the literature or they may be prepared by processes known in the art.

Compounds of formula (III) may be prepared according to Scheme 1:

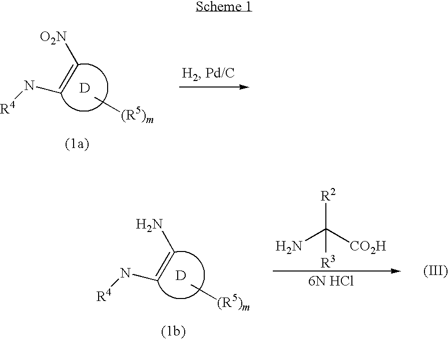

Compounds of formula (III) may also be prepared according to Scheme 2:

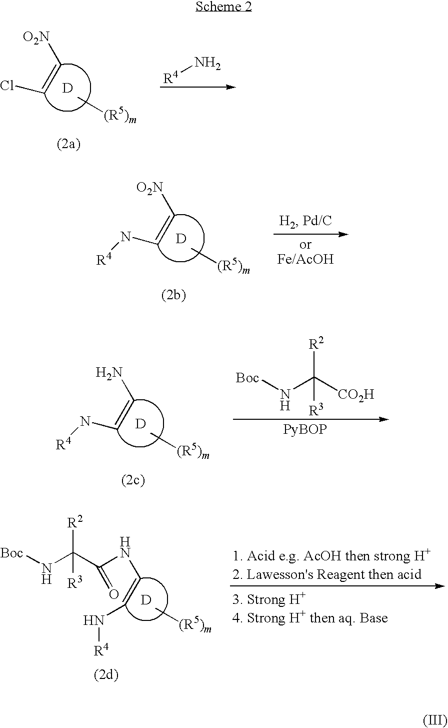

Compounds of formula (2b) may also be prepared according to Scheme 2a:

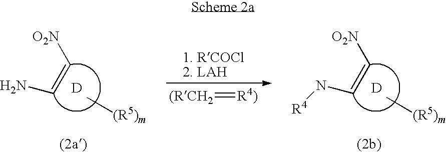

Compounds of formula (III) may also be prepared according to Scheme 3:

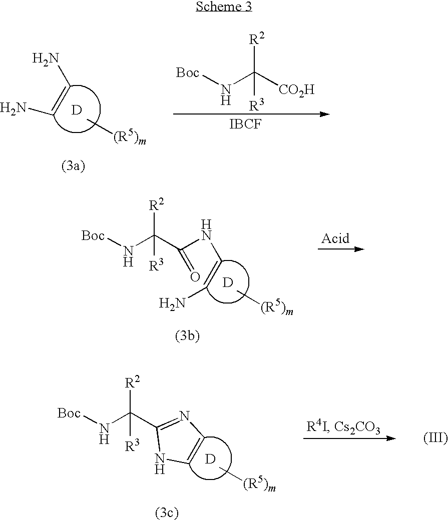

Compounds of formula (III) may also be prepared according to Scheme 4:

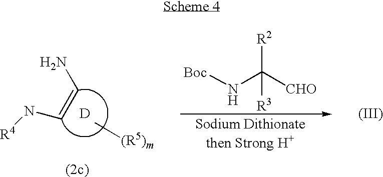

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

In order to use a compound of the formula (I), formula (Ia) or formula (Ib) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methylcellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

According to a further aspect of the present invention there is provided a compound of the formula (I) or (Ia), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their Edg-1 antagonistic properties. Accordingly the compounds of the present invention are expected to be useful in the treatment of diseases or medical conditions mediated alone or in part by Edg-1, i.e. the compounds may be used to produce an Edg-1 antagonistic effect in a warm-blooded animal in need of such treatment.

Thus the compounds of the present invention provide a method for treating cancer characterized by the antagonistic effect of Edg-1, i.e. the compounds may be used to produce an anti-cancer effect mediated alone or in part by the antagonistic effect of Edg-1.

Thus the compounds of the present invention provide a method for treating a variety of angiogenesis-related diseases that may be characterized by any abnormal, undesirable or pathological angiogenesis, for example tumor-related angiogenesis. The compounds may be used to produce an anti-cancer effect mediated alone or in part by antagonism of Edg-1.

Such a compound of the invention is expected to possess a wide range of activity in angiogenesis-related diseases including, but not limited to, non-solid tumours such as leukemia, multiple myeloma, hematologic malignancies or lymphoma, and also solid tumours and their metastases such as melanoma, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumours.

Excessive vascular growth also contributes to numerous non-neoplastic disorders for which the compounds of the invention may be useful in treating. These non-neoplastic angiogenesis-related diseases include: atherosclerosis, haemangioma, haemangioendothelioma, angiofibroma, vascular malformations (e.g. Hereditary Hemorrhagic Teleangiectasia (HHT), or Osler-Weber syndrome), waits, pyogenic granulomas, excessive hair growth, Kaposis' sarcoma, scar keloids, allergic oedema, psoriasis, dysfunctional uterine bleeding, follicular cysts, ovarian hyperstimulation, endometriosis, respiratory distress, ascites, peritoneal sclerosis in dialysis patients, adhesion formation result from abdominal surgery, obesity, rheumatoid arthritis, synovitis, osteomyelitis, pannus growth, osteophyte, hemophilic joints, inflammatory and infectious processes (e.g. hepatitis, pneumonia, glomerulonephritis), asthma, nasal polyps, liver regeneration, pulmonary hypertension, retinopathy of prematurity, diabetic retinopathy, age-related macular degeneration, leukomalacia, neovascular glaucoma, corneal graft neovascularization, trachoma, thyroiditis, thyroid enlargement, and lymphoproliferative disorders.

Thus according to this aspect of the invention there is provided a compound of the formula (I), formula (Ia) or formula (Ib) or a pharmaceutically acceptable salt thereof, as defined hereinbefore for use as a medicament.

According to a further aspect of the invention there is provided the use of a compound of the formula (I), formula (Ia) or formula (Ib) or a pharmaceutically acceptable salt thereof, as defined hereinbefore in the manufacture of a medicament for use in the production of a Edg-1 antagonistic effect in a warm-blooded animal such as man.

According to this aspect of the invention there is provided the use of a compound of the formula (I), formula (Ia) or formula (Ib) or a pharmaceutically acceptable salt thereof, as defined hereinbefore in the manufacture of a medicament for use in the production of an anti-cancer effect in a warm-blooded animal such as man.

According to a further feature of the invention, there is provided a compound of the formula (I), formula (Ia) or formula (Ib) or a pharmaceutically acceptable salt thereof, as defined herein before in the manufacture of a medicament for use in the treatment of pathologically angiogenic diseases, thrombosis, cardiac infarction, coronary heart diseases, arteriosclerosis, tumors, osteoporosis, inflammations or infections in a warm-blooded animal such as man.

According to a further feature of this aspect of the invention there is provided a method for producing a Edg-1 antagonistic effect in a warm-blooded animal, such as man, in need of such treatment which comprises administering to said animal an effective amount of a compound of formula (I), formula (Ia) or formula (Ib) or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating pathologically angiogenic diseases, thrombosis, cardiac infarction, coronary heart diseases, arteriosclerosis, tumors, osteoporosis, inflammations or infections, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a Edg-1 antagonistic effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of pathologically angiogenic diseases, thrombosis, cardiac infarction, coronary heart diseases, arteriosclerosis, tumors, osteoporosis, inflammations or infections in a warm-blooded animal such as man.

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:

1. antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
2. cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
3. agents which inhibit cancer cell invasion (for example metalloproteinase inhibitors like marimastat and inhibitors of urokinase plasminogen activator receptor function);
4. inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
5. antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
6. vascular damaging agents such as Combretastatin A4 and compounds disclosed in International Patent Applications WO 99/02166, WO 00/40529, WO 00/41669, WO 01/92224, WO 02/04434 and WO 02/08213;
7. antisense therapies, for example those which are directed to the targets listed above, such as ISIS 2503, an anti-ras antisense;
8. gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
9. immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

### Biological Activity

The following assays can be used to measure the effects of the compounds of the present invention as S1P1/Edg1 inhibitors.

I. In Vitro Cell Based Receptor Activation Assay-Transfluor Assay

This cell-based assay was designed to assess the ability of small molecule antagonists to inhibit activation of the GPCR S1P1 in the presence of its cognate ligand S1P. The assay used technology initially developed by Norak Biosciences (Xsira Pharmaceutical) and presently owned by Molecular Devices. A human osteogenic sarcoma (U2OS) cell line overexpressing the Edg-1/S1P1) receptor as well as a beta-arrestin/green fluorescent protein (GFP) construct hereafter termed Edg-1 Transfluor U2OS WT Clone #37 was employed.

Using a high content screening approach (Cellomics Arrayscan), receptor activity was measured by assessment of the relocalization of beta-arrestin GFP in response to stimulation of Edg-1 by SIP. Specifically, Edg-1 Transfluor U2OS WT Clone #37 cells were plated at a density of 6250 cells in 40 μL medium per well in 384 well plastic bottomed microtiter plates (BD Falcon) and incubated overnight at 37° C./5% CO₂. Prior to screening, compounds were dissolved in 100% dimethyl sulfoxide (DMSO) to a final stock concentration of 10 mM. Compounds were then serially diluted at 30× final concentration in Edg-1 Transfluor cell growth medium containing 30% DMSO using the Tecan Genesis instrument. These 30× plates were then diluted to 6× final concentration with Edg-1 Transfluor growth medium just prior to dosing. Cells were then dosed with 10 μL per well of 6× compound dilutions or 6% DMSO and pre-incubated for 15 minutes at room temperature. Cell plates were dosed with 10 μL per well 6× S1P Edg-1 Transfluor growth medium, then incubated for 45 minutes at 37° C./5% CO₂. Final concentration in the well of DMSO was 1%, compound was IX (3-fold, 9 point IC₅₀ dilutions starting at 100 μM final concentration), and either 375 nM or 750 DM SIP ligand. Cell plates were then fixed by adding 50 μL per well of 5% formaldehyde in 1× Dulbecco's phosphate buffered saline (DPBS) directly and incubating for 30 minutes at room temperature in darkness. Fixative was removed and replaced with 50 μL per well of 1×DPBS, after which cells were stained with 10 μg/mL final concentration of Hoechst 33342 (Molecular Probes) for 15 minutes at room temperature in darkness. Stain was then removed from the plates and replaced with 50 μL per well of 1×DPBS using the BioTek ExL405 plate washer. Plates were then sealed and analysed on the Cellomics Arrayscan using the GPCR signalling algorithm. EC₅₀ values were then calculated using IDBS ActivityBase software.

II. In Vitro Cell Based Receptor Activation Assay-Transfluor Assay

This cell-based assay was designed to assess the ability of small molecule antagonists to inhibit activation of the GPCR S1P1 in the presence of its cognate ligand S1P. The assay used technology initially developed by Norak Biosciences (Xsira Pharmaceutical) and presently owned by Molecular Devices (MDS Analytical Technologies). A human osteogenic sarcoma (U2OS) cell line overexpressing the Edg-1/S1P1) receptor as well as a beta-arrestin/green fluorescent protein (GFP) construct hereafter termed Edg-1 Transfluor U2OS Clone #3 was employed.

Using a high content screening approach (Molecular Devices Image Express), receptor activity was measured by assessment of the relocalization of beta-aiTestin GFP in response to stimulation of Edg-1 by S1P. Specifically, Edg-1 Transfluor U2OS Clone #3 cells were plated at a density of 6250 cells in 44 μL medium per well in 384 well plastic bottomed microtiter plates (BD Falcon) and incubated overnight at 37° C./5% CO₂. Prior to screening, compounds were dissolved in 100% dimethyl sulfoxide (DMSO) to a final stock concentration of 10 mM. Compounds were then serially diluted at 10× final concentration in Edg-1 Transfluor cell growth medium containing 6% DMSO using the BioMek instrument. Cells were then dosed with 6 μL per well of 10× compound dilutions or 6% DMSO and pre-incubated for 15 minutes at room temperature. Cell plates were dosed with 10 μL per well 6×S1P Edg-1 Transfluor growth medium, then incubated for 45 minutes at 37° C./5% CO₂. Final concentration in the well of DMSO was 1%, compound was 1× (3-fold, 9 point IC₅₀ dilutions starting at 3 μM final concentration), and 750 nM SIP ligand. Cell plates were then fixed by adding 50 μL per well of 5% formaldehyde in 1× Dulbecco's phosphate buffered saline (DPBS) directly and incubating for 30 minutes at room temperature in darkness. Fixative was removed and replaced with 50 μL per well of 1×DPBS, after which cells were stained with 10 μg/mL final concentration of Hoechst 33342 (Molecular Probes) for 15 minutes at room temperature in darkness. Stain was then removed from the plates and replaced with 50 μL per well of 1×DPBS using the BioTek ExL405 plate washer. Plates were then sealed and analysed on the Molecular Devices ImageXpress using the GPCR signalling algorithm. EC₅₀ values were then calculated using IDBS ActivityBase software.

Compounds of the invention generally exhibit EC₅₀ values <100 μM when tested in one or the other of the above two described assays. For example, the compound of Example 18 exhibited an EC₅₀ value of 0.896 μM; the compound of Example 19 exhibited an EC₅₀ value of 10.3 μM; and the compound of Example 21 exhibited an EC₅₀ value of 5.15 μM. The enantiomer of the compound of Example 102, 4-chloro-N-[(1S)-1-(1-ethyl-1H-imidazo[4,5-c]pyridin-2-yl)ethyl]benzenesulfonamide, did not show any measurable activity in these assays when the limit of detection was 33 μM.

It should be understood that for compounds of formula (I) which possess a chiral center, when resolved into the individual enantiomers, generally only one of the enantiomers possesses activity in the above-described assay.

Percentage inhibition values were also calculated using IDBS ActivityBase software and are indicated for each of the Examples in the experimental section below except for examples 1, 55, 70, 101, 111, 135, 163, 175 and 184. The % inhibition at the dose closest to 3.5 μM is reported with the exception of example 91 for which % inhibition is reported at 1 μM.

In most cases, compounds were initially dosed from a top concentration of 100 μM (final concentration in well). Careful attention was paid to obtaining an accurately fitting dose-response curve such that in some cases the top concentration was reduced to 10 μM or lower. Thus, the differences in absolute concentration for the compounds reflect differences in the top concentration for the corresponding dilution series. Hence, compounds listed as 3.70 μM were titrated from a top concentration of 100 μM, compounds listed as 3.50 μM were titrated from a top concentration of 10 μM and compounds listed as 3.30 μM were run with this as the top concentration. Example 91 listed as 1 μM was titrated with 3.50 μM as the top concentration.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous sodium sulphate; evaporation of solvent was carried out using a rotary evaporator under reduced pressure (600-4000 Pascals;
4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (M/Z);
(x) where a synthesis is described as being analogous to that described in a previous example the amounts used are the millimolar ratio equivalents to those used in the previous example; and
(xi) the following abbreviations have been used:
THF tetrahydrofuran;
BOC tert-butyloxycarbonyl;
DMF N,N-dimethylformamide;
EtOAc ethyl acetate;
RT room temperature;
DCM dichloromethane
DMSO dimethylsulphoxide
AcOH Acetic acid
IBCF isobutylchloroformate
PyBOP Benzotriazole-1-yl-oxy-tris-pyrrolidino-phosphonium hexafluorophosphate
LAH lithium aluminum hydride
TEA triethylamine; and
DIEA diisopropyl ethylamine
DIBAL Diisobutyl Aluminum Hydride
DAST Diethylaminosulfur trifluoride
NaBH(OAC)₃ Sodium triacetoxyborohydride
(DPPF)PdCl₂ Diphenylphosphinyl palladium chloride
(PPh₃)₄Pd Tetrakistriphenylphosphine Palladium (0)
CDI Carbonyl Diimidazole
HATU O-(7-Azabenotriazole-1-yl)-N,N,N′,N′-Tetramethyluronium Hexafluoro-Phosphate
SFC Super Critical Fluid Chromatography
Lawesson's reagent p-methoxyphenylthionophosphine sulfide dimer having the following structure:

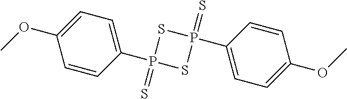

### Example 1

### 4-Chloro-N-[(1R)-1-(1-ethyl-1H-benzimidazol-2-yl)ethyl]benzenesulfonamide

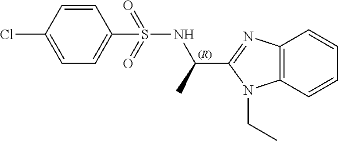

[(1R)-1-(1-ethyl-1H-benzimidazol-2-yl)ethyl]amine (Intermediate 1; 0.700 g, 3.70 mmol) and Et₃N (1.70 mL, 12.2 mmol) were dissolved in DCM (30 mL). After cooling to 0° C., a solution of 4-chlorobenzenesulfonyl chloride (0.820 g, 3.88 mmol) in DCM (5 mL) was added drop wise and the reaction mixture was stirred overnight at RT. The reaction mixture was diluted with DCM (70 mL) and washed with water (3×10 mL) and brine (10 mL). The organic layer was dried and concentrated in vacuo to give a dark brown semi-solid residue, which was purified by flash column chromatography using silica gel and CHCl₃/MeOH (98:2) to give the product as a light purple solid (1.30 g, 97% yield). ¹H NMR (300 MHz, CDCl₃) δ 7.64-7.57 (m, 3H), 7.26-7.16 (m, 5H), 6.18 (d, J=8.3 Hz, 1H), 4.85-4.75 (m, 1H), 4.24-3.99 (m, 2H), 1.56 (d, J=6.9 Hz, 3H), 1.35 (t, J=7.1 Hz, 3H). M/Z=363

The compounds of examples 2-182 were prepared by a procedure analogous to that of Example 1, using the appropriate sulfonyl chloride of formula (II) (see page 17) (which are commercially available except for those used in Examples 105-109, Exs. 114, 127, 140, 148, 151, 155, 160, 161, 172, 173, 177, 181 which were prepared as described below and shown in Table 7 and the appropriate Intermediate amine of formula (III) (see page 17), indicated as INT in Table 1, with the exception of Examples 68-78, Examples 8095, 101, 102, 106, 111, 128, 129, 133, 134, 141, 149, 152, 158, 163, 164, 165, 167, 168, 169, 170, 171, 178 and 182 which were synthesized from the Examples listed in Table 1 using the synthetic routes described at the end of table 1 Example 187 was prepared from appropriate Intermediate amine of formula (III) (see page 17) as described immediately following table 1. Procedures for the preparation of the Intermediates follow Table 1

TABLE 1

| Ex | Compound | NMR | M/Z | INT |
|---|---|---|---|---|
| 2 | 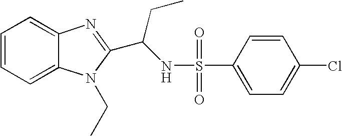 4-Chloro-N-[1-(1-ethyl-1H- benzoimidazol-2-yl)propyl]- benzenesulfonamide (94% inhibition at 3.50 μM) | ¹H NMR (300 MHz,) 8.60 (br s, 1H), 7.60 (d, J = 8.5 Hz, 2H), 7.49-7.43 (m, 2H), 7.33-7.30 (m, 2H), 7.22-7.12 (m, 2H), 4.55 (t, J = 7.1 Hz, 1H), 4.25-4.18 (m,2H), 1.99-1.87 (m, 1H), 1.84-1.70 (m, 1H), 1.27 (t, J = 7.1 Hz, 3H), 0.77 (t, J = 7.1 Hz, 3H) | 377 | 2 |
| 3 | 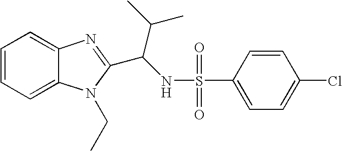 4-Chloro-N-[1-(1-ethyl-1H- benzimidazol-2-yl)-2-methyl- propyl]benzenesulfonamide (59% inhibition at 3.7 μM) | ¹H NMR (300 MHz) 8.59 (d, J = 8.3 Hz, 1H), 7.50-7.38 (m, 4H), 7.20-7.11 (m, 4H), 4.31-4.10 (m, 3H), 2.27-2.17 (m, 1H), 1.25 (t, J = 6.9 Hz, 3H), 1.01 (d, J = 6.6 Hz, 3H), 0.70 (d, J = 6.6 Hz, 3H) | 391 | 3 |
| 4 | 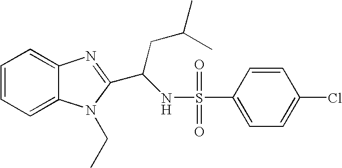 4-Chloro-N-[1-(1-ethyl-1H- benzimidazol-2-yl)-3-methyl- butyl}benzenesulfonamide (66.37% inhibition at 3.70 μM) | ¹H NMR (300 MHz,) 8.63 (br s, 1H), 7.58 (d, J = 8.5 Hz, 2H), 7.49-7.43 (m, 2H), 7.32 (d, J = 8.8 Hz, 2H), 7.22-7.13 (m, 2H), 4.61-4.56 (m, 1H), 4.24-4.14 (m, 2H), 1.79-1.60 (m, 2H), 1.56-1.47 (m, 1H), 1.28 (t, J = 7.1 Hz, 3H), 0.83 (d, J = 6.6 Hz, 3H), 0.77 (d, J = 6.3 Hz, 3H) | 405 | 4 |
| 5 | 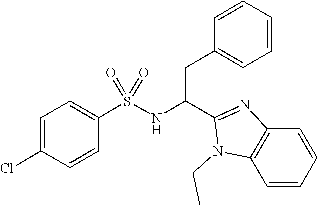 4-Chloro-N-[1-(1-ethyl-1H- benzimidazol-2-yl)-2-phenyl- ethyl]benzenesulfonamide (73.21% inhibition at 3.70 μM) | ¹H NMR (300 MHz) 8.89 (d, J = 8.5 Hz, 1H), 7.52-7.46 (m, 3H), 7.39-7.34 (m, 1H), 7.23-7.05 (m, 9H), 4.79-4.71 (m, 1H), 4.08-3.94 (m, 2H), 3.30 (dd, J = 12.9, 8.5 Hz, 1H), 3.09 (dd, J = 12.9, 6.6 Hz, 1H), 0.89 (t, J = 7.1 Hz, 3H) | 440 | 5 |
| 6 | 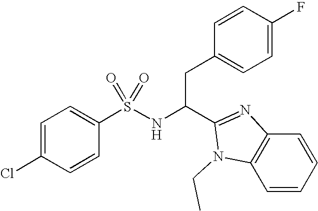 4-Chloro-N-[(4-chloro- phenyl)(1-ethyl-1H- benzimidazol-2-yl)methyl]- benzenesulfonamide (77.68% inhibition at 3.70 μM) | ¹H NMR (300 MHz, CDCl₃) 7.66-7.60 (m, 1H), 7.46-7.42 (m, 2H), 7.29-7.22 (m, 2H), 7.20-7.14 (m, 1H), 7.04-7.00 (m, 2H), 6.93-6.88 (m, 2H), 6.84-6.78 (m, 2H), 4.80 (br s, 1H), 3.95-3.82 (m, 1H), 3.72-3.59 (m, 1H), 3.38-3.24 (m, 2H), 1.85 (br s, 1H), 0.89 (t, J = 7.1 Hz, 3H) | 457 | 6 |
| 7 | 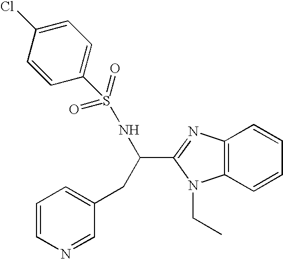 4-Chloro-N-[1-(1-ethyl-1H- benzimidazol-2-yl)-2- pyridin-3-yl-ethyl]benzene- sulfonamide (79.69% inhibition at 3.70 μM) | ¹H NMR (300 MHz) 8.91 (d, J = 8.0 Hz, 1H), 8.33-8.32 (m, 2H), 7.58-7.38 (m, 5H), 7.22-7.15 (m, 5H), 4.86-4.79 (m, 1H), 4.17-4.03 (m, 2H), 3.32-3.26 (m, 1H), 3.17-3.10 (m, 1H), 0.98 (t, J = 7.1 Hz, 3H) | 440 | 7 |
| 8 | 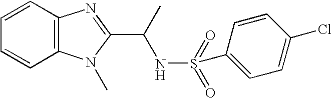 4-Chloro-N-[1-(1-methyl-1H- benzimidazol-2-yl)ethyl]- benzenesulfonamide (91.16% inhibition at 3.70 μM) | ¹H NMR (300 MHz, CDCl₃) 1.62 (d, 3H) 3.47-3.88 (m, 3H) 4.62-5.21 (m, 1H) 6.93-8.05 (m, 8H) | 350 | 8 |
| 9 | 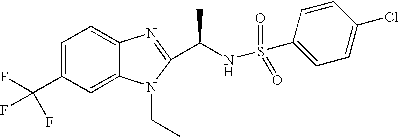 4-Chloro-N-{(1R)-1-[1-ethyl- 6-(trifluoromethyl)-1H- benzimidazol-2-yl]ethyl} benzene-sulfonamide (84.85% inhibition at 3.50 μM) | ¹H NMR (MeOH-d₆) 7.84 (s, 1H), 7.7 (d, 1H), 7.64 (d, 2H), 7.54 (d, 1H), 7.30 (d, 2H), 4.98 (q, 1H), 4.43 (q, 2H), 1.57 (d, 3H), 1.43 (t, 3H) | 341 | 9 |
| 10 | 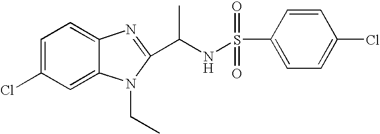 4-Chloro-N-[1-(6-chloro-1- ethyl-1H-benzimidazol-2- yl)ethyl]benzenesulfonamide (89.90% inhibition at 3.70 μM) | ¹H NMR (300 MHz, CDCl₃) 1.45 (t, 3H) 1.68 (d, 3H) 4.14 (m, 2H) 4.87 (m, 1H) 6.52 (m, 1H) 7.21 (d, 2H) 7.33 (m, 2H) 7.56 (d, 1H) 7.66 (d, 2H) | 398 | 10 |
| 11 | 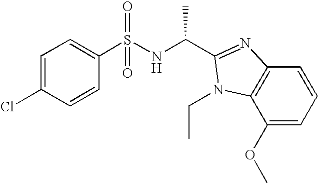 4-Chloro-N-[(1R)-1-(1-ethyl- 7-methoxy-1H-benzimidazol- 2-yl)ethyl]benzene- sulfonamide (82.03% inhibition at 3.70 μM) | ¹H NMR (300 MHz, CDCl₃) 1.48 (t, 3H) 1.68 (d, 3H) 3.97 (s, 3H) 4.58 (m, 2H) 4.96 (m, 1H) 6.84 (d, 1H) 7.05 (d, 2H) 7.15 (d, 1H) 7.34 (m, 1H) 7.55 (d, 2H) 9.24 (br d, 1H) | 393 | 11 |
| 12 | 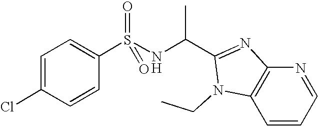 4-Chloro-N-[1-(1-ethyl-1H- imidazo[4,5-b]pyridin-2-yl)- ethyl]-benzenesulfonamide (93.34% inhibition at 3.50 μM) | ¹H NMR (300 MHz,) 1.30 (t, 3H) 1.40 (d, 3H) 4.30 (q, 2H) 4.81-4.95 (m, 1H) 7.18-7.27 (m, 1H) 7.45 (d, 2H) 7.70 (d, 2H) 7.92-8.00 (m, 1H) 8.32-8.39 (m, 1H) 8.65-8.73 (m, 1H) | 364 | 12 |
| 13 | 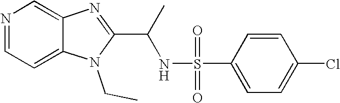 4-Chloro-N-[1-(1-ethyl-1H- imidazo[4,5-c]pyridin-2- yl)ethyl]benzene sulfonamide (72.77% inhibition at 3.70 μM) | ¹H NMR (CDCl₃) 8.8 (s, 3H), 8.3 (d, 1H), 7.7 (d, 2H), 7.6 (d, 1H), 7.3 (d, 2H), 4.4 (q, 2H), 1.6 (d, 3H), 1.4 (t, 3H) | 364 | 13 |
| 14 | 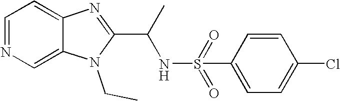 4-Chloro-N-[1-(3-ethyl-3H- imidazo[4,5-c]pyridin-2- yl)ethyl] benzene sulfonamide (72.77% inhibition at 3.70 μM) | ¹H NMR (MeOH-d₄) 8.86 (s, 1H), 8.33 (d, 1H), 7.75 (d, 2H), 7.6 (d, 1H), 7.34 (d, 2H), 5.01 (m, 1H), 4.45 (q, 2H0, 1.66 (d, 3H), 1.6 (t, 3H) | 364 | 14 |
| 15 | 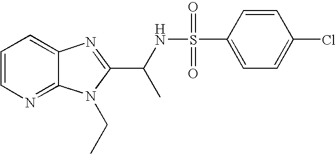 4-Chloro-N-[1-(3-ethyl-3H- imidazo[4,5-b]pyridin-2- yl)ethyl]benzenesulfonamide (61.55% inhibition at 3.70 μM) | ¹HNMR (MeOH-d₄) 8.45 (d, 1H), 7.88 (d, 1H), 7.64 (d, 2H), 7.28 (m, 2H), 7.41 (d,2H), 6.01 (d, 1H), 4.86 (m, 1H), 4.37 (q, 2H), 1.7 (s, 9H), 1.65 (d, 3H), 1.4 (t, 3H) | 364 | 15 |
| 16 | 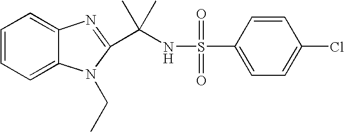 4-Chloro-N-[1-(1-ethyl-1H- benzoimidazol-2-yl)-1- methyl-ethyl]- benzenesulfonamide (96.23% inhibition at 3.50 μM) | ¹H NMR (300 MHz) 8.62 (br s, 1H), 7.62-7.57 (m, 3H), 7.45-7.37 (m, 3H), 7.24-7.15 (m, 2H), 4.32 (t, J = 7.1 Hz, 2H), 1.6 (br s, 6H), 1.38 (d, J = 6.9 Hz, 3H) | 377 | 16 |
| 17 | 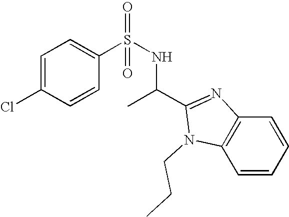 4-Chloro-N-[1-(1-propyl-1H- benzimidazol-2-yl)ethyl]- benzenesulfonamide (92.8% inhibition at 3.30 μM) | ¹H NMR (300 MHz, CDCl₃) 0.84 (t, 3H) 1.52 (d, 3H) 1.68 (m, 2H) 3.89 (m, 1H) 4.05 (m, 1H) 4.77 (m, 1H) 6.90 (d, 1H) 7.04 (d, 2H) 7.18 (m, 3H) 7.49 (d, 2H) 7.57 (m, 1H) | 377 | 17 |
| 18 | 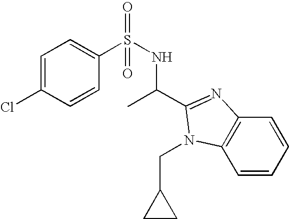 4-Chloro-N-{1-[1- (cyclopropylmethyl)-1H- benzimidazol-2-yl]ethyl}- benzenesulfonamide (100% inhibition at 3.70 μM) | ¹H NMR (300 MHz, CDCl₃) 0.25 (m, 2H) 0.47 (m, 2H) 0.99 (m, 1H) 1.49 (d, 3H) 3.84 (dd, 2H) 4.72 (m, 1H) 6.70 (d,1H) 6.95 (d,2H) 7.14 (m, 3H) 7.41 (d, 2H) 7.48 (m, 1H) | 389 | 18 |
| 19 | 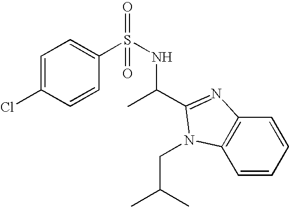 4-Chloro-N-[1-(1-isobutyl- 1H-benzimidazol-2-yl)ethyl]- benzenesulfonamide (16.4% inhibition at 3.70 μM) | ¹H NMR (300 MHz, CDCl₃) 0.89 (d, 3H) 0.90 (d, 3H) 1.62 (d, 3H) 2.07 (m, 1H) 3.77 (q, 1H) 4.02 (q, 1H) 4.86 (m, 1H) 7.11 (d, 2H) 7.24 (m, 3H) 7.42 (d, 1H) 7.57 (d, 2H) 7.68 (m, 1H) | 391 | 19 |
| 20 | 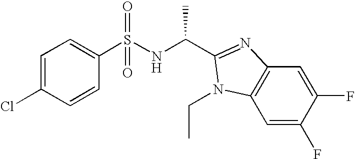 4-Chloro-N-[(1R)-1-(1-ethyl- 5,6-difluoro-1H- benzimidazol-2- yl)ethyl]benzenesulfonamide (79.5% inhibition at 3.70 μM) | ¹H NMR (300 MHz, CDCl₃) 1.44 (t, 3H) 1.66 (d, 3H) 4.12 (m, 1H) 4.24 (m, 1H) 4.86 (m, 1H) 6.33 (d, 1H) 7.15 (dd, 1H) 7.24 (d, 2H) 7.44 (dd, 1H) 7.68 (d, 2H) | 399 | 20 |
| 21 | 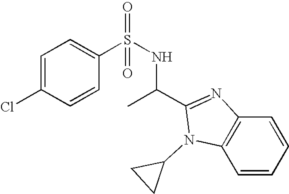 4-Chloro-N-[1-(1- cyclopropyl-1H- benzimidazol-2- yl)ethyl]benzenesulfonamide (39.05% inhibition at 3.70 μM) | ¹H NMR (300 MHz, CDCl₃) 1.00- 1.06 (m, 1H) 1.17-1.21 (m, 1H) 1.40 (m, 2H) 1.75 (d, 3H) 3.27 (m, 1H) 5.24 (m, 1H) 7.14 (d, 2H) 7.39 (m, 2H) 7.55-7.64 (m, 2H) 7.67 (d, 2H) | 375 | 21 |
| 22 | 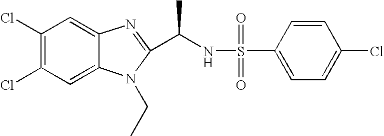 (R)-4-Chloro-N-[1-(5,6- dichloro-1-ethyl-1H- benzoimidazol-2-yl)ethyl]- benzenesulfonamide (63.53% inhibition at 3.70 μM) | ¹H NMR (300 MHz) 8.6 (d, J = 6.5 Hz, 1H), 7.9 (s, 1H), 7.7 (s, 1H), 7.6 (m 2H), 7.4 (m, 2H), 4.8 (m, 1H), 4.2 (m, 2H), 1.4 (d, J = 6.8 Hz, 3H), 1.3 (t, J = 9.4 Hz, 3H) | 432 | 22 |
| 23 | 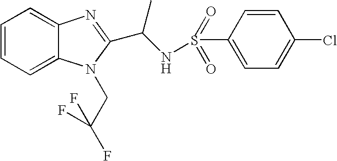 4-Chloro-N-{1-[1-(2,2,2- trifluoro-ethyl)-1H- benzimidazol-2-yl]-ethyl}- benzenesulfonamide (88.1% inhibition at 3.70 μM) | ¹H NMR (300 MHz, CDCl₃) 1.59 (d, 3H) 3.62-3.78 (m, 2H) 4.52-5.12 (m, 3H) 5.57 (d, 1H) 7.20 (dd, 2H) 7.25-7.36 (m, 4H) 7.60 (dd, 2H) | 418 | 23 |
| 24 | 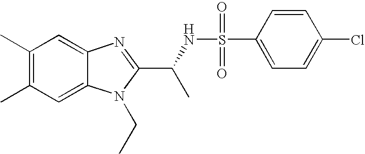 4-Chloro-N-[(1R)-1-(1-ethyl- 5,6-dimethyl-1H- benzimidazol-2- yl)ethyl]benzenesulfonamide (72.97% inhibition at 3.50 μM) | ¹H NMR (300 MHz) 1.30 (m, 6H) 2.30 (d, 6H) 4.19 (m, 2H) 4.79 (m, 1H) 7.25 (d, 2H) 7.47 (d,2H) 67.71 (d, 2H) 8.51 (d, 1H) | 391 | 24 |
| 25 | 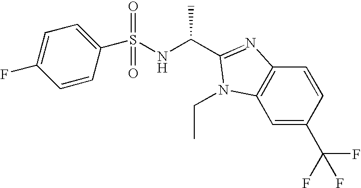 N-{(1R)-1-[1-Ethyl-6- (trifluoromethyl)-1H- benzimidazol-2-yl]ethyl}-4- fluorobenzenesulfonamide (89.95% inhibition at 3.50 μM) | ¹H NMR (300 MHz, CDCl₃) 7.78 (m, 3H) 7.48 (m, 2H) 7.13 (m, 1H) 6.98 (t, 2H) 4.90 (m, 1H) 4.29 (m, 1H) 4.12 (m, 1H) 1.54 (d, 3H) 1.39 (t, 3H) | 415 | 9 |
| 26 | 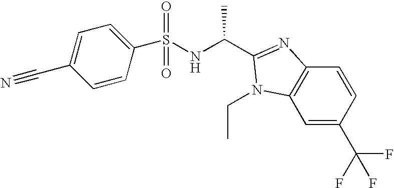 4-Cyano-N-{(1R)-1-[1-ethyl- 6-(trifluoromethyl)-1H- benzimidazol-2- yl]ethyl}benzenesulfonamide (87.38% inhibition at 3.50 μM) | ¹H NMR (300 MHz, CDCl₃) 7.87 (d, 2H) 7.75 (d, 1H) 7.51-7.59 (m, 4H) 7.25 (d, 1H) 4.95 (m, 1H) 4.31 (m, 1H) 4.15 (m, 1H) 1.58 (d, 3H) 1.43 (t, 3H) | 422 | 9 |
| 27 | 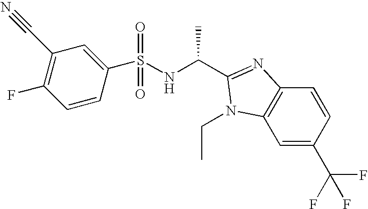 3-Cyano-N-{(1R)-1-[1-ethyl- 6-(trifluoromethyl)-1H- benzimidazol-2-yl]ethyl}-4- fluorobenzenesulfonamide (85.74% inhibition at 3.50 μM) | ¹H NMR (300 MHz, CDCl₃) 7.94-7.99 (m, 2H) 7.73 (d, 1H) 7.55 (m, 2H) 7.11 (t, 1H) 6.87 (bd, 1H) 4.94 (m, 1H) 4.32 (m, 1H) 4.21 (m, 1H) 1.63 (d,3H) 1.48 (t, 3H) | 440 | 9 |
| 28 | 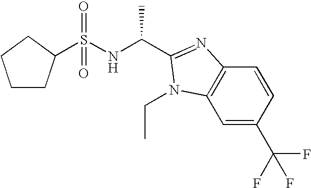 N-{(1R)-1-[1-Ethyl-6- (trifluoromethyl)-1H- benzimidazol-2-yl]ethyl} cyclopentanesulfonamide (66.06% inhibition at 3.70 μM) | ¹H NMR (300 MHz, CDCl₃) 7.92 (d, 1H) 7.70 (m, 1H) 7.19 (s, 1H) 5.08 (m, 1H) 4.30-4.51 (m, 2H) 3.36 (m, 1H) 1.90-2.01 (m, 4H) 1.81 (d, 3H) 1.70-1.75 (m, 2H) 1.55 (t, 5H) | 389 | 9 |
| 29 | 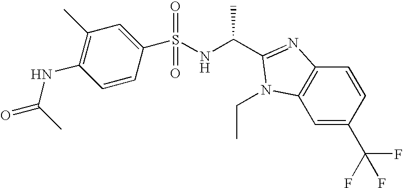 N-{4-[({(1R)-1-[1-Ethyl-6- (trifluoromethyl)-1H- benzimidazol-2- yl]ethyl}amino)sulfonyl]-2- methylphenyl}acetamide (21.84% inhibition at 3.70 μM) | ¹H NMR (300 MHz, CDCl₃) 7.83 (d, 1H) 7.75 (d, 1H) 7.45-7.58 (m, 4H) 6.65 (bs, 1H) 4.83 (m, 1H) 4.18 (m, 2H) 2.08 (s, 3H) 1.86 (s, 3H) 1.73 (d, 3H) 1.41 (t, 3H) | 468 | 9 |
| 30 | 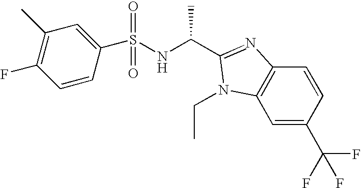 N-{(1R)-1-[1-Ethyl-6- (trifluoromethyl)-1H- benzimidazol-2-yl]ethyl}-4- fluoro-3- methylbenzenesulfonamide (94.56% inhibition at 3.50 μM) | ¹H NMR (300 MHz, CDCl₃) 7.80 (d, 1H) 7.68 (d, 2H) 7.57-7.63 (m, 2H) 6.87 (t, 1H) 4.99 (m, 1H) 4.44 (m, 1H) 4.30 (m, 1H) 2.08 (s, 3H) 1.79 (d, 3H) 1.56 (t,3H) | 429 | 9 |
| 31 | 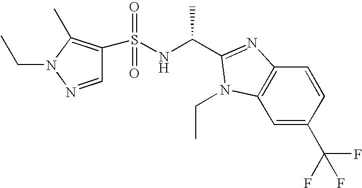 1-Ethyl-N-{(1R)-1-[1-ethyl-6- (trifluoromethyl)-1H- benzimidazol-2-yl]ethyl}-5- mthyl-1H-pyrazole-4- sulfonamide (78.6% inhibition at 3.70 μM) | ¹H NMR (300 MHz, CDCl₃) 7.98 (d, 1H) 7.84-7.80 (m, 3H) 7.40 (s, 1H) 5.12 (m, 1H), 4.59 (m, 1H) 4.45 (m, 1H) 3.90 (m, 2H) 2.57 (s, 3H) 1.90 (d, 3H) 1.67 (t, 3H) 1.22 (t, 3H) | 429 | 9 |
| 32 | 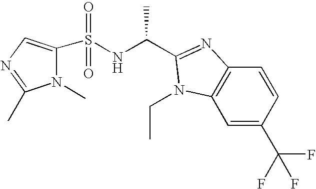 N-{(1R)-1-[1-Ethyl-6- (trifluoromethyl)-1H- benzimidazol-2-yl]ethyl}-1,2- dimethyl-1H-imidazole-5- sulfonamide (66.54% inhibition at 3.70 μM) | ¹H NMR (300 MHz, CDCl₃) 7.94 (d, 1H) 7.71-7.76 (m, 3H) 5.05 (m, 1H) 4.35-4.51 (m, 2H) 3.67 (s,3H) 2.39 (s, 3H) 1.80 (d, 3H) 1.58 (t, 3H) | 415 | 9 |
| 33 | 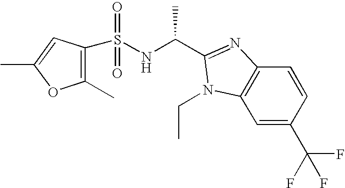 N-{(1R)-1-[1-Ethyl-6- (trifluoromethyl)-1H- benzimidazol-2-yl]ethyl}-2,5- dimethylfuran-3-sulfonamide (92.03% inhibition at 3.50 μM) | ¹H NMR (300 MHz, CDCl₃) 7.97 (d, 1H) 7.75-7.79 (m, 2H) 5.94 (s, 1H) 5.06 (m, 1H) 4.51 (m, 1H) 4.40 (m, 1H) 2.50 (s, 3H) 1.95 (s, 3H) 1.86 (d, 3H) 1.61 (t, 3H) | 415 | 9 |
| 34 | 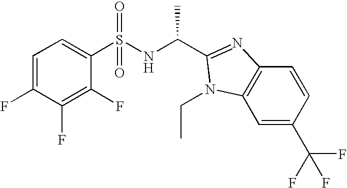 N-{(1R)-1-[1-Ethyl-6- (trifluoromethyl)-1H- benzimidazol-2-yl]ethyl}- 2,3,4- trifluoromethylbenzenesulfonamide (95.33% inhibition at 3.50 μM) | ¹H NMR (300 MHz, CDCl₃) 7.92 (d, 1H) 7.73-7.78 (m, 2H) 7.56-7.64 (m, 2H) 6.96 (m 1H) 5.25 (m, 1H) 4.52 (m, 1H) 4.41 (m, 1H) 1.86 (d, 3H) 1.62 (t, 3H) | 451 | 9 |
| 35 | 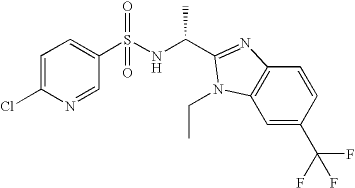 6-Chloro-N-{(1R)-1-[1-ethyl- 6-(trifluoromethyl)-1H-benz- imidazol-2-yl]ethyl}pyridine- 3-sulfonamide | ¹H NMR (300 MHz, CDCl₃) 8.00 (dd, 1H), 7.71 (d, 1H) 7.59-7.64 (m, 2H) 7.25-7.36 (m, 1H) 7.14 (d, 1H) 4.93 (m, 1H) 4.20-4.38 (m, 2H) 1.70 (d, 3H) 1.49 (t, 3H) | 433 | 9 |
| 36 | 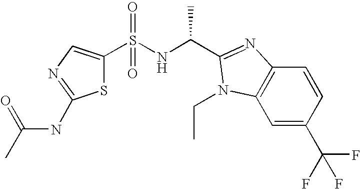 N-{5-[({(1R)-1-[1-Ethyl-6- (trifluoromethyl)-1H- benzimidazol-2- yl]ethyl}amino)sulfonyl]-1,3- thiazol-2-yl}acetamide (49.03% inhibition at 3.70 μM) | ¹H NMR (300 MHz, CDCl₃) 7.77 (d, 2H) 7.59-7.67 (m, 2H) 5.09 (m, 1H) 4.38 (m, 2H) 2.12 (s, 3H) 1.74 (d, 3H) 1.55 (t, 3H) | 461 | 9 |
| 37 | 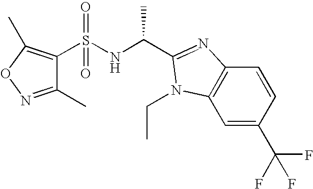 N-{(1R)-1-[1-Ethyl-6- (trifluoromethyl)-1H- benzimidazol-2-yl]ethyl}-3,5- dimethylisoxazole-4- sulfonamide (58.26% inhibition at 3.70 μM) | ¹H NMR (300 MHz, CDCl₃) 8.02 (d, 1H) 7.80-7.84 (m, 2H) 5.08 (m, 1H) 4.41-4.55 (m, 2H) 2.67 (s,3H) 2.46 (s, 3H) 1.85 (d, 3H) 1.65 (t, 3H) | 416 | 9 |
| 38 | 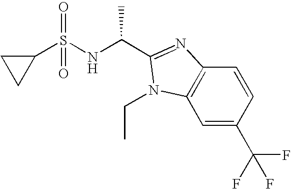 N-{(1R)-1-[1-Ethyl-6- (trifluoromethyl)-1H-benz- imidazol-2-yl]ethyl}cyclo- propanesulfonamide (35.1% at inhibition at 3.70 μM) | ¹H NMR (300 MHz, CDCl₃) 7.92 (d, 1H) 7.75 (s, 1H) 7.67 (d, 1H) 6.34 (bd, 1H) 5.09 (m, 1H) 4.34-4.52 (m, 2H) 2.33 (m, 1H) 1.85 (d, 3H) 1.59 (t, 3H) 1.21 (m, 1H) 1.11 (m, 1H) 0.97 (m, 1H) 0.83 (m, 1H) | 361 | 9 |
| 39 | 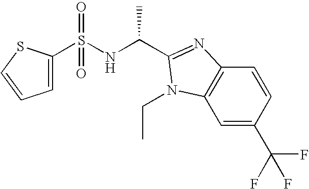 N-{(1R)-1-[1-Ethyl-6- (trifluoromethyl)-1H- benzimidazol-2- yl]ethyl}thiophene-2- sulfonamide (92.53% inhibition at 3.50 μM) | ¹H NMR (300 MHz, CDCl₃) 7.84 (d, 1H) 7.61-7.72 (m, 3H) 7.32 (d, 1H) 6.88 (d,1H) 5.06 (m, 1H) 4.45 (m, 1H) 4.34 (m, 1H) 1.78 (d, 3H) 1.57 (t, 3) | 403 | 9 |
| 40 | 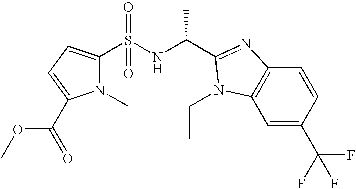 Methyl 5-[({(1R)-1-[1-ethyl- 6-(trifluoromethyl)-1H- benzimidazol-2- yl]ethyl}amino)sulfonyl]-1- methyl-1H-pyrrole-2- carboxylate (97.48% inhibition at 3.50 μM) | ¹H NMR (300 MHz, CDCl₃) 7.79 (d,1H) 7.66 (s, 1H) 7.62 (d, 1H) 7.17 (s, 1H) 6.74 (d, 1H) 6.31 (bs, 1H) 4.96 (m, 1H) 4.42 (m, 1H) 4.30 (m, 1H) 3.69 (s, 3H) 3.66 (s, 3H) 1.74 (d, 3H) 1.53 (t, 3H) | 458 | 9 |
| 41 | 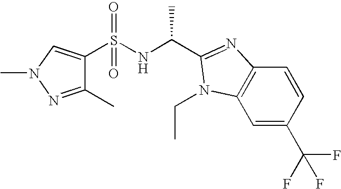 N-{(1R)-1-[1-Ethyl-6- (trifluoromethyl)-1H- benzimidazol-2-yl]ethyl}-1,3- dimethyl-1H-pyrazole-4- sulfonamide (61% inhibition at 3.70 μM) | ¹H NMR (300 MHz, CDCl₃) 7.89 (d, 1H) 7.69 (s, 1H) 7.62 (d, 1H) 6.67 (bs, 1H) 5.01 (m, 1H) 4.43 (m, 1H) 4.32 (m, 1H) 1.77 (d, 3H) 1.51 (t, 6H) 1.31 (s, 3H) 1.29 (s, 3H) | 429 | 9 |
| 42 | 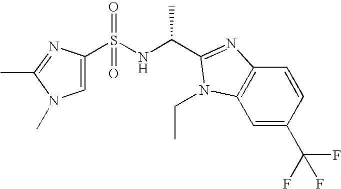 N-{(1R)-1-[1-Ethyl-6- (trifluoromethyl)-1H- benzimidazol-2-yl]ethyl}-1,2- dimethyl-1H-imidazole-4- sulfonamide (41.32% inhibition at 3.70 μM) | ¹H NMR (300 MHz, CDCl₃) 7.78 (d, 1H) 7.60 (s, 1H) 7.54 (d, 1H) 7.41 (s, 1H) 7.57 (bs, 1H) 4.76 (m, 1H) 4.18 (m, 2H) 3.60 (s, 3H) 1.85 (s, 3H) 1.65 (d, 3H) 1.42 (t, 3H) | 415 | 9 |
| 43 | 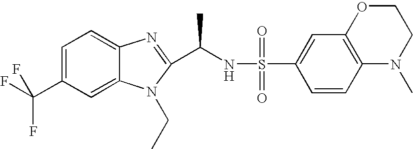 N-{(1R)-1-[1-Ethyl-6- (trifluoromethyl)-1H- benzimidazol-2-yl]ethyl}-4- methyl-3,4-dihydro-2H-1,4- benzoxamide-7-sulfonamide (93.34% inhibition at 3.50 μM) | ¹H NMR (300 MHz) 8.22-8.19 (m, 1H), 7.96 (brs, 1H), 7.74-7.70 (m, 1H), 7.48-7.46 (m, 1H), 7.00-6.96 (m, 1H), 6.86-6.85 (m, 1H) 6.68-6.65 (m, 1H), 4.82-4.72 (m, 1H), 4.42-4.24 (m, 2H), 4.19-4.11 (m, 2H), 3.14-3.11 (m, 2H), 2.74 (s,3H), 1.38 (d, J = 7.14 Hz, 3H), 1.28 (t, J = Hz, 3H) | 468 | 9 |
| 44 | 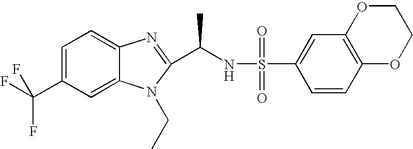 N-{(1R)-1-[1-Ethyl-6- (trifluoromethyl)-1H- benzimidazol-2-yl]ethyl}-2,3- dihydro-1,4-benzodioxine-6- sulfonamide (93.88% inhibition at 3.50 μM) | ¹H NMR (300 MHz) 8.38-8.35 (m, 1H), 7.97 (brs, 1H), 7.71-7.68 (m, 1H), 7.49-7.47 (m, 1H), 7.20-7.17 (m, 1H), 7.08-7.07 (m, 1H), 6.85-6.82 (m, 1H), 4.86-4.76 (m, 1H), 4.44-4.25 (m, 2H), 4.15-4.09 (m, 4H), 1.38 (d, J = 6.87 Hz, 3H), 1.30 (t, J = 7.14 Hz, 3H) | 455 | 9 |
| 45 | 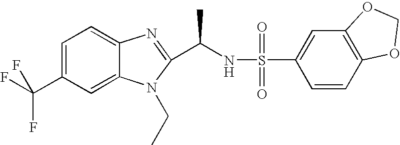 N-{(1R)-1-[1-Ethyl-6- (trifluoromethyl)-1H- benzimidazol-2-yl]ethyl}-1,3- benzodioxole-5-sulfonamide (95.96% inhibition at 3.50 μM) | ¹H NMR (300 MHz) 8.37 (brs, 1H), 7.97 (brs, 1H), 7.72-7.70 (m,1 H), 7.50-7.47 (m, 1H), 7.27-7.24 (m, 1H), 7.12-7.11 (m, 1H), 6.89-6.86 (m, 1H), 5.98 (d, J = 8.03 Hz, 2H), 4.87-4.78 (m, 1H), 4.43-4.26 (m, 2H), 1.38 (d, J = 6.87 Hz, 3H), 1.30 (t, J = 7.14 Hz, 3H) | 441 | 9 |
| 46 | 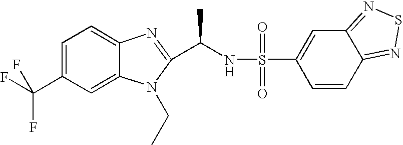 N-{(1R)-1-[1-Ethyl-6- (trifluoromethyl)-1H- benzimidazol-2-yl]ethyl}- 2,1,3-benzothiadiazole-5- sulfonamide (89.78% inhibition at 3.70 μM) | ¹H NMR (300 MHz) 8.96 (brs, 1H), 8.07-8.05 (m, 2H), 7.89-7.85 (m, 1H), 7.76 (brs, 1H), 7.34-7.18 (m, 2H), 5.01-4.91 (m, 1H), 4.37 (q, J = 7.14 Hz, 2H), 1.46 (d, J = 6.87 Hz, 3H), 1.30 (t, J = 7.14 Hz, 3H) | 455 | 9 |
| 47 | 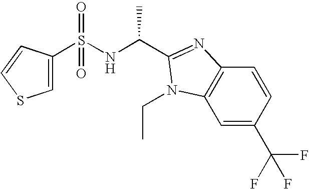 N-{(1R)-1-[1-Ethyl-6- (trifluoromethyl)-1H- benzimidazol-2- yl]ethyl}thiophene-3- sulfonamide (89.59% inhibition at 3.70 μM) | ¹H NMR (300 MHz, CDCl₃) 7.76 (dd, 1H) 7.68 (d, 1H) 7.52 (s, 1H) 7.48 (d, 1H) 7.12 (dd, 1H) 7.05 (m, 1H) 6.11 (bd, 1H) 4.82 (m, 1H) 4.21 (m, 1H) 4.12 (m, 1H) 1.58 (d, 3H) 1.36 (t, 3H) | 403 | 9 |
| 48 | 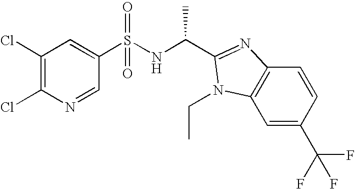 5,6-Dichloro-N-{(1R)-1-[1- ethyl-6-(trifluorometyhyl)-1H- benzimidazol-2- yl]ethyl}pyridine-3- sulfonamide (95.44% inhibition at 3.50 μM) | ¹H NMR (300 MHz, CDCl₃) 8.41 (d, 1H) 7.88 (d, 1H) 7.61 (d, 1H) 7.55 (s, 1H) 7.51 (d, 1H) 6.51 (bd, 1H) 4.85 (m, 1H) 4.20 (m, 2H) 1.64 (d, 3H) 1.43 (t, 3H) | 467 | 9 |
| 49 | 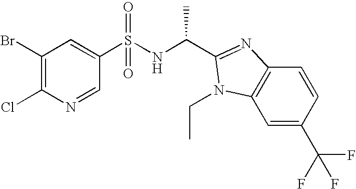 5-Bromo-6-chloro-N-{(1R)-1- [1-ethyl-6-(trifluoromethyl)- 1H-benzimidazol-2- yl]ethyl}pyridine-3- sulfonamide (92.11% inhibition at 3.70 μM) | ¹H NMR (300 MHz, CDCl₃) 8.46 (d, 1H) 8.00 (d, 1H) 7.61 (d, 1H) 7.54 (s, 1H) 7.51 (d, 1H) 6.39 (bd, 1H) 4.85 (m, 1H) 4.19 (m, 2H) 1.64 (d, 3H) 1.43 (t,3H) | 512 | 9 |
| 50 | 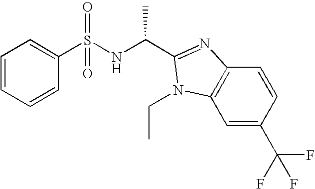 N-{(1R)-1-[1-Ethyl-6- (trifluoromethyl)-1H- benzimidazol-2- yl]ethyl}benzenesulfonamide (92.08% inhibition at 3.30 μM) | ¹H NMR (300 MHz, CDCl₃) 7.71-7.77 (m, 3H) 7.54-7.58 (m, 2H) 7.24-7.35 (m, 3H) 6.21 (bd, 1H) 4.90 (m, 1H) 4.30 (m, 1H) 4.19 (m, 1H) 1.66 (d, 3H) 1.44 (t, 3H) | 397 | 9 |
| 51 | 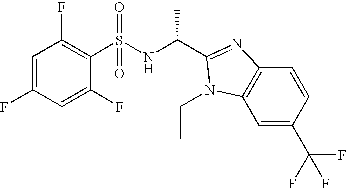 N-{(1R)-1-[1-Ethyl-6- (trifluoromethyl)-1H- benzimidazol-2-yl]ethyl}- 2,4,6- trifluorobenzenesulfonamide (92.81% inhibition at 3.70 μM) | ¹H NMR (300 MHz, CDCl₃) 7.61 (d, 1H) 7.53 (s, 1H) 7.46 (d, 1H) 6.32- 6.40 (m, 2H) 6.27 (bd, 1H) 5.02 (m, 1H) 4.35 (m, 1H) 4.17 (m, 1H) 1.67 (d, 3H) 1.39 (t, 3H) | 451 | 9 |
| 52 | 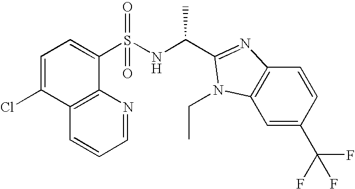 5-Chloro-N-{(1R)-1-[1-ethyl- 6-(trifluoromethyl)-1H- benzimidazol-2-yl]ethyl}- quinoline-8-sulfonamide (89.03% inhibition at 3.70 μM) | ¹H NMR (300 MHz, CDCl₃) 9.17 (dd, 1H) 8.49 (dd, 1H) 8.12 (d, 1H) 7.64 (dd, 1H) 7.52 (s, 1H) 7.46 (d, 3H) 5.17 (m, 1H) 4.51 (m, 1H) 4.22 (m, 1H) 1.66 (d, 3H) 1.48 (t, 3H) | 483 | 9 |
| 53 | 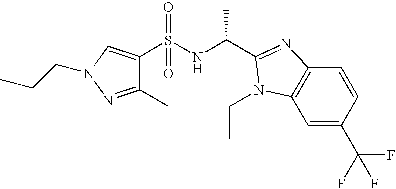 N-{(1R)-1-[1-Ethyl-6- (trifluoromethyl)-1H- benzimidazol-2-yl]ethyl}-3- methyl-1-propyl-1H- pyrazole-4-sulfonamide (84.48% inhibition at 3.70 μM) | ¹H NMR (300 MHz,CDCl₃) 7.87 (d, 1H) 7.67 (s, 1H) 7.63 (m, 2H) 6.28 (bd, 1H) 4.95 (m, 1H) 4.38 (m, 1H) 4.30 (m, 1H) 3.77 (t, 2H) 2.39 (s, 3H) 1.73 (d, 3H) 1.66 (m, 2H) 1.51 (t, 3H) 0.81 (t, 3H) | 443 | 9 |
| 54 | 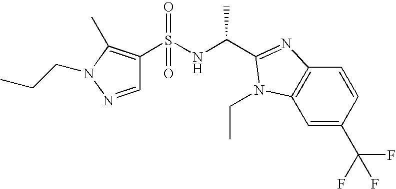 N-{(1R)-1-[1-Ethyl-6- (trifluoromethyl)-1H- benzimidazol-2-yl]ethyl}-5- methyl-1-propyl-1H- pyrazole-4-sulfonamide (77.47% inhibition at 3.70 μM) | ¹H NMR (300 MHz, CDCl₃) 7.71 (d, 1H) 7.49-7.53 (m, 3H) 6.17 (bd, 1H) 4.75 (m, 1H) 4.16 (m, 2H) 3.49 (m, 2H) 2.28 (s, 3H) 1.60 (d, 3H) 1.37 (t, 3H) 1.27 (m, 2H) 0.58 (t, 3H) | 443 | 9 |
| 55 | 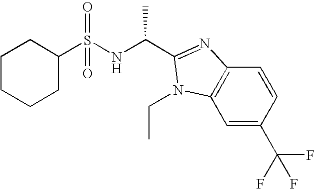 N-{(1R)-1-[1-Ethyl-6- (trifluoromethyl)-1H- benzimidazol-2- yl]ethyl}cyclohexanesulfon- amide | ¹H NMR (300 MHz) 8.06 (s, 1H), 7.94-7.92 (m, 1H), 7.81-7.79 (m, 1H), 7.54-7.51 (m, 1H), 4.97-4.90 (m, 1H), 4.46 (q, J = 7.14 Hz, 2H), 2.72-2.64 (m, 1H), 2.12-1.98 (m, 2H), 1.74 (brs, 2H), 1.58 (d, J = 6.87 Hz, 3H), 1.34 (t, J = 6.87 Hz, 3H), 1.29-1.00 (m, 6H) | 403 | 9 |
| 56 | 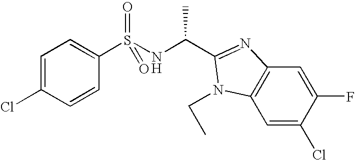 4-Chloro-N-[(1R)-1-(6- chloro-1-ethyl-5-fluoro-1H- benzimidazol-2- yl)ethyl]benzenesulfonamide (95.98% inhibition at 3.50 μM) | ¹H NMR (300 MHz( 8.6 (s, 1H), 7.8 (d, J = 6.6 Hz, 1H), 7.6 (dm, J = 8.5 Hz, 2H), 7.5 (d, J = 10.1 Hz, 1H), 7.4 (dm, J = 8.5 Hz, 2H), 4.8 (br, 1H), 4.2 (m, 2H), 1.4 (d, J = 6.8 Hz, 3H), 1.3 (t, J = 7.1 Hz, 3H) | 416 | 25 |
| 57 | 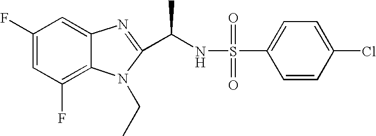 4-Chloro-N-[(1R)-1-(1-ethyl- 5,7-difluoro-1H- benzimidazol-2- yl)ethyl]benzenesulfonamide (84.47% inhibition at 3.70 μM) | ¹H NMR (300 MHz) 8.64 (s, 1H), 7.67 (d, J = 8.52 Hz, 2H), 7.45 (d, J = 8.52 Hz, 2H), 7.0-7.2 (m, 2H), 4.84 (d, J = 6.33 Hz, 2H), 4.2-4.3 (m, 1H), 1.31- 1.38 (m, 6H) | 399 | 26 |
| 58 | 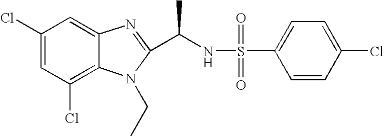 4-Chloro-N-[(1R)-1-(5,7- dichloro-1-ethyl-1H- benzimidazol-2- yl)ethyl]benzenesulfonamide (78.24% inhibition at 3.70 μM) | ¹H NMR (300 MHz) 8.68 (s, 1H), 7.67 (d, J = 8.49 Hz, 2H), 7.55 (d, J = 1.65 Hz, 1H), 7.43 (d, J = 8.52 Hz, 2H), 7.36 (d, J = 1.65 Hz, 1H), 4.8 (m, 1H), 4.45 (m, 2H), 1.33-1.39 (m, 6H) | 433 | 27 |
| 59 | 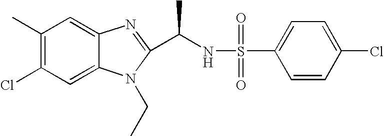 4-Chloro-N-[(1R)-1-(6- chloro-1-ethyl-5-methyl-1H- benzimidazol-2- yl)ethyl]benzenesulfonamide (78.24% inhibition at 3.70 μM) | ¹H NMR (300 MHz) 8.57 (d, J = 8.52 Hz, 1H), 7.66 (m, 3H), 7.44 (m, 3H), 4.80 (m, 1H), 4.2 (m, 2H), 2.38 (s, 3H), 1.35 (d, J = 6.87 Hz, 3H), 1.26 (t, J = 6.87 Hz, 3H) | 411 | 28 |
| 60 | 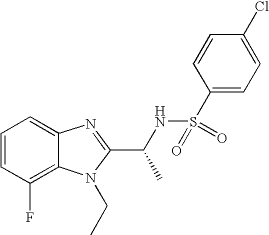 4-Chloro-N-[91R)-1-(1-ethyl- 7-fluoro-1H-benzimidazol-2- yl)ethyl]benzenesulfonamide (97.68% inhibition at 3.70 μM) | ¹H NMR (300 Mhz) 8.60 (d, J = 8.8 Hz, 1H), 7.70 (m, 2H), 7.48 (m, 2H), 7.35 (d, J = 8.0 Hz, 2H), 7.16-7.00 (m, 2H), 4.86 (m, 1H), 4.33 (m, 2H), 1.41- 1.33 (m, 6H) | 381 | 29 |
| 61 | 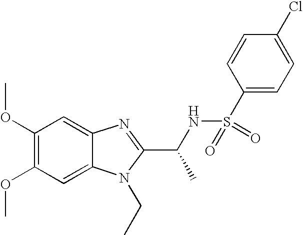 4-Chloro-N-[(1R)-1-(1-ethyl- 5,6-dimethoxy-1H- benzimidazol-2- yl)ethyl]benzenesulfonamide (54.94% inhibition at 3.70 μM) | ¹H NMR (300 MHz) 8.47 (bs, 1H), 7.68 (m, 2H), 7.45 (m, 2H), 7.04-7.03 (m, 2H), 4.76 (m, 1H), 4.18 (m, 2H), 3.81 (s, 3H), 3.75 (s, 3H), 1.33 (d, J = 6.6 Hz, 3H), 1.27 (t, J = 6.9 Hz, 3H) | 423 | 30 |
| 62 | 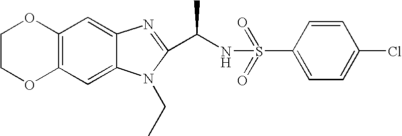 4-Chloro-N-[(1R)-1-(1-ethyl- 6,7-dihydro-1H-[1,4]dioxino- [2,3-f]benzimidazol-2-yl)- ethyl]benzenesulfonamide (52.20% inhibition at 3.70 μM) | ¹H NMR (300 Mhz) 8.48 (d, J = 8.25 Hz, 1H), 7.68 (d, J = 8.52 Hz, 2H), 7.46 (d, J = 8.25 Hz, 2H), 6.93 (d, J = 3.0 Hz, 2H), 4.74 (m, 1H), 4.21 (m, 6H), 1.31 (d, J = 6.3 Hz, 3H), 1.22 (t, J = 6.87 Hz, 3H). | 421 | 31 |
| 63 | 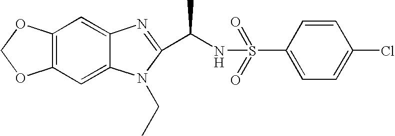 (R)-4-Chloro-N-[1-(5-Ethyl- 5H-[1,3]dioxolo[4′,5′;4,5]- benzo[1,2-d]imidazol-6-yl)- ethyl]benzenesulfonamide (93.94% inhibition at 3.50 μM) | ¹H NMR (300 MHz) 8.48 (brs, 1H), 7.70-7.66 (m, 2H), 7.49-7.45 (m, 2H), 7.10 (s, 1H), 6.99 (s, 1H), 5.98-5.97 (m, 2H), 4.78-4.70 (m, 1H), 4.15 (q, J = 7.14 Hz, 2H), 1.32 (d, J = 6.87 Hz, 3H), 1.23 (t, J = 7.14 Hz, 3H) | 407 | 32 |
| 64 | 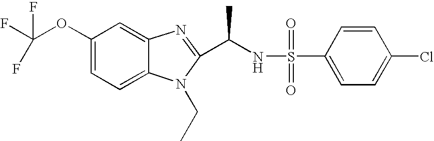 4-Chloro-N-{(1R)-1-[1-ethyl- 5-(trifluoromethoxy)-1H- benzimidazol-2- yl]ethyl}benzenesulfonamide (50.65% inhibition at 3.70 μM) | ¹H NMR (300 MHz, CDCl₃) 1.36 (t, J = 7.41 Hz, 3H), 1.56 (d, J = 6.87 Hz, 3H), 4.01-4.28 (m, 2H), 4.81 (brs, 1H), 6.13 (brs, 1H), 7.12-7.19 (m, 4H), 7.47 (br. s, 1H), 7.63 (d, J = 8.52 Hz, 2H) | 447 | 33 |
| 65 | 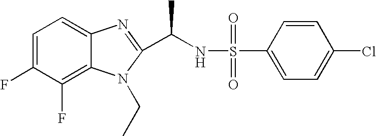 4-Chloro-N-[(1R)-1-(1-ethyl- 6,7-difluoro-1H- benzimidazol-2- yl)ethyl]benzenesulfonamide (84.17% inhibition at 3.70 μM) | ¹H NMR (300 MHz, CDCl₃) 1.39 (t, J = 7.14 Hz, 3H), 1.57 (d, J = 6.87 Hz, 3H), 4.01-4.29 (m, 2H), 4.76 (q, J = 6.60 Hz, 1H), 5.90 (d, J = 9.06 Hz, 1H), 7.01-7.08 (m, 1H), 7.27 (d, J = 8.52 Hz, 2H), 7.25-7.35 (m, overlap with CDCl₃, 1H), 7.63 (d, J = 8.52, Hz, 2H) | 399 | 34 |
| 66 | 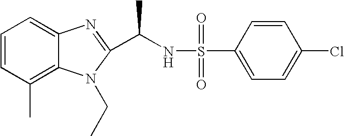 4-Chloro-N-[(1R)-1-(1-ethyl- 7-methyl-1H-benzimidazol-2- yl)ethyl]benzenesulfonamide (94.59% inhibition at 3.70 μM) | ¹H NMR (300 MHz, CDCl₃) 1.30 (t, J = 7.14 Hz, 3H), 1.57 (d, J = 6.87 Hz, 3H), 2.62 (s, 3H), 4.01-4.29 (m, 2H), 4.78 (q, J = 6.60 Hz, 1H), 5.97 (br. s, 1H), 6.96 (d, J = 7.14 Hz, 1H), 7.11 (t, J = 7.71 Hz, 1H), 7.18 (d, J = 8.52 Hz, 2H), 7.44 (d, J = 7.98 Hz, 1H), 7.63 (d, J = 8.52, Hz, 2H) | 377 | 35 |
| 67 | 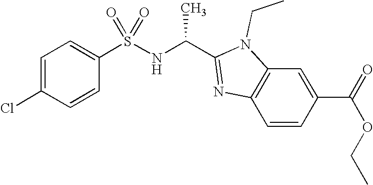 Ethyl 2-(1-{[(4- chlorophenyl)sulfonyl]amino}- ethyl)-1-ethyl-1H- benzimidazole-6-carboxylate (91.34% inhibition at 3.70 μM) | ¹H NMR (CDCl₃) 1.43 (t, J = 7.20 Hz, 6H) 1.65 (d, J = 6.82 Hz, 3H), 4.12- 4.23 (m, 1H) 4.24-4.34 (m, 1H) 4.42 (q, J = 7.07 Hz, 2H) 4.82-4.92 (m, 1H) 6.40 (broad s, 1H), 7.13- 7.20 (m, 2H), 7.56-7.65 (m, 3H) 8.01 (m, J = 8.59 Hz,1H), 8.05 (m, 1H) | 435 | 36 |
| 68 | 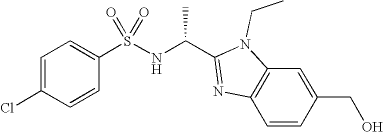 4-Chloro-N-{1-[1-ethyl-6- (hydroxymethyl)-1H- benzimidazol-2- yl]ethyl}benzenesulfonamide (94.38% inhibition at 3.50 μM) | ¹H NMR 1.29 (t, J = 7.20 Hz, 3H) 1.35 (d, J = 6.82 Hz, 3H) 4.20-4.31 (m, 2H) 4.58 (m, 2H) 4.79-4.90 (m, 1H) 5.12 (broad s, 1H) 7.20 (m, 1H) 7.44 (m, 1H) 7.46 (m, 1H) 7.52 (m, 2H) 7.75 (m, 2H) 8.55 (m, 1H) | 392 | Ex. 67 |
| 69 | 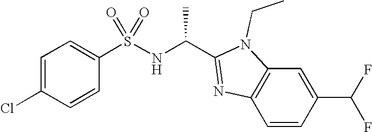 4-Chloro-N-{(1R)-1-[6- (difluoromethyl)-1-ethyl-1H- benzimidazol-2- yl]ethyl}benzenesulfonamide (85.16% inhibition at 3.50 μM) | ¹H NMR 1.30 (t, J = 6.95 Hz, 3H) 1.37 (d, J = 6.82 Hz, 3H) 4.32 (m, 2H) 4.82- 4.92 (m, 1H) 7.09 (t, J = 56.0 Hz, 1H) 7.36 (m, 1H) 7.45 (m, 2H) 7.62 (m, 1H) 7.70 (m, 2H) 7.75 (m, 1H) 8.60 (m, 1H) | 412 | Ex. 68 |
| 70 | 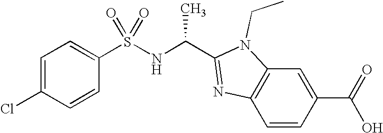 2-(1-{[(4- Chlorophenyl)sulfonyl]amino}- ethyl-1-ethyl-1H-benz- imidazole-6-carboxylic acid | ¹H NMR 1.30 (t, J = 7.20 Hz, 3H) 1.38 (d, J = 6.82 Hz, 3H) 4.28-4.38 (m, 2H) 4.82-4.91 (m, 1H) 7.42 (m, 2H) 7.55 (m, 1H) 7.68 (m, 2H) 7.78 (m, 1H) 8.08 (m, 1H) 8.62 (m, 1H) 12.79 (s, 1H) | 406 | Ex. 67 |
| 71 | 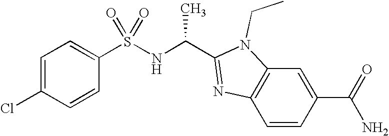 2-(1-{[(4- Chlorophenyl)sulfonyl]amino}- ethyl)-1-ethyl-1H-benz- imidazole-6-carboxamide (77.26% inhibition at 3.70 μM) | ¹H NMR) 1.32-1.42 (m, 6H) 4.33 (m, 2H) 4.89 (m, 1H) 7.34 (s, 1H) 7.48-7.57 (m, 2H) 7.58 (m, 1H) 7.68-7.77 (m, 2H) 7.79 (m, 1H) 7.93-8.05 (m, 1H) 8.12 (s, 1H) 8.66 (m, 1H) | 405 | Ex. 67 |
| 72 | 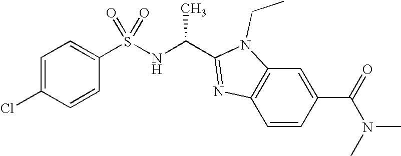 2-(1-{[(4- Chlorophenyl)sulfonyl]amino}- ethyl)-1-ethyl-N,N- dimethyl-1H-benzimidazole- 6-carboxamide (21.69% inhibition at 3.70 μM) | ¹H NMR 1.31 (t, J = 7.20 Hz, 3H) 1.40 (d, J = 6.82 Hz, 3H) 2.98 (s, 6H), 4.35 (m, 2H) 4.84-4.95 (m, 1H) 7.30 (m, 1H) 7.45-7.54 (m, 2H) 7.59 (m, 1H) 7.67-7.75 (m, 3H) 8.71 (m, 1H) | 433 | Ex. 67 |
| 73 | 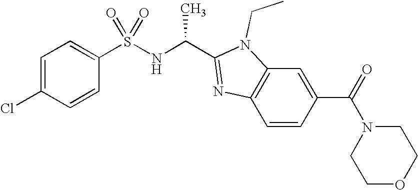 4-Chloro-N-{1-[1-ethyl-6- (morpholin-4-ylcarbonyl)- 1H-benzimidazol-2- yl]ethyl}benzenesulfonamide (8.88% inhibition at 3.70 μM) | ¹H NMR 1.31 (t, J = 7.20 Hz, 3H) 1.40 (d, J = 6.82 Hz, 3H) 3.47 (broad s, 4H) 3.61 (broad s, 4H) 4.34 (m, 2H) 4.84-4.95 (m, 1H) 7.28 (m, 1H) 7.48 (m, 2H) 7.59 (m, 1H) 7.71 (m, 3H) 8.70 (m, 1H) | 476 | Ex. 67 |
| 74 | 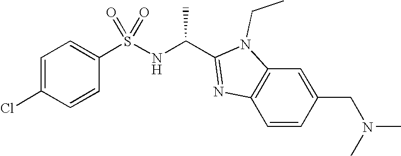 4-Chloro-N-((1R)-1-{6- [(dimethylamino)methyl]-1- ethyl-1H-benzimidazol-2- yl}ethyl)benzenesulfonamide (30.38% inhibition at 3.70 μM) | ¹H NMR (300 Mhz) 1.31-1.44 (m, 6H) 2.74 (d, J = 3.77 Hz, 6H) 4.31 (m, 2H) 4.39 (m, 2H) 4.90 (m, 1H) 7.33 (m, 1H) 7.49 (m, 2H) 7.63 (m, 1H) 7.71 (m, 3H) 8.70 (m, 1H) 9.88 (broad s, 1H) | 420 | Ex. 68 |
| 75 | 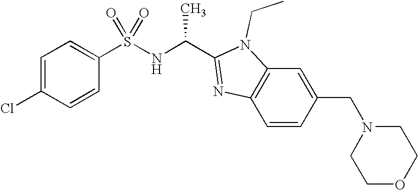 4-Chloro-N-{(1R)-1-[1-ethyl- 6-(morpholin-4-ylmethyl)- 1H-benzimidazol-2- yl]ethyl}benzenesulfonamide (37.25% inhibition at 3.70 μM) | ¹H NMR 1.32-1.42 (m, 6H) 3.14 (m, 2H) 3.25 (m, 2H) 3.64 (m, 2H) 3.95 (m, 2H) 4.26-4.37 (m, 2H) 4.46 (s, 2H) 4.84-4.95 (m, 1H) 7.33 (m, 1H) 7.46-7.53 (m, 2H) 7.62 (m, 1H) 7.69-7.76 (m, 3H) 8.65 (m, 1H) 10.21 (broad s, 1H) | 463 | Ex. 68 |
| 76 | 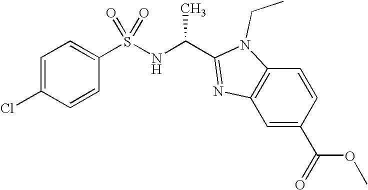 Methyl 2-(1-{[(4- chlorophenyl)sulfonyl]amino}- ethyl)-1-ethyl-1H- benzimidazole-6-carboxylate (52.94% inhibition at 3.70 μM) | ¹H NMR 1.31 (t, J = 7.20 Hz, 3H) 1.38 (d, J = 6.82 Hz, 3H) 3.85 (s, 3H) 4.25- 4.36 (m, 2H) 4.86 (m, 1H) 7.41- 7.49 (m, 2H) 7.61 (m, 1H) 7.65- 7.72 (m, 2H) 7.85 (m, 1H) 8.08 (m, 1H) 8.62 (d, J = 8.59 Hz, 1H) | 420 | 37 |
| 77 | 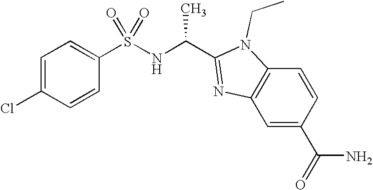 2-(1-{[(4- Chlorophenyl)sulfonyl]amino}- ethyl)-1-ethyl-1H-benz- imidazole-6-carboxamide (40.2% inhibition at 3.70 μM) | ¹H NMR 1.32 (t, J = 7.07 Hz, 3H) 1.40 (d, J = 6.82 Hz, 3H) 4.35 (m, 2H) 4.86- 4.97 (m, 1H) 7.34 (broad s, 1H) 7.52 (m, 2H) 7.67 (m, 1H) 7.74 (m, 2H) 7.84-7.95 (m, 1H) 8.00 (bs, 1H) 8.12 (m, 1H) 8.72 (m, 1H) | 405 | Ex. 76 |
| 78 | 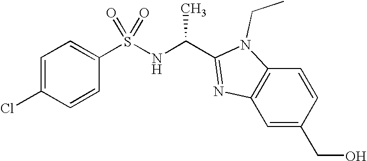 4-Chloro-N-{(1R)-1-[1-ethyl- 5-(hydroxymethyl)-1H- benzimidazol-2- yl]ethyl}benzenesulfonamide (58.42% inhibition at 3.70 μM) | ¹H NMR 1.29 (t, J = 7.20 Hz, 3H) 1.35 (d, J = 6.82 Hz, 3H) 4.20-4.31 (m, 2H) 4.58 (m, 2H) 4.79-4.90 (m, 1H) 5.12 (broad s, 1H) 7.20 (m, 1H) 7.44 (m, 1H) 7.46 (m, 1H) 7.52 (m, 2H) 7.75 (m, 2H) 8.55 (m, 1H) | 392 | Ex. 76 |
| 79 | 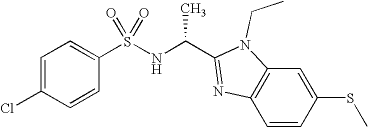 4-Chloro-N-{1-[1-ethyl-6- (methylthio)-1H- benzimidazol-2- yl]ethyl}benzenesulfonamide (94.94% inhibition at 3.70 μM) | ¹H NMR) 1.27 (t, J = 7.20 Hz, 3H) 1.34 (d, J = 6.82 Hz, 3H) 2.52 (s, 3H) 4.23 (m, 2H) 4.80 (m, 1H) 7.10 (m, 1H) 7.40 (m, 1H) 7.41-7.51 (m, 3H) 7.67-7.76 (m, 2H) 8.53 (m, 1H) | 408 | 38 |
| 80 | 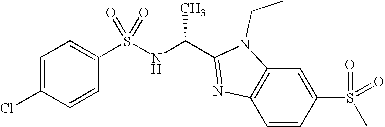 4-Chloro-N-{1-[1-ethyl-6- (methylsulfonyl)-1H- benzimidazol-2- yl]ethyl}benzenesulfonamide (22.57% inhibition at 3.70 μM) | ¹H NMR 1.33 (t, J = 7.20 Hz, 3H), 1.39 (d, J = 6.82 Hz, 3H) 3.22 (s, 3H) 4.38 (m, 2H) 4.85-4.94 (m, 1H) 7.41 (m, 2H) 7.66 (m, 2H) 7.69 (m, 2H) 8.10 (m, 1H) 8.65 (m, 1H) | 440 | Ex. 79 |
| 81 | 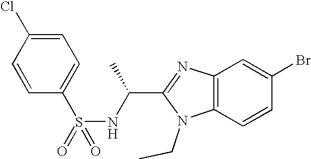 N-[(1R)-1-(5-Bromo-1-ethyl- 1H-benzimidazol-2-yl)ethyl]- 4-chlorobenzenesulfonamide (91.61% inhibition at 3.70 μM) | ¹H NMR 1.27 (t, 3H), 1.33 (d, 3H) 4.25 (m, 2H), 4.82 (m, 1H), 7.34 (m, 1H), 7.46 (dd, 3H), 7.67 (m, 2H), 8.61 (d, 1H) | 441 | 39 |
| 82 | 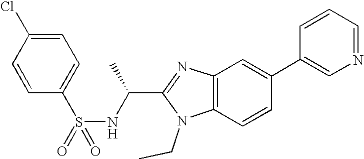 4-Chloro-N-[(1R)-1-(1-ethyl- 5-pyridin-3-yl-1H- benzimidazol-2- yl)ethyl]benzenesulfonamide (10.27% inhibition at 3.70 μM) | ¹H NMR 1.31 (t, 3H), 1.38 (d, 3H), 4.30 (m, 2H), 4.85 (m, 1H), 7.47 (m, 2H), 7.58 (m, 2H), 7.69 (d, 2H), 7.82 (s, 1H), 8.08 (d, 1H), 8.53 (d, 1H), 8.61 (d, 1H), 8.91 (s, 1H) | 440 | 40 |
| 83 | 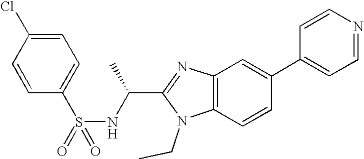 4-Chloro-N-[(1R)-1-(1-ethyl- 5-pyridin-4-yl-1H- benzimidazol-2- yl)ethyl]benzenesulfonamide (16.38% inhibition at 3.70 μM) | ¹H NMR 1.31 (t, 3H), 1.38 (d, 3H), 4.31 (m, 2H), 4.86 (m, 1H), 7.45 (d,m 2H), 7.65 (m, 2H), 7.69 (s, 1H), 7.73 (m, 2H), 7.94 (s, 1H), 8.60 (d, 3H) | 440 | 41 |
| 84 | 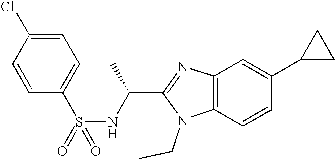 4-Chloro-N-[(1R)-1-(5- cyclopropyl-1-ethyl-1H- benzimidazol-2- yl)ethyl]benzenesulfonamide (59.34% inhibition at 3.70 μM) | ¹H NMR (300 MHz) 0.65 (m, 2H) 0.84-0.96 (m, 2H) 1.26 (t, J = 7.16 Hz, 3H) 1.32 (d, J = 6.78 Hz, 3H) 1.98 (m, 1H) 4.21 (m, 2H) 4.73-4.87 (m, 1H) 6.95 (m, 1H) 7.19 (m, 1H) 7.34 (m, 1H) 7.47 (m, 2H) 7.70 (m, 2H) 8.52 (m, 1H) | 403 | 42 |
| 85 | 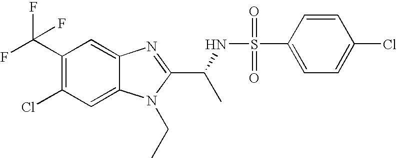 4-Chloro-N-{(1R)-1-[6chloro- 1-ethyl-5-(trifluoromethyl)-1H- benzimidazol-2- yl]ethyl}benzenesulfonamide (44.93% inhibition at 3.70 μM) | ¹H NMR (300 MHz) 1.30 (t, 3H) 1.41 (d, 3H) 4.37 (m, 2H) 4.87 (m, 1H) 7.38 (d, 2H) 7.62 (d, 2H) 7.79 (s, 1H) 8.09 (s, 1H) 8.71 (d, 1H) | 465 | 43 |
| 86 | 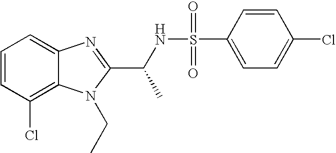 (R)-4-Chloro-N-[1-(7-chloro- 1-ethyl-1H-benzoimidazol-2- yl)ethyl]- benzenesulfonamide (51.70% inhibition at 3.70 μM) | ¹H NMR (300 MHz) 8.63 (brs, 1H), 7.71-7.67 (m, 2H), 7.51-7.43 (m, 3H), 7.26-7.13 (m, 2H), 4.89-4.83 (m, 1H), 4.55-4.39 (m, 2H), 1.39-1.35 (m, 6H) | 397 | 44 |
| 87 | 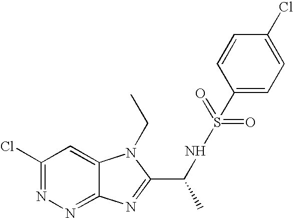 4-Chloro-N-[(1R)-1-(3- chloro-5-ethyl-5H- imidazo[4,5-c]pyridazin-6- yl)ethyl]benzenesulfonamide (62.35% inhibition at 3.70 μM) | ¹H NMR 8.85 (1H, d); 8.23 (1H, s); 7.70 (2H, d), 7.47 (2H, d), 4.94 (1H, m); 4.31 (2H, m); 1.42 (3H, d), 1.32 (3H, t) | 399 | 45 |
| 88 | 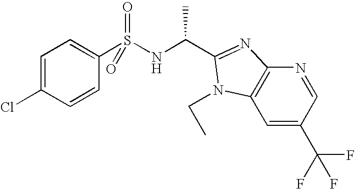 4-Chloro-N-{(1R)-1-[1-ethyl- 6-(trifluoromethyl)-1H- imidazo[4,5-b]pyridin-2- yl]ethyl}benzenesulfonamide (82.60% inhibition at 3.55 μM) | ¹H NMR (300 MHz, CDCl₃) 1.35-1.43 (t, 3H) 1.54-1.62 (d, 3H) 4.05-4.35 (m, 2H) 4.74-4.86 (s, 1H) 6.18-6.27 (d, 1H) 7.14-7.19 (dd, 2H) 7.60-7.67 (dd, 2H) 7.80 (s, 1H) 8.74 (s, 1H) | 433 | 46 |
| 89 | 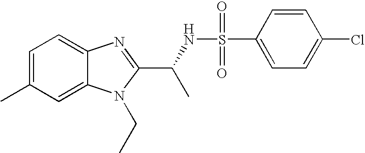 4-Chloro-N-[(1R)-1-(1-ethyl- 6-methyl-1H-benzimidazol-2- yl)ethyl]benzenesulfonamide (90.69% inhibition at 3.50 μM) | ¹H NMR (300 MHz) 1.28 (t, 3H) 1.34 (d, 3H) 2.43 (s, 3H) 4.22 (q, 2H) 4.81 (m, 1H) 6.98 (d, 1H) 7.27 (s,1H) 7.39 (d, 1H) 7.49 (d, 2H) 7.73 (d, 2H) 8.53 (d, 1H) | 377 | 47 |
| 90 | 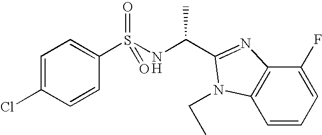 4-Chloro-N-[(1R)-1-(1-ethyl- 4-fluoro-1H-benzimidazol-2- yl)ethyl]benzenesulfonamide (93.41% inhibition at 3.50 μM) | ¹H NMR (300 MHz, CDCl₃) 1.33 (t, 3H) 1.56 (d,3H) 3.99-4.07 (m, 1H) 4.09-4.18 (m, 1H) 4.71-4.82 (m, 1H) 6.07 (d, 1H) 6.88-6.96 (m, 1H) 7.00-7.04 (m, 1H) 7.14 (m, 4H) 7.53 (dd, 2H) | 382 | 48 |
| 91 | 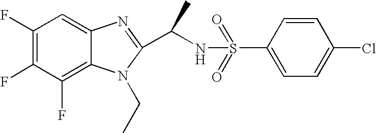 (R)-4-Chloro-N-[1-(1-ethyl- 5,6,7-trifluoro-1H- benzoimidazol-2-yl)-ethyl]- benzenesulfonamide (91.78% inhibition at 1 μM) | ¹H NMR (300 MHz) 8.63 (brs, 1H), 7.67-7.64 (m, 2H), 7.46-7.43 (m, 3H), 4.88-4.81 (m, 1H), 4.30 (q, J = 7.14 Hz, 2H), 1.39-1.33 (m, 6H) | 417 | 49 |
| 92 | 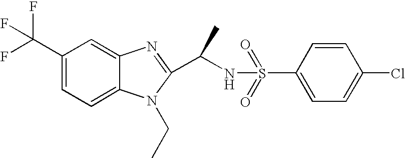 4-Chloro-N-{(1R)-1-[1-ethyl- 5-(trifluoromethyl)-1H- benzimidazol-2- yl]ethyl}benzenesulfonamide (86.06% inhibition at 3.50 μM) | ¹H NMR (300 MHz, CDCl₃) 1.36-1.44 (t, 3H) 1.55-1.61 (d, 3H) 1.65 (s, 3H) 4.06-4.31 (m, 2H) 4.78-4.88 (m, 1H) 5.90-5.96 (m, 1H) 7.18-7.23 (ddm 2H) 7.32-7.37 (d,1H) 7.49-7.54 (m, 1H) 7.60-7.65 (dd, 2H) 7.87 (s,1H) | 432 | 50 |
| 93 | 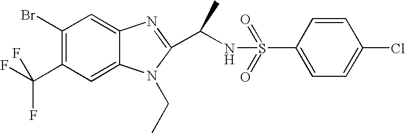 N-{(1R)-1-[5-Bromo-1-ethyl- 6-(trifluoromethyl)-1H- benzimidazol-2-yl]ethyl}-4- chlorobenzenesulfonamide (62.3% inhibition at 3.70 μM) | ¹HNMR (300 MHz, CDCl₃) 1.36-1.44 (t, 3H) 1.60-1.66 (d, 3H) 4.10-4.33 (m, 2H) 4.77-4.89 (m, 2H) 6.50 (s, 1H) 7.12-7.18 (dd, 2H) 7.56-7.62 (dd, 2H) 7.93 (s, 1H) | 512 | 51 |
| 94 | 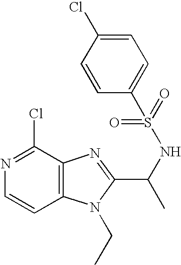 4-Chloro-N-[1-(4-chloro-1- ethyl-1H-imidazo[4,5- c]pyridin-2- yl)ethyl]benzenesulfonamide (91.61% inhibition at 3.70 μM) | ¹H NMR 8.73 (1H, d); 8.11 (1H, d); 7.67 (2H, d), 7.44 (2H, d), 4.91 (1H, m); 4.33 (2H, m); 1.38 (3H, d), 1.30 (3H, t) | 399 | 52 |
| 95 | 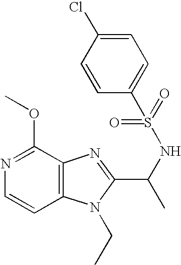 4-Chloro-N-[1-(1-ethyl-4- methoxy-1H-imidazo[4,5- c]pyridin-2-yl)ethyl] benzenesulfonamide (47.67% inhibition at 3.50 μM) | ¹H NMR (300 MHz, CDCl₃) 7.91 (1H, d), 7.57 (2H, d), 7.15 (2H, d), 6.86 (1H, d5.93 (1H, m), 4.80 (1H, m), 4.11 (3H, s), 4.10 (2H, m), 1.56 (3H, d), 1.37 (3H, t) | 395 | Ex. 94 |
| 96 | 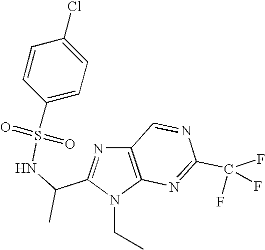 4-Chloro-N-{1-[9-ethyl-2- (tri-fluoromethyl)-9H-purin- 8-yl]ethyl}benzene- sulfonamide (81.67% inhibition at 3.70 μM) | ¹H NMR 9.2 (1H, s); 8.84 (1H, d); 7.67 (2H, d), 7.44 (2H, d), 4.99 (1H, m); 4.36 (2H, m); 1.44 (3H, d), 1.38 (3H, t) | 433 | — |
| 97 | 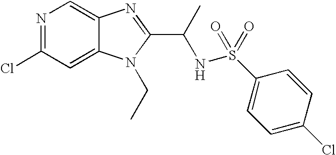 4-Chloro-N-[(1R)-1-(4- chloro-7-erthyl-3,7,9- triazabicyclo[4.3.0]nona- 1,3,5,8-tetraen-8- yl)ethyl]benzenesulfonamide (88.94% inhibition at 3.70 μM) | ¹H NMR (300 MHz) 1.29 (t, J = 7.16 Hz, 3H), 1.38 (d, J = 6.78 Hz, 3H), 4.28 (m, 2H), 4.88 (m, 1H), 7.49 (d, J = 8.67 Hz, 2H), 7.70 (d, J = 8.67 Hz, 2H), 7.78 (s, 1H), 8.61 (s, 1H), 8.69 (d, J = 8.67 Hz, 1H) | 398 | — |
| 98 | 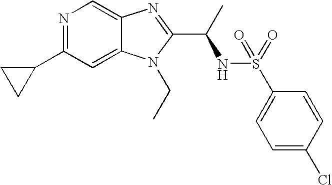 4-Chloro-N-[(1R)-1-(6- cyclopropyl-1-ethyl-1H- imidazo[4,5-c]pyridin-2- yl)ethyl]benzenesulfonamide (88.22% inhibition at 3.50 μM) | ¹H NMR (300 MHz) 0.92 (m, 4H), 1.32 (m, 6H), 2.15 (s, 1H), 4.25 (m, 2H), 4.85 (d, J = 6.78 Hz, 1H), 7.43 (s, 1H), 7.50 (d, J = 8.48 Hz, 2H), 7.72 (d, J = 8.48 Hz, 2H), 8.62 (m, 2H) | 404 | — |
| 99 | 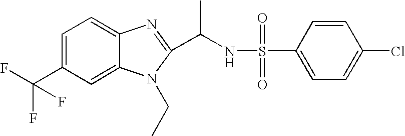 4-Chloro-N-{1-[1-ethyl-6- (trifluoromethyl)-1H- benzimidazol-2- yl]ethyl}benzenesulfonamide (94.50% inhibition at 3.70 μM) | ¹H NMR (MeOH-d₄) 7.84 (s, 1H), 7.7 (d, 1H), 7.64 (d, 2H), 7.54 (d, 1H), 7.30 (d, 2H), 4.98 (q, 1H), 4.43 (q, 2H), 1.57 (d, 3H), 1.43 (t, 3H) | 341 | 9a |
| 100 | 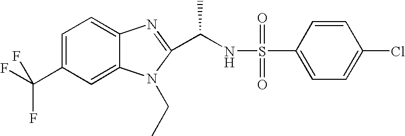 4-Chloro-N-{(1S)-1-[1-ethyl- 6-(trifluoromethyl)-1H- benzimidazol-2- yl]ethyl}benzenesulfonamide (84.59% inhibition at 3.50 μM) | ¹HNMR (MeOH-d₄) 7.84 (s, 1H), 7.7 (d, 1H), 7.64 (d, 2H), 7.54 (d,1H), 7.30 (d, 2H), 4.98 (q, 1H), 4.43 (q, 2H), 1.57 (d, 3H), 1.43 (t,3H) | 341 | 9b |
| 101 | 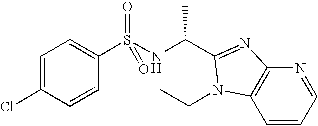 4-Chloro-N-[(1R)-1-(1-ethyl- 1H-imidazo[4,5-b]pyridin-2- yl)ethyl]benzenesulfonamide | ¹H NMR (300 MHz) 1.30 (t, 3H) 1.40 (d, 3H) 4.30 (q, 2H) 4.81-4.95 (m, 1H) 7.18-7.27 (m, 1H) 7.45 (d, 2H) 7.70 (d, 2H) 7.92-8.00 (m, 1H) 8.32- 8.39 (m, 1H) 8.65-8.73 (m, 1H) | 364 | Ex. 12 |
| 102 | 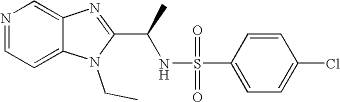 4-Chloro-N-[(1R)-1-(1-ethyl- 1H-imidazo[4,5-c]pyridin-2- yl)ethyl]benzenesulfonamide (93.75% inhibition at 3.50 μM) | ¹H NMR (CDCl₃) 8.8 (s, 1H), 8.3 (d, 1H), 7.7 (d, 2H), 7.6 (d, 1H), 7.3 (d, 2H), 4.4 (q, 2H), 1.6 (d, 3H), 1.4 (t, 3H) | 364 | Ex. 13 |
| 103 | 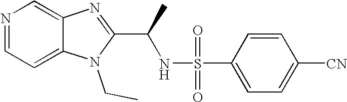 4-Cyano-N-[(1R)-1-(1-ethyl- 1H-imidazol[4,5-c]pyridin-2- yl)ethyl]benzenesulfonamide (89.44% inhibition at 3.50 μM) | ¹H NMR 8.91 (s, 1H), 8.8 (s, 1H), 8.30 (d, 2H), 7.85 (d, 1H), 7.84 (s, 1H), 7.6 (d, 1H), 4.98 (m, 1H), 4.31 (q, 2H), 1.44 (d, 3H), 1.31 (t, 6H) | 355 | — |
| 104 | 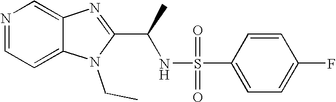 4-Fluoro-N-[(1R)-1-(1-ethyl- 1H-imidazo[4,5-c]pyridin-2- yl)ethyl]benzenesulfonamide (90.30% inhibition at 3.50 μM) | ¹H NMR (MeOH-d₄): 8.7 (s, 1H), 8.29 (d, 2H), 7.78 (dd, 2H), 7.55 (d, 1H), 7.05 (dd, 1H), 4.80 (m, 1H), 4.25 (q, 2H), 1.44 (d, 3H), 1.33 (t, 6H) | 348 | — |
| 105 | 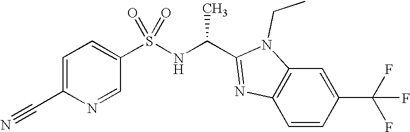 (R)-6-Cyano-N-(1-(1-ethyl-6- (trifluoromethyl)-1H- benzo[d]imidazol-2-yl)ethyl) pyridine-3-sulfonamide (93.08% inhibition at 3.50 μM) | ¹H NMR 1.29 (t, J = 7.07 Hz, 3H) 1.48 (d, J = 6.82 Hz, 3H) 4.36 (m, 2H) 4.93-5.03 (m, 1H) 7.41-7.52 (m, 1H) 7.57 (m, 1H) 7.84 (m, 1H) 7.94 (m, 1H) 8.12 (m, 1H) 8.81 (m, 1H) 9.17 (m, 1H). | 423 | 9 |
| 106 | 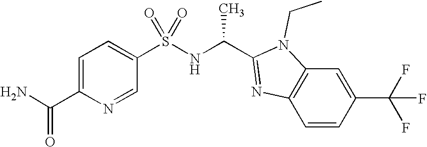 (R)-5-(N-(1-(1-Ethyl-6- (trifluoromethyl)-1H- benzo[d]imidazol-2-yl)ethyl) sulfamoyl)picolinamide (93.35% inhibition at 3.50 μM) | ¹H NMR 1.,30 (t, J = 7.07 Hz, 3H) 1.41 (d, J = 6.82 Hz, 3H) 4.38 (m, 2H) 4.94-5.04 (m, 1H) 7.41 (m, 1H) 7.61 (m, 1H) 7.76 (m, 1H) 7.92- 8.00 (m, 2H) 8.04 (m, 1H) 8.18 (m, 1H) 8.82 (m, 1H) 8.96 (m, 1H). | 441 | Ex. 105 |
| 107 | 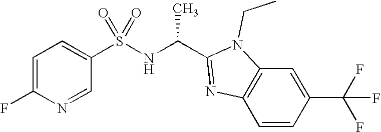 (R)-N-(1-(1-Ethyl-6- (trifluoromethyl)-1H- benzo[d]imidazol-2-yl)ethyl)- 6-fluoropyridine-3- sulfonamide (92.55% inhibition at 3.50 μM) | ¹H NMR (t, J = 7.07 Hz, 3H) 1.43 (d, J = 6.57 Hz, 3H) 4.37 (m, 2H) 4.92- 5.03 (m, 1H) 7.10-7.20 (m, 1H) 7.44-7.54 (m, 1H) 7.63 (m, 1H) 7.96 (m, 1H) 8.13-8.24 (m, 1H) 8.44 (m, 1H) 8.88 (m, 1H). | 416 | 9 |
| 108 | 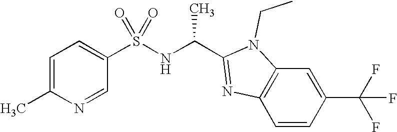 (R)-N-(1-(1-Ethyl-6- (trifluoromethyl)-1H- benzo[d]imidazol-2-yl)ethyl)- 6-methylpyridine-3- sulfonamide (91.76% inhibition at 3.50 μM) | ¹H NMR 1.29 (t, J = 7.07 Hz, 3H) 1.41 (d, J = 6.82 Hz, 3H) 2.30 (s, 3H) 4.30-4.41 (m, 2H) 4.90 (m, 1H) 7.15 (m, 1H) 7.46 (m, 1H) 7.64 (m, 1H) 7.85 (m, 1H) 7.94 (m, 1H) 8.63 (m, 1H) 8.70 (m, 1H) | 412 | 9 |
| 109 | 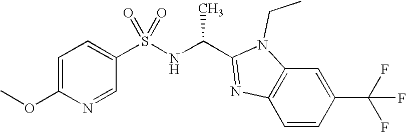 (R)-N-(1-(1-Ethyl-6- (trifluoromethyl)-1H- benzo[d]imidazol-2-yl)ethyl)- 6-methoxypyridine-3- sulfonamide (95.57% inhibition at 3.50 μM) | ¹H NMR 1.29 (t, J = 7.07 Hz, 3H) 1.43 (d, J = 6.82 Hz, 3H) 3.69 (s, 3H) 4.36 (m, 2H) 4.83-4.93 (m, 1H) 6.72 (m, 1H) 7.44 (m, 1H) 7.62 (m, 1H) 7.86 (m, 1H) 7.94 (m, 1H) 8.27 (m, 1H) 8.60 (m, 1H) | 428 | 9 |
| 110 | 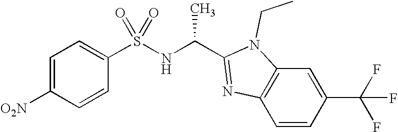 (R)-N-(1-(1-Ethyl-6- (trifluoromethyl)-1H- benzo[d]imidazol-2-yl)ethyl)- 4-nitrobenzenesulfonamide (92.41% inhibition at 3.50 μM) | ¹H NMR 1.29 (t, J = 7.16 Hz, 3H) 1.45 (d, J = 6.78 Hz, 3H) 4.28-4.43 (m, 2H) 4.87-5.02 (m, 1H) 7.37 (m, 1H) 7.56 (m, 1H) 7.80 (m, 2H) 7.90 (m, 1H) 8.04 (m, 2H) 8.98 (m, 1H) | 442 | 9 |
| 111 | 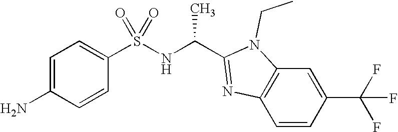 (R)-4-Amino-N-(1-(1-ethyl-6- (trifluoromethyl)-1H- benzo[d]imidazol-2- yl)ethyl)benzenesulfonamide | ¹H NMR 1.27-1.37 (m, 6H) 4.34- 4.45 (m, 2H) 4.71 (m, 1H) 5.92 (s, 2H) 6.56 (m, 2H) 7.44 (m, 2H) 7.48 (m, 1H) 7.76 (m, 1H) 7.87 (m, 1H) 7.99 (m, 1H) | 412 | Ex. 110 |
| 112 | 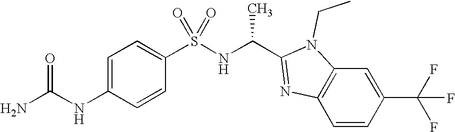 (R)-N-(1-(1-Ethyl-6- (trifluoromethyl)-1H- benzo[d]imidazol-2-yl)ethyl)- 4-ureidobenzenesulfonamide (Inactive at 3.50 μM) | ¹H NMR δ ppm 1.28-1.36 (m, 6H) 4.32-4.43 (m, 2H) 4.80 (m, 1H) 6.04 (s, 2H) 7.50 (m, 3H) 7.64 (m, 2H) 7.75 (m, 1H) 7.98 (m, 1H) 8.21 (m, 1H) 8.93 (m, 1H) | 455 | 9 |
| 113 | 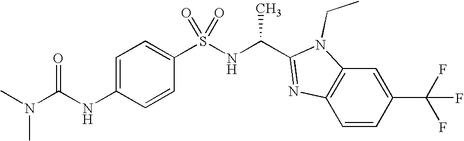 (R)-4-(3,3-Dimethylureido)- N-(1-(1-ethyl-6- (trifluoromethyl)-1H- benzo[d]imidazol-2- yl)ethyl)benzenesulfonamide (Inactive at 3.50 μM) | ¹H NMR 1.27-1.36 (m, 6H) 2.92 (s, 6H) 4.31-4.41 (m, 2H) 4.80 (m, 1H) 7.41-7.51 (m, 1H) 7.54-7.60 (m, 2H) 7.60-7.65 (m, 2H) 7.74 (m, 1H) 7.96 (m, 1H) 8.22 (m, 1H) 8.62 (m, 1H) | 483 | 9 |
| 114 | 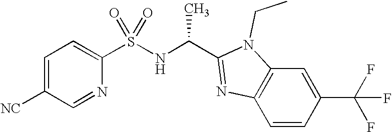 (R)-5-Cyano-N-(1-(1-ethyl-6- (trifluoromethyl)-1H- benzo[d]imidazol-2- yl)ethyl)pyridine-2- sulfonamide (72.04% inhibition at 3.50 μM) | ¹H NMR 1.26-1.36 (t, 3H) 1.50 (d, J = 6.82 Hz, 3H) 4.39 (m, 2H) 4.97- 5.07 (m, 1H) 7.46 (m, 1H) 7.61 (m, 1H) 7.88 (m, 1H) 7.96-8.03 (m, 1H) 8.29 (m, 1H) 8.83 (m, 1H) 9.06 (m, 1H) | 423 | 9 |
| 115 | 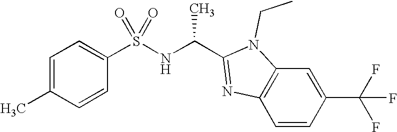 (R)-N-(1-(1-Ethyl-6- (trifluoromethyl)-1H- benzo[d]imidazol-2-yl)ethyl)- 4-methylbenzenesulfonamide (85.31% inhibition at 3.50 μM) | ¹H NMR 1.29 (t, J = 7.07 Hz, 3H) 1.35 (d, J = 6.82 Hz, 3H) 2.21 (s, 3H) 4.28-4.40 (m, 2H) 4.82 (m, 1H) 7.17 (m, 2H) 7.43-7.53 (m, 1H) 7.58 (m, 2H) 7.69 (m, 1H) 7.94 (m, 1H) 8.39 (m, 1H) | 411 | 9 |
| 116 | 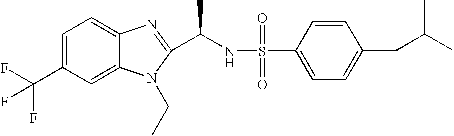 N-{(1R)-1-[1-Ethyl-6- (trifluoromethyl)-1H- benzimidazol-2-yl]ethyl}-4- isobutylbenzenesulfonamide (64.14% inhibition at 3.50 μM) | ¹H NMR (300 MHz, CDCl₃) δ ppm 7.63 (d, 1H) 7.53 (d, 2H) 7.46 (s, 1H) 7.44 (s, 1H) 6.89 (d, 2H) 6.07 (br, 1H) 4.79 (m, 1H) 4.13-4.26 (m, 1H) 3.99-4.11 (m, 1H) 2.19 (d, 2H) 1.57 (d, 3H) 1.48-1.60 (m, 1H) 1.33 (t, 3H) 0.66 (d, 3H) | 454 | 9 |
| 117 | 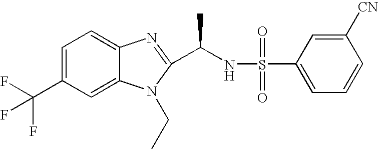 N-{(1R)-1-[1-Ethyl-6- (trifluoromethyl)-1H- benzimidazol-2-yl]ethyl}-3- isocyanobenzenesulfonamide (82.81% inhibition at 3.50 μM) | ¹H NMR (300 MHz, CDCl₃) δ ppm 7.95 (d, 1H) 7.83 (s, 1H) 7.62 (m, 2H) 7.54 (d, 1H) 7.43 (d, 1H) 7.35 (m, 1H) 6.90 (br, 1H) 4.91 (m, 1H) 4.27- 4.39 (m, 1H) 4.14-4.27 (m, 1H) 1.65 (d, 3H) 1.46 (t, 3H) | 422 | 9 |
| 118 | 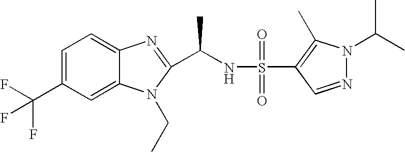 N-{(1R)-1-[1-Ethyl-6- (trifluoromethyl)-1H- benzimidazol-2-yl]ethyl}-1- isopropyl-5-methyl-1H- pyrazole-4-sulfonamide (77.99% inhibition at 3.50 μM) | ¹ H NMR (300 MHz, CDCl₃) δ ppm 7.70 (d,1H) 7.55 (s, 1H) 7.51 (d, 1H) 7.46 (s, 1H) 6.38 (br, 1H) 4.80 (m, 1H) 4.09-4.31 (m, 2H) 3.96-4.04 (m, 1H) 2.31 (s, 3H) 1.62 (d, 3H) 1.40 (t, 3H) 1.19 (d, 3H) 0.80 (d, 3H) | 443 | 9 |
| 119 | 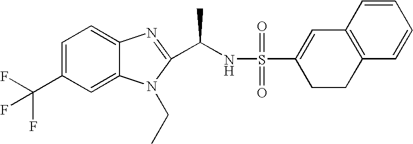 M-{(1R)-1-[1-Ethyl-6- (trifluoromethyl)-1H- benzimidazol-2-yl]ethyl}-3,4- diyhydronaphthalene-2- sulfonamide (71.72% inhibition at 3.30 μM) | ¹H NMR (300 MHz, CDCl₃) δ ppm 7.65 (d, 1H) 7.47 (s, 1H) 7.39 (d, 1H) 6.97-7.11 (m, 3H) 6.89 (d, 1H) 6.81 (d, 1H) 6.18 (br, 1H) 4.85 (m, 1H) 4.31-4.40 (m, 1H) 4.12-4.25 (m, 1H) 2.60-2.73 (m, 1H) 2.46-2.55 (m, 3H) 1.70 (d, 3H) 1.41 (t, 3H) | 449 | 9 |
| 120 | 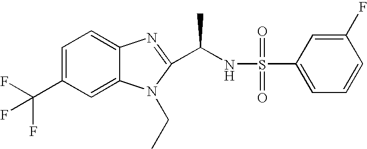 N-{(1R)-1-[1-Ethyl-6- (trifluoromethyl)-1H- benzimidazol-2-yl]ethyl}-3- fluorobenzenesulfonamide (90.89% inhibition at 3.50 μM) | ¹H NMR (300 MHz, CDCl₃) δ ppm 7.63 (d, 1H) 7.44-7.52 (m, 3H) 7.34 (m, 1H) 7.15 (m, 1H) 6.90 (m, 1H) 6.28 (br d, 1H) 4.83 (m, 1H) 4.06- 4.31 (m, 2H) 1.58 (d, 3H) 1.38 (t, 3H) | 415 | 9 |
| 121 | 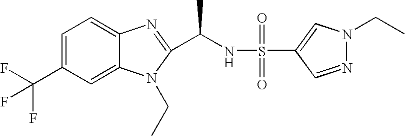 1-ethyl-N-{(1R)-1-[1-Ethyl-6- (trifluoromethyl)-1H- benzimidazol-2-yl]ethyl}-1H- pyrazole-4-sulfonamide (73.98% inhibition at 3.30 μM) | ¹H NMR (300 MHz, CDCl₃) δ ppm 7.86 (d, 1H) 7.71 (s, 1H) 7.61 (s, 1H) 7.63 (d, 1H) 7.59 (s,1H) 6.65 (br d, 1H) 4.97 (m, 1H) 4.22-4.40 (m, 2H) 3.84 (q, 2H) 1.74 (d, 3H) 1.50 (t, 3H) 1.21 (t, 3H) | 415 | 9 |
| 122 | 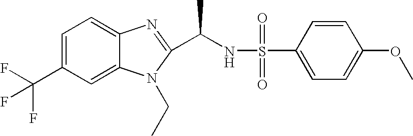 N-{(1R)-1-[1-Ethyl-6- (trifluoromethyl)-1H- benzimidazol-2-yl]ethyl}-4- methoxybenzenesulfonamide (91.36% inhibition at 3.50 μM) | ¹H NMR (300 MHz, CDCl₃) δ ppm 7.78 (d, 1H) 7.63-7.68 (m, 4H) 6.63 (d,2H) 4.94 (m, 1H) 4.39 (m, 1H) 4.25 (m, 1H) 3.62 (s, 3H) 1.77 (d, 3H) 1.52 (t, 3H) | 427 | 9 |
| 123 | 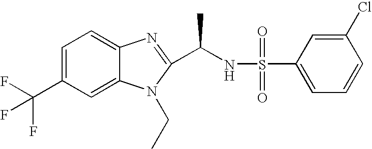 3-Chloro-N-{(1R)-1-[1-ethyl- 6-(trifluoromethyl)-1H- benzimidazol-2- yl]ethyl}benzenesulfonamide (96.66% inhibition at 3.50 μM) | ¹H NMR (300 MHz, CDCl₃) δ ppm 7.69 (d, 1H) 7.64 (m, 2H) 7.60 (s, 1H) 7.55 (d, 1H) 7.20 (m, 2H) 6.27 (br d, 1H) 4.91 (m, 1H) 4.14-4.40 (m, 2H) 1.68 (d, 3H) 1.48 (t, 3H) | 432 | 9 |
| 124 | 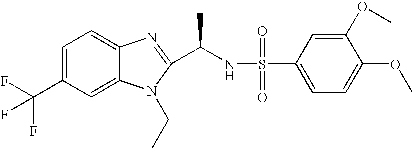 N-{(1R)-1-[1-Ethyl-6- (trifluoromethyl)-1H-benz- imidazol-2-yl]ethyl}-3,4-di- methoxybenzenesulfonamide (79.37% inhibition at 3.50 μM) | ¹H NMR (300 MHz, CDCl₃) δ ppm 7.72 (d, 1H) 7.60 (s, 1H) 7.58 (d, 1H) 7.29 (s, 1H) 7.19 (d, 1H) 6.45 (d, 1H) 6.25 (br, 1H) 4.89 (m, 1H) 4.32 (m, 1H) 4.18 (m, 1H) 3.89 (s, 3H) 3.69 (m, 3H) 1.70 (d, 3H) 1.47 (t, 3H) | 457 | 9 |
| 125 | 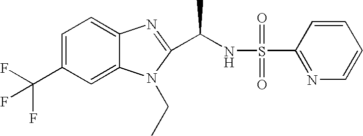 N-{(1R)-1-[1-Ethyl-6- (trifluoromethyl)-1H- benzimidazol-2- yl]ethyl}pyridine-2- sulfonamide (14.90% inhibition at 3.50 μM) | ¹H NMR (300 MHz, CDCl₃) δ ppm 8.32 (d, 1H) 7.90 (d, 1H) 7.65-7.75 (m, 2H) 7.44 (d, 1H) 7.22 (m, 1H) 7.13 (d, 1H) 5.20 (m, 1H) 4.42 (m, 1H) 4.23 (m, 1H) 1.65 (d, 3H) 1.49 (t, 3H) | 398 | 9 |
| 126 | 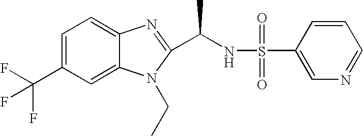 (R)-N-(1-(1-Ethyl-6- (trifluoromethyl)-1H- benzo[d]imidazol-2- yl)ethyl)pyridine-3- sulfonamide (92.75% inhibition at 3.50 μM) | ¹H NMR (300 MHz, CDCl₃) δ ppm 1.40 (t, 3H) 1.58 (d, 3H) 4.06-4.36 (m, 2H) 4.87-4.98 (m, 1H) 6.96- 7.05 (m, 1H) 7.14-7.21 (m, 1H) 7.44- 7.51 (m, 2H) 7.68-7.74 (m, 1H) 7.96-8.02 (m, 1H) 8.51-8.55 (m, 1H) 8.98-9.02 (m, 1H) | 398 | 9 |
| 127 | 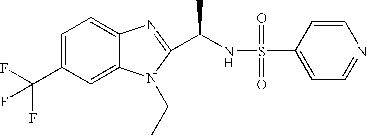 (R)-N-(1-(1-Ethyl-6- (trifluoromethyl)-1H- benzo[d]imidazol-2- yl)ethyl)pyridine-4- sulfonamide (86.42% inhibition at 3.50 μM) | ¹H NMR (300 MHz, CDCl₃) δ ppm 1.41 (t, 3H) 1.57 (d, 3H) 1.86-1.95 (m, 1H) 4.07-4.36 (m, 2H) 4.93 (d, 1H) 7.46-7.54 (m, 2H) 7.59 (dd, 2H) 7.68-7.74 (m, 1H) 8.63 (dd, 2H) | 398 | 9 |
| 128 | 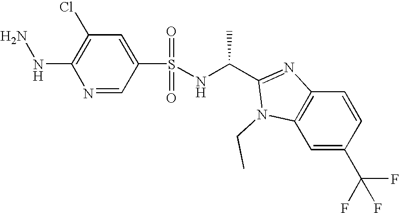 R-5-Chloro-N-(1-(1-ethyl-6- (trifluoromethyl)-1H- benzo[d]imidazol-2-yl)ethyl)- 6-hydrazinylpyridine-3- sulfonamide (94.50% inhibition at 3.50 μM) | ¹H NMR (300 MHz, MeOH-d₄) δ ppm 1.33 (t, 3H) 1.46-1.52 (m, 4H) 3.19- 3.24 (m, 4H) 4.33 (q, 1H) 4.85 (q, 1H) 7.36-7.56 (m, 3H) 7.72-7.76 (m, 1H) 8.09 (d, 1H) | 462 | Ex 48 |
| 129 | 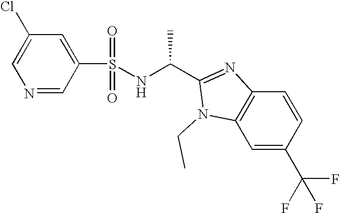 R-5-Chloro-N-(1-(1-ethyl-6- (trifluoromethyl)-1H- benzo[d]imidazol-2- yl)ethyl)pyridine-3- sulfonamide (90.28% inhibition at 3.50 μM) | ¹H NMR (300 MHz, CDCl₃) δ ppm 1.39 (t, 3H) 1.58 (d, 3H) 2.50-2.70 (m, 1H) 4.04-4.30 (m, 2H) 4.79- 4.92 (m, 1H) 6.38 (d, 1H) 7.41-7.51 (m, 2H) 7.58-7.64 (m, 1H) 7.79-7.83 (m, 1H) 8.27-8.34 (m, 3H) 8.68-8.75 (m, 1H) | 434 | Ex 128 |
| 130 | 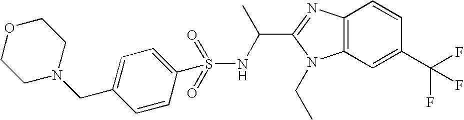 N-(1-(1-Ethyl-6- (trifluoromethyl)-1H- benzo[d]imidazol-2-yl)ethyl)- 4-(morpholinomethyl)- benzenesulfonamide (10.79% inhibition at 3.50 μM) | ¹H NMR (300 MHz, CDCl₃) δ ppm 1.38 (t, 3H) 1.58 (d, 3H) 2.24-2.32 (m, 4H) 3.27-3.31 (m, 2H) 3.61- 3.68 (m, 4H) 4.03-4.34 (m, 2H) 4.79-4.92 (m, 1H) 6.20 (d, 1H) 7.20 (d, 2H) 7.43-7.54 (m, 2H) 7.60- 7.71 (m, 3H) | 496 | 9 |
| 131 | 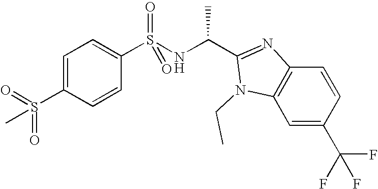 (R)-N-(1-(1-Ethyl-6- (trifluoromethyl)-1H- benzo[d]imidazol-2-yl)ethyl)- 4-(methylsulfonyl)- benzenesulfonamide (94.95% inhibition at 3.50 μM) | ¹H NMR (300 MHz, CDCl₃) δ ppm 1.36 (t, 3H) 1.54 (d, 2H) 2.75-2.80 (m, 3H) 4.03-4.28 (m, 2H) 4.79- 4.91 (m, 1H) 6.58 (d, 1H) 7.40-7.50 (m, 2H) 7.60-7.66 (m, 1H) 7.76 (dd, 2H) 7.86 (dd, 2H) | 475 | 9 |
| 132 | 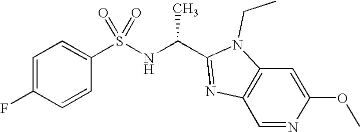 (R)-N-(1-(1-Ethyl-6- methoxy-1H-imidazo[4,5- c]pyridin-2-yl)ethyl)-4- fluorobenzenesulfonamide (86.15% inhibition at 3..50 μM) | ¹H NMR 1.26 (t, J = 7.07 Hz, 3H) 1.31 (d, J = 6.57 Hz, 3H) 3.86 (s, 3H) 4.19 (m, 2H) 4.81 (m, 1H) 6.87 (ms, 1H) 7.30 (m, 2H) 7.73-7.82 (m, 2H) 8.41 (m, 1H) 8.51 (m, 1H). | 378 | 53 |
| 133 | 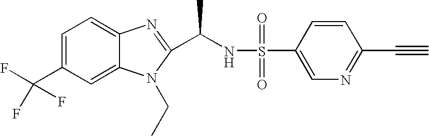 (R)-N-(1-(1-Ethyl-6- (trifluoromethyl)-1H- benzo[d]imidazol-2-yl)ethyl)- 6-ethynylpyridine-3- sulfonamide (86.39% inhibition at 3.50 μM) | ¹H NMR (300 MHz, MeOH-d₄) δ 8.75 (s, 1H) 7.96 (d, 1H) 7.87 (s, 1H) 7.65 (d, 1H) 7.55 (d, 1H) 7.40 (d, 1H), 5.0 (m, 1H) 4.44 (m, 2H) 3.96 (s, 1H) 1.59 (d,3H) 1.40 (t, 3H) | 422 | Ex. 35 |
| 134 | 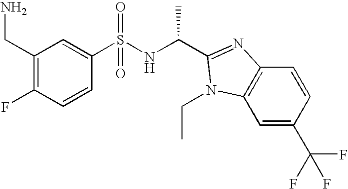 3-(Aminomethyl)-N-{(1R)-1- [1-ethyl-6-(trifluoromethyl)- 1H-benzimidazol-2-yl]ethyl}- 4-fluorobenzenesulfonamide (95% inhibition at 3.50 μM) | ¹H NMR (MeOH-d₄) δ 7.95 (m, 1H) 7.81 (m, 1H) 7.74 (m, 1H) 7.65 (d, 1H) 7.55 (d, 1H) 7.13 (t, 1H) 4.95 (m, 1H) 4.44 (m, 2H) 3.99 (m, 2H) 1.48 (d, 3H) 1.40 (s, 3H). | 444 | Ex. 27 |
| 135 | 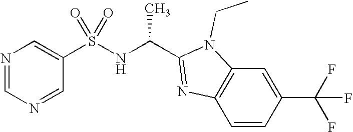 (R)-N-(1-(1-Ethyl-6- (trifluoromethyl)-1H- benzo[d]imidazol-2- yl)ethyl)pyrimidine-5- sulfonamide | ¹ H NMR 1.30 (t, J = 7.20 Hz, 3H) 1.45 (d, J = 6.82 Hz, 3H) 4.35-4.45 (m, 2H) 5.04 (m, 1H) 7.46 (m, 1H) 7.59 (m, 1H) 7.99 (s, 1H) 8.94 (s, 2H) 9.10 (m, 1H). | 399 | 9 |
| 136 | 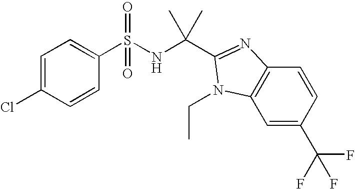 4-Chloro-N-{1-[1-ethyl-6- (trifluoromethyl)-1H- benzimidazol-2-yl]-1- methylethyl}benzenesulfon- amide (82.9% inhibition at 3.50 μM) | ¹H NMR (300 MHz, CDCl₃) δ 7.85 (d, 1H) 7.67 (d, 2H) 7.54 (d, 1H) 7.53 (s, 1H) 7.29 (d, 2H) 5.97 (br, 1H) 4.35 (q, 2H) 1.82 (s, 6H) 1.50 (t, 3H) | 446 | 54 |
| 137 | 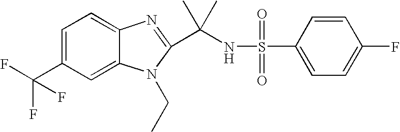 N-[1-(1-Ethyl-6- trifluoromethyl-1H- benzoimidazol-2-yl)-1- methyl-ethyl]-4-fluoro- benzenesulfonamide (88.80% inhibition at 3.50 μM) | ¹H NMR (300 MHz, DMSO-d₆) δ: 8.65 (brs, 1H), 7.84 (s, 1H), 7.78 (d, J = 8.81 Hz, 1H), 7.69-7.74 (m, 2H), 7.49 (d, J = 8.53 Hz, 1H), 7.23-7.17 (m, 2H), 4.46 (q, J = 6.88 Hz, 2H), 1.61 (s, 6H), 1.39 (t, J = 7.15 Hz, 3H) | 429 | 54 |
| 138 | 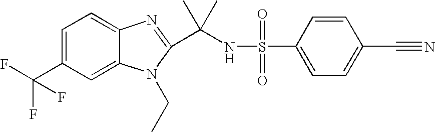 4-Cyano-N-[1-(1-ethyl-6- trifluoromethyl-1H- benzoimidazol-2-yl)-1- methyl-ethyl]- benzenesulfonamide (78.44% inhibition at 3.50 μM) | ¹H NMR (300 MHz, DMSO-d₆) δ: 8.95 (brs, 1H), 7.81-7.70 (m, 6H), 7.49 (d, J = 8.52 Hz, 1H), 4.41 (q, J = 6.88 Hz, 2H), 1.64 (s, 6H), 1.37 (t, J = 7.15 Hz, 3H) | 436 | 54 |
| 139 | 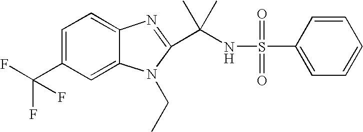 N-{(1R)-1-[1-Ethyl-6- (trifluoromethyl)-1H- benzimidazol-2-yl]-1-methyl- ethyl}benzenesulfonamide (88.32% inhibition at 3.50 μM) | ¹ H NMR (300 MHz, MeOH-d₄) δ: 7.36-7.82 (m, 8H), 4.45 (q, 2H), 1.76 (s, 6H), 1.55 (t, 3H) | 411 | 54 |
| 140 | 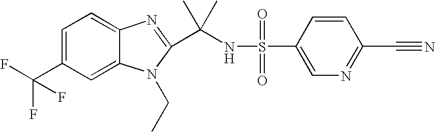 6-Cyano-N-(2-(1-ethyl-6- (trifluoromethyl)-1H-0 benzo[d]imidazol-2- yl)propan-2-yl)pyridine-3- sulfonamide (82.93% inhibition at 3.50 μM) | ¹H NMR (300 MHz, MeOH-d₄) δ 8.75 (s, 1H) 8.4 (d, 1H) 8.01 (d, 1H) 7.76 (d, 1H) 7.7 (s, 1H) 7.55 (d, 1H), 4.55 (m, 1H) 4.44 (m, 2H) 1.85 (s, 6H) 1.8 (s, 6H) 1.40 (t, 3H) | 437 | 54 |
| 141 | 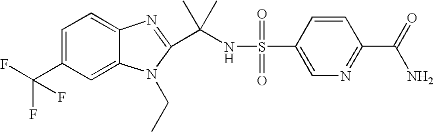 5-(N-(2-(1-Ethyl-6- (trifluoromethyl)-1H- benzo[d]imidazol-2- yl)propan-2- yl)sulfamoyl)picolinamide (82.56% inhibition at 3.50 μM) | ¹ H NMR (300 MHz, MsOH-d₄) δ 8.75 (s, 1H) 8.4 (d, 1H) 8.01 (d, 1H) 7.76 (d, 1H) 7.7 (s, 1H) 7.55 (d, 1H), 4.55 (m, 1H) 4.44 (m, 2H) 1.85 (s, 6H) 1. (d, 3H) 1.40 (t, 3H) | 455 | Ex. 140 |
| 142 | 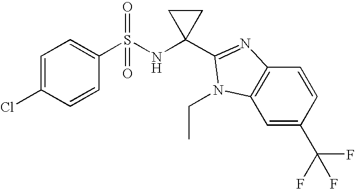 4-Chloro-N-{1-[1-ethyl-6- (trifluoromethyl)-1H- benzimidazol-2- yl]cyclopropyl}benzenesulfon amide (80.52% inhibition at 3.50 μM) | ¹ H NMR (300 MHz, MeOH-d₄) δ 7.95 (m, 1H) 7.81 (m, 1H) 7.74 (m, 1H) 7.65 (d, 1H) 7.55 (d, 1H) 7.13 (t, 1H) 4.95 (m, 1H) 4.44 (m, 2H) 3.99 (mn, 2H) 1.48 (d, 3H) 1.40 (t, 3H). | 444 | 55 |
| 143 | 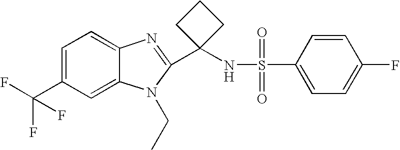 N-[1-(1-Ethyl-6- trifluoromethyl-1H- benzoimidazol-2-yl)- cyclobutyl]-4-fluoro- benzenesulfonamide (84.67% inhibition at 3.50 μM) | ¹ H NMR (300 MHz, DMSO-d₆): 8.87 (brs, 1H), 7.82 (d, J = 8.53 Hz, 1H), 7.76 (s, 1H), 7.53-7.49 (m, 3H), 7.09- 7.03 (m, 2H), 4.16 (q, J = 7.15 Hz, 2H), 2.76-2.68 (m, 2H), 2.42-2.33 (m, 2H), 1.93-1.82 (m,1 H), 1.73-1.61 (m, 1H), 1.31 (t, J = 7.15 Hz, 3H | 441 | 56 |
| 144 | 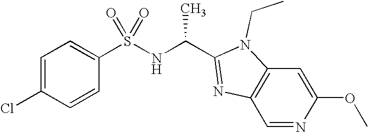 (R)-4-Chloro-N-(1-(1-ethyl-6- methoxy-1H-imidazo[4,5- c]pyridin-2- yl)ethyl)benzenesulfonamide (90.66% inhibition at 3.50 μM) | ¹ H NMR (MeOH-d₄) 1.37 (t, J = 7.20 Hz, 3H) 1.49 (d, J = 6.82 Hz, 3H) 3.94 (s, 3H) 4.26 (m, 2H) 4.85-4.92 (m, 1H) 6.81 (m, 1H) 7.32-7.41 (m, 2H) 7.66-7.76 (m, 2H) 8.33 (m, J = 10.1 Hz, 1H). | 394 | — |
| 145 | 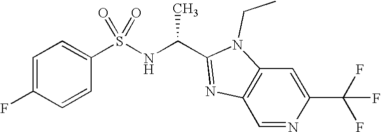 (R)-N-(1-(1-Ethyl-6- (trifluoromethyl)-1H- imidazo[4,5-c]pyridin-2- yl)ethyl)-2- fluorobenzenesulfonamide (92.46% inhibition at 3.50 μM) | ¹H NMR 1.32 (t, J = 7.20 Hz, 3H) 1.39 (d, J = 6.82 Hz, 3H) 4.35-4.47 (m, 2H) 4.92 (m, 1H) 7.23 (m, 2H) 7.76 (m, 2H) 8.22 (m, 1H) 8.64 (m, 1H) 8.93 (m, 1H) | 416 | 57 |
| 146 | 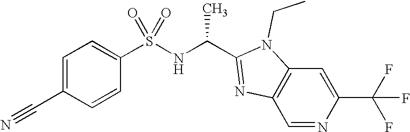 (R)-4-Cyano-N-(1-(1-ethyl-6- (trifluoromethyl)-1H- imidazo[4,5-c]pyridin-2- yl)ethyl)benzenesulfonamide (91.85% inhibition at 3.50 μM) | ¹H NMR 1.31 (t, J = 7.07 Hz, 3H) 1.42 (d, J = 6.82 Hz, 3H) 4.33-4.45 (m, 2H) 4.98 (m, 1H) 7.75-7.85 (m, 4H) 8.20 (m, 1H) 8.90 (m, 1H) 8.97 (m, 1H) | 423 | 57 |
| 147 | 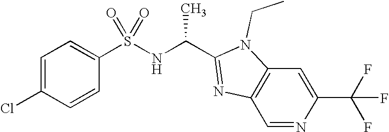 (R)-4-Chloro-N-(1-(1-ethyl-6- (trifluoromethyl)-1H- imidazo[4,5-c]pyridin-2- yl)ethyl)benzenesulfonamide (95.91% inhibition at 3.50 μM) | ¹H NMR 1.31 (t, J = 7.20 Hz, 3H) 1.41 (d, J = 6.57 Hz, 3H) 4.33-4.45 (m, 2H) 4.91 (m, 1H) 7.35-7.45 (m, 2H) 7.66 (m, 2H) 8.20 (m, 1H) 8.72 (m, 1H) 8.92 (m, 1H). | 432 | 57 |
| 148 | 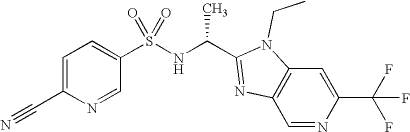 (R)-6-Cyano-N-(1-(1-ethyl-6- (trifluoromethyl)-1H- imidazo[4,5-c]pyridin-2- yl)ethyl)pyridine-3- sulfonamide (93.03% inhibition at 3.50 μM) | ¹H NMR 1.32 (t, J = 7.07 Hz, 3H) 1.46 (d, J = 6.82 Hz, 3H) 4.36-4.46 (m, 2H) 5.05 (m, 1H) 8.00 (m, 1H) 8.18- 8.24 (m, 2H) 8.86 (m, 1H) 8.87 (m, 1H) 9.25 (m, 1H). | 424 | 57 |
| 149 | 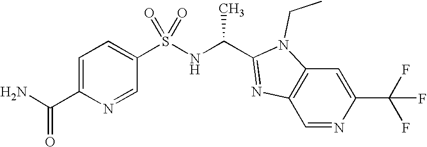 (R)-5-(N-(1-(1-Ethyl-6- (trifluoromethyl)-1H- imidazo[4,5-c]pyridin-2- yl)ethyl)sulfamoyl)picolinamide (93.31% inhibition at 3.50 μM) | ¹ H NMR 1.32 (t, J = 7.07 Hz, 3H) 1.41 (d, J = 6.57 Hz, 3H) 4.37-4.48 (m, 2H) 5.03 (m, 1H) 7.79 (broad s, 1H) 8.01 (m, 1H) 8.10 (broad s, 1H) 8.16- 8.26 (m, 2H) 8.81-8.90 (m, 2H) 9.05 (m, 1H). | 442 | Ex. 148 |
| 150 | 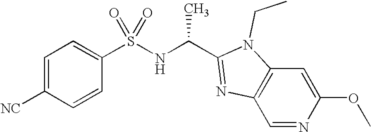 (R)-4-Cyano-N-(1-(1-ethyl-6- methoxy-1H-imidazo[4,5- c]pyridin-2- yl)ethyl)benzenesulfonamide (91.59% inhibition at 3.50 μM) | ¹H NMR 1.25 (t, J = 7.20 Hz, 3H) 1.37 (d, J = 6.82 Hz, 3H) 3.87 (s, 3H) 4.17 (m, 2H) 4.81-4.90 (m, 1H) 6.84 (s, 1H) 7.79-7.87 (m, 4H) 8.36 (s, 1H) 8.83 (m, 1H). | 385 | 53 |
| 151 | 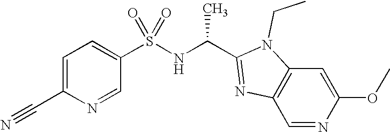 (R)-6-Cyano-N-(1-(1-ethyl-6- methoxy-1H-imidazo[4,5- c]pyridin-2- yl)ethyl)pyridine-3- sulfonamide (91.69% inhibition at 3.50 μM) | ¹ H NMR 1.25 (t, J = 7.20 Hz, 3H) 1.43 (d, J = 6.82 Hz, 3H) 3.87 (s, 3H) 4.18 (m, 2H) 4.92 (m, 1H) 6.85 (s, 1H) 7.98 (m, 1H) 8.18 (m, 1H) 8.30 (m, 1H) 8.86 (m, 1H) 9.11 (m, 1H). | 386 | 53 |
| 152 | 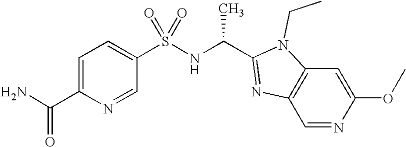 (R)-6-Cyano-N-(1-(1-ethyl-6- methoxy-1H-imidazo[4,5- c]pyridin-2- yl)ethyl)pyridine-3- sulfonamide (91.77% inhibition at 3.50 μM) | ¹ H NMR 1.27 (t, J = 7.07 Hz, 3H) 1.37 (d, J = 6.82 Hz, 3H) 3.86 (s, 3H) 4.16- 4.26 (m, 2H) 4.92 (m, 1H) 6.86 (s, 1H) 7.81 (m, 1) 8.02 (m, 1H) 8.11 (m, 1H) 8.21 (m, 1H) 8.34 (m, 1H) 8.86 (m, 1H) 8.92 (m, 1H). | 404 | Ex. 151 |
| 153 | 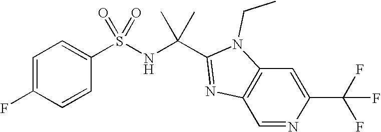 N-(2-(1-Ethyl-6- (trifluoromethyl)-1H- imidazo[4,5-c]pyridin-2- yl)propan-2-yl)-4- fluorobenzenesulfonamide (82.02% inhibition at 3.50 μM) | ¹H NMR 1.40 (t, J = 7.07 Hz, 3H), 1.61 (s, 6H) 4.51 (q, J = 7.07 Hz, 2H) 7.23 (m, 2H) 7.65-7.75 (m, 2H) 8.11 (m, 1H) 8.71 (m, 1H) 9.00 (m, 1H). | 430 | 58 |
| 154 | 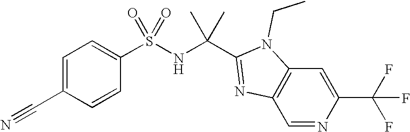 4-Cyano-N-(2-(1-ethyl-6- (trifluoromethyl)-1H- imidazo[4,5-c]pyridin-2- yl)propan-2- yl)benzenesulfonamide (77.31% inhibition at 3.50 μM) | ¹H NMR 1.39 (t, J = 7.07 Hz, 3H) 1.63 (s, 6H) 4.47 (d, J = 7.07 Hz, 2H) 7.73- 7.80 (m, 2H) 7.81-7.88 (m, 2H) 8.11 (m, 1H) 9.01 (m, 2H). | 437 | 58 |
| 155 | 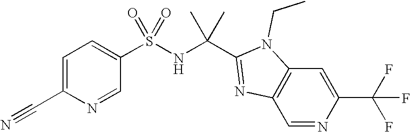 6-Cyano-N-(2-(1-ethyl-6- (trifluoromethyl)-1H- imidazo[4,5-c]pyridin-2- yl)propan-2-yl)pyridine-3- sulfonamide (78.91% inhibition at 3.50 μM) | ¹H NMR 1.40 (t, J = 7.20 Hz, 3H) 1.66 (s, 6H) 4.44-4.54 (d, J = 7.20 Hz, 2H) 8.06 (m, 1H) 8.15 (m, 1H) 8.23 (m, 1H) 8.87 (m, 1H) 9.02 (m, 1H) 9.26 (m, 1H). | 438 | 58 |
| 156 | 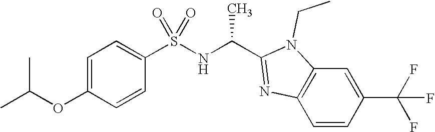 R)-N-(1-(1-Ethyl-6- (tyrifluoromethyl)-1H- benzo[d]imidazol-2-yl)ethyl)- 4-isopropoxybenzene- sulfonamide (80.66% inhibition at 3.50 μM) | ¹H NMR 1.18 (m, 6H) 1.25-1.32 (m, 3) 1.37 (d, J = 6.82 Hz, 3H) 4.28- 4.40 (m, 2H) 4.52 (m, 1H) 4.80 (m, 1H) 6.76-6.86 (m, 2H) 7.45 (m, 1H) 7.53-7.60 (m, 2H) 7.66-7.76 (m, 1H) 7.93 (m, 1H) 8.30 (m, 1H). | 455 | 9 |
| 157 | 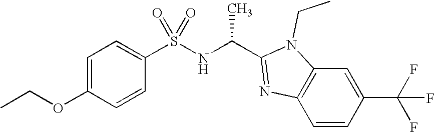 (R)-4-Ethoxy-N-(1-(1-ethyl- 6-(trifluoromethyl)-1H- benzo[d]imidazol-2- yl)ethyl)benzenesulfonamide (88.37% inhibition at 3.50 μM) | ¹H NMR 1.24-1.32 (m, 6H) 1.36 (d, J = 6.82 Hz, 3H) 3.85-3.96 (m, 2H) 4.27-4.39 (m, 2H) 4.79 (m, 1H) 6.80-6.89 (m, 2H) 7.43-7.49 (m, 1H) 7.59 (m, 2H) 7.68 (m, 1H) 7.93 (m, 1H) 8.30 (m, 1H). | 441 | 9 |
| 158 | 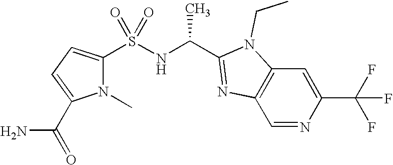 (R)-5-(N-(1-(1-Ethyl-6- (trifluoromethyl)-1H- benzo[d]imidazol-2- yl)ethyl)sulfamoyl)-1-methyl- 1H-pyrrole-2-carboxamide (93.56% inhibition at 3.50 μM) | ¹ H NMR 1.29 (t, J = 7.20 Hz, 3H) 1.45 (d, J = 6.82 Hz, 3H) 3.67 (s, 3H) 4.32- 4.43 (m, 2H) 4.72-4.83 (m, 1H) 7.06 (m, 1H) 7.12 (broad s, 1H) 7.29 (m, 1H) 7.48 (m, 1H) 7.68 (broad s, 1H) 7.74 (m, 1H) 7.98 (m, 1H) 8.03 (m, 1H). | 443 | Ex. 40 |
| 159 | 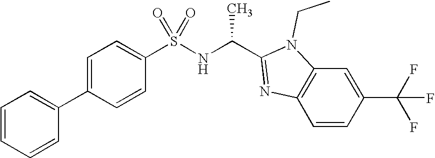 (R)-N-(1-(1-Ethyl-6- (trifluoromethyl)-1H- benzo[d]imidazol-2- yl)ethyl)biphenyl-4- sulfonamide (19.75% inhibition at 3.50 μM) | ¹H NMR 1.29 (t, J = 7.20 Hz, 3H) 1.42 (d, J = 6.82 Hz, 3H) 4.30-4.41 (m, 2H) 4.84-4.94 (m, 1H) 7.37-7.46 (m, 5H) 7.48-7.51 (m, 2H) 7.59 (m, 2H) 7.65 (m, 1H) 7.73 (m, 2H) 7.92 (s, 1H) 8.58 (m, 1H). | 473 | 9 |
| 160 | 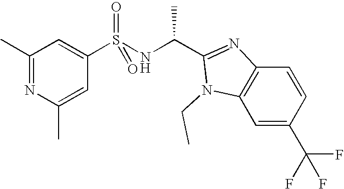 (R)-N-(1-(1-Ethyl-6- (trifluoromethyl)-1H- benzo[d]imidazol-2-yl)ethyl)- 2,6-dimethylpyridine-4- sulfonamide (90.33% inhibition at 3.50 μM) | ¹H NMR (300 MHz, CDCl₃) δ ppm 1.41 (t,3H) 1.61 (d, 3H) 1.85-1.90 (m, 1H) 2.31-2.36 (m, 6H) 4.05- 4.35 (m, 2H) 4.82-4.95 (m, 1H) 6.30-6.37 (m, 1H) 7.13-7.17 (m, 2H) 7.48-7.56 (m, 2H) 7.66-7.71 (m, 1H) | 427 | 9 |
| 161 | 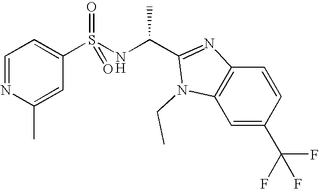 (R)-N-(1-(1-Ethyl-6- (trifluoromethyl)-1H- benzo[d]imidazol-2-yl)ethyl)- 2-methylpyridine-4- sulfonamide (91.37% inhibition at 3.50 μM) | ¹H NMR (300 MHz, CDCl₃) δ ppm 1.36 (t, 3H) 1.57 (d, 3H) 2.27-2.31 (m, 3H) 2.72-2.94 (m, 1H) 4.02- 4.29 (m, 2H) 4.78-4.90 (m, 1H) 6.32 (d, 1H) 7.23-7.27 (m, 1H) 7.29- 7.34 (m, 1H) 7.44-7.53 (m, 2H) 7.60-7.66 (m, 1H) 8.39-8.44 (m, 1H) | 413 | 9 |
| 162 | 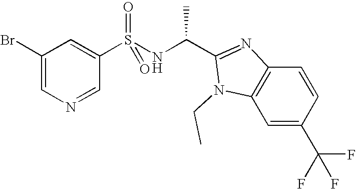 5-Bromo-N-(1-(1-ethyl-6- (trifluoromethyl)-1H- benzo[d]imidazol-2- yl)ethyl)pyridine-3- sulfonamide (90.32% inhibition at 3.50 μM) | ¹H NMR (300 MHz, CDCl₃) δ ppm 1.45 (t, 3H) 1.62-1.68 (m, 3H) 4.10- 4.34 (m, 2H) 4.85-4.94 (m, 1H) 5.98-6.18 (m, 1H) 7.47-7.57 (m, 2H) 7.61-7.67 (m, 1H) 7.96-7.99 (m, 1H) 8.42-8.44 (m, 1H) 8.79-8.81 (m, 1H) | 477 | 9 |
| 163 | 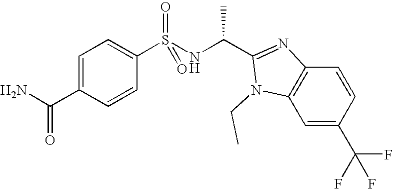 (R)-4-(N-(1-(1-Ethyl-6- (trifluoromethyl)-1H- benzo[d]imidazol-2-yl)ethyl)- sulfamoyl)benzamide | ¹ H NMR (300 MHz, MeOH-d₄) δ ppm 1.30 (t, 3H) 1.44 (d, 3H) 4.29 (q, 2H) 4.88 (q, 1H) 7.33-7.39 (m, 1H) 7.48-7.54 (m, 1H) 7.61-7.71 (m, 3H) 7.72-7.79 (m, 2H) | 442 | Ex. 26 |
| 164 | 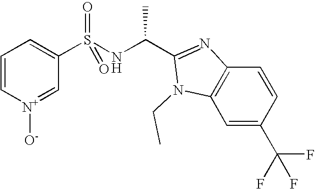 (R)-3-(N-(1-(1-Ethyl-6- (trifluoromethyl)-1H- benzo[d]imidazol-2- yl)ethyl)sulfamoyl)pyridine 1-oxide (19.47% inhibition at 3.50 μM) | ¹ H NMR (300 MHz, MeOH-d₄) δ ppm 1.35 (t, 3H) 1.50 (d, 3H) 4.35 (q, 2H) 4.98 (q, 1H) 7.17-7.23 (m, 1H) 7.38-7.43 (m, 1H) 7.51-7.59 (m, 3H) 7.76-7.78 (m, 1H) 7.99-8.03 (m, 1H) 8.41-8.44 (m, 1H) | 415 | Ex. 126 |
| 165 | 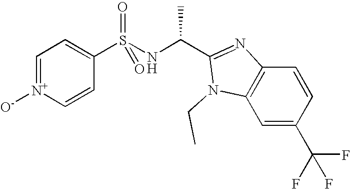 (R)-4-(N-(1-(1-Ethyl-6- (trifluoromethyl)-1H- benzo[d]imidazol-2- yl)ethyl)sulfamoyl)pyridine 1-oxide (75.6% inhibition at 3.50 μM) | ¹H NMR (300 MHz, MeOH-d₄) δ ppm 1.47 (t, 3H) 1.61 (d, 3H) 4.48 (q, 2H) 5.08 (q, 1H) 7.52-7.57 (m, 1H) 7.62-7.70 (m, 2H) 7.89-7.92 (m, 1H) 8.10-8.15 (m, 2H) | 416 | Ex. 127 |
| 166 | 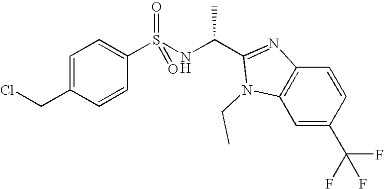 (R)-4-(Chloromethyl)-N-(1- (1-ethyl-6-(trifluoromethyl)- 1H-benzo[d]imidazol-2- yl)ethyl)benzenesulfonamide (86.04% inhibition at 3.50 μM) | ¹ H NMR (300 MHz, CDCl₃) δ ppm 1.32 (t, 3H) 1.54 (d, 3H) 2.25-2.44 (m, 1H) 3.95-4.24 (m, 2H) 4.28 (d, 2H) 4.71-4.83 (m, 1H) 6.18 (d, 1H) 7.14 (d, 2H) 7.40-7.47 (m, 2H) 7.61 (d, 2H) | 446 | 9 |
| 167 | 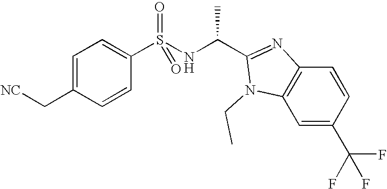 (R)-4-(Cyanomethyl)-N-(1- (1-ethyl-6-(trifluoromethyl)- 1H-benzo[d]imidazol-2- yl)ethyl)benzenesulfonamide (86.04% inhibition at 3.50 μM) | ¹ H NMR (300 MHz, CDCl₃) δ ppm 1.42 (t, 3H) 1.61 (d, 3H) 2.06-2.16 (m, 1H) 3.55-3.59 (m, 2H) 4.05- 4.35 (m, 2H) 4.81-4.94 (m, 1H) 6.35 (d, 1H) 7.20 (d, 2H) 7.49-7.56 (m, 2H) 7.68-7.75 (m, 2H) | 437 | Ex. 166 |
| 168 | 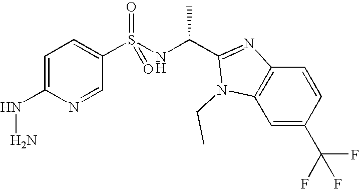 (R)-N-(1-(1-Ethyl-6- (trifluoromethyl)-1H- benzo[d]imidazol-2-yl)ethyl)- 6-hydrazinylpyridine-3- sulfonamide (77.06% inhibition at 3.50 μM) | ¹H NMR (300 MHz, MeOH-d₄) δ ppm 1.45 (t, 3H) 1.57 (d, 3H) 4.45 (q, 2H) 4.91-4.97 (m, 1H) 6.56 (d, 1H) 7.50-7.56 (m, 1H) 7.61-7.72 (m, 3H) 7.85-7.88 (m, 1H) 8.25-8.28 (m, 1H) | 429 | Ex. 35 |
| 169 | 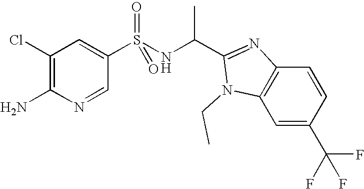 6-Amino-5-chloro-N-(1-(1- ethyl-6-(trifluoromthyl)-1H- benzo[d]imidazol-2- yl)ethyl)pyridine-3- sulfonamide (87.06% inhibition at 3.50 μM) | ¹⁻H NMR (300 MHz, MeOD-d₄) δ ppm 1.44 (t, 3H) 1.60 (d, 3H) 4.43 (q, 2H) 4.91-4.99 (m, 1H) 7.49-7.54 (m, 1H) 7.62 (d, 1H) 7.67-7.68 (m, 1H) 7.69-7.71 (m, 1H) 7.84-7.86 (m, 1H) 8.15 (d, 1H) | 448 | Ex. 48 |
| 170 | 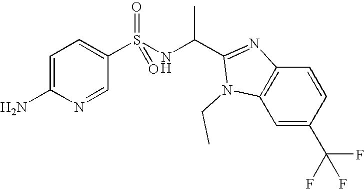 6-Amino-N-(1-(1-ethyl-6- (trifluoromethyl)-1H- benzo[d]imidazol-2- yl)ethyl)pyridine-3- sulfonamide (83.12% inhibition at 3.50 μM) | ¹H NMR (300 MHz, MeOH-d₄) δ ppm 1.44 (t, 3H) 1.57 (d, 3H) 4.44 (q, 2H) 4.92-4.97 (m, 1H) 6.33-6.38 (m, 1H) 7.50-7.55 (m, 1H) 7.59-7.64 (m, 1H) 7.69-7.74 (m, 1H) 7.85- 7.87 (m, 1H) 8.21-8.23 (m, 1H) | 415 | Ex. 35 |
| 171 | 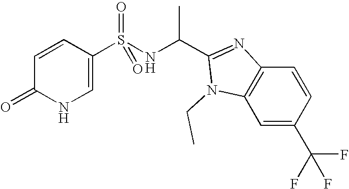 N-(1-(1-Ethyl-6- (trifluoromethyl)-1H- benzo[d]imidazol-2-yl)ethyl)- 6-oxo-1,6-dihydropyridine-3- sulfonamide (73.33% inhibition at 3.50 μM) | ¹H NMR (300 MHz, MeOH-d₄) δ ppm 1.47 (t, 3H) 1.62 (d, 3H) 4.43-4.52 (m, 2H) 4.93-5.01 (m, 1H) 6.34- 6.40 (m, 1H) 7.52-7.57 (m, 1H) 7.64-7.66 (m, 1H) 7.67-7.69 (m, 1H) 7.70-7.71 (m, 1H) 7.72-7.76 (m, 2H) | 415 | Ex. 35 |
| 172 | 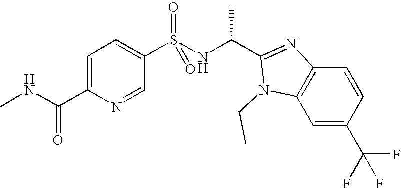 (R)-5-[1-(1-Ethyl-6- trifluoromethyl-1H- benzoimidazol-2-yl)- ethylsulfamoyl]-pyridine-2- carboxylic acid methylamide (69.11% inhibition at 3.50 μM) | ¹H NMR (300 MHz, DMSO-d₆) δ ppm 8.99 (br s., 2H), 8.78 (d, J = 2.2 Hz, 1H), 8.65-8.71 (m, 1H), 8.18 (dd, J = 8.3, 2.2 Hz, 1H), 7.89-7.97 (m, 2H), 7.61 (d, J = 8.5 Hz, 1H), 7.41 (dd, J = 8.5, 1.4 Hz, 1H), 4.98 (q, J = 6.7 Hz, 1H), 4.33-4.42 (m, 2H), 2.78 (d,J = 4.7 Hz, 3H), 1.43 (d, J = 6.9 Hz, 3H), 1.31 (t, J = 7.2 Hz, 3H). | 455 | 9 |
| 173 | 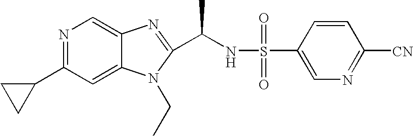 6-Cyano-N-[(1R)-1-(6- cyclopropyl-1-ethyl-1H- imidazo[4,5-c]pyridin-2- yl)ethyl]pyridine-3- sulfonamide (81.86% inhibition at 3.50 μM) | ¹H NMR (300 MHz, DMSO-d₆) 9.14 (s, 1H), 8.85 (d, J = 2.2 Hz, 1H), 8.51 (s, 1H), 8.17 (dd, J = 8.3, 2.2 Hz, 1H), 7.95 (d, J = 8.0 Hz, 1H), 7.42 (s, 1H), 4.95 (q, J = 6.9 Hz, 1H), 4.25 (q, J = 7.6 Hz, 2H), 2.03-2.28 (m, 1H), 1.44 (d, J = 6.9 Hz, 3H), 1.30 (t, J = 7.0 Hz, 3H), 0.89-0.98 (m, 4H). | 396 | — |
| 174 | 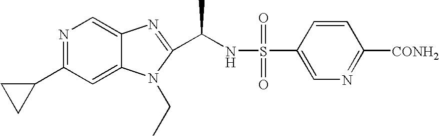 5-({[(1R)-1-(6-Cyclopropyl-1- ethyl-1H-imidazo[4,5- c]pyridin-2- yl)ethyl]amino}sulfonyl)pyr- idine-2-carboxamide (87.78% inhibition at 3.50 μM) | ¹H NMR (300 MHz, DMSO-d₆) δ ppm 8.94 (br. s., 1H), 8.86 (d, J = 2.2 Hz, 1H), 8.56 (s, 1H), 8.22 (dd, J = 8.3, 2.2 Hz, 1H), 8.14 (s, 1H), 8.03 (d, J = 8.3 Hz, 1H), 7.82 (s, 1H), 7.43 (s, 1H), 4.88-5.06 (m, 1H), 4.19- 4.38 (m, 2H), 2.06-2.23 (m, 1H), 1.37 (d, J= 6.9 Hz, 3H), 1.31 (t, J = 7.2 Hz, 3H), 0.88-0.94 (m, 4H). | 414 | — |
| 175 | 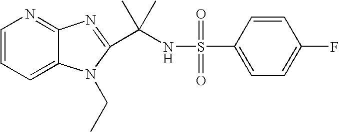 N-[1-(1-Ethyl-1H- imidazo[4,5-b]pyridin-2-yl)- 1-methylethyl]-4- fluorobenzenesulfonamide | ¹H NMR (300 MHz) 8.63 (br. s., 1H), 8.38 (dd, J = 4.68, 1.38 Hz, 1H), 7.92 (dd, J = 8.26, 1.65 Hz, 1H), 7.75-7.68 (m, 2H), 7.29-7.21 (m, 3H), 4.42 (q, J = 7.15 Hz, 2H), 1.60 (s, 6H), 1.40 (t, J = 7.15 Hz, 3H). | 362 | 59 |
| 176 | 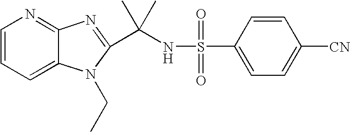 4-Cyano-N-[1-(1-ethyl-1H- imidazo[4,.5-b]pyridin-2-yl)- 1-methylethyl] benzenesulfonamide (36.28% inhibition at 3.50 μM) | ¹H NMR (300 MHz) 8.93 (br. s., 1H), 8.39-8.38 (m, 1H), 7.90-7.76 (m, 5H), 7.25-7.21 (m, 1H), 4.36 (q, J = 6.88 Hz, 2H), 1.62 (s, 6H), 1.38 (t, J = 6.88 Hz, 3H). | 369 | 59 |
| 177 | 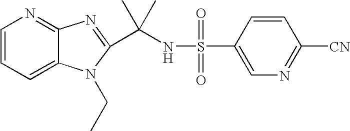 6-Cyano-N-[1-(1-ethyl-1H- imidazo[4,5-b]pyridin-2-yl)- 1-methylethyl]pyridine-3- sulfonamide (3.55% inhibition at 3.50 μM) | ¹H NMR (MeOH-d₄) δ ppm 8.89 (s, 1H), 8.45 (d, 1H), 8.24 (d, 1H), 8.0 (d, 1H), 7.91 (d, 1H), 7.34 (dd, 1H), 4.66 (q, 2H), 1.82 (s, 6H), 1.55 (t, 3H) | 370 | 59 |
| 178 | 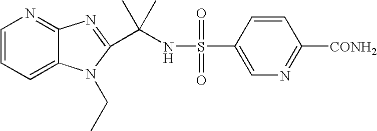 5-({[1-(1-Ethyl-1H-imidazo- [4,5-b]pyridin-2-yl)-1- methylethyl]amino}sulfonyl) pyridine-2-carboxamide (8.27% inhibition at 3.50 μM) | ¹H NMR 8.90 (s, 1H), 8.79 (s, 1H), 8.40 (d, 1H), 8.2 (m, 3H), 8.05 (d, 1H), 7.91 (d, 1H), 7.7 (bs,. 1H), 7.34 (dd, 1H), 4.45 (q, 2H), 1.68 (s, 6H), 1.45 (t, 3H) | 388 | Ex. 177 |
| 179 | 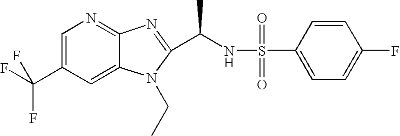 N-{(1R)-1-[1-Ethyl-6- (trifluoromethyl)-1H- imidazo[4,5-b]pyridin-2- yl]ethyl}-4-fluorobenzene sulfonamide (90.47% inhibition at 3.50 μM) | ¹H NMR (MeOH-d₄) δ ppm 8.7 (s, 1H), 8.35 (s,1H), 7.8 (dd, 2H) 7.05 (dd, 2H), 5.01 (q, 2H), 4.46 (q, 2H), 1.62 (d, 3H), 1.45 (t, 3H) | 416 | 46′ |
| 180 | 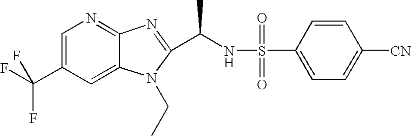 4-Cyano-N-{(1R)-1-[1-ethyl- 6-(trifluoromethyl)-1H- imidazo[4,5-b]pyridin-2- yl]ethyl}benzenesulfonamide (91.07% inhibition at 3.50 μM) | ¹H NMR (MeOH-d₄) δ ppm 8.7 (s, 1H), 8.4 (s, 1H), 7.81 (d,2H) 7.59 (d, 1H), 5.05 (q, 1H), 4.5 (q, 2H), 1.60 (d, 3H), 1.52 (t, 3H) | 423 | 46′ |
| 181 | 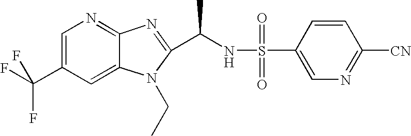 6-Cyano-N-{(1R)-1-[1-ethyl- 6-(trifluoromethyl)-1H- imidazo[4,5-b]pyridin-2- yl]ethyl}pyridine-3- sulfonamide (90.6% inhibition at 3.50 μM) | ¹H NMR (MeOH-d₄) δ ppm 8.85 (s, 1H), 8.72 (s, 1H), 8.45 (s, 1H), 8.15 (d, 1H) 7.7 (d, 1H), 5.1 (q, 2H), 4.55 (q, 2H), 1.63 (d, 3H), 1.55 (t, 3H) | 424 | 46′ |
| 182 | 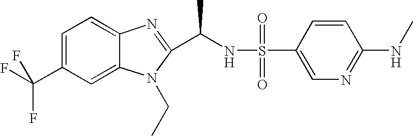 N-{(1R)-1-[1-Ethyl-6- (trifluoromethyl)-1H- benzimidazol-2-yl]ethyl}-6- (methylamino)pyridine-3- sulfonamide (92.53% inhibition at 3.50 μM) | ¹H NMR (MeOH-d₄) δ ppm 8.2 (s, 1H), 7.8 (s, 1H), 7.7 (d, 1H), 7.5 (d, 2H), 6.2 (d, 1H), 4.4 (q, 2H), 2.7 (s, 3H), 1.6 (d, 3H), 1.4 (t, 3H) | 427 | Ex. 35 |
| 183 | 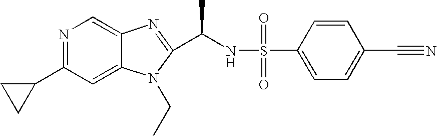 4-Cyano-N-[1-(6- cyclopropyl-1-ethyl-1H- imidazo[4,5-c]pyridin-2-yl)- ethyl]-benzenesulfonamide (83.41% inhibition at 3.50 μM) | ¹H NMR (300 MHz, DMSO-d₆) δ ppm 8.86 (br. s., 1H), 8.58 (s, 1H), 7.80- 7.88 (m, 4H), 7.41 (s, 1H), 4.83- 4.96 (m, 1H), 4.19-4.28 (m, 2H), 2.07-2.24 (m, 1H), 1.38 (d, J = 6.9 Hz, 3H), 1.30 (t, J = 7.2 Hz, 3H), 0.87- 0.99 (m, 4H) | 395 | 60 |
| 184 | 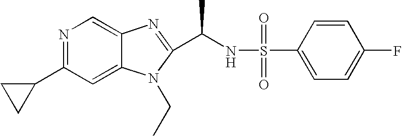 N-[(1R)-1-(6-Cyclopropyl-1- ethyl-1H-imidazo[4,5- c]pyridin-2-yl)ethyl]-4- fluorobenzenesulfonamide | ¹H NMR (300 MHz, DMSO-d₆) δ ppm 8.63 (s, 1H), 8.53 (br. s., 1H), 7.77- 7.84 (m, 1H), 7.81 (dd, J = 8.8, 5.2 Hz, 1H), 7.44 (s, 1H), 7.30 (t, J = 8.8 Hz, 2H), 4.84 (q, J = 6.8 Hz, 1H), 4.21- 4.31 (m, 2H), 2.11-2.20 (m, 1H), 1.27-1.37 (m, 6H), 0.87-0.99 (m, 4H) | 388 | 60 |
| 185 | 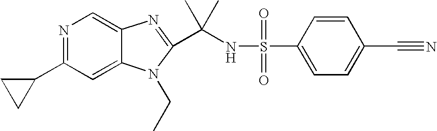 4-Cyano-N-[1-(6- cyclopropyl-1-ethyl-1H- imidazo[4,5-c]pyridin-2-yl)- 1-methyl-ethyl]- benzenesulfonamide (65.01% inhibition at 3.50 μM) | ¹H NMR (300 MHz, DMSO-d₆) δ ppm 8.91 (br. s., 1H), 8.66 (s, 1H), 7.82 (d, 2H), 7.74 (d, 2H), 7.31 (s, 1H), 4.28-4.32 (m, 2H), 2.09-2.24 (m, 1H), 1.60 (s, 6H), 1.37 (t, J = 7.0 Hz, 3H), 0.90-0.97 (m, 4H) | 409 | 61 |
| 186 | 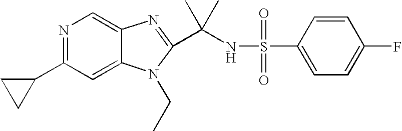 4-Fluoro-N-[1-(6- cyclopropyl-1-ethyl-1H- imidazo[4,5-c]pyridin-2-yl)- 1-methyl-ethyl]- benzenesulfonamide (56.47% inhibition at 3.50 μM) | ¹H NMR (300 MHz, DMSO-d₆) δ ppm 8.91 (br. s., 1H), 8.66 (s, 1H), 7.82 (d,2H), 7.74 (d, 2H), 7.31 (s, 1H), 4.28-4.32 (m, 2H), 2.09-2.24 (m, 1H), 1.60 (s, 6H), 1.37 (t, J = 7.0 Hz, 3H), 0.90-0.97 (m, 4H). | 402 | 61 |
| 187 | 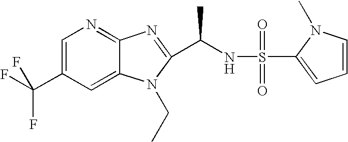 (R)-N-(1-(1-Ethyl-6- (trifluoromethyl)-1H- benzo[d]imidazol-2-yl)ethyl)- 1-methyl-1H-pyrrole-2- sulfonamide (86.24% inhibition at 3.50 μM) | ¹H NMR 1.27-1.36 (m, 6H), 3.77 (s, 3H) 4.28-4.34 (m, 1H) 4.36-4.42 (m, 1H) 4.72 (m, 1H) 5.94 (m, 1H) 6.62 (m, 1H) 6.93 (m, 1H) 7.48 (m, 1H) 7.76 (m, 1H) 8.00 (m, 1H) 8.42 (m, 1H). | 400 | 9 |

Example 101 was generated from Example 12 by enantio-resolution on a normal phase chiral HPLC (chiral pak AD-H, 250×21 mm, 5μ) using 40% methanol, 0.1% dimethylethylamine. Example 102 was similarly generated from Example 13 using 50% hexanes, 50% (1:1) ethanol:methanol, 0.1% DIEA as a modifier. Example 88 may also be generated by enantio-resolution of the racemic version of Example 88. The racemate of Example 88 was prepared by using Boc-DL-Ala-OH as the commercially available starting material by a method analogous to that described for Example 88. The enantio-resolution was accomplished on a normal phase chiral HPLC (chiral pak AD-H, 250×21 mm, 5 g) using 30% isopropanol as the modifier. Examples 173 anad 174 were generated by enantio-resolution of their corresponding racemates after carrying out their syntheses as outlined below. Examples 179-181 were generated by separation of their respective mixture predominantly rich in the desired isomer by super critical fluid chromatography [SFC] (methanol/CO₂). Examples 183 and 184 were generated by enantioresolution of their racemates synthesized by the methods described.

### Example 68

### 4-Chloro-N-{1-[1-ethyl-6-(hydroxymethyl)-1H-benzimidazol-2-yl]ethyl}benzenesulfonamide

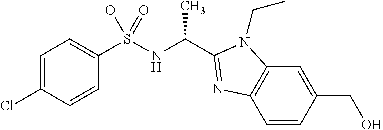

A 250 mL round bottom flask containing crude ethyl 2-(1-{[(4-chlorophenyl)sulfonyl]-amino}ethyl)-1-ethyl-1H-benzimidazole-6-carboxylate (Example 67, 12.5 mmol) was evacuated and back filled with N₂ (3×). Anhydrous THF (30 mL) was added, and the resulting solution was cooled to 0° C. DIBAL (1.0 M solution in THF; 50 mL, 50 mmol) was added drop wise. After 1.5 h at 0° C., additional DIBAL (14 mL, 14 mmol) was added. The resulting solution was allowed to stir at room temperature overnight. The reaction was cautiously quenched with aqueous MeOH (until gas evolution ceased), and the layers of the biphasic mixture were separated. The aqueous layer was extracted with EtOAc (2×), and the combined organics were washed with H₂O, brine, dried (MgSO₄), filtered, and concentrated. The crude material was purified by silica gel chromatography (gradient elution; R_{f} in 100% EtOAc=0.27) to give a colorless to pale yellow solid (3.32 g, 68%).

Application of the above procedure to Example 76 yielded Example 78.

### Example 69

### 4-Chloro-N-{1-[6-(difluoromethyl)-1-ethyl-1H-benzimidazol-2-yl]ethyl}benzenesulfonamide

Example 69 was prepared in two steps from Example 68:

### Step 1: 4-Chloro-N-[1-(1-ethyl-6-formyl-1H-benzimidazol-2-yl)ethyl]benzenesulfonamide

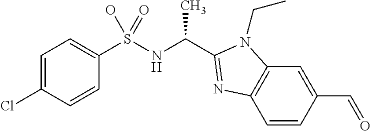

A 100 mL round bottom flask was charged with 4-chloro-N-{1-[1-ethyl-6-(hydroxymethyl)-1H-benzimidazol-2-yl]ethyl}benzenesulfonamide (Example 68, 1.18 g, 3.00 mmol) and activated MnO₂ (85%; 1.56 g, 15.3 mmol). Acetone (15 mL) was added, and the suspension was allowed to stir at room temperature over the weekend. The mixture was suction-filtered through a pad of diatomaceous earth, and the filter cake was thoroughly washed with acetone. Concentration of the filtrate afforded the title compound as a foam (1.18 g, 100%). M/Z=391. ¹H NMR (CDCl₃) δ ppm 1.46 (t, J=7.33 Hz, 3H) 1.66 (d, J=6.82 Hz, 3H) 4.15-4.26 (m, 1H) 4.31 (m, 1H) 4.89 (m, 1H) 6.35 (m, 1H) 7.11-7.21 (m, 2H) 7.63 (m, 2H) 7.73 (m, 1H) 7.83 (m, 1H) 7.90 (m, 1H) 10.08 (s, 1H).

### Step 2

### 4-Chloro-N-{1-[6-(difluoromethyl)-1-ethyl-1H-benzimidazol-2-yl]ethyl}benzenesulfonamide

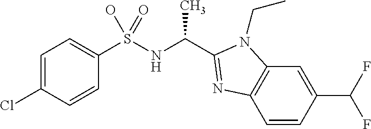

A 50 mL round bottom flask was charged with 4-chloro-N-[1-(1-ethyl-6-formyl-1H-benzimidazol-2-yl)ethyl]benzenesulfonamide (obtained from step 1, 232 mg, 0.59 mmol) and CHCl₃ (4 mL). Freshly distilled DAST (180 mL, 1.36 mmol) was added, and the resulting solution was heated at 60° C. overnight. On cooling, the reaction mixture was partitioned between CH₂Cl₂ and H₂O. The aqueous layer was extracted with CH₂Cl₂, and the combined organics were washed with brine, dried (MgSO₄), filtered and concentrated. The crude material was purified by silica gel chromatography (gradient elution; R_{f} in 50:50 hexanes:EtOAc=0.26) to an orange-colored oil. Crystallization from CH₂Cl₂/hexanes afforded the product as a pale yellow solid (34 mg, 14%).

### Example 70

### 2-(1-{[(4-Chlorophenyl)sulfonyl]amino}ethyl)-1-ethyl-1H-benzimidazole-6-carboxylic acid

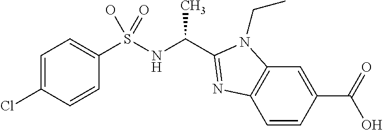

A 250 mL round bottom flask was charged with ethyl 2-(1-{[(4-chlorophenyl)sulfonyl]-amino}ethyl)-1-ethyl-1H-benzimidazole-6-carboxylate (Example 67, 1.76 g, 4.04 mmol) and dioxane (10 mL). A solution of NaOH (732 mg, 18.3 mmol) in H₂O (6 mL) was added, and the mixture was heated to 50° C. After 5 h, the reaction was allowed to cool and was diluted with H₂O (20 mL). Concentrated HCl was added (˜3 mL) dropwise, precipitating a colorless solid that was isolated by suction filtration, washed with H₂O, and dried in air to give the title compound (1.14 g, 69%). M/Z=407. ¹H NMR 5 ppm 1.30 (t, J=7.20 Hz, 3H) 1.38 (d, J=6.82 Hz, 3H) 4.28-4.38 (m, 2H) 4.82-4.91 (m, 1H) 7.42 (m, 2H) 7.55 (m, 1H) 7.68 (m, 2H) 7.78 (m, 1H) 8.08 (m, 1H) 8.62 (m, 1H) 12.79 (s, 1H).

### Preparation of Examples 71-73 from Example 70

Examples 71-73 were prepared from Example 70 by the general procedure outlined below. A test tube equipped with a stir bar was charged with 2-(1-{[(4-Chlorophenyl)sulfonyl]-aminoethyl)-1-ethyl-1H-benzimidazole-6-carboxylic acid (Example 70, 0.33 mmol) and PyBOP (0.37 mmol). Diisopropylethylamine (70 μL, 0.39 mmol) and CH₂Cl₂ (1.0 mL) were added, and the solutions were allowed to stir at room temperature for 30 min. The desired amine (˜2 equiv) was then added, and the mixtures were allowed to stir at room temperature for 2 h. The reactions were diluted with H₂O (10 mL) and extracted with CH₂Cl₂ (2×). The combined organics were washed with brine, dried (MgSO₄), filtered, and concentrated. The crude amides were purified by reverse-phase HPLC (5-95% 0.1% TFA in MeCN/0.1% TFA in H₂O; Atlantis 19×100 column; 10 min run).

Application of this procedure to Example 76 yielded Example 77.

### Preparation of Examples 74 and 75

Examples 74 and 75 were prepared by reductive amination of 4-Chloro-N-[1-(1-ethyl-6-formyl-1H-benzimidazol-2-yl)ethyl]benzenesulfonamide (generated in step 1 of Example 69, above) with the appropriate amine by the general method described below:

General procedure for reductive amination to prepare Examples 74 and 75:

A 25 mL round bottom flask was charged with 4-chloro-N-[1-(1-ethyl-6-formyl-1H-benzimidazol-2-yl)ethyl]benzenesulfonamide (example 69, 1 equiv), the corresponding amine (1.5 equiv), and THF (˜4 mL per mmol aldehyde). NaBH(OAc)₃ (2 equiv) was added, and the reaction was allowed to stir at room temperature overnight. The reaction was partitioned between EtOAc and H₂O, and the aqueous layer was further extracted with EtOAc. The combined organics were washed with brine, dried (MgSO₄), filtered, and concentrated. The crude products were purified by reverse-phase HPLC to give the desired compounds as their TFA salts.

### Example 80

### 4-Chloro-N-{1-[1-ethyl-6-(methylsulfonyl)-1H-benzimidazol-2-yl]ethyl}benzenesulfonamide

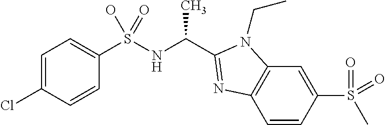

A 250 mL round bottom flask containing 4-chloro-N-{1-[1-ethyl-6-(methylthio)-1H-benzimidazol-2-yl]ethyl}benzenesulfonamide (Example 79, 659 mg, 1.61 mmol) was treated with MeOH (10 mL). The solution was cooled to 0° C., and then Oxone (2.25 g, 7.32 mmol of oxidant) and H₂O were added. The mixture was allowed to warm to room temperature. After 4 hours, the reaction was diluted with H₂O (40 mL) and the mixture was extracted with CH₂Cl₂ (2×). The combined organics were washed with brine, dried (MgSO₄), filtered, and concentrated. The crude material was purified by silica gel chromatography (gradient elution; R_{f} in 25:75 hexanes:EtOAc=0.29) to give a colorless solid (459 mg, 64%).

### Example 95

### 4-chloro-N-[1-(1-ethyl-4-methoxy-1H-imidazo[4,5-c]pyridin-2-yl)ethyl]benzenesulfonamide

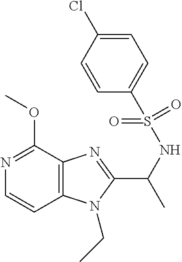

A solution of 4-chloro-N-[1-(4-chloro-1-ethyl-1H-imidazo[4,5-c]pyridin-2-yl)ethyl]benzenesulfonamide (Example 94, 20 mg) in MeOH (1 mL) was treated with 20 molar equivalents of sodium methoxide. The resulting solution was refluxed for 5 h. The reaction mixture was concentrated to yield a viscous glue, which was purified on reverse phase HPLC to yield 4-chloro-N-[1-(1-ethyl-4-methoxy-1H-imidazo[4,5-c]pyridin-2-yl)ethyl]benzenesulfonamide.

### Example 96

### 4-chloro-N-{1-[9-ethyl-2-(trifluoromethyl)-9H-purin-8-yl]ethyl}benzenesulfonamide

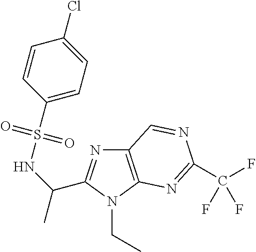

Example 96 was prepared from commercially available 2-amino-2-cyanoacetamide in 7 steps

### Step 1

### 5-amino-1-ethyl-1H-imidazole-4-carboxamide

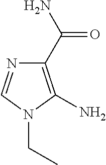

To a solution of 2-amino-2-cyanoacetamide (10 g) in CH₃CN was added triethyl ortho-formate and heated to reflux for 2 h. To the cooled solution was added ethylamine (61 mL, 2M in THF). The solution was stirred overnight. A precipitate formed and was collected to yield the desired product, 5-amino-1-ethyl-1H-imidazole-4-carboxamide as a solid (11 g). M/Z 154.

### Step 2

### 9-ethyl-2-(trifluoromethyl)-1,9-dihydro-6H-purin-6-one

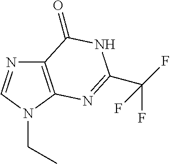

To a solution of 5-amino-1-ethyl-1H-imidazole-4-carboxamide (2 g) in EtOH (71 mL) was added ethyl trifluoroacetate (15.44 mL) followed by sodium ethoxide. The reaction mixture was heated to reflux for 24 hours. The crude mixture was dilute with a saturated aqueous solution of ammonia chloride and extracted with EtOAc. The organic layers were concentrated and used directly in the next step. M/Z 232.

### Step 3

### 6-chloro-9-ethyl-2-(trifluoromethyl)-9H-purine

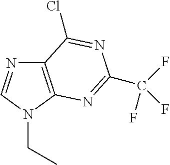

A solution of 5-amino-1-ethyl-1H-imidazole-4-carboxamide (approximately 2 g) in POCl₃ (10 mL) was heated to reflux for overnight. The reaction mixture was concentrated under vacuum and purified using silica chromatography to yield 6-chloro-9-ethyl-2-(trifluoromethyl)-9H-purine as a solid. M/Z 250.

### Step 4

### 1-[6-chloro-9-ethyl-2-(trifluoromethyl)-9H-purin-8-yl]ethanone

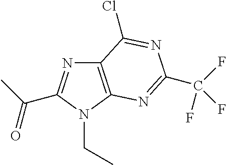

A solution of 6-chloro-9-ethyl-2-(trifluoromethyl)-9H-purine (200 mg) in THF was added LDA 90.7 mL, 1.8 M) at −78° C. and stirred for 35 min before N-methoxy-N-methylacetamide (252.8 uL) was added. After stirring for 0.5 h, the reaction was quenched with water and extracted with EtOAc. The organic layers were concentrated. The mixture was purified using silica chromatography to yield 1-[6-chloro-9-ethyl-2-(trifluoromethyl)-9H-purin-8-yl]ethanone. M/z 292.

### Step 5

### 1-[9-ethyl-2-(trifluoromethyl)-9H-purin-8-yl]ethanol

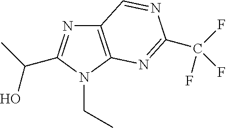

To a solution of 1-[6-chloro-9-ethyl-2-(trifluoromethyl)-9H-purin-8-yl]ethanone (50 mg) in EtOAc (1 mL) was added triethyl amine followed by palladium on carbon (50 mg). The reaction mixture was placed under H₂ (1 atm.) for 5 h. The reaction mixture was filtered through a pad of Diatomaceous earth and concentrated to yield 1-[9-ethyl-2-(trifluoromethyl)-9H-purin-8-yl]ethanol (40 mg). M/Z 260.

### Step 6

### tert-butyl[(4-chlorophenyl)sulfonyl]{1-[9-ethyl-2-(trifluoromethyl)-9H-purin-8-yl]ethyl}carbamate

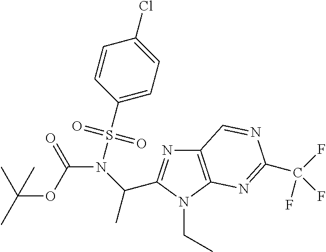

To a solution of 1-[9-ethyl-2-(trifluoromethyl)-9H-purin-8-yl]ethanol (30 mg) in THF (1 mL) was added triphenyl phosphine, tert-butyl[(4-chlorophenyl)sulfonyl]carbamate (100 mg) and diisopropylazodicarboxylate (70 ul). The reaction was stirred for overnight. The reaction mixture was concentrated and purified using silica column to yield tert-butyl[(4-chlorophenyl)sulfonyl]{1-[9-ethyl-2-(trifluoromethyl)-9H-purin-8-yl]ethyl}carbamate. M/Z 533.

### Step 7

### 4-chloro-N-{1-[9-ethyl-2-(trifluoromethyl)-9H-purin-8-yl]ethyl}benzenesulfonamide

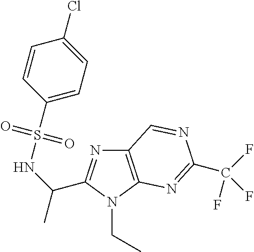

A solution of tert-butyl[(4-chlorophenyl)sulfonyl]{1-[9-ethyl-2-(trifluoromethyl)-9H-purin-8-yl]ethyl}carbamate in MeOH was treated with HCl (4 mL, 4N in dooxane). The reaction mixture was stirred for 48 h and was concentrated and purified on reverse phase HPLC to yield 4-chloro-N-{1-[9-ethyl-2-(trifluoromethyl)-9H-purin-8-yl]ethyl}benzenesulfonamide (20 mg).

Examples 97, 98, 103, 104 and 144 were directly generated from Amide Starting Material (SM2) 2ab, 2ac, 2e′ and 2ad respectively, and the appropriate commercially available sulfonyl chloride, by the method represented below for Example 97. Examples 103 and 144, were generated by resolution of the enantiomers by super critical fluid chromatography (MeOH/CO₂). Example 104 was prepared similarly from 2e′ and the appropriate commercially available sulfonyl chloride except the reaction was carried out at room temperature rather than in the microwave as described below and the desired product was separated from the unreacted starting material by column chromatography to generate the desired product.

### Example 97

### 4-chloro-N-[(1R)-1-(6-chloro-1-ethyl-1H-imidazo[4,5-c]pyridin-2-yl)ethyl]benzenesulfonamide

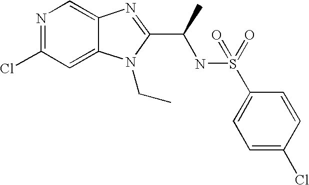

### Step 1

### (2R)—N-(6-chloro-4-ethylamino-pyridin-3-yl)-2-[(4-chlorophenyl)sulfonylamino]propanamide

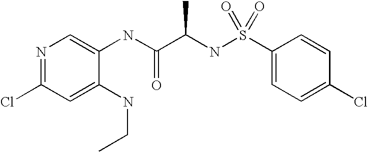

To anhydrous MeOH (20 mL) was added tert-butyl N-[(1R)-1-[(6-chloro-4-ethylamino-pyridin-3-yl)carbamoyl]ethyl]carbamate (SM 2ab, 1.3 g, 3.8 mmol), and HCl in dixoane (20 mL, 4 M solution) was added. The reaction was kept at rt for 1 h. Concentration removed the solvents. To the residue was added DCM (15 mL), and the mixture was cooled to −15° C. p-chlorobenzenesulfonyl chloride (0.8 g, 3.9 mmol) and Et₃N (1.5 mL, 11.4 mmol) were added. The reaction was warmed up to rt for 1 h. After addition of water (10 mL), extraction with DCM (2×15 mL), drying (Na₂SO₄), and concentration, the product was collected as a solid (1.1 g, 60% yield). M/Z 416.

### Step 2

### 4-chloro-N-[(1R)-1-(6-chloro-1-ethyl-1H-imidazo[4,5-c]pyridin-2-yl)ethyl]benzenesulfonamide

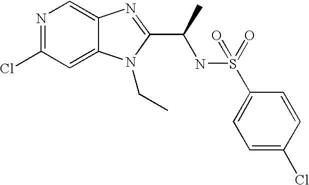

In a microwave tube equipped with a magnetic stirring bar was placed (2R)—N-(6-chloro-4-ethylamino-pyridin-3-yl)-2-[(4-chlorophenyl) sulfonylamino]propanamide (0.3 g, 0.72 mmol), and AcOH (5 mL) was added. The reaction was heated using a microwave at 150° C. for 0.5 h. Concentration removed AcOH, and the resulting residue was dissolved in EtOAc (15 mL), treated with sat. NaHCO₃ and extracted with EtOAc (2×10 mL). Silica gel chromatography purification afforded the title compound as a white solid (0.16 g, 55% yield).

Examples 141, 149 and 152 were generated from appropriate examples as indicated in Table 1 in a manner analogous to that described below for Ex. 106. All of these compounds may also be generated by direct sulfonamidation using SC 10 with the appropriate intermediates as indicated in Table 1 by a standard sulfonamidation procedure described above for Ex. 1. Ex 178 was prepared from Ex. 177 using the method described below.

### Example 106

### (R)-5-(N-(1-(1-Ethyl-6-(trifluoromethyl)-1H-benzo[d]imidazol-2-yl)ethyl)sulfamoyl)picolinamide

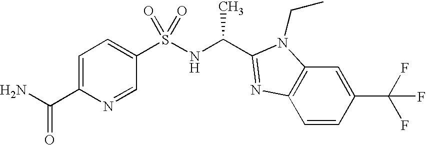

A test tube equipped with a stir bar was charged with (R)-6-cyano-N-(1-(1-ethyl-6-(trifluoromethyl)-1H-benzo[d]imidazol-2-yl)ethyl)pyridine-3-sulfonamide (Ex 105, 82 mg, 0.19 mmol) and concentrated sulfuric acid (1 mL). The resulting mixture was allowed to stir at room temperature overnight. The reaction mixture was poured onto to 20 mL crushed ice, and the resulting mixture was treated with K₂CO₃ until the mixture was basic. The mixture was extracted with CH₂Cl₂ (3×), and the combined organic extracts were washed with brine, dried (MgSO₄), filtered, and concentrated to a colorless solid. This was dried in vacuo to give 77 mg (90%) of analytically pure material.

The sulfonyl chlorides listed in Table 6 were used to generate examples 106 and 108-110.

### Example 111

### (R)-4-Amino-N-(1-(1-ethyl-6-(trifluoromethyl)-1H-benzo[d]imidazol-2-yl)ethyl)benzenesulfonamide

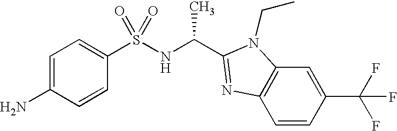

A 250 mL round bottom flask containing (R)—N-(1-(1-ethyl-6-(trifluoromethyl)-1H-benzo[d]imidazol-2-yl)ethyl)-4-nitrobenzenesulfonamide (Ex. 110, 1.25 g, 2.83 mmol) was charged with tin(II) chloride dihydrate (2.72 g, 12.05 mmol) and EtOAc (15 mL). The resulting mixture was heated using a 80° C. oil bath. After 1 hour at reflux, the reaction was allowed to cool to room temperature and was diluted with H₂O (25 mL). Saturated NaHCO₃ was added, causing gas evolution and precipitation of a solid material. The resulting mixture was suction-filtered through a pad of diatomaceous earth, and the reaction flask and filter cake were thoroughly washed with EtOAc and H₂O. The filtrate layers were separated, and the aqueous layer was extracted with EtOAc. The combined organics were washed with brine, dried (MgSO₄), filtered, and concentrated to a viscous oil. The product was precipitated from CH₂Cl₂/hexanes to give 973 mg (84%) of colorless to pale yellow solid.

### Example 128

### R-5-chloro-N-(1-(1-ethyl-6-(trifluoromethyl)-1H-benzo[d]imidazol-2-yl)ethyl)-6-hydrazinylpyridine-3-sulfonamide

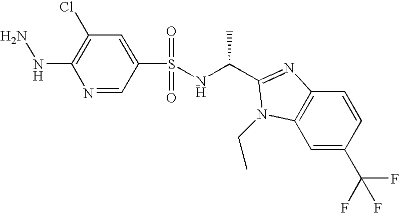

Under a nitrogen purge, R-5,6-dichloro-N-(1-(1-ethyl-6-(trifluoromethyl)-1H-benzo[d]imidazol-2-yl)ethyl)pyridine-3-sulfonamide (Ex. 48, 0.472 g, 0.00101 mol) was added to ethanol (15 mL) in a 50 mL 3-neck round-bottomed flask, providing a suspension. In a single portion, hydrazine hydrate (0.102 g, 0.00204 mol; 0.1 mL) was added. Upon heating to reflux, the solids all dissolved. After 90 min at reflux, turbidity was noted, and some solids formed. After another 1 h, LC/MS indicated some starting material remained and another 0.1 mL hydrazine hydrate was added. After refluxing another 2 h, the reaction mixture was cooled and the solvent was removed under reduced pressure to provide a solid. The solid was partitioned between ethyl acetate and water, filtered and the organic layer was washed twice with water, then once with saturated sodium chloride. After drying over MgSO₄, the solvent was removed under reduced pressure to provide the desired product as a white solid, 0.41 g (88%).

### Example 133

### (R)—N-(1-(1-ethyl-6-(trifluoromethyl)-1H-benzo[d]imidazol-2-yl)ethyl)-6-ethynylpyridine-3-sulfonamide

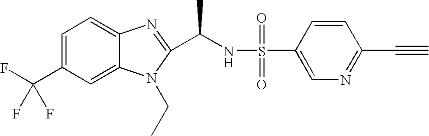

All the solid reactants namely, (R)-6-chloro-N-(1-(1-ethyl-6-(trifluoromethyl)-1H-benzo[d]imidazol-2-yl)ethyl)pyridine-3-sulfonamide (Ex. 35, 200 mg, 0.46 mmols), were charged in a reaction vessel under nitrogen. To this reaction mixture, DMF (0.5 mL) and triethylamine (0.322 mL) were added following which trimethylsilylacetylene (0.30 mL) was added. The resultant mixture was heated at 60° C. for 3 h at which point all the starting material had been consumed. The reaction mixture was cooled to room temperature and tetrabutylammonium fluoride (1 mL, 1M in THF) was added and the resultant mixture was stirred at room temperature for 30 minutes. The mixture was concentrated to remove THF and then subjected to column chromatography using a gradient of ethyl acetate and hexanes (20% to 100%) to isolate the desired product (65 mg, 33.3%).

### Example 129

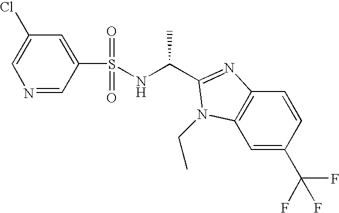

### R-5-chloro-N-(1-(1-ethyl-6-(trifluoromethyl)-1H-benzo[d]imidazol-2-yl)ethyl)pyridine-3-sulfonamide

Under a nitrogen, R-5-chloro-N-(1-(1-ethyl-6-(trifluoromethyl)-1H-benzo[d]imidazol-2-yl)ethyl)-6-hydrazinylpyridine-3-sulfonamide (Ex. 128, 0.347 mg, 0.75 mmol) was dissolved in acetic acid (9 mL) in a 50 mL 3-neck round-bottomed flask, remaining in solution with the addition of water (3 mL). The solution was heated to reflux, whereupon a solution of copper(II) sulfate pentahydrate (0.412 mg, 0.165 mmol) dissolved in 5 mL water was added dropwise from an addition funnel. After the addition was complete, the solution was refluxed another 75 min. The solution was cooled, and solvent removed in vacuo. The residue was partitioned between ethyl acetate and dilute (1 part:4 parts water) ammonium hydroxide solution. The organic layer was washed with diluted ammonium hydroxide solution, then a 50:50 (water:saturated EDTA) solution. The organic layer was then washed with saturated sodium chloride solution, and dried over MgSO₄. Removal of solvent under reduced pressure provided a brownish semi-solid. Trituration of the semi-solid with methylene chloride provided 0.15 g of an off-white solid by filtration. The filtrate was concentrated under reduce pressure and chromatographed by medium pressure chromatography (ethanol in dichloromethane; 5% conc. ammonium hydroxide in ethanol) to obtain another 0.020 g of material, 0.017 g combined total. (52%) constituting a mixture of R and S enantiomers in 9:1 ratio which was further resolved by chiral HPLC.

### Example 130

### N-(1-(1-ethyl-6-(trifluoromethyl)-1H-benzo[d]imidazol-2-yl)ethyl)-4-(morpholinomethyl)benzenesulfonamide

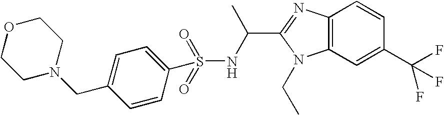

The hydrochloride salt of intermediate 9 (0.296 g, 0.00101 mol) was suspended in THF (30 mL) and cooled in ice-acetone bath. N,N-Diisopropylethylamine (0.528 mL, 0.00303 mol) was added to the suspension in a single portion. 4-(Bromomethyl)benzenesulfonyl chloride (0.272 g, 0.00101 mol) was dissolved in THF (5 mL) and added dropwise to the mixture. The reaction mixture was stirred 30 min in the ice-acetone bath, then warmed to room temperature and stirred another 30 min. The suspension was again cooled in ice-acetone bath and morpholine (0.528 mL, 0.00606 mol) was added in a single portion. The reaction mixture was then stirred for 30 min while cooling in the ice-acetone bath. After warming to room temperature, the reaction mixture was refluxed for 6. The resulting suspension was cooled, and solvent was removed under reduced pressure. The residue was partitioned between ethyl acetate and water. The organic was washed twice with water, and once with saturated sodium chloride solution. After drying over MgSO₄, solvent was removed under reduced pressure. The resulting oil was purified by flash chromatography (ethanol/dichloromethane; 5% conc. NH4OH in the ethanol) to yield the desired product. Solvent was removed under reduced pressure to obtain the desired product as a white solid, 0.17 g (32% theory). This material was determined to be about 90% R-enantiomer.

### Example 134

### 3-(Aminomethyl)-N-[(1R)-1-[1-ethyl-6-(trifluoromethyl)-1H-benzimidazol-2-yl]ethyl]-4-fluorobenzenesulfonamide

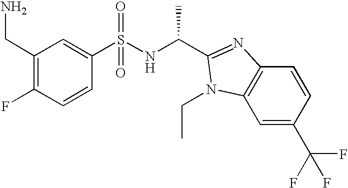

A 25 ml round bottom flask was charged with 3-cyano-N-{(1R)-1-[1-ethyl-6-(trifluoromethyl)-1H-benzimidazol-2-yl]ethyl}-4-fluorobenzenesulfonamide (Ex. 27, 37.4 mg, 0.08 mmol) and THF (2 mL). The solution was cooled to 0° C. and LAH (0.160 mL, 0.32 mmol) was added. The solution turned red, was warmed to room temperature and stirred for 5 hr. Ice and EtOAc (5 mL) were added into the reaction mixture and stirred for 10 mins. The aqueous phase was extracted with EtOAc (3 mL×2) and the combined organic phases were washed with brine. The organic layer was filtered, the solvent was evaporated, and the resulting solid was purified by semi-prep HPLC to afford the desired product (11.5 mg, 30%).

### Example 135

### (R)—N-(1-(1-Ethyl-6-(trifluoromethyl)-1H-benzo[d]imidazol-2-yl)ethyl)pyrimidine-5-sulfonamide

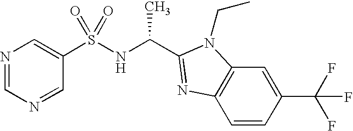

Ex. 135 was prepared in two steps from intermediate 9 as follows:

### Step 1

### (R)—N-(1-(1-Ethyl-6-(trifluoromethyl)-1H-benzo[d]imidazol-2-yl)ethyl)-2-(ethylthio)pyrimidine-5-sulfonamide

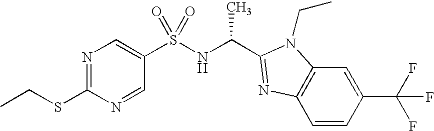

A 50 mL round bottom flask was charged with Intermediate 9 (323 mg, 0.90 mmol) and to which was added CH₂Cl₂ (5 mL) and triethyl amine (650 μL, 4.66 mmol). A separate 50 mL round bottom flask was charged with 2-chloropyrimidine-5-sulfonyl chloride (217 mg, 1.02 mmol) and CH₂Cl₂ (3 mL), and the suspension was cooled to 0° C. The solution of intermediate 9 with CH₂Cl₂ and triethyl amine was added dropwise, followed by an additional 2×1 mL CH₂Cl₂ to rinse in the remaining reagent. The mixture was allowed to stir at 0° C. After 1 hour, ethanethiol (200 μL, 2.70 mmol) was added, followed by additional triethylamine (200 μL, 6.10 mmol total). After 3 hours, an additional 200 μL of ethanethiol (5.40 mmol total) was added, and the reaction was allowed to stir overnight. The mixture was partitioned between CH₂Cl₂ and H₂O, and the aqueous layer was extracted with CH₂Cl₂. The combined organics were washed with brine, dried (MgSO₄), filtered, and concentrated. The crude foam was purified by silica gel chromatography (gradient elution; R_{f} in 50:50 hexanes:EtOAc=0.31) to give a colorless oil that crystallized on standing in vacuo (99 mg, 24%). ¹H NMR (400 MHz, DMSO-D6) δ ppm 1.15 (t, J=7.33 Hz, 3H) 1.29 (t, J=7.20 Hz, 3H) 1.49 (d, J=6.82 Hz, 3H) 2.81-2.91 (m, 2H) 4.37 (m, 2H) 4.95 (m, 1H) 7.44 (m, 1H) 7.57 (m, 1H) 7.96 (s, 1H) 8.58 (s, 2H) 9.00 (m, 1H). M/Z=459.

### Step 2

### (R)—N-(1-(1-Ethyl-6-(trifluoromethyl)-1H-benzo[d]imidazol-2-yl)ethyl)pyrimidine-5-sulfonamide

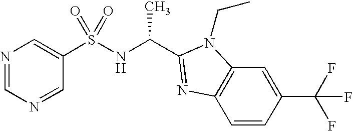

A 50 mL round bottom flask containing (R)—N-(1-(1-ethyl-6-(trifluoromethyl)-1H-benzo[d]imidazol-2-yl)ethyl)-2-(ethylthio)pyrimidine-5-sulfonamide (step 1, 79 mg, 0.17 mmol) was treated with a slurry of Raney Ni (excess) in EtOH (˜5 mL). The resulting mixture was heated to reflux. After 2 hours, LC-MS showed complete conversion of the sulfide to the desired molecular weight. The mixture was allowed to cool and was filtered through a short plug of diatomaceous earth. The flask and filter were washed with MeOH, and the combined filtrates were concentrated to a solid residue. This was purified by silca gel chromatography (gradient elution; R_{f} in 20:80 hexanes:EtOAc=0.18) to give a colorless oil that was lyophilized from a CH₃CN/H₂O solution to give a colorless solid (12 mg, 17%). Example 135 may also be prepared by the following two-step procedure:

### Step 1

### (R)-2-Chloro-N-(1-(1-ethyl-6-(trifluoromethyl)-1H-benzo[d]imidazol-2-yl)ethyl)pyrimidine-5-sulfonamide

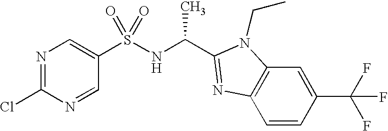

A 100 mL round bottom flask containing (R)-tert-butyl 1-(1-ethyl-6-(trifluoromethyl)-1H-benzo[d]imidazol-2-yl)ethylcarbamate (777 mg, 2.17 mmol) was treated with 4N HCl/dioxane (˜8 mL). After stirring at room temperature for 1 hour, the volatiles were evaporated under reduced pressure. The residue was dissolved in CH₂Cl₂ (10 mL) and NEt₃ (1.50 mL, 10.8 mmol). The mixture was cooled to 0° C., and 2-chloropyrimidine-5-sulfonyl chloride (Beta Pharma, New Haven, Conn.; 513 mg, 2.41 mmol) was added in one portion. The resulting mixture was allowed to stir at 0° C. After 1 hour, the reaction was partitioned between CH₂Cl₂ and H₂O. The aqueous layer was extracted with CH₂Cl₂, and the combined organics were washed with brine, dried (MgSO₄), filtered, and concentrated. The crude material was purified by silica gel chromatography (gradient elution; R_{f} in 60:40 hexanes:EtOAc=0.20) to give a colorless oil that slowly solidified on standing (690 mg). M/Z=433.

### Step 2

### (R)—N-(1-(1-Ethyl-6-(trifluoromethyl)-1H-benzo[d]imidazol-2-yl)ethyl)pyrimidine-5-sulfonamide

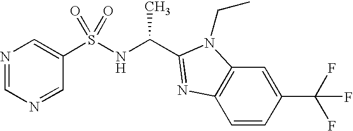

A 50 mL round bottom flask was charged with (R)-2-chloro-N-(1-(1-ethyl-6-(trifluoromethyl)-1H-benzo[d]imidazol-2-yl)ethyl)pyrimidine-5-sulfonamide (generated in step 1, 0.44 mmol), MgO (112 mg, 2.78 mmol), and 10% Pd/C (92 mg). The flask was evacuated and backfilled with H₂ (using a filled balloon), and then MeOH (2 mL) was added. The resulting mixture was allowed to stir at room temperature under 1 atm H₂. After 4 hours, the reaction was suction-filtered through a pad of Celite, and the reaction flask and filter cake were washed well with MeOH. The combined filtrates were concentrated, and the residue was purified by silica gel chromatography (gradient elution; R_{f} in 20:80 hexanes:EtOAc=0.18) to give a colorless oil. The product was precipitated from CH₂Cl₂/hexanes to give a colorless solid (67 mg, 38%).

### Example 158

### (R)-5-(N-(1-(1-Ethyl-6-(trifluoromethyl)-1H-benzo[d]imidazol-2-yl)ethyl)sulfamoyl)-1-methyl-1H-pyrrole-2-carboxamide

### Step 1

### (R)-5-(N-(1-(1-Ethyl-6-(trifluoromethyl)-1H-benzo[d]imidazol-2-yl)ethyl)sulfamoyl)-1′-methyl-1H-pyrrole-2-carboxylic acid

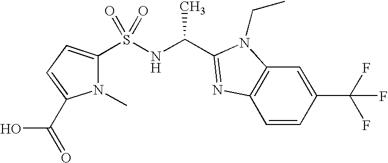

A 50 mL round bottom containing (R)-methyl 5-(N-(1-(1-ethyl-6-(trifluoromethyl)-1H-benzo[d]imidazol-2-yl)ethyl)sulfamoyl)-1-methyl-1H-pyrrole-2-carboxylate (Ex. 40, 465 mg, 1.01 mmol) was charged with LiOH (195 mg, 8.14 mmol). THF (4 mL) and H₂O (2 mL) were added, and the resulting mixture was stirred vigorously at room temperature. After stirring overnight, the mixture was diluted with H₂O (5 mL) and was treated with conc. HCl (˜1 mL) to give a mixture of pH ˜1. The mixture was partitioned between EtOAc and H₂O, and the aqueous layer was extracted with EtOAc (2×). The combined organics were washed with brine, dried (MgSO₄), filtered, and concentrated to give a colorless to tan solid (389 mg, 87%). This was used without any further purification.

### Step 2

### (R)-5-(N-(1-(1-Ethyl-6-(trifluoromethyl)-1H-benzo[d]imidazol-2-yl)ethyl)sulfamoyl)-1-methyl-1H-pyrrole-2-carboxamide

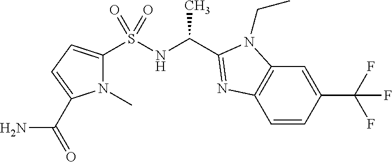

A 100 mL round bottom flask containing (R)-5-(N-(1-(1-ethyl-6-(trifluoromethyl)-1H-benzo[d]imidazol-2-yl)ethyl)sulfamoyl)-1-methyl-1H-pyrrole-2-carboxylic acid (generated in step 1, 389 mg, 0.88 mmol) was charged with HATU (369 mg, 0.97 mmol). Anhydrous DMF (3.0 mL) was added, followed by 4-methylmorpholine (0.15 mL, 1.4 mmol), and the resulting solution was allowed to stir at room temperature. After 45 minutes, 7N NH₃/MeOH (1.0 mL, 7.0 mmol) was added, followed by additional MeOH (1 mL). The mixture was allowed to stir at room temperature. After 3 hours, the reaction was diluted with H₂O (25 mL) and, was extracted with EtOAc (3×). The combined organics were washed with brine, dried (MgSO₄), filtered, and concentrated. The crude material was purified by silica gel chromatography (gradient elution; R_{f} in 100% EtOAc=0.22) to give a colorless solid (259 mg, 67%).

### Example 163

### R)-4-(N-(1-(1-ethyl-6-(trifluoromethyl)-1H-benzo[d]imidazol-2-yl)ethyl)sulfamoyl)benzamide

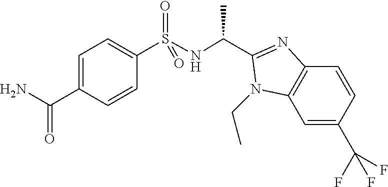

(R)-4-cyano-N-(1-(1-ethyl-6-(trifluoromethyl)-1H-benzo[d]imidazol-2-yl)ethyl)benzenesulfonamide (Ex. 163, 0.21 g, 0.50 mmol) dioxane (3 mL) in a 20 mL pressure tube to get a clear solution. Addition of 4N HCl in dioxane (3 mL) caused a two-phase system to form. The tube was sealed, and the contents were heated at 90° C. for 16 h, resulting in complete conversion. The reaction mixture was cooled, and volatiles removed under reduced pressure. The resulting semi-solid was dissolved in acetonitrile (40 mL) and powder potassium carbonate (0.077 g; 1.1 equiv.) was added the suspension was refluxed for 30 min. Volatiles were removed from the suspension and the residue was partitioned between ethyl acetate and just sufficient water to dissolve the inorganic salts (pH of the water layer approximately 8). The organic layer was separated, and washed once with water and once with saturated sodium chloride solution. After drying over magnesium sulfate, solvent was removed under reduced pressure to obtain a white solid (0.16 g). This was recrystallized from acetonitrile to obtain pure product. (0.08 g, 36.2%).

Ex. 164 was prepared from Ex. 126 as described below. Example 165 was similarly prepared from Ex 127.

### Example 164

### (R)-3-(N-(1-(1-ethyl-6-(trifluoromethyl)-1H-benzo[d]imidazol-2-yl)ethyl)sulfamoyl)pyridine 1-oxide

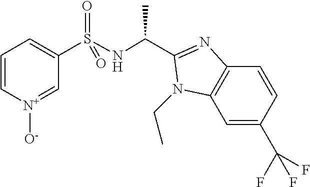

Under a nitrogen purge, (R)—N-(1-(1-ethyl-6-(trifluoromethyl)-1H-benzo[d]imidazol-2-yl)ethyl)pyridine-3-sulfonamide (Ex. 126, 0.200 g, 0.5 mmol) in acetic acid (10 mL) and the solution was heated to 85° C. 50% hydrogen peroxide (0.046 mL, 0.75 mmol) in an addition funnel, along with acetic acid (5 mL). This solution was added dropwise over 15 min and the reaction mixture was stirred for an additional 75 minutes. The reaction mixture was cooled, and solvent and excess reagent were removed under reduced pressure. The resulting residue was partitioned between ethyl acetate and water. The organic layer was washed twice with water, followed by saturated sodium chloride solution. The resultant was dried over magnesium sulfate, filtered and the solvent was removed under reduced pressure to provide a white semi-solid. This was purified by flash chromatography (5 to 20% ethanol/dichloromethane; 5% v:v concentrated ammonium hydroxide in ethanol) to obtain the desired product as a white powder (0.13 g, 62.8%)

### Example 166

### (R)-4-(Chloromethyl)-N-(1-(1-ethyl-6-(trifluoromethyl)-1H-benzo[d]imidazol-2-yl)ethyl)benzenesulfonamide

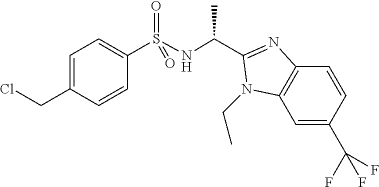

(R)-tert-Butyl 1-(1-ethyl-6-(trifluoromethyl)-1H-benzo[d]imidazol-2-yl)ethylcarbamate (Boc protected INT 9, 0.543 g, 0.00152 mol) in dioxane (15 mL) in a 100 mL round-bottomed flask. (Some heating with a heat gun was necessary to get clear solution, which was then cooled back to room temperature). In a single portion, 4N HCl in dioxane (5 mL) was added, causing a two phase solution to form. The reaction mixture was stirred rapidly at ambient for 3 h. Solvent was removed under reduced pressure, to obtain a white semi-solid. A small portion of ether was added, and the suspension was subjected to ultrasonication. The ether was decanted from the solid, and then the process was repeated twice more. The while solid was placed under vacuum to remove volatiles. This solid was then suspended in tetrahydrofuran (20 mL), and the reaction was cooled in an ice/acetone bath. Diisopropylethylamine (1.059 mL, 6.08 mmol), diluted with tetrahydrofuran (5 mL) was added dropwise, forming a thicker suspension. 4-(bromomethyl)benzene-1-sulfonyl chloride (0.410 g, 0.00152 mol) was dissolved in tetrahydrofuran (5 mL) and placed in an addition funnel. After the contents were added dropwise, the reaction mixture was allowed to warm to ambient and stirred 16 h longer. The suspension was heated to reflux for 1 h, causing the solids to dissolve. Solvent was removed under reduced pressure, and the residue was partitioned between ethyl acetate and water. The organic layer was washed twice with water, then once with saturated sodium chloride solution. After drying over magnesium sulfate, solvent was removed under reduced pressure to obtain a semi-solid. The crude was purified by flash column chromatography using a gradient of 50% ethyl acetate in hexanes to 100% ethyl acetate to obtain the pure product. (0.34 g, 50%).

### Example 167

### (R)-4-(cyanomethyl)-N-(1-(1-ethyl-6-(trifluoromethyl)-1H-benzo[d]imidazol-2-yl)ethyl)benzenesulfonamide

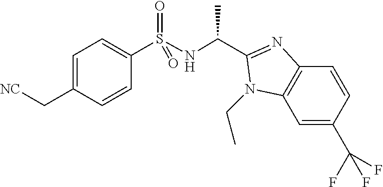

Under a nitrogen purge, (R)-4-(chloromethyl)-N-(1-(1-ethyl-6-(trifluoromethyl)-1H-benzo[d]imidazol-2-yl)ethyl)benzenesulfonamide (Ex. 166, 0.22 g, 0.49 mmol) was dissolved in acetonitrile (22 mL) Trimethylsilyl cyanide (0.132 ml, 0.99 mmol) was added from a syringe, washed in with a bit more acetonitrile. Then tetrabutylammonium fluoride (980 ml, 0.98 mol; 1 M in THF) was added from an addition funnel. The reaction mixture was then heated to reflux, and stirred for 3 h, resulting in complete consumption of the starting material. The reaction mixture was cooled, and solvent was removed under reduced pressure. The residue was partitioned between ethyl acetate and water, and the organic layer washed with water, then brine. After drying over magnesium sulfate, solvent was removed under reduced pressure and the residue was purified by flash column chromatography using a gradient of 50% to 100% ethyl acetate in hexanes to obtain the product as a white solid (0.2 g, 93.5%).

### Example 168

### (R)—N-(1-(1-ethyl-6-(trifluoromethyl)-1H-benzo[d]imidazol-2-yl)ethyl)-6-hydrazinylpyridine-3-sulfonamide

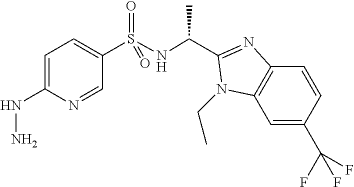

(R)-6-chloro-N-(1-(1-ethyl-6-(trifluoromethyl)-1H-benzo[d]imidazol-2-yl)ethyl)pyridine-3-sulfonamide (Ex. 35, 0.26 g; 0.0006 mol) was dissolved in ethanol (15 mL) in a 50 mL 3-neck round-bottomed flask under a nitrogen purge. Hydrazine hydrate (0.030 g, 0.00060 mol) was added in a single portion, washed in with a bit more ethanol, and the reaction mixture was heated to reflux and maintained for 6 h. Solvent was removed under reduced pressure and the resulting residue was recrystallized from 2-propanol to obtain desired product (0.095 g, 36.9%).

### Example 169

### 6-amino-5-chloro-N-(1-(1-ethyl-6-(trifluoromethyl)-1H-benzo[d]imidazol-2-yl)ethyl)pyridine-3-sulfonamide

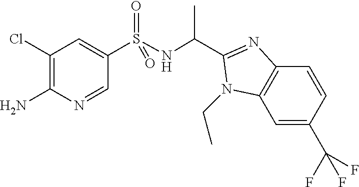

5,6-dichloro-N-(1-(1-ethyl-6-(trifluoromethyl)-1H-benzo[d]imidazol-2-yl)ethyl)pyridine-3-sulfonamide (Ex. 48, 0.238 g, 0.00051 mol), acetamide (0.030 g, 0.00051 mol), potassium carbonate (0.141 g, 0.001 mol) and 18-crown-6 (0.013 g, 0.00005 mol) were added together to 1.5 mL acetonitrile in a 5 mL microwave tube. The tube was sealed and heated to 160° C. for 3 h. 7 Solvent was removed under reduced pressure and the resulting residue was partitioned between ethyl acetate and water. The organic layer was washed twice with water, then once with saturated sodium chloride solution. After drying over magnesium sulfate, solvent was removed in vacuo. The resulting residue was purified by flash chromatography using a gradient of 100% DCM to 15% ethanol in DCM with 5% volume:volume conc. ammonium hydroxide in ethanol to obtain desired product as a beige solid (0.045 g, 19.7%).

### Example 170

### 6-amino-N-(1-(1-ethyl-6-(trifluoromethyl)-1H-benzo[d]imidazol-2-yl)ethyl)pyridine-3-sulfonamide

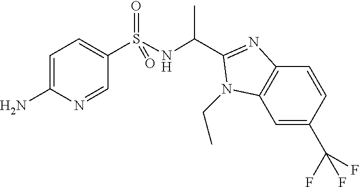

6-Chloro-N-{(1R)-1-[1-ethyl-6-(trifluoromethyl)-1H-benzimidazol-2-yl]ethyl}pyridine-3-sulfonamide (Ex. 35, 0.221 g, 0.00051 mol) and ammonia (1.5 mL, 10.50 mmol; 7N in methanol) were taken in a 5 mL microwave tube. Initially, the reaction was heated to 120° C. and maintained for 2 h, resulting in very little conversion to the desired amine. Heating to 140° C. for another 2 h improved conversion. After heating for 16 h at 160° C., the reaction was cooled, and solvent was removed under reduced pressure. The residue was partitioned between ethyl acetate and water. The organic layer was extracted twice with water, and then with saturated sodium chloride solution. After drying over magnesium sulfate, solvent was removed in vacuo. The residue was purified by flash chromatography using a gradient of 100% dichloromethane to 15% ethanol in dichloromethane; 5% v:v concentrated ammonium hydroxide in the ethanol) to obtain the desired product (0.045 g, 21.4%)

### Example 171

### N-(1-(1-ethyl-6-(trifluoromethyl)-1H-benzo[d]imidazol-2-yl)ethyl)-6-oxo-1,6-dihydropyridine-3-sulfonamide

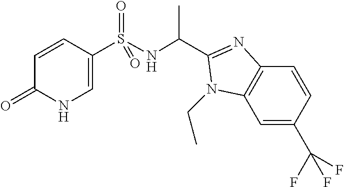

6-Chloro-N-{(1R)-1-[1-ethyl-6-(trifluoromethyl)-1H-benzimidazol-2-yl]ethyl}pyridine-3-sulfonamide (Ex. 35, 0.212 g, 0.00049 mol) was dissolved in dioxane (1.5 mL) in a 5 mL pressure tube. 5N sodium hydroxide solution (1 mL; 0.005 mol) was added in a single portion, and the tube was sealed. After heating at 150° C. for 4 h, LC/MC shows almost no conversion. Upon heating the reaction to 170° C. for another 4 h. some conversion to the desired product had occurred. After heating at 170° C. for another 8 h, most of the starting material was consumed. The suspension was neutralized by the addition of acetic acid and then the volatiles were removed under reduced pressure. The residue was extracted with portions of hot ethanol, filtering off the solids. Solvent was removed under reduced pressure, and the residue was partitioned between ethyl acetate and a small portion of water. The organic layer was washed with water, then saturated sodium chloride solution. After drying over magnesium sulfate, solvent was removed under reduced pressure. The residue was purified flash chromatography using a gradient of 100% dichloromethane to 15% ethanol in dichloromethane; 5% v:v concentrated ammonium hydroxide in the ethanol) to obtain the desired product (15 mg, 7%).

### Examples 173 and 174

SM 2ac′ was converted to Ex. 173 and 174 as described below. The product of step 1 was subjected to step 2 and step 2′ to generate racemates of Ex. 173 and 174 respectively which were further resolved on chiral HPLC using the conditions described for Ex. 97 to obtain Ex. 173 and Ex. 174.

### Step 1

### 2-(6-Cyano-pyridine-3-sulfonylamino)-N-(6-cyclopropyl-4-ethylamino-pyridin-3-yl)-propionamide

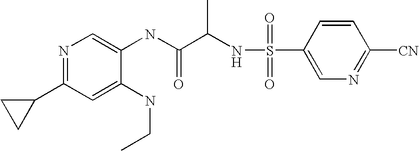

Hydrogen chloride in dixoane (15 mL, 4 M solution) was added to tert-butyl N-[-1-[(6-cyclopropyl-4-ethylamino-pyridin-3-yl)carbamoyl]ethyl]carbamate (SM 2ac′, 420 mg, 1.2 mmol) at room temperature (within 10 minute white solid precipitated). The resulting mixture was allowed to stir at room temperature for 2 h. The reaction mixture was concentrated. The residue was added DCM (20 mL), followed by triethyl amine (1.0 mL, 7.2 mmol) at an ice bath temperature. 6-Cyano-pyridine-3-sulfonyl chloride (290 mg, 1.4 mmol) solution in DCM (5 mL) was added drop wise. The reaction mixture was allowed warm to room temperature for 2 h. The mixture was diluted with ethyl acetate, washed with sat. aq. NaHCO₃ solution, water and brine. The organic extract was dried (Na₂SO₄), filtered, and concentrated. The residue was dissolved in DCM and purified by flash chromatography (gradient of 0-4% MeOH in chloroform) to afford the title compound as a light brown solid (226 mg, 46% yield). ¹H NMR (301 MHz, DMSO-d₆) δ ppm 9.36 (bs, 1H), 9.10 (d, J=2.2 Hz, 1H), 8.85 (d, J=7.4 Hz, 1H), 8.42 (dd, J=8.3, 2.2 Hz, 1H), 8.32 (s, 1H), 8.25 (d, J=8.0 Hz, 1H), 7.71 (s, 1H), 6.53 (s, 1H), 4.14 (t, J=7.0 Hz, 1H), 3.19-3.30 (m, 2H), 1.96-2.08 (m, 1H), 1.31 (d, J=6.9 Hz, 3H), 1.18 (t, J=7.2 Hz, 3H), 0.93-1.02 (m, 4H). M/z 414.

### Step 2

### 6-Cyano-pyridine-3-sulfonic acid [1-(6-cyclopropyl-1-ethyl-1H-imidazo[4,5-c]pyridin-2-yl)-ethyl]-amide

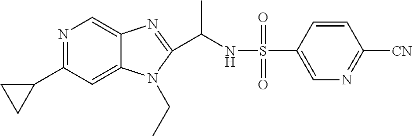

2-(6-Cyano-pyridine-3-sulfonylamino)-N-(6-cyclopropyl-4-ethylamino-pyridin-3 yl)-propionamide (Step 1, 226 mg, 0.55 mmol) was dissolved in THF:MeOH (6:1, 7 mL) and heated in microwave at 120° C. for 15 h (5 h intervals). The reaction mixture was concentrated. The residue was purified by flash chromatography eluting with 5-10% MeOH in chloroform to give the titled product as a white solid (97 mg, 45% yield). ¹H NMR (301 MHz, DMSO-d₆) δ ppm 9.14 (s, 1H), 8.85 (d, J=2.2 Hz, 1H), 8.51 (s, 1H), 8.17 (dd, J=8.3, 2.2 Hz, 1H), 7.95 (d, J=8.0 Hz, 1H), 7.42 (s, 1H), 4.95 (q, J=6.9 Hz, 1H), 4.25 (q, J=7.6 Hz, 2H), 2.03-2.28 (m, 1H), 1.44 (d, J=6.9 Hz, 3H), 1.30 (t, J=7.0 Hz, 3H), 0.89-0.98 (m, 4H). M/z 396. Enatioresolution of the product on a chiral HPLC afforded Ex. 173.

### Step 2′

### 5-[1-(6-Cyclopropyl-1-ethyl-1H-imidazo[4,5-c]pyridin-2-yl)-ethylsulfamoyl]-pyridine-2-carboxylic acid amide

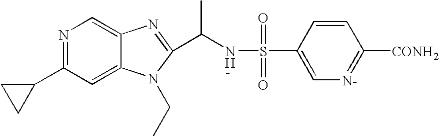

2-(6-Cyano-pyridine-3-sulfonylamino)-N-(6-cyclopropyl-4-ethylamino-pyridin-3-yl)-propionamide (Step 1, 220 mg, 0.54 mmol) was dissolved in MeOH (4 mL) and aqueous ammonia. The mixture was heated in microwave at 120° C. for 1 h (5 h intervals). The reaction mixture was concentrated. The residue was purified by flash chromatography eluting with 5-10% MeOH in chloroform followed by trituration with ether/hexanes to give the titled product as a white solid (146 mg, 68% yield). ¹H NMR (301 MHz, DMSO-d₆) δ ppm 8.94 (br. s., 1H), 8.86 (d, J=2.2 Hz, 1H), 8.56 (s, 1H), 8.22 (dd, J=8.3, 2.2 Hz, 1H), 8.14 (s, 1H), 8.03 (d, J=8.3 Hz, 1H), 7.82 (s, 1H), 7.43 (s, 1H), 4.88-5.06 (m, 1H), 4.19-4.38 (m, 2H), 2.06-2.23 (m, 1H), 1.37 (d, J=6.9 Hz, 3H), 1.31 (t, J=7.2 Hz, 3H), 0.88-0.94 (m, 4H). M/z 414.

Enatioresolution of the product on a chiral SFC (Methanol/CO₂) afforded Ex. 174.

### Example 178

### 5-({[1-(1-Ethyl-1H-imidazo[4,5-b]pyridin-2-yl)-1-methylethyl]amino}sulfonyl)pyridine-2-carboxamide

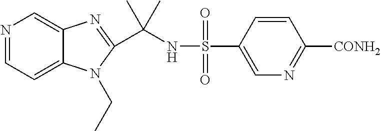

6-cyano-N-(2-(1-ethyl-1H-imidazo[4,5-b]pyridin-2-yl)propan-2-yl)pyridine-3-sulfonamide (Ex. 177, 142 mg, 0.38 mmols) was taken in a round bottom flask and 4M HCl in dioxane (10 mL) was added to it. The resultant was stirred at room temperature and was found to be converting to the desired product only slowly when an additional 4M HCL in dioxane (10 mL) was added and the resultant was stirred over the weekend. After a total of 4 days of stirring at room temperature, the reaction mixture was concentrated and the resultant was carefully neutralized and the product extracted into the ethyl acetate layer (3×30 mL). The organics were dried over anhydrous sodium sulfate, filtered and concentrated. The resultant material was purified using a gradient for 50% ethyl acetate in hexanes to 15% methanol in ethyl acetate to obtain the desired product (26 mg, 17.46%).

### Example 187

### (R)—N-(1-(1-Ethyl-6-(trifluoromethyl)-1H-benzo[d]imidazol-2-yl)ethyl)-1-methyl-1H-pyrrole-2-sulfonamide

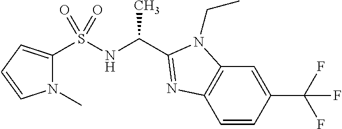

### Step 1

### Lithium 1-methyl-1H-pyrrole-2-sulfinate

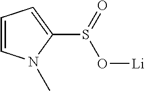

An oven-dried 100 mL round bottom flask was evacuated while hot and was allowed to cool under N₂. The flask was charged with 1-methyl-1H-pyrrole (1.00 mL, 11.3 mmol) and anhydrous THF (12 mL), and the resulting solution was cooled to −78° C. n-BuLi (2.5 M in hexane; 5.0 mL, 12.5 mmol) was added dropwise, and the resulting mixture was allowed to stir at −78° C. for 5 minutes and was then allowed to warm to room temperature. After stirring at room temperature overnight, the mixture was cooled back to −78° C., and SO₂ (excess) was introduced. The resulting mixture was allowed to stir at −78° C. for 5 minutes and was then allowed to warm to room temperature. After 5 hours at room temperature, the volatile components were evaporated under reduced pressure. The residue was triturated with ether and was then dried in vacuo to give a tan solid. 1.76 g of material was collected. This was used directly without any further purification.

### Step 2

### (R)—N-(1-(1-Ethyl-6-(trifluoromethyl)-1H-benzo[d]imidazol-2-yl)ethyl)-1-methyl-1H-pyrrole-2-sulfonamide

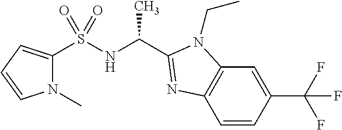

A 50 mL round bottom flask was charged with (R)-tert-butyl 1-(1-ethyl-6-(trifluoromethyl)-1H-benzo[d]imidazol-2-yl)ethylcarbamate (Boc protected Int 9, 236 mg, 0.66 mmol) and 4N HCl/dioxane (3 mL). The resulting solution was allowed to stir at room temperature for 1 hour and was then concentrated under reduced pressure. The resulting residue was dissolved in CH₂Cl₂ (3 mL) and NEt₃ (500 μL, 3.6 mmol). Meanwhile, a separate 25 mL round bottom flask was charged with lithium 1-methyl-1H-pyrrole-2-sulfinate (189 mg, 1.25 mmol), CH₂Cl₂ (3 mL), and H₂O (3 mL). This biphasic mixture was cooled to 0° C. with vigorous stirring, and then N-chlorosuccinimide (164 mg, 1.23 mmol) was added. The mixture was allowed to warm to room temperature with vigorous stirring. After 30 minutes, the reaction was transferred to a separatory funnel, and the organic layer was drained into the flask containing the deprotected amine. This mixture was allowed to stir at room temperature. After stirring at room temperature overnight, the mixture was partitioned between CH₂Cl₂ and H₂O, and the aqueous layer was extracted with CH₂Cl₂. The combined organics were dried (MgSO₄), filtered, and concentrated. The crude material was purified by silica gel chromatography (gradient elution; R_{f} in 60:40 hexanes:EtOAc=0.19) to give a pale yellow oil. The product was precipitated from MeOH/H₂O to give a colorless solid (25 mg, 9%). M/Z=400. ¹H NMR (400 MHz, DMSO-D6) δ ppm 1.27-1.36 (m, 6H) 3.77 (s, 3H) 4.28-4.34 (m, 1H) 4.36-4.42 (m, 1H) 4.72 (m, 1H) 5.94 (m, 1H) 6.62 (m, 1H) 6.93 (m, 1H) 7.48 (m, 1H) 7.76 (m, 1H) 8.00 (m, 1H) 8.42 (m, 1H).

### 5-({[1-(1-ethyl-1H-imidazo[4,5-b]pyridin-2-yl)-1-methylethyl]amino}sulfonyl)pyridine-2-carboxamide

### Intermediate 1

### [(1R)-1-(1-ethyl-1H-benzimidazol-2-yl)ethyl]amine

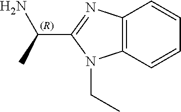

N-Ethyl-1,2-phenylenediamine (Starting Material (SM) 1 h, 14.7 mmol) and D-Alanine (2.2 g, 22.0 mmol) were taken into 6N HCl (15.0 mL) and the mixture was refluxed for 6 d. The reaction mixture was cooled in ice-bath, basified using 2N NaOH and extracted with EtOAc (3×50 mL). The organic layer was washed with brine (10 mL), dried and concentrated in vacuo to give a dark brown viscous glue, which was purified by flash column chromatography using silica gel and CHCl₃/MeOH (95:5) as eluent to give Intermediate 1 as a brown oil in 81% yield. ¹H NMR (300 MHz, CDCl₃) δ 7.78-7.72 (m, 1H), 7.37-7.21 (m, 3H), 4.36-4.17 (m, 3H), 1.81 (br s, 2H), 1.61 (d, J=6.9 Hz, 3H), 1.45 (t, J=7.1 Hz, 3H). M/Z=189 Starting Material 1 h

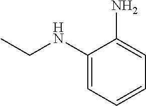

### N-Ethyl-1,2-phenylenediamine

To a solution of 1-ethyl-2-nitroaniline (5.0 g, 30.0 mmol) in EtOH (100 mL) was added 10% Pd on carbon (1.24 g). The mixture was reacted in a Parr apparatus under 50 psi of H₂ gas for 2 h. The mixture was filtered through diatomaceous earth. The diatomaceous earth was washed with EtOAc, and the combined organic solvents were concentrated in vacuo to give the product as brown oil (4.0 g, 100% yield), which was used directly in the next step. ¹H NMR (300 MHz, CDCl₃) δ 6.72-6.68 (m, 4H), 3.19-3.12 (m, 5H), 1.28 (t, J=7.1 Hz, 3H). M/Z 136.

Intermediates 2-7 shown in Table 2 were prepared in an analogous manner to Intermediate 1, using the appropriate commercially available amino acid.

TABLE 2

| INT | Compound | NMR | M/Z |
|---|---|---|---|
| 2 | 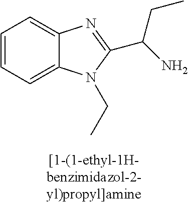 | ¹H NMR (300 MHz, CDCl₃) δ 7.73(d, J = 4.4 Hz, 1 H), 7.26-7.23(m, 3 H), 4.28-4.05(m, 3 H), 1.90-1.42(m, 7 H), 0.99(t, J = 7.1 Hz, 3 H) | 203 |
| 3 | 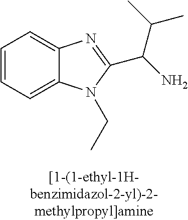 | ¹H NMR (300 MHz, CDCl₃) δ 7.75-7.73(m, 1 H), 7.27-7.23(m, 3 H), 4.26(q, J = 7.1 Hz, 2 H), 3.83(d, J = 3.8 Hz, 1 H), 2.21-2.16(m, 1 H), 1.80(br s, 2 H), 1.44(t, J = 7.4 Hz, 3 H), 1.08(d, J = 6.9 Hz, 3 H), 0.93(d, J = 6.9 Hz, 3 H). | 217 |
| 4 | 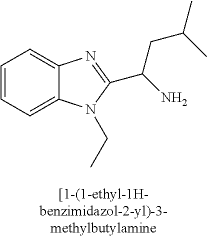 | ¹H NMR (300 MHz, CDCl₃) δ 7.75(d, J = 4.9 Hz, 1 H), 7.26-7.23(m, 3 H), 4.29-4.26(m, 3 H), 1.81-1.70(m, 5 H), 1.48(t, J = 7.1 Hz, 3 H), 0.98-0.97(m, 6 H) | 231 |
| 5 | 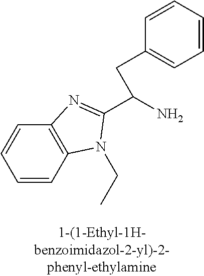 | ¹H NMR (300 MHz, CDCl₃) δ 7.81-7.75(m, 1 H), 7.32-7.12(m, 8 H), 4.34-4.29(m 1 H), 4.15-3.92(m, 2 H), 3.35(dd, J = 13.2, 6.6 Hz, 1 H), 3.19(dd,J = 13.2, 8.0 Hz, 1 H), 2.16(br s, 2 H), 1.24(t, J = 7.1 Hz, 3 H). | 265 |
| 6 | 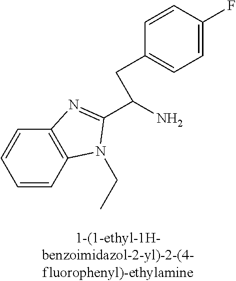 | ¹H NMR (300 MHz, CDCl₃) δ 7.77-7.75(m, 1 H), 7.29-6.92(m, 7 H), 4.26-4.00(m, 3 H), 3.31-3.17(m, 2 H), 1.79(br s, 2 H), 1.23(t, J = 7.1 Hz, 3 H). | 283 |
| 7 | 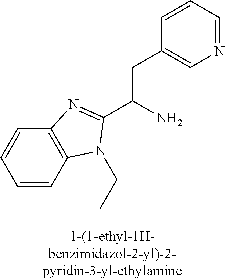 | ¹H NMR (300 MHz, CDCl₃) δ 8.72-8.43(m, 2 H), 7.74-6.91(m, 6 H), 4.26-4.00(m, 3 H), 3.34-3.18(m, 2 H), 1.92(br s, 2 H), 1.22(t, J = 7.1 Hz, 3 H). | 266 |

### Intermediate 8

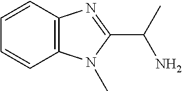

### 1-(1-Methyl-1H-benzimidazol-2-yl)ethylamine

Intermediate 8 was prepared in an analogous manner as that used for preparing Intermediate 1 but using N-Methyl-1,2-phenylenediamine (Starting Material Ii) and BOC-Ala-OH. ¹H NMR (300 MHz, CDCl₃) 1.57 (d, 3H) 3.79 (s, 3H) 4.07-4.52 (m, 1H) 4.81 (s, 2H) 7.10-7.69 (m, 3H) 7.57-7.98 (m, 1H). M/Z 176.

### Starting Material 1i

### N-Methyl-1,2-phenylenediamine

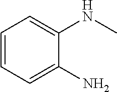

N-methyl-2-nitroaniline (3.0 g; 0.05 mol) was dissolved in ca. 120 mL ethanol to a clear, yellow solution. Cyclohexene (40 mL; 0.4 mol) and 10% palladium-on-carbon (2.65 g; 5 mol %) were sequentially added as single portions. The resulting suspension was heated to reflux and maintained for 16 h. The reaction mixture was filtered hot through a pad of diatomaceous earth and the filter cake washed with a few portions of hot ethanol. The filtrate was concentrated under reduced pressure to yield the product as a red-brown oil, which was used directly in the subsequent step. ¹H NMR (300 MHz, CDCl₃): δ 2.84 (s, 3H) 3.32 (s, 3H) 6.49-6.82 (m, 3H) 6.78-7.01 (m, 1H) M/Z=123.

Intermediates 9-16 and 25-52 were generated as indicated generally in scheme 2, above, by reacting the appropriate amide Starting Material 2 (SM 2) with the appropriate cyclization agent by one of four methods: (1: AcOH; 2: Lawesson's Reagent, 3: 4M HCl/Dioxane or 4: Sodium dithionate/aldehyde.

### Method 1: AcOH

### Intermediate 11:

### Step I

### tert-butyl[(1R)-1-(1-ethyl-7-methoxy-1H-benzimidazol-2-yl)ethyl]carbamate

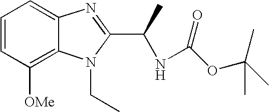

tert-butyl((1R)-2-{[2-(ethylamino)-3-methoxyphenyl]amino}-1-methyl-2-oxoethyl) carbamate (SM 2c) (2.75 mmols) obtained from the previous step was dissolved in AcOH (6 mL) and heated to 65° C. for 2 h. The reaction mixture was concentrated under reduced pressure and purified by flash chromatography (30% ethyl acetate in hexanes) to afford the desired product as a white powder (180 mg, 22%). The title product was carried on to the next step after LC-MS characterization.

### Step II

### [(1R)-1-(1-ethyl-7-methoxy-1H-benzimidazol-2-yl)ethyl]amine hydrochloride (Intermediate 11)

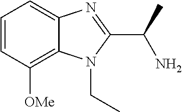

tert-butyl[(1R)-1-(1-ethyl-7-methoxy-1H-benzimidazol-2-yl)ethyl]carbamate (0.18 g, 0.56 mmol) and 4M HCl/dioxane (3 mL) were taken in a round bottom flask equipped with a stir bar and a rubber septum. The reaction mixture was stirred at room temperature for 40 minutes then concentrated under reduced pressure and dried in vacuo to afford 0.148 g of title compound. M/Z 219.

### Method 2: Lawesson's Reagent

### Intermediate 15:

### Step I

### tert-butyl (2-{[2-(ethylamino)pyridin-3-yl]amino}-1-methyl-2-oxoethyl)carbamate (SM 2g)

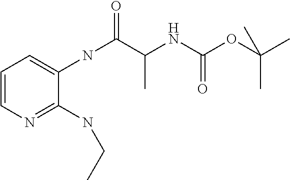

Boc-Ala-OH (0.367 g, 1.93 mmols) was placed in a round bottom flask equipped with a stir bar and DCM (2 mL) was added. To the resulting homogeneous solution, DIEA (0.34 mL, 1.93 mmols) and PYBOP (1.0 g, 1.93 mmols) were added. The resultant mixture was stirred for 15 minutes and then added slowly to another round bottom flask containing N²-ethylpyridine-2,3-diamine (SM Ig) (0.24 g, 1.75 mmols) and DCM (2 mL). The resultant mixture was stirred at room temperature overnight. The reaction mixture was concentrated to a thick syrup. A solution was reconstituted using ethyl acetate and washed with water followed by brine. The organic layer was dried over sodium sulfate (anhydrous), filtered and concentrated to a solid. The solid crude product thus obtained was purified via column chromatography using a gradient of 10% ethyl acetate in hexanes to 100% ethyl acetate followed by 5% methanol in ethyl acetate to obtain the desired product in the form of an off-white powder (0.305 mg, 60%), ¹H NMR (MeOH-d₄): δ 7.96 (d, 1H), 7.84 (bs, 1H), 7.54 (d, 1H), 6.60 (dd, 1H), 5.04 (q, 1H), 3.42 (q, 2H), 1.50 (s, 9H), 1.48 (d, 3H), 1.28 (t, 3H). M/Z 308

### Step II

### [1-(3-ethyl-3H-imidazo[4,5-b]pyridin-2-yl)ethyl]amine dihydrochloride (Intermediate 15)

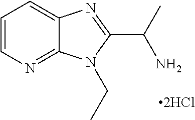

tert-butyl (2-{[2-(ethylamino)pyridin-3-yl]amino}-1-methyl-2-oxoethyl)carbamate (0.168 g, 0.54 mmols), dioxane (3 mL) and Lawesson's reagent (0.109 g, 0.27 mmols) were placed in a microwave tube equipped with a stir bar and the resultant mixture was heated in a microwave at 150° C. for 2 h. This resulting mixture was taken in a round bottom flask, concentrated to a dark brown solid which was then reconstituted in dioxane, filtered, concentrated and subject to the deprotection of the BOC group using 4M HCl/dioxane (5 mL). The crude reaction mixture was stirred at room temperature for 2 h and concentrated under reduced pressure to yield the amine hydrochloride which was used without further purification. M/Z 190.

### Method 3: 4M HCl/dioxane

### Intermediate 16

### [1-(1-ethyl-1H-benzimidazol-2-yl)-1-methylethyl]amine hydrochloride

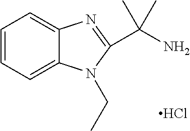

[1-(2-Ethylamino-phenylcarbamoyl)-1-methylethyl]-carbamic acid tert-butyl ester (SM 2h) (0.600 g, 1.86 mmol) was refluxed in 4 M HCl/dioxane (5.0 mL) overnight. The solvent was evaporated to obtain the title compound (0.576 g), which was used in the next step without further purification. For NMR analysis, a small amount (0.020 g) of the crude product was basified using 2N NaOH and extracted with EtOAc. The organic layer was concentrated in vacuo to give the product as a free base.

¹H NMR (free base) (300 MHz, CDCl₃) δ 7.76-7.73 (m, 1H), 7.36-7.22 (m, 3H), 4.66 (q, J=7.1 Hz, 2H), 3.50-3.48 (m, 2H), 1.70 (br s, 3H), 1.57 (br s, 3H), 1.48 (t, J=7.1 Hz, 3H). M/Z 203.

Method 3′: 2M HCl in Ethanol and 4M HCl in dioxane

### Intermediate 59

### 1-(1-Ethyl-1H-imidazo[4,5-b]pyridin-2-yl)-1-methyl-ethylamine; hydrochloride

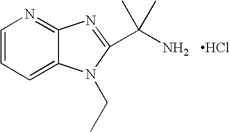

The [1-(3-ethylamino-pyridin-2-ylcarbamoyl)-1-methyl-ethyl]-carbamic acid tert-butyl ester (SM2ai, 1.93 g, 6 mmol) was suspended in 4M HCl in dioxane (50 mL) and 2.5 M HCl in ethanol (15 mL) under N₂ atm and refluxed for 16 h. The solvent was evaporated and the resulting solid triturated with ether, filtered and dried under vacuum to obtain the desired product (1.4 g, 99%).

Method 4: Sodium Dithionate, Aldehyde

### Intermediate 30

### (R)-1-(1-Ethyl-5,6-dimethoxy-1H-benzoimidazol-2-yl)-ethylamine

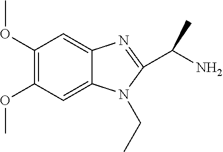

### Step 1

### (R)-[1-(1-Ethyl-5,6-dimethoxy-1H-benzoimidazol-2-yl)-ethyl]-carbamic acid tert-butyl ester

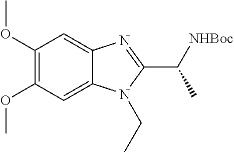

A solution or suspension of (4,5-Dimethoxy-2-nitro-phenyl)ethylamine (0.23 g, 1 mmol) and Boc-D-Ala-CHO (SM 1ae, 0.17 g, 1 mmol) in EtOH (4 mL) was treated with freshly prepared 1M aq. Na₂S₂O₄ (3 mmol, 3 mL). After heating the reaction mixture at 70° C. for 5-12 h, it was cooled to rt and treated dropwise with 5N aq NH₄OH. The resulting residue was extracted twice with EtOAc. The combined organic layers were washed with brine and dried over anhydrous MgSO₄ and concentrated. The resulting product was then purified by flash chromatography on silica gel using a 35-80% EtOAc/hexane as eluent. Yield: 0.37 g (60%). ¹H NMR (300 MHz, DMSO-d₆) δ: 7.42 (d, J=8.5 Hz, 1H), 7.14 (s, 1H), 7.10 (s, 1H), 4.97 (m, 1H), 4.22 (m, 2H), 3.81 (s, 3H), 3.76 (s, 3H), 1.48 (d, J=6.9 Hz, 3H), 1.38 (s, 9H). (M/Z=349.

### Step 2

### (R)-1-(1-Ethyl-5,6-dimethoxy-1H-benzoimidazol-2-yl)-ethylamine (Intermediate 30)

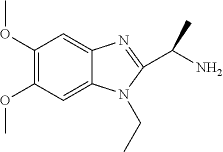

The reaction of (R)-[1-(1-Ethyl-5,6-dimethoxy-1H-benzoimidazol-2-yl)-ethyl]carbamic acid tert-butyl ester (Step 1, 0.34 g, 0.58 mmol) with 4M HCl in dioxane (5 mL, 20.0 mmol) gave the product as hydrochloride salt. Yield: 0.28 g. M/Z 249. When Step 2 was carried out using TFA/DCM (1:1, v/v) in place of 4M HCl/dioxane, the corresponding trifluoroacetate salt was obtained (as in the case of Intermediate 32)

Method 5: 4N HCl/Dioxane and Sodium hydroxide, ethanol

### Intermediate 53

### (R)-1-(1-ethyl-6-methoxy-1H-imidazo[4,5-c]pyridin-2-yl)ethanamine

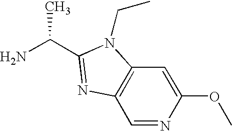

A 100 mL round bottom flask containing crude (R)-tert-butyl 1-(4-(ethylamino)-6-methoxypyridin-3-ylamino)-1-oxopropan-2-ylcarbamate 2.13 mmol maximum, (SM 2ad′, 0.719 g) was charged with 4N HCl/dioxane (6 mL), and the mixture was allowed to stir at room temperature. After 2 hours, the dioxane and excess HCl were evaporated under reduced pressure. The residue was dissolved in absolute EtOH (8 mL), and solid NaOH (406 mg, 10.2 mmol) was added. The mixture was placed in an 80° C. oil bath. After 90 minutes, an additional 440 mg NaOH (21.2 mmol total) was added, and heating was continued. After another 3 hours, the reaction was allowed to cool. The mixture was concentrated under reduced pressure, and the residue was partitioned between CH₂Cl₂ and H₂O. The aqueous layer was extracted with CH₂Cl₂, and the combined organics were washed with brine, dried (MgSO₄), filtered, and concentrated to a dark red oil. This material was used without further purification.

Intermediate 60 was prepared from starting material 2ac′ by method described below. Application of method 5′ described below to starting material 2a yielded intermediate 61.

### Method 5′ (Microwave Conditions)

### Intermediate 60

### 1-(6-Cyclopropyl-1-ethyl-1H-imidazo[4,5-c]pyridin-2-yl)-1-methyl-ethylamine

To [1-(6-Cyclopropyl-4-ethylamino-pyridin-3-ylcarbamoyl)-ethyl]-carbamic acid tert-butyl ester (SM 2ac′, 696 mg, 2.0 mmol) was added 4M HCl in dioxane (20 mL) under N₂ atm. It was allowed to stir at room temperature for 2 h. The solvent was removed by evaporation and the residue dried under vacuo. The residue was dissolved in 10% aq. NaOH (4 mL) and EtOH (10 mL). The mixture was heated at 80° C. for 2 h in a microwave reactor. The reaction mixture was concentrated. The residue was partitioned between water/chloroform, and extracted with chloroform. The organic layer was dried (Na₂SO₄), filtered and concentrated to obtain 1-(6-Cyclopropyl-1-ethyl-1H-imidazo[4,5-c]pyridin-2-yl)-ethylamine as a light brown oil. Intermediates 9, 10 and 12-14 were prepared by one of the three methods described above for Intermediates 11, 15 and 16 using the specific amide Starting Material (SM2) indicated in Table 3.

TABLE 3

| INT | Structure and Name | 1H NMR | M/Z | SM2 | Method |
|---|---|---|---|---|---|
| 9 | 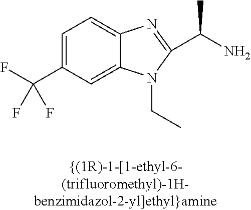 | — | 256 | 2a | 1 |
| 9a | 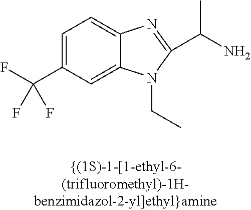 | — | 256 | 2a′ | 1 |
| 9b | 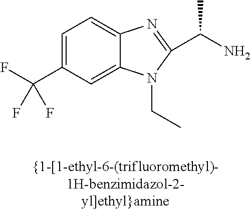 | — | 256 | 2a″ | 1 |
| 10 | 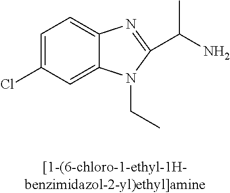 | — | 323 | 2b | 1 |
| 12 | 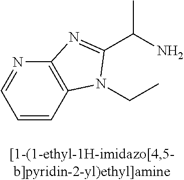 | — | 190 | 2d | 1 |
| 13 | 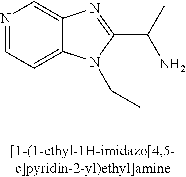 | — | 190 | 2e | 2 |
| 14 | 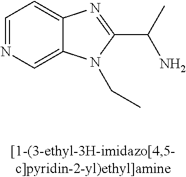 | ¹H(NMR (MeOH-d₄) δ 9.53(s, 1 H), 8.55 (d, 1 H), 8.28(d, 1 H), 5.10(q, 1 H), 4.65(q, 2 H), 1.70(d, 3 H), 1.52(t, 3 H) | 190 | 2f | 1 |
| 25 | 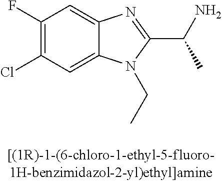 | — | 242 | 2i | 3 |
| 26 | 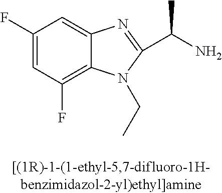 | — | 225 | 2j | 3 |
| 27 | 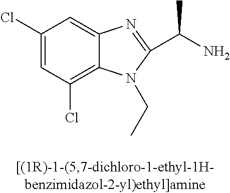 | — | 258 | 2k | 3 |
| 28 | 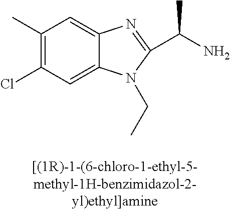 | — | 238 | 2l | 3 |
| 29 | 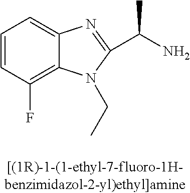 | — | 207 | 2m | 3 |
| 30 | 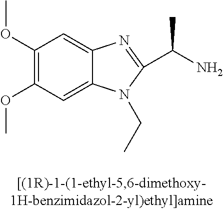 | ¹H NMR (300 MHz, DMSO-d₆) δ: 8.89 (bs, 2 H), 7.36(s, 1 H), 7.22(s, 1 H), 4.95(bs, 1 H), 4.40 (m, 2 H), 3.87(s, 3 H), 3.82(s, 3 H), 1.66(d, J = 6.9 Hz, 3 H), 1.34 (t, J = 6.9 Hz, 3 H) | 249 | — | 4 |
| 31 | 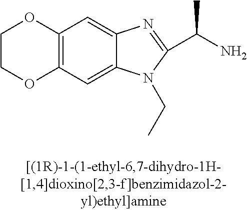 | — | 248 | — | 4 |
| 32 | 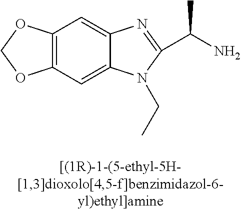 | ¹H NMR(300 MHz, DMSO-d₆) δ: 8.52 (brs, 3 H), 7.29(s, 1 H), 7.17(s, 1 H), 6.03(s, 2 H), 4.32- 4.16(m, 3 H), 1.53 (d, J = 6.87 Hz, 3 H), 1.29(t, J = 6.87 Hz, 3 H) | 233 | — | 4 |
| 33 | 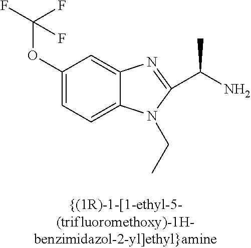 | — | 273 | 2n | 3 |
| 34 | 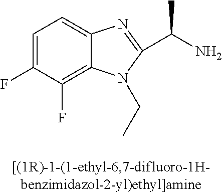 | — | 225 | 2o | 3 |
| 35 | 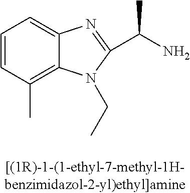 | — | 203 | 2p | 3 |
| 36 | 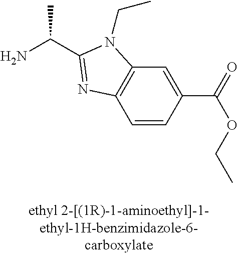 | — | 261 | 2q | 1 |
| 37 | 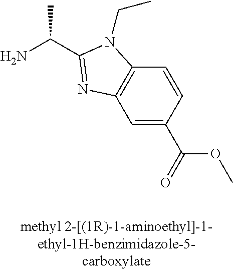 | — | 247 | 2r | 1 |
| 38 | 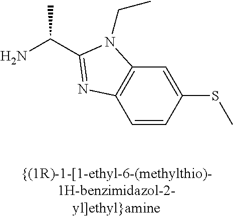 | — | 235 | 2s | 1 |
| 39 | 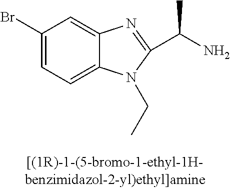 | — | 268 | 2t | 1 |
| 40 | 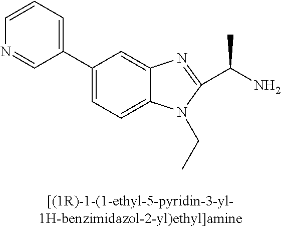 | — | 266 | 2t | 1 |
| 41 | 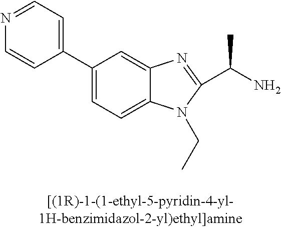 | — | 266 | 2t | 1 |
| 42 | 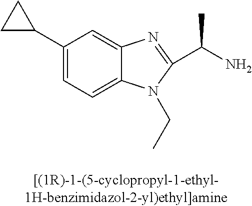 | — | 229 | 2t | 1 |
| 43 | 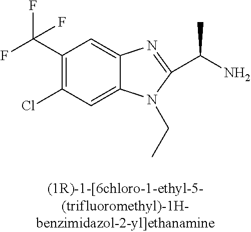 | ¹H NMR (300 MHz, DMSO-D₆) δ ppm 1.35(t, 3 H) 1.59(d, 3 H) 4.43(m ,2 H) 4.93(m, 1 H) 8.03(s, .5 H) 8.12(s, .5 H) 8.21(s, .5 H) 8.30(s, .5 H) 8.87(s, 3 H) | 292 | 2u | 1 |
| 44 | 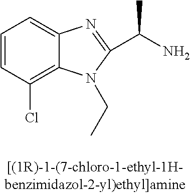 | ¹H NMR (300 MHz, DMSO-d₆) δ: 8.81 (brs, 3 H), 7.68-7.65 (m, 1 H), 7.37-7.24 (m, 2 H), 4.94-4.84 (m, 1 H), 4.68-4.42 (m, 2 H), 1.59(d, J = 6.87 Hz, 3 H), 1.40(t, J = 6.87 Hz, 3 H). | 223 | — | 4 |
| 45 | 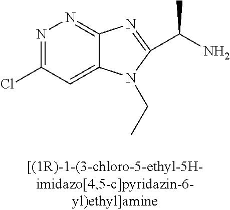 | — | 226 | 2v | 1 |
| 46 | 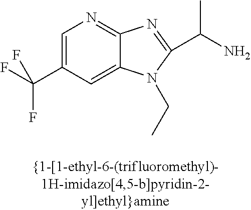 | — | 258 | 2w | 1 |
| 46′ | 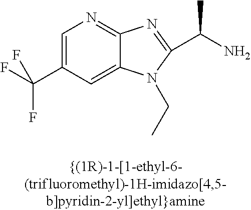 | — | 258 | 2w′ | 1 |
| 47 | 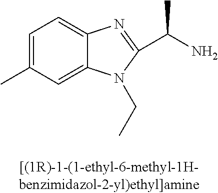 | ¹H NMR (300 MHz, DMSO-D₆) δ ppm 1.37(q, 3 H) 1.63(d, 3 H) 2.47(s, 3 H) 4.36(m, 2 H) 4.94 (m, 1 H) 7.18(d, 1 H) 7.55(s, 1 H) 7.60 (d, 1 H) 8.34(s, 2 H) 8.89(s, 3 H) | 203 | 2x | 1 |
| 48 | 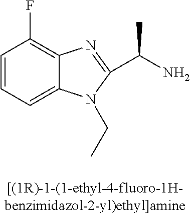 | — | 207 | 2y | 1 |
| 49 | 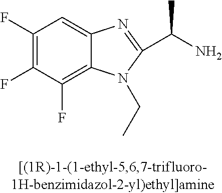 | ¹H NMR (300 MHz, DMSO-d₆) δ: 8.83 (brs, 3 H), 7.69-7.66 (m, 1 H), 4.96-4.86 (m, 1 H), 4.48-4.27 (m, 2 H), 1.57(d, J = 6.87 Hz, 3 H) ,1.39(t, J = 6.87 Hz, 3 H). | 243 | — | 4 |
| 50 | 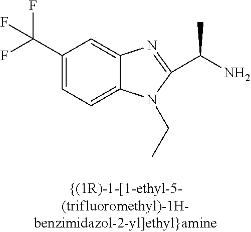 | — | 257 | 2z | 1 |
| 51 | 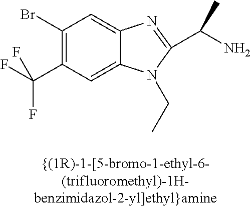 | — | 336 | 2aa | 1 |
| 52 | 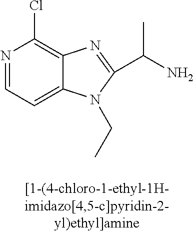 | — | 225 | — | 1 |
| 53 | 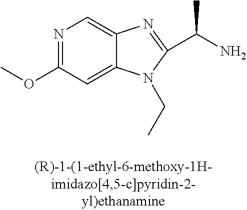 | — | 220 | 2ad′ | 5 |
| 54 | 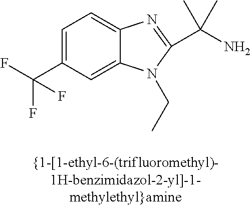 | ¹H NMR (300 MHz, DMSO-d₆) δ: 9.00 (brs, 3 H), 8.13(s, 1 H), 7.88(d, J = 8.26 Hz, 1 H), 7.60(d, J = 9.91 Hz, 1 H), 4.56 (q, J = 6.88 Hz, 2 H), 1.82(s, 6 H), 1.36(t, J = 6.88 Hz, 3 H). | 271 | 2ae | 1, 3 |
| 55 | 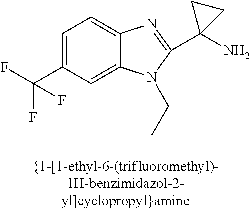 | — | 269 | 2af | 1 |
| 56 | 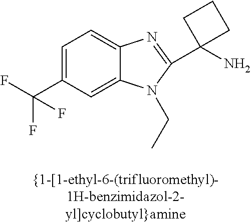 | ¹H NMR (300 MHz, DMSO-d₆) δ: 9.33 (brs, 3 H), 8.16(s, 1 H), 7.91(d, J = 8.26 Hz, 1 H), 7.60(d, J = 7.43 Hz, 1 H), 4.46 (q, J = 6.88 Hz, 2 H), 3.05-2.95(m, 2 H), 2.79-2.69(m, 2 H), 2.43-2.28(m ,1 H), 1.99-1.89(m, 1 H), 1.40(t, J = 6.88 Hz, 3 H) | 283 | 2ag | 1, 3 |
| 57 | 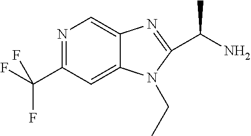 | — | 258 | 2ah | 5 |
| 58 | 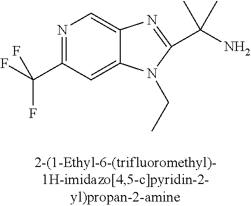 | ¹H NMR (400 MHz, DMSO-D6) δ ppm 1.38(t, J = 7.07 Hz, 3 H) .159(s, 6 H) 2.27 (broad s, 2 H) 4.84 (q, J = 7.07 Hz, 2 H) 8.16(s, 1 H) 8.98(s, 1 H). | 272 | 2ai | 5 |
| 59 | 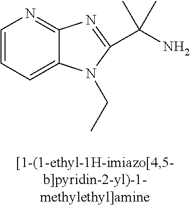 | ¹H NMR (300 MHz, DMSO-d₆) δ: 9.09 (br s., 3 H), 8.59(d, J = 5.23 Hz, 1 H), 8.44 (d, J = 7.15 Hz, 1 H), 7.56-7.51(m, 1 H), 4.56(q, J = 7.15 Hz, 2 H), 1.84(s, 6 H), 1.39(t, J = 6.87 Hz, 3 H). | 204 | 2aj | 3′ |
| 60 | 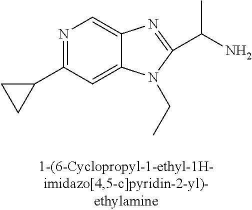 | — | 230 | | 5′ |
| 61 | 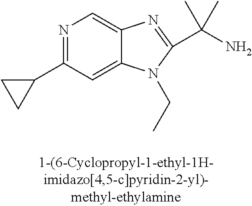 | — | 244 | | 5′ |

Intermediates 17-23 were generated by a method outlined below for Intermediate 17.

### Intermediate 17

### [1-(1-ethyl-1H-benzimidazol-2-yl)propyl]amine hydrochloride

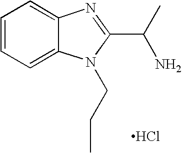

A 25 ml round bottom flask was charged with above tert-butyl [1-(1H-benzimidazol-2-yl)ethyl]carbamate (Starting Material 3; 0.045 g, 0.17 mmol) and THF (5 mL). The solution was treated with cesium carbonate (0.25 g, 0.75 mmol) and n-propyl iodide (18 μL, 0.19 mmol), and allowed to stir at room temperature overnight. The volatile components were evaporated under reduced pressure. The resulting residue was purified by silica gel chromatography (EtOAc/Hexane 40:60) to afford 40 mg of desired product (77.65%). This was subsequently dissolved in 4N HCl/dioxane (1.5 mL) and the solution was allowed to stir for 1.5 hours. Evaporation of the solvent followed by drying under high vacuum yielded the title compound in quantitative yield.

### Preparation of Starting Material 3

### tert-butyl[1-(1H-benzimidazol-2-yl)ethyl]carbamate

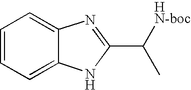

A solution of Boc-Ala-OH (1.56 g, 8.24 mmol) and 4-methylmorpholine (0.91 ml, 8.24 mmol) in DMF (15 ml) was treated at −20° C. with isobutyl chloroformate (1.08 ml, 8.24 mmol). After 10 min at −20° C., o-phenylendiamine (0.89 g, 8.24 mmol) was added. The reaction mixture was allowed to stir while slowly warming to room temperature (1 h) and was then stirred for 3 h. The solvent was evaporated, and the residue was partitioned between EtOAc and H₂O. The EtOAc layer was washed with 5% NaHCO₃ and brine and dried. The solution was filtered, the solvent was evaporated, and the residue was dissolved in glacial AcOH (15 ml). The solution was heated at 65° C. for 1 h. The solvent was evaporated and the residue was purified by silica gel chromatography (EtOAc/Hexane 50:50) to afford a pale white solid (750 mg, 38%). ¹H NMR (300 MHz, DMSO-d₆) δ 1.40 (s, 9H), 1.47 (d, 3H), 3.17 (d, 1H), 4.86 (m, 1H), 7.13 (m, 2H), 7.49 (m, 2H). M/Z=261.

Intermediates 18-20, shown in Table 4, were prepared in a similar manner to that of Intermediate 17 using Starting Material 3 for Intermediates 18 and 19 and Starting Material 4 for Intermediate 20 and the appropriate commercially available alkyl halide.

### Preparation of Starting Material 4 (SM4)

### tert-butyl[(1R)-1-(5,6-difluoro-1H-benzimidazol-2-yl)ethyl]carbamate

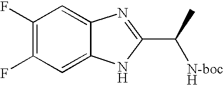

was prepared in an analogous manner to Starting Material 3 except that 1,2-diamine-4,5-difluorbenzene was used in place of the o-phenylendiamine to obtain SM4 as a white solid (107 mg). ¹H NMR (300 MHz, CDCl₃): δ1.47 (s, 9H), 1.76 (t, 3H), 5.01 (m, 1H), 5.36 (d, 1H), 7.36-7.41 (t, 2H). M/Z=297.

TABLE 4

| INT | Compound | M/Z |
|---|---|---|
| 18 | 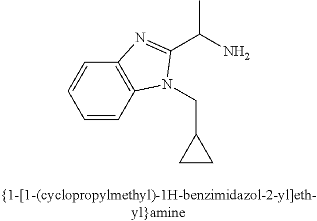 | 215 |
| 19 | 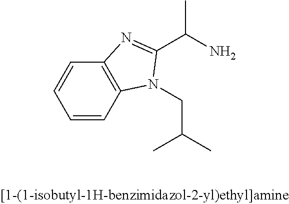 | 217 |
| 20 | 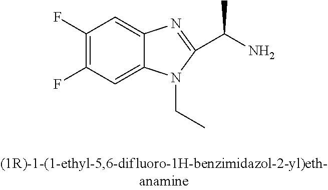 | 225 |
| 22 | 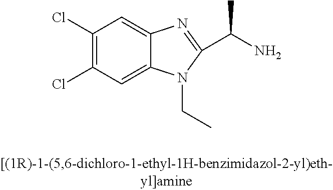 | 257 |
| 23 | 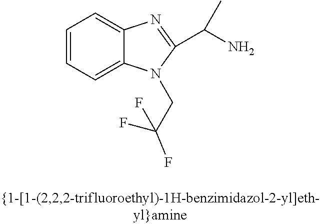 | 243 |
| 24 | 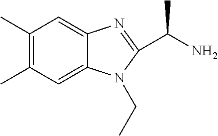 | 217 |

### Intermediate 21

### [1-(1-cyclopropyl-1H-benzimidazol-2-yl)ethyl]amine

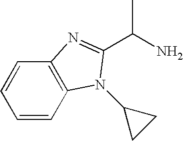

### Step I

### tert-butyl[2-([(1E)-1-[(1E)-1-aminoprop-1-en-1-yl]buta-1,3-dien-1-yl]amino)-1-methyl-2-oxoethyl]carbamate

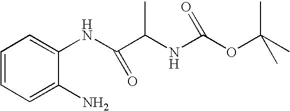

A solution of Boc-Ala-OH (1.56 g, 8.24 mmol) and 4-methylmorpholine (0.91 ml, 8.24 mmol) in N,N-dimethylformamide (DMF, 15 ml) was treated at −20° C. with isobutyl chloroformate (1.08 ml, 8.24 mmol). After 10 min at −20° C., o-phenylendiamine (0.89 g, 8.24 mmol) was added. The reaction mixture was allowed to stir while slowly warming to room temperature (1 h) and was then stirred for 3 h. The solvent was evaporated, and the residue was partitioned between ethyl acetate and H₂O. The EtOAc layer was washed with 5% NaHCO₃ and brine and dried over Na₂SO₄. The solution was filtered, the solvent was evaporated, and the residue was recrystallized from EtOAc to afford tert-butyl[2-({(1E)-1-[(1E)-1-aminoprop-1-en-1-yl]buta-1,3-dien-1-yl}amino)-1-methyl-2-oxoethyl]carbamate which was carried on to the next step without further purification. M/Z 279.

### Step II

### tert-butyl (2-{[2-(cyclopropylamino)phenyl]amino}-1-methyl-2-oxoethyl)carbamate

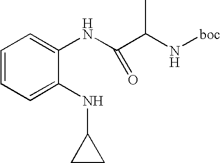

The amine from Step I, i.e. (tert-butyl[2-({(1E)-1-[(1E)-1-aminoprop-1-en-1-yl]buta-1,3-dien-1-yl}amino)-1-methyl-2-oxoethyl]carbamate, 2.07 g, 7.42 mmol), AcOH ((1.20 ml, 29.68 mmol) and MeOH (12 ml) were placed in a 100 ml round bottom flask. [(1-ethoxycyclopropyl)oxy]-trimethylsilane (1.78 g, 29.68 mmol) was added drop wise at room temperature and the reaction mixture was refluxed at 67-69° C. for 3 h under N₂ atmosphere. The resulting mixture was concentrated in vacuo using a rotary evaporator to obtain tert-butyl [2-({2-[(1-ethoxycyclopropyl)amino]phenyl}amino)-1-methyl-2-oxoethyl]carbamate 2.69 g), (M/Z 363). Into a 100 ml round bottom flask was fed NaBH₄ (0.56 g, 14.83 mmol) and anhydrous THF (20 ml). After cooling to 5° C. and adding BF₃.Et₂O complex (2011 g, 14.83 mmol) drop wise, the mixture was stirred under N2 atmosphere for 1 h at 5° C. Into this flask, the crude product (tert-butyl[2-({2-[(1-ethoxycyclopropyl)amino]phenyl}amino)-1-methyl-2-oxoethyl]carbamate) dissolved in THF (10 ml) was added drop wise at 5-10° C. in a time period of 20 mins. After stirring at room temperature for 5 h, at reflux for 2 h, and recovering THF by distillation, the mixture was cooled to room temperature and poured into water (50 m). The resulting mixture was extracted with Et₂O (2×50 ml). The Et₂O layer was washed with water (2×50 ml) and dried over anhydrous Na₂SO₄ followed by the removal of Et₂O using a rotary evaporator to obtain the title compound (1.2 g). M/Z 319.

The product is used in the next cyclization step without further purification.

### Step III

### [1-(1-cyclopropyl-1H-benzimidazol-2-yl)ethyl]amine hydrochloride (Intermediate 21)

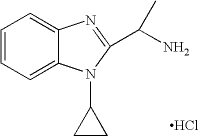

tert-butyl (2-{[2-(cyclopropylamino)phenyl]amino}-1-methyl-2-oxoethyl)carbamate (1.2 g, 7.42 mmols) was dissolved in 4M HCl/dioxane (10 mL) and stirred at room temperature for 1 h to remove the BOC group. The reaction mixture was concentrated under reduced pressure and dried in vacuo to obtain the title compound (0.43 g), M/Z 201.

Intermediates 40, 41 and 43 were prepared from BOC protected intermediate 39 as described below for intermediate 40

### Intermediate 43

### [(1R)-1-(1-ethyl-5-pyridin-3-yl-1H-benzimidazol-2-yl)ethyl]amine

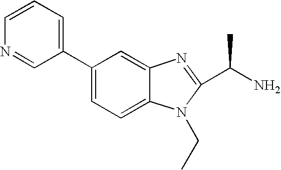

### Step 1

### tert-butyl[(1R)-1-(1-ethyl-5-pyridin-3-yl-1H-benzimidazol-2-yl)ethyl]carbamate

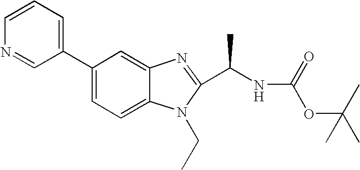

tert-butyl[1-(5-bromo-1-ethyl-1H-benzimidazol-2-yl)ethyl]carbamate (Boc protected intermediate 39, 367 mg, 1 mmol), pyridyl boronic acid (180 mg, 1.5 mmol), potassium carbonate (483 mg, 3.5 mmol), and (DPPF)PdCl₂ (42 mg, 0.05 mol) were combined in a septum-capped test tube under nitrogen. Dioxane (2 mL) and water (0.5 mL) were added and the mixture was stirred at 90° C. for 12 hours. The mixture was partitioned between dichloromethane and water and the aqueous layer was extracted with dichloromethane. The combined organic layers were washed with brine and dried over magnesium sulfate. The solvent was removed in vacuo and the material was purified by flash chromatography. ¹H NMR (DMSO-d6): 1.32 (t, 3H), 1.38 (s, 9H), 1.52 (d, 3H), 4.30 (m, 2H), 5.05 (m, 1H), 7.47 (m, 1H), 7.57 (d, 1H), 7.65 (d, 1H), 7.94 (s, 1H), 8.09 (d, 1H), 8.53 (d, 1H), 8.92 (s, 1H)

### Step 2

### [(1R)-1-(1-ethyl-5-pyridin-3-yl-1H-benzimidazol-2-yl)ethyl]amine

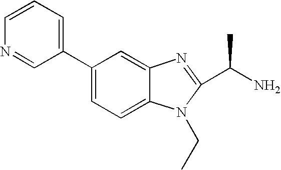

Pyridyl benzimidazole carbamate (170 mg, 0.46 mmol) was stirred at room temperature in 4N HCl in dioxane (3 mL). After one hour, solvent was removed in vacuo to yield crude product as an HCl salt. M/Z 266.

### Intermediate 52

### 1-(4-chloro-1-ethyl-1H-imidazo[4,5-c]pyridin-2-yl)ethanamine[1-(4-chloro-1-ethyl-1H-imidazo[4,5-c]pyridin-2-yl)ethyl]amine

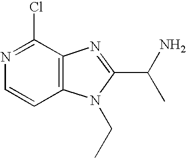

### Step 1

### 4-chloro-1-ethyl-1H-imidazo[4,5-c]pyridine

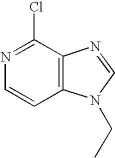

To a solution of 2-chloro-N⁴-ethylpyridine-3,4-diamin (SM 1ac, 2 g) in triethyl orthoformate (30 mL) was added HCl (12N, 1.3 mL). The reaction was stirred for 12 h at rt. The reaction mixture was concentrated under vacuum. The mixture was purified using silica gel chromatography to yield 4-chloro-1-ethyl-1H-imidazo[4,5-c]pyridine (900 mg). M/Z 181.

### Step 2

### 4-chloro-1-ethyl-1H-imidazo[4,5-c]pyridine-2-carbaldehyde

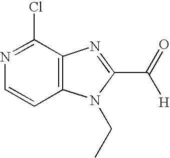

To a solution of 4-chloro-1-ethyl-1H-imidazo[4,5-c]pyridine (500 mg) in THF (14 mL) was added n-BuLi (2.4 mL, 2.5 M in hexane) at −78° C. The reaction mixture was stirred for 45 min at this temperature and then DMF (1.10 mL, 14.3 mmol) was added. The resulting solution was quenched with water then extracted with chloroform (2×20 mL). The combined organic layers were dried over Na₂SO₄ and concentrated to yield crude product 4-chloro-1-ethyl-1H-imidazo[4,5-c]pyridine-2-carbaldehyde as yellow solid, which was used directly in next step. M/Z=209.

### Step 3

### N-[(1E)-(4-chloro-1-ethyl-1H-imidazo[4,5-c]pyridin-2-yl)methylene]-2-methylpropane-2-sulfinamide

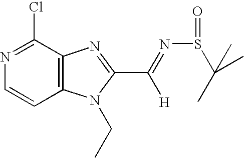

A solution of 4-chloro-1-ethyl-1H-imidazo[4,5-c]pyridine-2-carbaldehyde (prepared above) in dichloromethane was treated with 2-methylpropane-2-sulfinamide (518 mg, 4.28 mmol) and copper sulfate (4 g). The resulting solution was stirred for 18 h at room temperature. The reaction mixture was diluted with dichloromethane, filtered and concentrated to yield N-[(1E)-(4-chloro-1-ethyl-1H-imidazo[4,5-c]pyridin-2-yl)methylene]-2-methylpropane-2-sulfinamide (380 mg), which was used directly in next step. M/Z=312.

### Step 4

### N-[1-(4-chloro-1-ethyl-1H-imidazo[4,5-c]pyridin-2-yl)ethyl]-2-methylpropane-2-sulfinamide

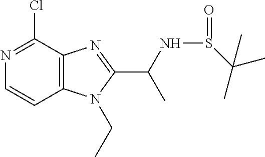

A solution of N-[(1E)-(4-chloro-1-ethyl-1H-imidazo[4,5-c]pyridin-2-yl)methylene]-2-methylpropane-2-sulfinamide (prepared above) in THF was treated with methyl magnesium bromide (2.4 mL, 1M in THF) at −78° C. The resulting solution was stirred for overnight and slowly warmed to room temperature. The reaction mixture was poured into a saturated solution of ammonia chloride (20 mL) slowly and extracted with DCM (2×30 mL). The combined organic layers were dried over Na₂SO₄ and concentrated to yield N-[1-(4-chloro-1-ethyl-1H-imidazo[4,5-c]pyridin-2-yl)ethyl]-2-methylpropane-2-sulfinamide as a yellow solid, which was used directly in next step. M/Z=328.

### Step 5

### 1-(4-chloro-1-ethyl-1H-imidazo[4,5-c]pyridin-2-yl)ethanamine[1-(4-chloro-1-ethyl-1H-imidazo[4,5-c]pyridin-2-yl)ethyl]amine

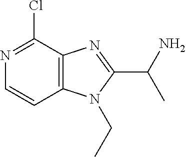

A solution N-[1-(4-chloro-1-ethyl-1H-imidazo[4,5-c]pyridin-2-yl)ethyl]-2-methylpropane-2-sulfinamide (prepared above) in MeOH (2 mL) was treated with hydrochloride acid (1.8 mL, 4M). The resulting solution was stirred overnight. The reaction mixture was concentrated to yield product as a viscous glue. To this material was added a solvent mixture of MeOH/Et₂O (V/V=1:3, around 10 mL). 1-(4-chloro-1-ethyl-1H-imidazo[4,5-c]pyridin-2-yl)ethanamine[1-(4-chloro-1-ethyl-1H-imidazo[4,5-c]pyridin-2-yl)ethyl]amine was precipitated from solution as a white solid. M/Z=224.

### Preparation of Amide Starting Materials (SM) 2a-2z and 2aa-2aj

### Starting Material 2a

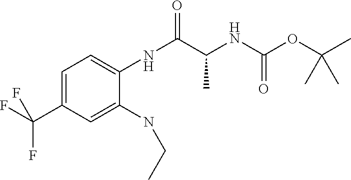

Boc-D-Ala-OH (1.78 g, 9.4 mmols) was taken in a round bottom flask equipped with a stir bar and DCM (10 mL) was added to it. To the homogeneous solution obtained, DIEA (3.3 mL, 19 mmols) and PYBOP (4.9 g, 9.4 mmols) were added. The resultant mixture was stirred for 15 minutes and then added slowly to another round bottom flask containing N-ethyl-4-aminobenzotrifluoride (Starting Material 1, 1.74 g, 8.5 mmols) and DCM (10 mL). The resultant mixture was stirred at room temperature overnight. The reaction mixture was concentrated to a thick syrup and dried in vacuo and used in its crude form for the next step. M/Z 375. The S isomer (Starting Material 2a″) as well as the racemate (Starting Material 2a′) of 2a were prepared following the above procedure and reacting Starting Material 1 with commercially available Boc-L-Ala-OH and Boc-DL-Ala-OH, respectively.

Starting Materials 2b-2g were prepared in a similar fashion to Starting Material 2a starting from the appropriate Starting Material 1b-1g as indicated in Table 5. Starting Material 2h was analogously prepared from the appropriate commercially available BOC protected amino acid and Starting Material 1 h. Generation of racemates and L-isomers was affected by using Boc-Ala-OH of appropriate chirality.

Starting material 2ah and 2ai were prepared as described below. Starting material 2w was prepared either by the method described above for 2a using racemic Boc-Ala-OH or below for 2ah using Boc-D-ala-OH to generate 2w′.

Starting Material 2ah:

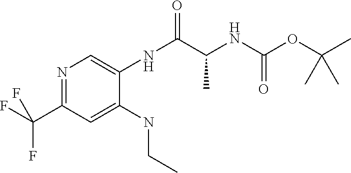

A 50 mL round bottom flask was charged with BOC-D-Ala-OH (480 mg, 2.54 mmol) and N,N′-carbonyldiimidazole (411 mg, 2.53 mmol). CH₂Cl₂ (3 mL) was added, and the resulting solution was allowed to stir at room temperature. After 75 minutes, the mixture was transferred to a separate 50 mL round bottom-flask containing crude N⁴-ethyl-6-(trifluoromethyl)pyridine-3,4-diamine (Starting material 1aj, prepared as described in WO2002050062; 446 mg, 2.17 mmol). An additional 4×1 mL CH₂Cl₂ was used to rinse in the remaining reagent, and the resulting mixture was placed in a 45° C. oil bath. After stirring at 45° C. for 60 hours, the reaction was allowed to cool. The mixture was partitioned between CH₂Cl₂ and H₂O, and the aqueous layer was extracted with CH₂Cl₂. The combined organics were washed with brine, dried (MgSO₄), filtered, and concentrated. The crude material was purified by silica gel chromatography (gradient elution; R_{f} in 40:60 hexanes:EtOAc=0.33) to give tert-Butyl 1-(4-(ethylamino)-6-(trifluoromethyl)pyridin-3-ylamino)-1-oxopropan-2-ylcarbamate as a colorless to pale yellow solid (397 mg, 49% yield). In the case of starting material 2ai, the reaction mixture was heated at 60° C. for a total of 72 hours instead and an additional 2-(tert-butoxycarbonylamino)-2-methylpropanoic acid (822 mg, 4.04 mmol) and CDI (665 mg, 4.10 mmol) in CHCl₃ (8 mL) was added at the end of 48 hours and the reaction mixture was continued to heat for another 24 hours. At the end of this time, the same work up as that described for 2ah yields 2ai.

Starting material 2aj was prepared from 1d as described below:

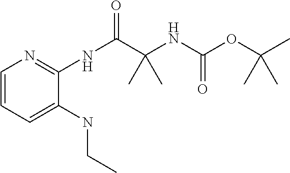

To a solution of 2-tert-butoxycarbonylamino-2-methyl-propionic acid (15.5 g, 76.4 mmol) in DMF (100 mL) was added diisopropyl ethylamine (39.7 mL, 229.2 mmol) under N₂ atm at 0° C. After stirring for 5 min, HATU (32 g, 84 mmol) was added. After stirring for 30 min at this temperature, N³-Ethyl-pyridine-2,3-diamine (10.5 g, 76.39 mmol) in DMF (100 mL) was added with cannula to the reaction mixture. The ice bath was removed and further stirred at rt for 4 days. The reaction mixture was concentrated, diluted with EtOAc, washed with aq. NaHCO₃, water, brine and dried over MgSO₄. The solution was filtered and evaporated and the residue was purified on flash column chromatography on silica gel using 80% EtOAc/hexanes to EtOAc as eluent to afford the product 6.7 g (27.3%).

TABLE 5

| SM2 | Compound | NMR | M/Z | SM1 |
|---|---|---|---|---|
| 2b | 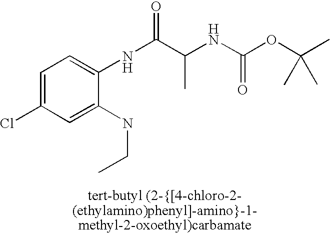 | — | 341 | 1b |
| 2c | 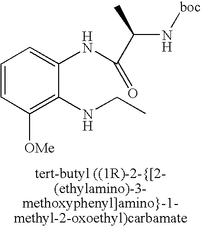 | — | 337 | 1c |
| 2d | 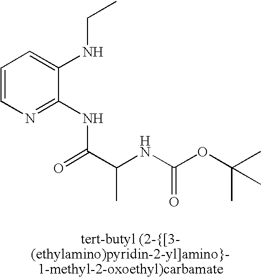 | ¹H NMR (300 MHz, CDCl3) δ ppm 1.45 (s, 12 H) 3.87 (s, 2 H) 4.20-4.34 (m, 1 H) 5.06 (s, 1 H) 6.76 (d, 2 H) 7.00-7.09 (m, 1 H) 7.20-7.29 (m, 2 H) 7.97 (s, 1 H) 7.51-7.75 (m, 1 H) | 308 | 1d |
| 2e | 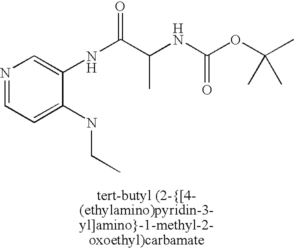 | ¹H NMR (CDCl3): δ 8.16 (d, 1 H), 7.86 (s, 1 H), 6.72 (d, 1 H), 4.05 (m, 1 H), 3.68 (m, 1 H), 3.28 (m, 2 H), 1.50 (s, 9 H), 1.41 (d, 3 H), 1.28 (t, 3 H) | 308 | 1e |
| 2e′ | 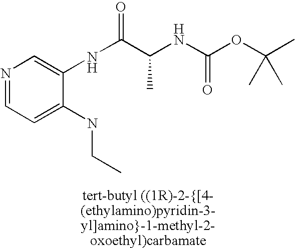 | — | 308 | 1e′ |
| 2f | 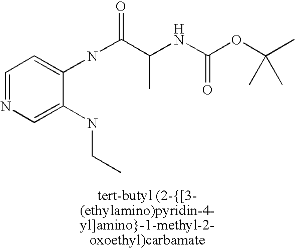 | ¹H(NMR (CDCl3): δ 8.44 (d, 1 H), 7.85 (d, 1 H), 7.71 (s, 1 H), 4.46 (m, 1 H), 3.68 (m, 1 H), 3.2 (m, 2 H), 1.44 (s, 9 H), 1.39 (d, t, 6 H). | 308 | 1f |
| 2g | 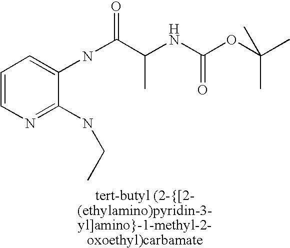 | ¹H NMR (MeOH-d4): δ 7.96 (d, 1 H), 7.84 (bs, 1 H), 7.54 (d, 1 H), 6.60 (dd, 1 H), 5.04 (q, 1 H), 3.42 (q, 2 H), 1.50 (s, 9 H), 1.48 (d, 3 H), 1.28 (t, 3 H). | 308 | 1g |
| 2h | 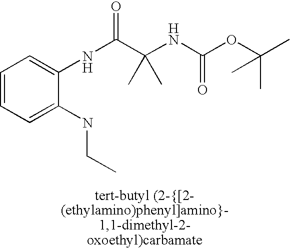 | ¹H NMR (300 MHz, CDCl₃) δ 7.72 (br s, 1 H), 7.26-7.13 (m, 2 H), 6.69-6.67 (m, 2 H), 4.89 (br s, 1 H), 4.56 (br s, 1 H), 3.16-3.11 (m, 2 H), 1.58 (s, 6 H), 1.46 (s, 9 H), 1.27 (t, J = 7.1 Hz, 3 H). | 321 | 1h |
| 2i | 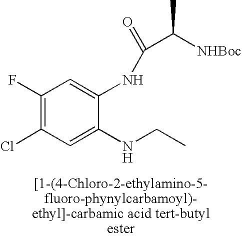 | ¹H NMR (300 MHz, DMSO-d₆) δ: 9.3 (s, 1 H), 7.3 (d, J = 10.4 Hz, 1 H), 7.2 (d, J = 6.3 Hz, 1 H), 6.7 (d, J = 7.4 Hz, 1 H), 4.9 (t, J = 4.9 Hz, 1 H), 3.0 (m, 2 H), 1.3 (s, 9 H), 1.2 (t, J = 7.1 Hz, 3 H). | — | 1i |
| 2j | 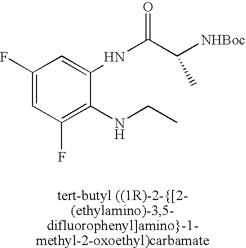 | ¹H NMR (300 MHz, DMSO-d₆) δ: 8.64 (s, 1 H), 7.67 (d, J = 8.52 Hz, 2 H), 7.45 (d, J = 8.52 Hz, 2 H), 7.0-7.2 (m, 2 H), 4.84 (d, J = 6.33 Hz, 2 H), 4.2-4.3 (m, 1 H), 1.31-1.38 (m, 6 H) | 399 | 1j |
| 2k | 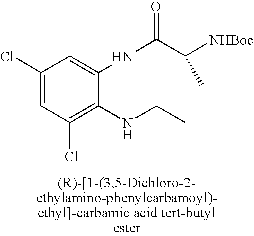 | ¹H NMR (300 MHz, CDCl₃) δ: 8.90 (s, 1 H), 8.33 (d, J = 2.1 Hz, 1 H), 7.0 (d, J = 2.1 Hz, 1 H), 4.95 (m, 1 H), 4.33 (m, 1 H), 3.07 (m, 1 H), 2.89 (m, 2 H), 1.37-1.46 (m, 12 H), 1.21 (t, J = 7.1 Hz, 3 H). | 375 | 1k |
| 2l | 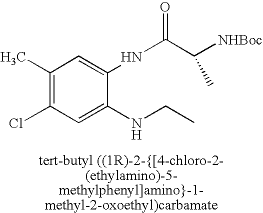 | ¹H NMR (300 MHz, CDCl₃) δ: 7.65 (s, 1 H), 7.14 (s, 1 H), 6.68 (s, 1 H), 4.95 (d, J = 6.8 Hz, 1 H), 3.96 (brs, 2 H), 3.0 (m, 2 H), 2.24 (s, 3 H), 1.39-1.49 (m, 12 H), 1.25 (t, J = 7.1 Hz, 3 H). | 355 | 1l |
| 2m | 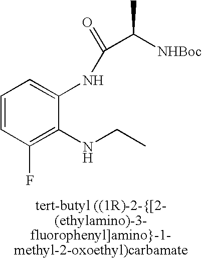 | — | 325 | 1m |
| 2n | 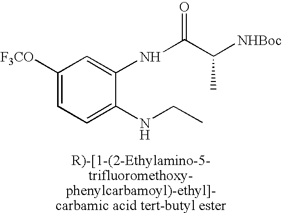 | ¹H NMR (300 MHz, CDCl₃, ppm) δ 1.26 (t, J = 7.14 Hz, 3 H), 1.40- 1.48 (m, 12 H), 3.09-3.17 (m, 2 H), 3.99 (br S, 1 H), 4.22 (quint., J = 6.87 Hz, 1 H), 4.99 (d, J = 6.03 Hz, 1 H), 6.64 (d, J = 7.13 Hz, 1 H), 6.96 (dd, J = 2.46, 8.79 Hz, 1 H), 7.33 (d, J = 2.19, Hz, 1 H), 7.99 (s, 1 H) | 391 | 1n |
| 2o | 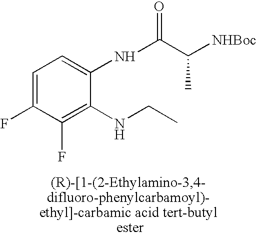 | ¹H NMR (300 MHz, CDCl₃, ppm) δ 1.171.21 (m, 6 H), 1.48 (s, 9 H), 3.07-3.10 (m, 2 H), 4.06-4.12 (m, 1 H), 4.20-4.29 (m, 1 H), 4.97 (br. s, 1 H), 6.69-6.79 (m, 1 H), 7.68- 7.76 (m, 1 H), 8.27 (brs, 1 H) | 343 | 1o |
| 2p | 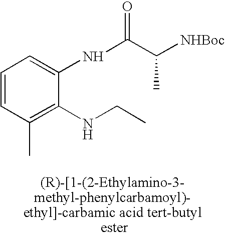 | — | 322 | 1p |
| 2q | 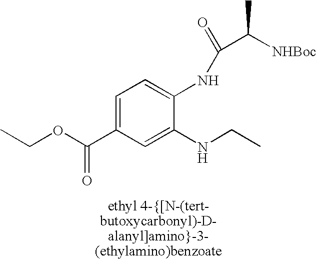 | — | 379 | 1q |
| 2r | 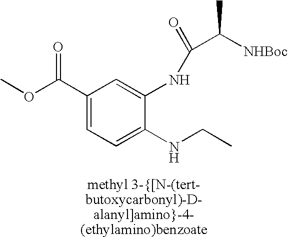 | — | 365 | 1r |
| 2s | 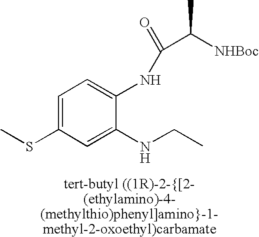 | — | 353 | 1s |
| 2t | 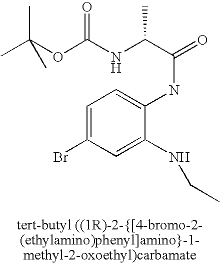 | — | — | 1t |
| 2u | 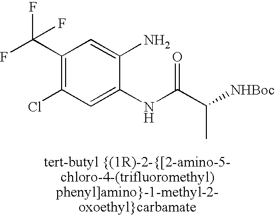 | — | | 1u |
| 2v | 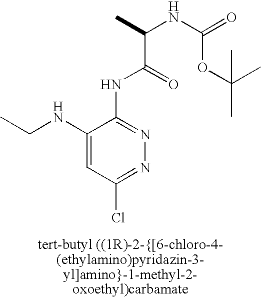 | — | 343 | 1v |
| 2w | 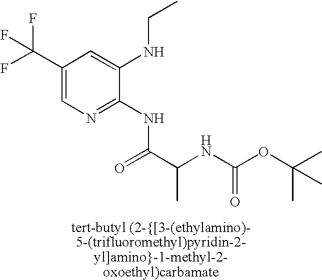 | — | 378 | 1w |
| 2w′ | 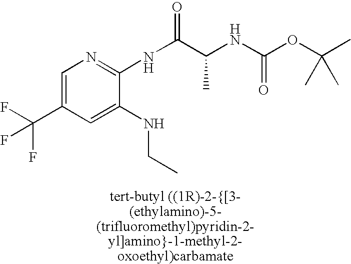 | | 378 | 1w |
| 2x | 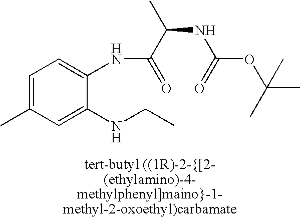 | — | 321 | 1x |
| 2y | 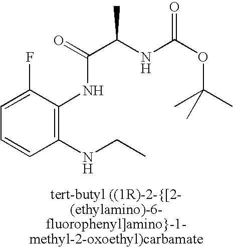 | — | 326 | 1y |
| 2z | 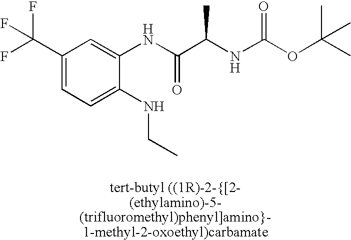 | ¹H NMR (300 MHz, CDCl₃) δ ppm 1.26 (t, 3 H) 1.47 (s, 9 H) 3.12-3.27 (m, 2 H) 4.17-4.27 (m, 1 H) 4.59 (br s, 1 H) 5.06 (d, 1 H) 6.68 (d, 1 H) 7.36 (m, 1 H) 7.45 (m, 1 H) 7.79 (s, 1 H) | 376 | 1z |
| 2aa | 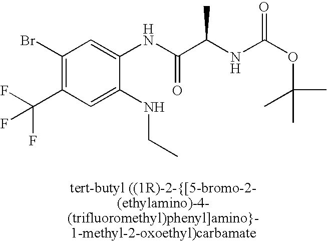 | — | 455 | 1aa |
| 2ab | 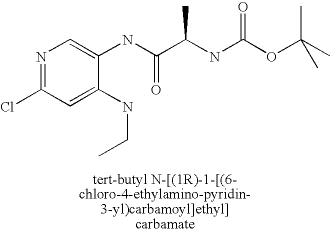 | — | 343 | 1ab |
| 2ac | 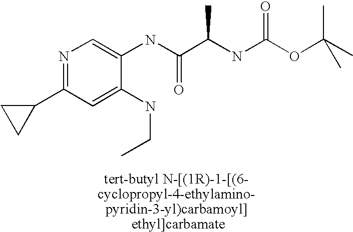 | — | 348 | 1ac |
| 2ac′ | 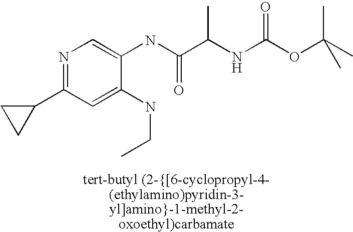 | ¹H NMR (300 MHz, DMSO-D6) δ ppm: 9.56 (s, 1 H), 8.20 (bs, 1 H), 8.00 (s, 1 H), 7.12 (s, 1 H), 6.55 (s, 1 H), 4.04 (m, 1 H), 3.62 (m, 2 H), 2.10 (m, 1 H), 1.40 (s, 9 H), 1.30-1.14 (m, 10 H). | 348 | 1ac′ |
| 2ad | 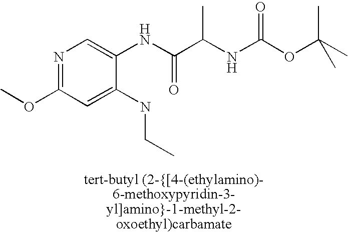 | — | 338 | 1ai |
| 2ad′ | 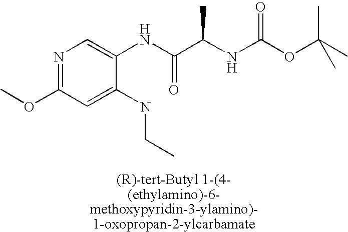 | — | 338 | 1ai′ |
| 2ae | 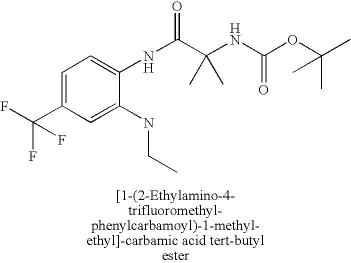 | ¹H NMR (300 MHz, DMSO-d₆) δ: 9.24 (s, 1 H), 7.34 (s, 1 H), 7.03 (d, J = 7.98 Hz, 1 H), 6.81 (d, J = 7.98 Hz, 1 H), 6.74 (s, 1 H), 5.36 (t, J = 3.58 Hz, 1 H), 3.15-3.06 (m, 2 H), 1.40 (s, 9 H), 1.35 (s, 6 H), 1.19 (t, J = 6.87 Hz, 3 H) | 389 | 1a |
| 2af | 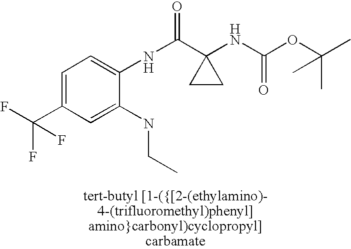 | — | 387 | 1a |
| 2ag | 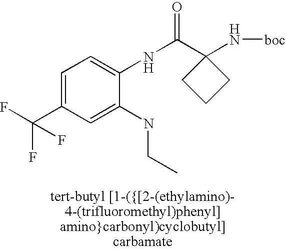 | ¹H NMR (300 MHz, DMSO-d₆) δ: 9.12 (s, 1 H), 7.79 (s, 1 H), 7.12 (d, J = 6.88 Hz, 1 H), 6.84 (d, J = 8.26 Hz, 1 H), 6.76 (s, 1 H), 5.14 (t, J = 3.58 Hz, 1 H), 3.14-3.05 (m, 2 H), 2.56-2.53 (m, 2 H), 2.13-2.04 (m, 2 H), 1.92-1.70 (m, 2 H), 1.41 (s, 9 H), 1.18 (t, J = 7.15 Hz, 3 H) | 401 | 1a |
| 2ah | 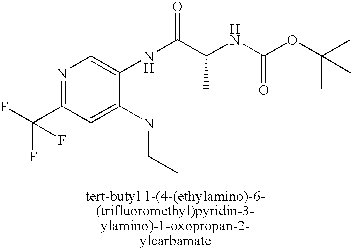 | ¹H NMR (400 MHz, DMSO-D6) δ ppm 1.17 (t, J = 7.20 Hz, 4 H) 1.27 (d, J = 7.07 Hz, 3 H) 1.39 (s, 9 H) 3.19-3.28 (m, 2 H) 3.99- 4.10 (m, 1 H) 6.05 (m, 1 H) 6.95 (m, 1 H) 7.30 (m, 1 H) 8.04-8.13 (m, 1 H) 9.48 (m, 1 H). | 376 | 1aj |
| 2ai | 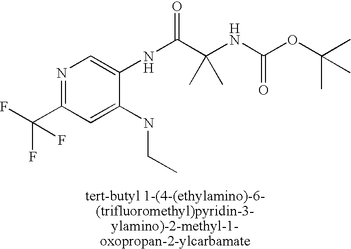 | — | 390 | 1aj |
| 2aj | 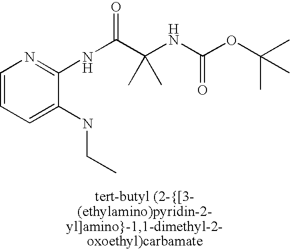 | ¹H NMR (300 MHz, DMSO-d₆) δ: 9.48 (br. s., 1 H), 7.59-7.58 (m, 1 H), 7.27 (br. s., 1 H), 7.10-7.06 (m, 1 H), 6.03-6.90 (m, 1 H), 5.23- 5.19 (m, 1 H), 3.08-3.04 (m, 2 H), 1.41 (s, 9 H), 1.35 (s, 6 H), 1.18 (t, J = 7.15 Hz, 3 H). | 322 | 1d |
| 2ak | 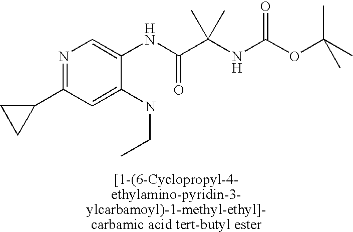 | ¹H NMR (301 MHz, DMSO-d₆) δ ppm 9.29 (s, 1 H), 7.72 (s, 1 H), 7.55 (br. s., 1 H), 6.48 (s, 2 H), 3.20-3.30 (m, 2 H), 1.90-2.11 (m, 1 H), 1.41 (s, 9 H), 1.35 (s, 6 H), 1.19 (t, J = 7.2 Hz, 3 H), 1.01- 1.07 (m, 4 H) | 362 | 1ac |

Starting Materials 1a-1z and 1aa-1ad were prepared from commercially available materials listed in Table 6 and starting material 1aj was prepared as described in WO2002050062.

TABLE 6

| SM1 | Compound | NMR | M/Z | Precursor |
|---|---|---|---|---|
| 1a | 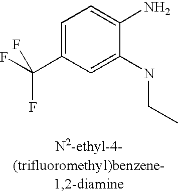 | — | 204 | 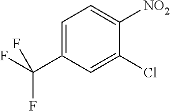 |
| 1b | 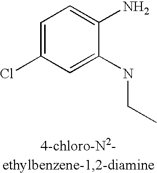 | — | 170 | 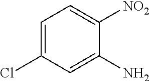 |
| 1c | 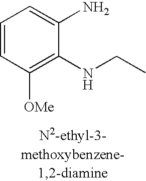 | — | 166 | 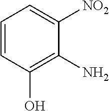 |
| 1d | 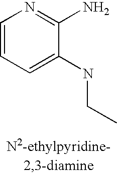 | — | 137 | 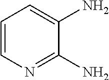 |
| 1e | 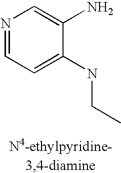 | ¹HNMR (MeOH-d₄): δ 7.67 (s, 1 H), 7.66 (d, 1 H), 6.35 (d, 1 H), 3.26 (q, 2 H), 1.22 (t, 3 H). | 137 | 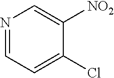 |
| 1f | 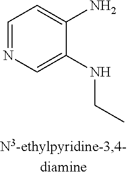 | ¹H NMR (MeOH-d₄): δ 7.55 (d, 1 H), 7.5 (s, 1 H), 6.55 (d, 1 H), 3.1 (t, 2 H), 1.3 (t, 3 H). | 137 | 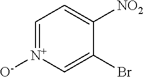 |
| 1g | 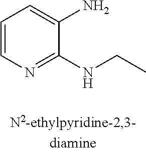 | ¹H NMR (300 MHz, DMSO-d6) δ 7.35 (dd, 1 H), 6.65 (dd, 1 H), 6.32 (dd, 1 H), 5.46 (t, 1 H), 4.65 (s, 2 H), 3.32 (m, 2 H), 1.15 (t, 3 H) | 137 | 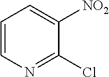 |
| 1h | 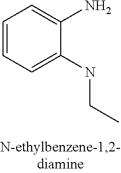 | ¹H NMR (300 MHz, CDCl₃) δ 6.72-6.68 (m, 4 H), 3.19-3.12 (m, 5 H), 1.28 (t, J = 7.1 Hz, 3 H) | 136 | 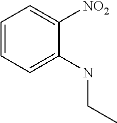 |
| 1i | 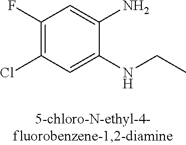 | ¹H NMR (300 MHz, DMSO-d₆) δ: 6.5 (d, J = 11.3 Hz, 1 H), 6.3 (d, J = 7.1 Hz, 1 H), 5.0 (s, 2 H), 4.5 (t, J = 5.0 Hz, 1 H), 3.0 (m, 2 H), 1.2 (t, J = 7 Hz, 3 H) | — | 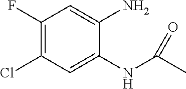 |
| 1j | 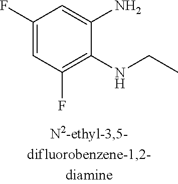 | ¹H NMR (300 MHz, CDCl₃) δ: 6.20-6.23 (m, 2 H), 4.1 (brs, 2 H), 2.92- 2.94 (m, 2 H), 2.8 (brs, 1 H), 1.14 (t, J = 7.1 Hz, 3 H) | 172 | 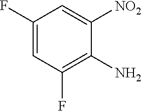 |
| 1k | 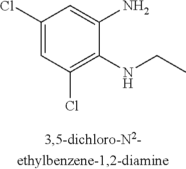 | ¹H NMR (300 MHz, CDCl₃) δ: 6.74 (d, J = 2.19 Hz, 1 H), 6.57 (d, J = 2.19 Hz, 1 H), 4.1 (brs, 2 H), 3.1 (brs, 1 H), 2.89- 2.96 (m, 2 H), 1.14 (t, J = 7.1 Hz, 3 H) | 204 | 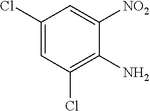 |
| 1l | 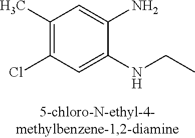 | ¹H NMR (300 MHz, CDCl₃) δ: 6.6 (s, 1 H), 6.55 (s, 1 H), 3.2 (brs, 2 H), 3.10 (m, 3 H), 2.2 (s, 3 H), 1.28 (t, J = 6.87 Hz, 3 H) | 184 | 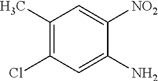 |
| 1m | 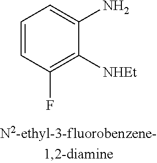 | ¹H NMR (300 MHz, CDCl₃) δ: 6.78 (m, 1 H), 6.50-6.43 (m, 2 H), 3.04- 2.90 (m, 3 H), 1.15 (t, J = 6.9 Hz, 3 H) | 154 | 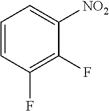 |
| 1n | 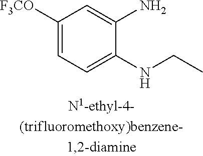 | ¹H NMR (300 MHz, CDCl₃, ppm) δ 1.32 (t, J = 9.06 Hz, 3 H), 3.13 (q, J = 7.14 Hz, 2 H), 3.43 (brs, 2 H), 6.54-6.71 (m, 3 H) | 220 | 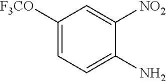 |
| 1o | 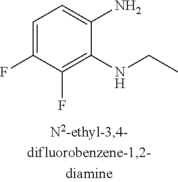 | ¹H NMR (300 MHz, CDCl₃, ppm) δ 1.17 (t, J = 7.14 Hz, 3 H), 1.58 (brs, 1 H), 3.09 (q, J = 7.17 Hz, 2 H), 3.64 (brs, 2 H), 6.32- 6.39 (m, 1 H), 6.58-6.67 (m, 1 H) | 172 | 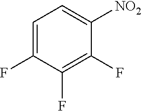 |
| 1p | 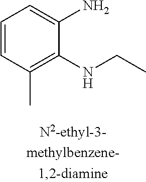 | — | 150 | 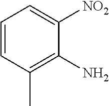 |
| 1q | 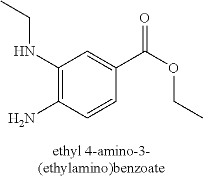 | ¹H NMR δ ppm 1.20- 1.28 (m, 6 H) 3.05 (m, 2 H) 4.15-4.23 (m, 2 H) 4.53 (m, 1 H) 5.38 (s, 2 H) 6.53 (m, 1 H) 6.69 (m, 1 H) 7.14 (m, 1 H) | 208 | 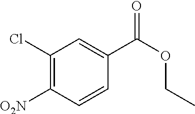 |
| 1r | 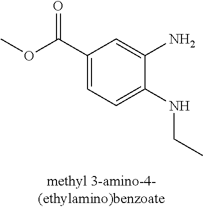 | Bioorg. Med. Chem. 2005, 13, 1587 | 194 | 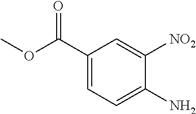 |
| 1s | 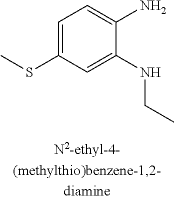 | ¹H NMR δ ppm 1.19 (t, J = 7.20, 3 H) 2.32 (s, 3 H) 2.97-3.07 (m, 2 H) 4.44 (broad s, 1 H) 4.55 (broad s, 2 H) 6.36 (m, 1 H) 6.40- 6.44 (m, 1 H) 6.46- 6.50 (m, 1 H) | 182 | 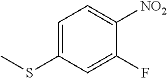 |
| 1t | 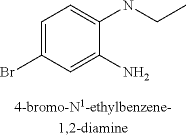 | ¹H NMR 1.18 (t, 3 H), 1.85 (s, 3 H), 2.99 (qua, 2 H), 4.46 (br s, 2 H), 4.81 (broad s), 6.29 (d, 1 H), 6.56 (dd, 1 H), 6.65 (d, 1 H). | — | 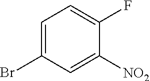 |
| 1u | 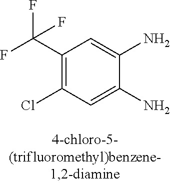 | ¹H NMR (300 MHz, DMSO-D₆) δ ppm 4.95 (s, 2 H) 5.41 (s, 2 H) 6.63 (s, 1 H) 6.85 (s, 1 H) | — | 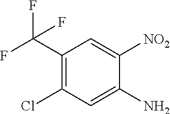 |
| 1v | 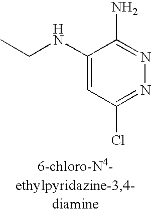 | — | 172 | 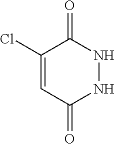 |
| 1w | 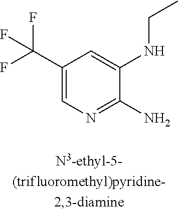 | ¹H NMR (300 MHz, CDCl₃) δ ppm 1.33 (t, 3 H) 1.77 (br s, 1 H) 3.10- 3.22 (m, 2 H) 4.55 (br s, 2 H) 6.90 (d, 1 H) 7.83-7.90 (m, 1 H) | 206 | 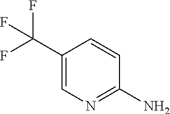 |
| 1x | 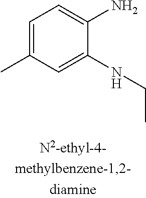 | — | 150 | 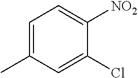 |
| 1y | 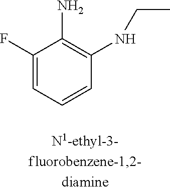 | — | 155 | 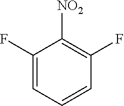 |
| 1z | 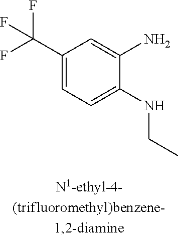 | ¹H NMR (300 MHz, CDCl₃) δ ppm 1.31 (t, 3 H 3.18 (q, 2 H) 3.41 (br s, 2 H), 6.62 (d, 1 H) 6.92 (d, 1 H) 7.05-7.12 (m, 1 H) | 205 | 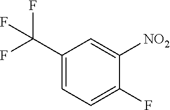 |
| 1aa | 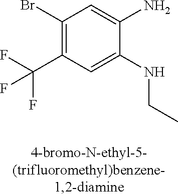 | ¹H NMR (300 MHz, CDCl₃) δ ppm 1.31 (t, 3 H) 3.14 (q, 2 H) 3.61 (br s, 2 H), 6.88 (s, 1 H) 6.94 (s, 1 H) | 283 | 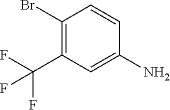 |
| 1ab | 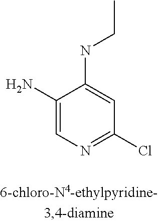 | ¹H NMR (300 MHz, DMSO-D₆) δ ppm 1.19 (t, J = 7.16 Hz, 3 H), 3.10 (dt, J = 12.25, 7.06 Hz, 2 H), 4.75 (s, 2 H), 5.64 (s, 1 H), 6.28 (s, 1 H), 7.37 (s, 1 H). | 171 | 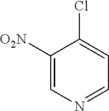 |
| 1ac | 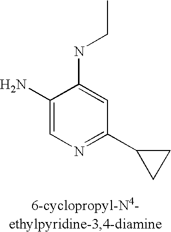 | ¹H NMR (300 MHz, DMSO-D₆) δ ppm 0.72 (s, 2 H), 0.75 (d, J = 3.58 Hz, 2 H), 1.20 (t, J = 7.16 Hz, 3 H), 1.82 (s, 1 H), 3.13 (dt, J = 12.39, 7.09 Hz, 2 H), 4.43 (s, 2 H), 5.40 (s, 1 H), 6.25 (s, 1 H), 7.46 (s, 1 H). | — | 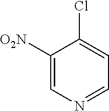 |
| 1ad | 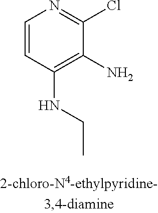 | — | 161 | 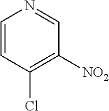 |
| 1ae | 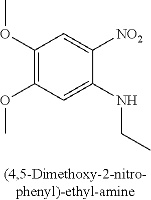 | ¹H NMR (300 MHz, MeOH-d₃) δ(~90% pure): 8.47 (bs, 1 H), 7.44 (s, 1 H), 6.37 (s, 1 H), 3.91 (s, 3 H), 3.73 (s, 3 H), 3.42 (m, 2 H), 1.25 (t, J = 7.14 Hz, 3 H). | 226 | 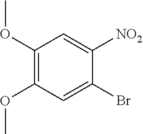 |
| 1af | 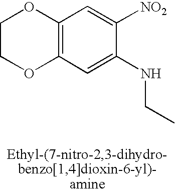 | ¹H NMR (300 MHz, CDCl₃) δ: 7.88 (brs, 1 H), 7.73 (m, 1 H), 6.22 (m, 1 H), 4.33 (m, 2 H), 4.22 (m, 2 H), 3.25 (m, 2 H), 1.33 (t, J = 7.14 Hz, 3 H). | 224 | 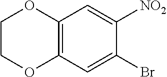 |
| 1ag | 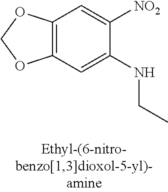 | ¹H NMR (300 MHz, CDCl₃) δ: 8.69 (brs, 1 H), 7.59 (s, 1 H), 6.28 (s, 1 H), 5.98 (s, 2 H), 3.33-3.29 (m, 2 H), 1.36 (t, J = 7.14 Hz, 3 H). | 210 | 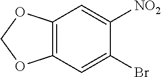 |
| 1ai | 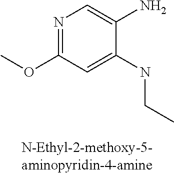 | ¹H NMR (400 MHz, DMSO-D6) δ ppm 1.17 (t, J = 7.20 Hz, 3 H) 3.05 (dd, J = 7.07, 5.31 Hz, 2 H) 3.64 (s, 3 H) 3.72 (s, 1 H) 4.11 (broad s, 2 H) 5.32 (m, 1 H) 5.70 (s, 1 H) 7.20 (s, 1 H). | 167 | 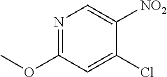 |
| 1aj | 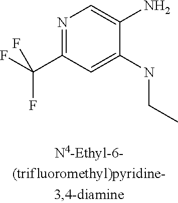 | — | 237 | 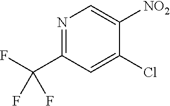 |

### Preparation of Starting Material 1a

### Step I

### N-ethyl-4-nitrobenzotrifluoride

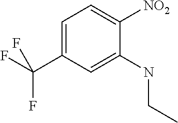

3-chloro-4-nitrobenoztrifloride (1 g, 4.43 mmols) and ethylamine (2M in THF, 12 mL) were taken in a microwave tube equipped with a stir bar. The contents were stirred, sealed and heated in a microwave at 100° C. for 2 hours. The reaction mixture was then transferred into a round bottom flask and concentrated to obtain a bright orange solid. The solid was partitioned between ethyl acetate (300 mL) and water (50 mL). The organic layer was washed with brine, dried with sodium sulfate (anhydrous), filtered and dried in vacuo to obtain 1.45 g (94.66%) of desired product.

### Step II

### N-ethyl-4-aminobenzotrifluoride (starting material 1a)

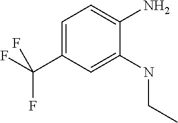

N-ethyl-4-nitrobenzotrifluoride (1.94 g, 8.29 mmols), ethanol (25 mL), 10% Pd/C (3 g) and cyclohexane (20 mL) were taken in a round bottom flask equipped with a stir bar and a reflux condenser. The resultant mixture was heated to 80° C. for 3 h when the reaction was judged to have reached completion based on LC-MS monitoring. The reaction mixture was cooled to room temperature and was filtered through a pad of diatomaceous earth. The filtrate was concentrated in vacuo to obtain an off-white solid, which was used for the next reaction after LC-MS characterization.

Starting Materials 1e-1h, 1m, 1o, 1q-1s, 1t, 1y, 1z, 1ae, 1af, 1ag were prepared in a manner analogous to that described for Starting Material 1a above starting from the commercially available precursor as indicated in Table 6 except that step II for 1f was carried out by treatment with Fe/AcOH, and step II for 1m, 1o using Zn/ammonium chloride as follows:

### Step II in the Preparation of 1f:

### N³-ethylpyridine-3,4-diamine (Starting material 1f)

N³-ethyl-4-nitro-pyridine-N-oxide (1.11 g, 6 mmols), acetic acid (30 mL, 0.2 M) and Fe powder (2 g, 36 mmols) were taken in a flask equipped with a stir bar and heated to 80° C. for 4 hours. The reaction mixture was cooled and acetic acid was evaporated on a rotary evaporator, neutralized with ammonia/methanol (2M). After evaporating methanol, the resulting material was partitioned between ethyl acetate and 50% aq. ammonium hydroxide. The organic layer was washed with brine, dried over Na₂SO₄ (anhydrous), filtered and concentrated under reduced pressure to obtain the title compound (420 mg, 51%), which was used for the next step without further purification.

### N-2-Ethyl-3-fluoro-benzene-1,2-diamine (Starting Material 1m)

Ammonium chloride 93.57 g, 66.12 mmol), zinc powder (4.30 g, 66.12 mmol) was added to a solution of ethyl-(2-fluoro-6-nitro-phenyl)amine (1.21 g, 6.61 mmol) in ethanol (50 mL). The mixture was allowed to stir at room temperature overnight. The reaction mixture was filtered through a diatomaceous earth pad, and washed with ethanol. The filtrate was concentrated. The residue was purified by flash chromatography eluting with gradient of 0.10% EtOAc in hexanes to obtain the product as a black gum (0.54 g, 52% yield). ¹H NMR (300 MHz, CDCl₃) δ: 6.78 (m, 1H), 6.50-6.43 (m, 2H), 3.04-2.90 (m, 3H), 1.15 (t, J=6.9 Hz, 3H). M/Z 154.

### Preparation of Ic

### N²-ethyl-3-methoxybenzene-1,2-diamine (Starting Material 1c)

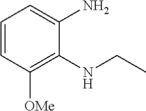

A solution of N-(2-amino-6-methoxyphenyl)acetamide (0.68 g, 3.76 mmol) in dry THF (50 ml) was cooled to 0° C. and LAH (15.03 mmol) was carefully added. After refluxing for 30 mins, the mixture was cooled to room temperature and then to 0° C. and hydrolyzed with a minimum amount of EtOAc and ice. The organic layer was separated by filtration and the solid residue was washed with EtOAc. The EtOAc layer was washed with brine and dried over Na₂SO₄, filtered and evaporated. The product was used in next coupling step without further purification. M/Z 166.

### N-(2-amino-6-methoxyphenyl)acetamide

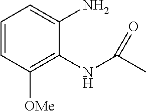

N-(2-nitro-6-methoxyphenyl)acetamide (1.0 g, 4.74 mmol) was dissolved in 50 ml AcOH, then iron (1.59 g, 28.43 mmol) was added into the solution. The reaction mixture was stirred overnight at room temperature. The mixture was diluted with 50 ml EtOAc, filtered and evaporated. The residue was partitioned between ethyl acetate and H₂O. The EtOAc layer was washed with 1N NaOH until pH 9. The EtOAc layer was washed with brine and dried over Na₂SO₄. The solution was filtered, and the solvent was evaporated. The resulting product was used in LAH reduction without further purification. M/Z 180.

### N-(2-nitro-6-methoxyphenyl)acetamide

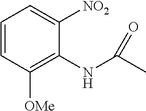

2-amino-3-nitro-6-methoxybenzene (0.80 g, 4.74 mmol) was dissolved in 30 ml toluene then acetyl chloride (0.74 g, 9.48 mmol) was added into the reaction mixture. The mixture was heated at 80° C. overnight. The residue was partitioned between EtOAc and H₂O. The EtOAc layer was washed with brine and dried over Na₂SO₄. The solution was filtered, and the solvent was evaporated. The crude product thus obtained was carried on to the next step to generate N-(2-amino-6-methoxyphenyl)acetamide. M/Z 210.

In the case of SM 1c an additional step needed to be carried out as shown below:

### 2-amino-3-nitro-6-methoxybenzene

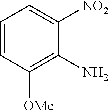

Into a 250 ml round bottom flask was fed 2-amino-3-nitrophenol (2.04 g, 13.24 mmol) and 50 ml anhydrous THF. Cesium carbonate (18.98 g, 58.26 mmol) was added into the solution followed by methyl iodide (2.07 g, 14.56 mmol). The mixture was stirred 6 days at room temperature. The resulting mixture was filtered and washed with DCM. The solvent was removed on a rotary evaporator. The crude material was purified by silica gel chromatography (gradient elution; EtOAc:Hexane 20%) to afford title compound (1.1 g, 50%) M/Z 168.

Starting Material 1b was made in a similar manner to Ic starting from the commercially available precursor listed in Table 6.

Starting material 1d was prepared by a two-step procedure described below:

### Step I

### N-(2-Amino-pyridin-3-yl)-acetamide

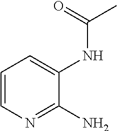

N-(2-Amino-pyridin-3-yl)-acetamide was prepared from pyridine-2,3-diamine and acetic anhydride, following the method of Mazzini, C; Lebreton, J; Furstoss, R; Heterocycles; 45(6); 1161 (1997).

### Step II

### N³-Ethyl-pyridine-2,3-diamine (Starting material 1d)

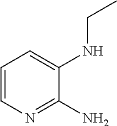

N³-Ethyl-pyridine-2,3-diamine was prepared from N-(2-amino-pyridin-3-yl)-acetamide and lithium aluminum hydride, following the method of Mazzini, C; Lebreton, J; Furstoss, R; Heterocycles; 45(6); 1161 (1997).

Starting Materials 1i-1i, 1n, 1p were prepared in a manner analogous to that described for Starting Material 1a above starting from the commercially available precursor as indicated in Table 6. For starting material 1i, only step 2 (reduction of the acetamide) was necessary

### Preparation of Starting Material 1j

### N-(2,4-Difluoro-6-nitro-phenyl)-acetamide

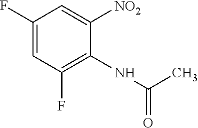

2,4-Difluoro-6-nitro-phenylamine (3.0 g, 17.1 mmol) was dissolved in anhydrous THF, to this was added pyridine (2.6 mL, 32.4 mmol) followed by acetyl chloride (2.63 mL, 37.0 mmol). This was stirred overnight at rt under a current of N_{2(g)}. Next day TLC indicated completion of the reaction. The reaction mixture was concentrated, diluted with EtOAc, and washed with water/HCl_{(aq)}/H₂O/brine, dried under Na₂SO₄ and concentrated. The residue was subjected to flash chromatography on silica gel. Yield: 3.9 g (96%). ¹H NMR (300 MHz, CDCl₃) δ: 8.0 (brs, 1H), 7.5-7.6 (m, 1H), 7.23-7.59 (m, 1H), 2.28 (s, 3H). (M+1)/Z=217.1.

### Step 2

### (2,4-Difluoro-6-nitro-phenyl)-ethyl-amine

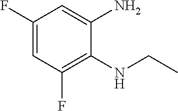

N-(2,4-Difluoro-6-nitro-phenyl)-acetamide (3.56 g, 16.4 mmol) was dissolved in anhydrous THF and cooled to 0° C. To the resulting mixture was slowly added LAH (2.49 g, 65.6 mmol). This was then refluxed at 80° C. for 30 min, after which it was quenched by adding a few drops of EtOAc and ice at 0° C. This mixture was then filtered over diatomaceous earth. The residue was washed with EtOAc and concentrated and subjected to flash chromatography on silica gel. Yield: 1.0 g (35%). ¹H NMR (300 MHz, CDCl₃) δ: 6.20-6.23 (m, 2H), 4.1 (brs, 2H), 2.92-2.94 (m, 2H), 2.8 (brs, 1H), 1.14 (t, J=7.1 Hz, 3H). (M+1)/Z=173.1.

### Starting Material 1u

### 4-chloro-5-(trifluoromethyl)benzene-1,2-diamine

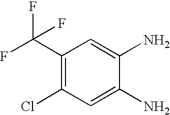

5-chloro-2-nitro-4-(trifluoromethyl)aniline was combined with 5% iron(III) chloride on silica gel (6.692 g, 0.2063 mmol), activated carbon (3.2 g, 2×wt. of SM), and 20 mL MeOH. This reaction mixture was stirred and heated at 80° C. for 10 min. Hydrazine monohydrate was then added (4.0 mL, 82.51 mmol) slowly, drop wise at first to avoid foaming, then quickly. The reaction was stirred and heated for an additional 15 min, then filtered hot. The solids were washed with MeOH and EtOAc. The filtrates were combined and concentrated to yield 1.4276 g product as a light yellow solid. ¹H NMR (300 MHz, DMSO-D₆) 4.95 (s, 2H) 5.41 (s, 2H) 6.63 (s, 1H) 6.85 (s, 1H).

### Starting Material 1v

### 6-chloro-N⁴-ethylpyridazine-3,4-diamine

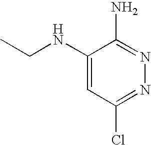

SM 1v was prepared in 5 steps from commercially available 3-chlorofuran-2,5-dione

### Step 1

### 4-chloro-1,2-dihydropyridazine-3,6-dione

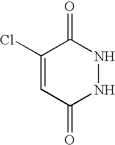

To a solution of 3-chlorofuran-2,5-dione (10 g) in EtOH (200 mL) was added hydrazine monohydrate (4 mL) and refluxed for 10 h. The reaction mixture was concentrated to yield 4-chloro-1,2-dihydropyridazine-3,6-dione, which was used directly in the next step. M/Z 146.

### Step 2

### 3,4,6-trichloropyridazine

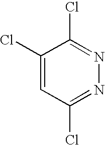

A solution of 4-chloro-1,2-dihydropyridazine-3,6-dione in POCl₃ (100 mL) was refluxed for 10 h. The reaction mixture was concentrated and purified to yield 3,4,6-trichloropyridazine, which was used directly in the next step. M/Z 182.

### Step 3

### 3,6-dichloro-N-ethylpyridazin-4-amine

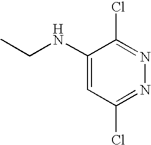

To 3,4,6-trichloropyridazine was added ethylamine (35 mL, 70% water solution). The reaction mixture was stirred for 2 h and was extracted with ethyl acetate. The combined organic layers were concentrated to yield 3,6-dichloro-N-ethylpyridazin-4-amine, which was used directly in the next step. M/Z 191.

### Step 4

### 6-chloro-N-ethyl-3-hydrazinopyridazin-4-amine

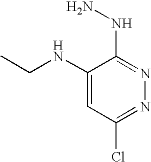

To 3,6-dichloro-N-ethylpyridazin-4-amine (3.6 g) was added hydrazine (16 mL). The reaction mixture was refluxed for 2 h and was diluted with water (10 mL). A precipitate formed and was collected to yield 6-chloro-N-ethyl-3-hydrazinopyridazin-4-amine which was used directly in the next step. M/Z 187.

### Step 5

### 6-chloro-N⁴-ethylpyridazine-3,4-diamine

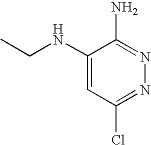

To a solution of 6-chloro-N-ethyl-3-hydrazinopyridazin-4-amine (500 mg) in EtOH was added Raney nickel (0.2 g). The reaction mixture was placed under a hydrogen atmosphere for 2 h. The reaction mixture was filtered through diatomaceous earth. The organic filtrate was concentrated to yield 6-chloro-N⁴-ethylpyridazine-3,4-diamine, which was used directly in the next step. M/Z 172.

Starting material 1w was prepared in 4 steps as described below

### Starting Material 1w

### N³-Ethyl-5-trifluoromethyl-pyridine-2,3-diamine

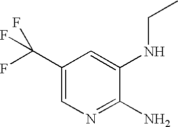

### Step 1

### N-(5-Trifluoromethylpyridin-2-yl)methanesulfonamide

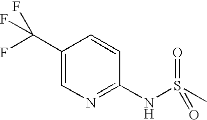

Under a nitrogen atmosphere, 2-chloro-5-trifluoromethyl-pyridine (9.07 g; 0.05 mol) was added to ca. 30 mL dimethylsulfoxide in a 100 mL round-bottomed flask. Methanesulfonamide (5.3 g; 0.5 g; 0.06 mol) and potassium carbonate (325 mesh powder; 13.9 g; 0.10 mol) were added sequentially in single portions and washed with another ca. 30 mL dimethylsulfoxide. After heating to 120° C. for 8 h, the reaction mixture was poured onto ice/water, causing small amount of precipitate to form. The aqueous phase was extracted twice with ether, leaving a clear yellow solution. Cautious addition of concentrated hydrochloric acid to the aqueous layer until pH ca. 4, provided a white solid. This solid was collected by filtration, and washed with portions of water. After drying on the filter, the solid was recrystallized from 2-propanol. M/Z=241 (300 MHz, CHLOROFORM-D) δ ppm 3.26 (s, 3H) 7.38 (d, 1H) 7.94 (m, 1H), 8.65 (s, 1H)

### Step 2

### N-(3-Nitro-5-trifluoromethyl-pyridin-2-yl)-methanesulfonamide

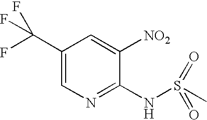

N-(5-Trifluoromethyl-pyridin-2-yl)-methanesulfonamide (4.8 g; 0.02 mol) was suspended in ca. 15 mL acetic acid in a 50 mL round-bottomed flask. Heating to ca 110° C., caused most of the material to dissolve to a somewhat turbid solution. Nitric acid (fuming 90%; 2.1 mL) was added drop wise from an addition funnel, immediately causing the remainder of solid to dissolve. After the addition was complete, the reaction was heated 7 h longer, and then cooled. The yellow solution was poured onto ice/water causing a solid to form. The solid was filtered, and the filter cake was washed with portions of water to obtain a white solid. The solid was recrystallized from 2-propanol. Evaporation of the filtrate, and recrystallization of the residue provided a second crop. M/Z=285 (300 MHz, CDCl₃) δ ppm 3.58 (s, 3H) 8.82 (m, 1H) 8.91 (m, 1H) 10.10 (br s, 1H)

### Step 3

### N-(3-Ethylamino-5-trifluoromethyl-pyridin-2-yl)-methanesulfonamide

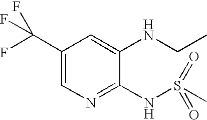

N-(3-Nitro-5-trifluoromethyl-pyridin-2-yl)-methanesulfonamide (1.1 g; 0.004 mol) was suspended in ca. 15 mL methanol in a 50 mL round-bottomed flask. Acetonitrile (2 mL; 0.04 mol), ammonium acetate (0.31 g; 0.004 mol) and then the 10% palladium-on-carbon (0.22 g; mol %) were added sequentially in single portions, and washed in with a bit more methanol. A hydrogen-filled balloon was attached to the flask, and the flask was alternatively placed under vacuum, and then under hydrogen atmosphere. After the third cycle, the reaction was left under a hydrogen atmosphere for 16 h, after which time all the starting material was consumed. The reaction mixture was filtered through a pad of diatomaceous earth, which was washed with a few portions of methanol. Two products were in evidence: the desired product, along with a similar amount of N-(3-amino-5-trifluoromethyl-pyridin-2-yl)-methanesulfonamide (the product of simple reduction without alkylation). Medium pressure chromatography (ethyl acetate/hexanes) provided pure desired product. M/Z=284 (300 MHz, CDCl₃) 1.33 (t, 3H) 1.64 (br s, 1H) 3.11 (s, 3H) 3.14-3.25 (m, 2H) 5.29 (br m, 1H) 6.46 (m, 1H) 7.17 (s, 1H)

### Step 4

### N³-Ethyl-5-trifluoromethyl-pyridine-2,3-diamine

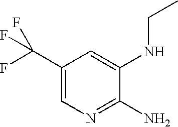

N-(3-Ethylamino-5-trifluoromethyl-pyridin-2-yl)-methanesulfonamide (0.57 g; 0.002 mol) was added to a 15 mL round-bottomed flask. Addition of ca 1.0 mL concentrated sulfuric acid caused much of the material to dissolve. Upon heating to 110° C., the entire solid dissolved to a clear, yellowish solution, which darkened somewhat with time. After ca 45 min, LC/MS of an aliquot showed complete disappearance of the starting material, and the reaction mixture was cooled to room temperature. Ca. 7 g sodium carbonate was added to ca 20 mL water in a 250 mL flask; most of the solid dissolved. Ca. 125 mL ether was then added as the upper layer. The reaction mixture was cautiously added portion wise to the rapidly stirred two-phase system. The layers were separated and the aqueous phase was extracted twice with ether. The combined organic layer was washed with a small portion of water, and then with a small portion of saturated sodium chloride solution. Upon drying over magnesium sulfate, removal of solvent under reduced pressure provided the product as an off-white solid. M/Z 206 (300 MHz, CDCl₃) δ ppm 1.33 (t, 3H) 1.77 (br s, 1H) 3.10-3.22 (m, 2H) 4.55 (br s, 2H) 6.90 (d, 1H) 7.83-7.90 (m, 1H)

### Starting Material 1aa

### 4-Bromo-N¹-ethyl-5-trifluoromethyl-benzene-1,2-diamine

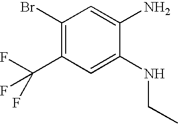

### Step 1

### N-(4-bromo-2-nitro-5-trifluoromethyl-phenyl)-acetamide

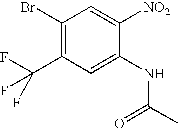

Following the method of Ognyanov, V. I., et. al; J. Med. Chem.; 49(12); 3719 (2006), under a nitrogen purge, 4-bromo-3-trifluoromethylaniline (7.2 g; 0.03 mol) was added to acetic anhydride (30 mL) in a 100 mL round-bottomed flask. After stirring for 16 h at room temperature, solvent was removed under reduced pressure to obtain a white solid, which was used as-is for the nitration step.

Concentrated sulfuric acid (32.5 mL) was added to the solid N-(4-bromo-3-trifluoromethyl-phenyl)-acetamide and the round-bottomed flask cooled in an ice bath. Nitric acid (ca 90%, 4.1 mL) was placed in an addition funnel and added dropwise. After stirring another 30 min at ice temperature, the reaction was allowed to warm to room temperature and stir another 3 h. The reaction was poured onto ice, and solid sodium bicarbonate was cautiously added until the aqueous phase measured slightly basic. The reaction was extracted with ethyl acetate three times. The combined organic layer was washed with water twice, and once with saturated sodium chloride solution. After drying over magnesium sulfate, solvent was removed under reduced pressure. The resulting crude product was purified by recrystallization from methylcyclohexane. M/Z=328 (300 MHz, CDCl₃) δ ppm 2.33 (s, 3H) 8.53 (s, 1H) 9.29 (s, 1H) 10.22 (br s, 1H)

### Step 2

### 4-Bromo-N¹-ethyl-5-trifluoromethyl-benzene-1,2-diamine

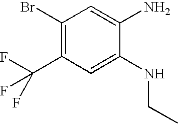

Zirconium borohydride was prepared by dissolving zirconium chloride (11.6 g; 0.05 mol) in THF (200 mL) in a 500 mL round-bottomed flask and then adding solid sodium borohydride (7.6 g; 0.2 mol). The suspension was stirred 40 h under a nitrogen atmosphere. As needed, the required volume of the supernatant (nominally 0.1 molar borohydride) was decanted from the settled solids and filtered into an addition funnel.

Under a nitrogen purge, N-(4-bromo-2-nitro-5-trifluoromethyl-phenyl)-acetamide (0.65 g: 0.002 mol) was dissolved in ca. 30 THF in a 100 mL round-bottomed flask. Supernatant zirconium borohydride solution (20 mL of 0.1 M borohydride; 0.002 mol) was withdrawn and then filtered through a syringe filter into an addition funnel. The solution was added drop wise to the reaction, causing an exotherm. The reaction was stirred 16 h at room temperature, at which time an aliquot revealed reduction of both the amide and nitro groups. Volatiles were removed under reduced pressure. Ice and ethyl acetate were cautiously added to the resulting residue. The layers were separated, and the aqueous phase was extracted twice with ethyl acetate and the combined organic layer was washed twice with water and once with saturated sodium chloride solution. After drying over magnesium sulfate, solvent was removed under reduced pressure to provide the desired compound, which was employed in the next step without further purification. M/Z=283 (300 MHz, CDCl₃) δ ppm 1.31 (t, 3H) 3.14 (q, 2H) 3.61 (br s, 2H) 6.88 (s, 1H) 6.94 (s, 1H)

### Starting Material 1ab

### 6-chloro-N′-ethyl-pyridine-3,4-diamine

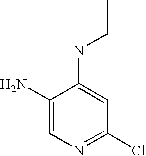

### Step 1

### 4-chloro-5-nitropyridin-2-ol

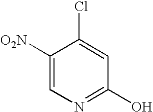

THF (50 mL) was cooled to −78° C., and NH₃ (gas, about 30 mL) was condensed into the THF solution. Potassium tert-butoxide (9.3 g, 79.1 mmol) was added and the mixture was warmed, and kept at −35° C. In a separate flask, 4-chloro-3-nitropyridine was dissolved in THF (40 mL) and cooled to 0° C. To the 4-chloro-3-nitropyridine solution was added t-BuOOH (7.0 mL of 5 M solution in decane, 35.0 mmol). The solution was added drop wise to KOt-Bu solution over 30 min. The reaction was stirred at −35° C. for 0.5 h, and it was cooled to −78° C. before quenching slowly with a saturated NH₄Cl solution (20 mL). The mixture was kept at rt and allowed to vent overnight. The mixture was concentrated, filtered, and the resulting solid was washed with cold water (3×10 mL). Vacuum drying afforded the title compound as a yellow solid (2.7 g, 60% yield). ¹H NMR (300 MHz, DMSO-D₆) δ ppm 5.95 (s, 1H), 8.80 (s, 1H). M/Z 174 (M-H observed).

### Step 2

### 2,4-dichloro-5-nitro-pyridine

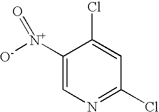

At rt, 4-chloro-5-nitropyridin-2-ol (2.7 g, 15.6 mmol) was suspended in toluene (60 mL), and POCl₃ (14.2 mL, 156 mmol) was added to the mixture. The reaction was refluxed for 6 h, and heated at 60° C. over weekend. After cooling the mixture down, concentration removed the solvent and excess POCl₃. To the residue was added toluene (2×20 mL) and concentration was reapplied to remove excess POCl₃. The residue was slowly added to sat. K₂CO₃ (30 mL), and the mixture was extracted with EtOAc (2×20 mL). Drying of the organic phase (Na₂SO₄), filtering through a short silica gel pad (50/50 EtOAc/hexane), and concentration afforded the title compound as a brown solid (2.2 g, 75% yield). ¹H NMR (300 MHz, DMSO-D₆) δ ppm 8.23 (s, 1H), 9.18 (s, 1H). M/Z 192.

### Step 3

### 2-chloro-N-ethyl-5-nitro-pyridin-4-amine

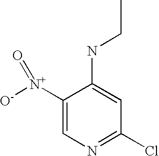

At 0° C., 2,4-dichloro-5-nitro-pyridine (1.7 g, 8.8 mmol) was dissolved in THF (10 mL) and ethylamine (20 mmol, 2 M solution in THF) was slowly added. Concentration afforded the product. ¹H NMR (300 MHz, DMSO-D₆) δ ppm 1.17 (m, 3H), 3.44 (dq, J=6.97, 6.78 Hz, 2H), 7.10 (s, 1H), 8.51 (s, 1H), 8.87 (s, 1H).

### Step 4

### 6-chloro-N′-ethyl-pyridine-3,4-diamine

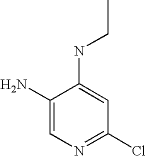

In MeOH (80 mL) was dissolved the 2-chloro-N-ethyl-5-nitro-pyridin-4-amine (1.6 g, 8.0 mmol) from step C, and to the solution was added FeCl₃ (1.6 mmol, 5% in silica gel) and active carbon (3.0 g). The mixture was heated to 72° C. before hydrazine hydrate (4.0 mL, 80 mmol) was slowly added. The reaction was refluxed for 1 h. The mixture was filtered through a short pad of diatomaceous earth, and the residue was washed with MeOH (4×20 mL). The filtrate was concentrated, and to the liquid residue was added water (20 mL). Extraction with EtOAC (2×15 mL), drying (Na₂SO₄), and concentrated afforded the title compound as a pale yellow solid (1.2 g, 80% yield over two steps). ¹H NMR (300 MHz, DMSO-D₆) δ ppm 1.19 (t, J=7.16 Hz, 3H), 3.10 (dt, J=12.25, 7.06 Hz, 2H), 4.75 (s, 2H), 5.64 (s, 1H), 6.28 (s, 1H), 7.37 (s, 1H). M/Z 171.

### Starting Material 1ad

### 6-cyclopropyl-N′-ethyl-pyridine-3,4-diamine

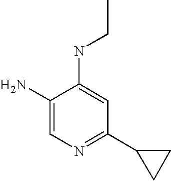

### Step A

### 2-cyclopropyl-N-ethyl-5-nitro-pyridin-4-amine

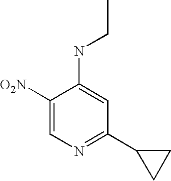

In a 100-mL flask was added 2-chloro-N-ethyl-5-nitro-pyridin-4-amine (0.6 g, 3.0 mmol) (generated in step 3 of SM lab), cyclopropylboronic acid (0.65 g, 7.5 mmol) and K₃PO₄ (1.92 g, 15.0 mmol). To the mixture was added solvent (50 mL, toluene/water=20:1), and N₂ was bubbled into the mixture for 20 min before Pd(PPh₃)₄ (0.85 g, 0.75 mmol) was added. The reaction was heated to 108° C. overnight. After cooling down, to the mixture was added water (30 mL), and extracted with EtOAc (3×20 mL). The organic phase was combined, dried (Na₂SO₄), and concentrated. Silica gel chromatography (EtOAc/hexane, 0-80% gradient) gave the title compound as a solid (0.3 g, 45% yield). ¹H NMR (300 MHz, DMSO-D₆) δ ppm 0.97 (s, 4H), 1.21 (s, 3H), 2.10 (s, 1H), 3.43 (s, 2H), 6.92 (s, 1H), 8.30 (s, 1H), 8.90 (s, 1H). M/Z 207.

### Step 2

### 6-cyclopropyl-N′-ethyl-pyridine-3,4-diamine

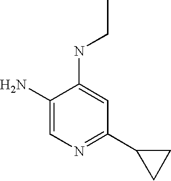

The title compound (0.25 g, 100% yield) was prepared using the protocol described in step 4 of SM lab. ¹H NMR (300 MHz, DMSO-D₆) 8 ppm 0.72 (s, 2H), 0.75 (d, J=3.58 Hz, 2H), 1.20 (t, J=7.16 Hz, 3H), 1.82 (s, 1H), 3.13 (dt, J=12.39, 7.09 Hz, 2H), 4.43 (s, 2H), 5.40 (s, 1H), 6.25 (s, 1H), 7.46 (s, 1H).

### Starting Material 1ad

### 2-Chloro-N⁴-ethylpyridine-3,4-diamine

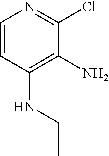

### Step 1

### N-ethyl-3-nitropyridin-4-amine

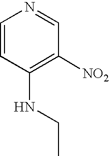

To a solution of 4-chloro-3-nitropyridine (12 g) was added ethylamine (150 mL, 70% in water) and stirred for 10 min at 0° C. The Reaction mixture was extracted with dichloromethane (2×50 ml). The organic layers were concentrated to yield N-ethyl-3-nitropyridin-4-amine, which was used directly in the next step. M/Z 158.

### Step 2

### 2-chloro-N⁴-ethylpyridine-3,4-diamine

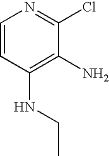

A solution of N-ethyl-3-nitropyridin-4-amine (10 g) in hydrochloride acid (12N, 50 Ml) was heated to 90° C. To this solution was slowly added stannous dichloride (57 g) and the resulting mixture was kept at this temperature for 1 h. The reaction was cooled to rt and water (10 mL) was added. A precipitate was formed and collected to yield 2-chloro-N⁴-ethylpyridine-3,4-diamine (12 g). M/Z 161.

### Starting Material 1ah

### Boc-(R)-Ala-aldehyde

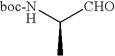

To a solution of commercially available (R)-(+)-2-(tert-Butoxy-carbonylamine)-1-propanol (3.17 g, 18.13 mmol) in dry CH₂Cl₂ (46 mL) at 0° C. under a N₂ atm was added Dess-Martin periodinane (10 g, 23.57 mmol, in two portions, (one, after 2 min, the other portion). It was stirred between 0° C.-10° C. for 45 min and further at rt. Upon completion of the reaction (2.5 h) as indicated by TLC analysis, the mixture was diluted with EtOAc (250 mL). To this 5N NaOH (70 mL) was added and stirred at rt for 20 min. The organic layer was separated, washed with water, brine and dried over anhydrous MgSO₄. The solution was filtered, evaporated and dried to afford the product as an oily solid. The product was stored in the freezer. Yield: 2.49 g (76%). ¹H NMR (300 MHz, CDCl₃) δ: 9.55 (s, 1H), 5.09 (brs, 1H), 4.25-4.10 (m, 1H), 1.44 (s, 9H), 1.33 (d, J=7.14 Hz, 3H).

### Starting Material 1ai

### Step 1

### N-Ethyl-2-methoxy-5-nitropyridin-4-amine

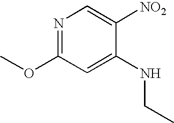

A 250 mL round bottom flask containing 2-chloro-N-ethyl-5-nitropyridin-4-amine was charged with MeOH (25 mL) and NaOMe (1.12 g, 20.7 mmol). The mixture was placed in a 65° C. oil bath. After stirring at 65° C. overnight, the reaction was allowed to cool and the MeOH was removed under reduced pressure. The residue was partitioned between EtOAc and H₂O, and the aqueous layer was extracted with EtOAc. The combined organics were washed with brine, dried (MgSO₄), filtered, and concentrated to a pale yellow solid (827 mg, 86%).

### Step 2

### N⁴-Ethyl-6-methoxypyridine-3,4-diamine

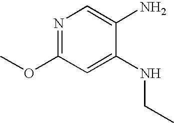

A 250 mL round bottom flask containing N-ethyl-2-methoxy-5-nitropyridin-4-amine (827 mg, 4.19 mmol) was charged with tin (II) chloride dihydrate (3.82 g, 16.93 mmol) and EtOAc (15 mL). The resulting mixture was heated to 80° C. After 4 hours, the mixture was allowed to cool and was treated with aqueous NaHCO₃, precipitating a colorless solid. The mixture was suction-filtered through a pad of diatomaceous earth, and the reaction flask and filter were thoroughly washed with H₂O and EtOAc. The filtrate layers were separated, and the aqueous layer was further extracted with EtOAc. The combined organics were washed with brine, dried (MgSO₄), filtered, and concentrated to a dark red solid (465 mg, 66%).

### Starting Material 1aj

### N⁴-Ethyl-6-trifluoromethyl-pyridine-3,4-diamine

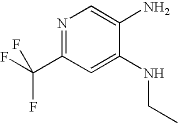

### Starting Material 1aj can be Prepared in the Following Two Procedures:

### Procedure 1:

Starting material 1aj may be prepared by following the procedure as outlined in WO2002050062

### Procedure 2:

Alternatively, it may be prepared as described below:

### Step 1

### 5-bromo-2-trifluoromethyl-pyridin-4-yl-ethylamine

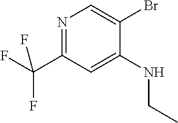

To a solution of 2.0 M LDA in THF/heptane/ethylbenzene (41.77 mL, 83.54 mmol) was added 5-bromo-2-(trifluoromethyl)pyridine (18.8 g, 83.54 mmol) in dry THF (100 mL) drop wise with dropping funnel over 25 min at −78 to −80° C. under N₂ atm. Stirred for 2 h at −78° C., followed by drop wise addition of 12 (21.62 g, 85.21 mmol) in THF (100 mL) over 55 min. After stirring for 15 min at the same temperature, the solution was poured into a mixture of 2.0 M Na₂S₂O₃.5H₂O (200 mL) and diethyl ether (300 mL). It was stirred for 10 min. The phases were separated and the aqueous phase was extracted with diethyl ether. The combined organic layers were washed with brine, dried (Na₂SO₄), evaporated and dried under vacuum to give the crude product, 5-bromo-4-iodo-2-(trifluoromethyl)pyridine, 28.44 g (96.7% recovery, ¹H NMR shows the ratio 88:12 (product and impurity).

The above crude product 5-bromo-4-iodo-2-(trifluoromethyl)pyridine (28.44 g, 79.6 mmol) and 2.0M C₂H₅NH₂ in THF (240 mL, 478 mmol) was heated in a sealed tube at 70-75° C. for 2 days. The reaction mixture was cooled to rt, concentrated and the residue was partitioned between ethyl acetate and water. The organic layer was separated, washed with brine and dried (Na₂SO₄). The solution was filtered, evaporated and the residue purified by flash column on silica gel using 1-2% EtOAc/hexane as eluent. Yield: 15.15 g (67.2% in two steps). ¹H NMR (300 MHz, CDCl₃) J: 8.41 (s, 1H), 6.80 (s, 1H), 4.96 (brs, 1H), 3.34-3.29 (m, 2H), 1.35 (t, J=7.15 Hz, 3H). M/Z=268.08

### Step 2

### N⁴-Ethyl-6-trifluoromethyl-pyridine-3,4-diamine

### Starting Material 1aj

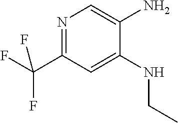

An oven-dried sealed tube equipped with magnetic stir bar and rubber septum was cooled under N₂. The sealed tube was charged with Pd₂ dba₃ (5.55 g, 6 mmol, 20 mol %), rac-BINAP (7.55 g, 12.12 mmol, 40 mol %) and toluene (200 mL). The mixture was degassed, the rubber septum was replaced with a Teflon screw cap, and the mixture was heated at 110° C. in an oil bath for 30 min. The solution was then allowed to cool to rt, and benzophenone imine (6.61 mL, 39.5 mmol), 5-bromo-2-trifluoromethyl-pyridin-4-yl-ethylamine (8.16 g, 30.32 mmol) in toluene (100 mL), and sodium tert-butoxide (3.8 g, 39.5 mmol) were added. The mixture was degassed, the rubber septum was replaced with Teflon cap, and the mixture was heated between 135-140° C. for 16 h. The solution was then allowed to cool to rt, diluted with ether, filtered through a pad of Celite, and concentrated to give the imine adduct as a dark green-brown oil, 27.24 g. To this crude imine adduct in THF (242 mL) was added aqueous 2.0 M HCl (80 mL) and stirred at rt for 20 h. The solvent was concentrated and the reaction mixture was partitioned between EtOAc (1 L) and 2.0 M HCl (120 mL). The aqueous layer was separated, cooled to 0° C. and basified with NaOH to pH=14. The reaction mixture was extracted with EtOAc (2×500 mL) washed with brine and dried (Na₂SO₄). The solution was filtered, evaporated and dried under vacuum to give crude diamine as off-white solid 4.09 g (64.4%).

Another reaction was carried out with the Pd₂ dba₃ (7.25 g, 7.92 mmol, 20 mol %), rac-BINAP (9.86 g, 15.84 mmol), toluene (200 mL), benzophenone imine (8.64 mL, 51.5 mmol), 5-bromo-2-trifluoromethyl-pyridin-4-yl-ethylamine (10.66 g, 39.61 mmol) in toluene (100 mL), and sodium tert-butoxide (4.94 g, 51.5 mmol) to afford the crude imine adduct 36.47 g. This was treated with THF (325 mL) and 2.0M HCl (110 mL) gave the crude diamine 5.13 g (63.17%).

The above crude diamines (9.22 g) were combined and purified by flash column on silica gel using 35-40% EtOAC/hexane as eluent to obtain 7.55 g (52% in two steps). ¹H NMR (300 MHz, DMSO-d₆), 7.69 (s, 1H), 6.65 (s, 1H), 5.65 (t, J=4.68 Hz, 1H), 5.23 (brs, 2H), 3.21-3.12 (m, 2H), 1.21 (t, J=7.15 Hz, 3H). M/Z=205.0.

### Preparation of Sulfonyl Chlorides (SC):

General procedure for synthesis of 4-substituted-3-pyridyl sulfonyl chlorides from aminopyridines (SC 1-SC4): A 250 mL round bottom flask is charged with water (30 mL) and is cooled to 0° C. Thionyl chloride (6.0 mL, 82.3 mmol) is added dropwise over a period of 2 hours. The mixture is slowly allowed to warm to room temperature overnight. CuCl (72 mg, 0.73 mmol) is added, and the yellow solution is cooled to 0° C. Meanwhile, a separate 100 mL round bottom flask is charged with a 3-aminopyridine derivative (15.0 mmol) and concentrated HCl (20 mL). The solution is cooled to 0° C., and then a solution of sodium nitrite (1.49 g, 21.6 mmol) in H₂O (15 mL) is added dropwise over 10 minutes. This mixture is allowed to stir at 0° C. for an additional 15 minutes, and is then added dropwise (keeping the bulk of the diazonium mixture at 0° C.) to the water/thionyl chloride solution over 10 minutes. After 1 hour at 0° C., the mixture is extracted with CH₂Cl₂ (2×), and the combined organics are washed with brine, dried (MgSO₄), filtered, and concentrated to give the sulfonyl chloride which is used without any further purification.

SC 5 was prepared as described in J. Chem. Soc., 1948, 1939-1945. SC 6 was prepared as follows:
Pyridine-4-sulfonyl chloride (SC 6)

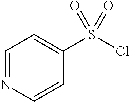

Pyridine-4-thiol (1.101 g, 0.01 mol) was dissolved in concentrated hydrochloric acid (7.5 mL)+water (2 mL) in a 50 mL 3-neck round-bottomed flask and cooled in dry ice/acetone bath to −10 C. Chlorine gas was introduced into the solution through a sparge tube for 45 min, dry ice being added to the acetone bath as necessary in order to maintain a temperature of −10° C. Once the addition of chlorine gas was complete, calcium carbonate (1 g) was slowly added to the reaction mixture The reaction mixture was then transferred into ca 20 mL chloroform, cooled to −10° C. More calcium chloride (7 g) was added portionwise. After the addition was complete, the organic layer was decanted from the semi-solid inorganics. The gum was then washed twice with chloroform (two portions, 20 mL each). The combined organic layer was dried over sodium sulfate. This chloroform solution was used as is. SC 7 was prepared in two steps from commercially available 2,6-dimethylpyridin-4(1H)-one as described below:

### 2,6-dimethylpyridine-4-sulfonyl chloride

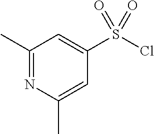

### Step 1

### 2,6-dimethylpyridine-4(1H)-thione

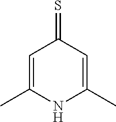

Under a nitrogen purge, 2,6-dimethylpyridin-4(1H)-one (5.00 g, 0.04060 mol) toluene (150 mL) in a 500 mL 3-neck round bottom flask. Lawesson's Reagent (16.8 g, 0.04 mol), was sifted into the reaction, washing in with a bit more toluene. The suspension was heated to reflux, and maintained 16 h. Solvent was decanted from the gummy yellow solid, which was then further extracted with a few more portions of hot toluene. The remaining gummy yellow solid was dissolved with difficulty in hot acetonitrile (ca 400 mL), and then pre-absorbed onto silica. Flash chromatography using a gradient of 100% dichloromethane to 20% ethanol in dichloromethane; 5% v:v concentrated ammonium hydroxide in the ethanol to obtain the desired product which was further purified by recrystallization from hot water to provide light yellow crystals (2.1 g, 37%). ¹H NMR (300 MHz, MeOH-d₄) δ ppm 2.32-2.36 (m, 6H) 7.14-7.17 (m, 2H) M/z=140

### Step 2

### 2,6-dimethylpyridine-4-sulfonyl chloride

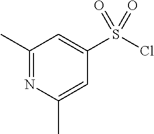

2,6-dimethylpyridine-4(1H)-thione (Step 1, 1.403 g, 0.01008 mol) concentrated hydrochloric acid (7.5 mL) and water (2.0 mL) in a 50 mL 3-neck round-bottomed flask and cooled in dry ice/acetone bath to −10° C. Chlorine gas was introduced through a sparge tube for about 45 min, dry ice was added as necessary to keep the temperature −10° C.+/−5° C. Afterwards, nitrogen was sparged through the system for 15 min. Calcium carbonate (1 g) was added to the reaction mixture and the reaction mixture was then transferred into chloroform (20 mL, pre-cooled to −5° C.). More calcium carbonate (7 g) was added in small portions. After the addition was complete, the organic layer was decanted from the semi-solid gum which was washed twice with cold chloroform (2×20 mL). The combined organic layer was dried over sodium sulfate and filtered. The filtrate was used as is without further purification. SC 8 was prepared as described below:

### Step 1

### 2-methylpyridine-4(1H)-thione

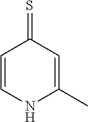

Sodium hydrosulfide hydrate (5.49 g, 97.98 mmol) 10 mL water in a 20 mL pressure tube, along with benzyltriethylammonium bromide (0.267 g, 0.98 mmol). 4-chloro-2-methylpyridine (2.5 g, 19.60 mmol) subjected to microwave irradiation, 140° C. for 6 h. This resulted in a mostly clear, yellow solution, along with some dark solid at the bottom of the tube. The clear yellow liquid was decanted, and then cooled in an ice bath, causing a solid to precipitate. The yellow solid was filtered, and then washed with a small portion of ice-cold water. After air-drying on the filter, this material was used as-is in the subsequent step. ¹H NMR (300 MHz, MeOH-d₄) δ ppm 2.34-2.38 (m, 3H) 7.28-7.35 (m, 2H) 7.53-7.57 (m, 1H) M/z=125

### Step 2

### 2-methylpyridine-4-sulfonyl chloride

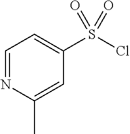

2-methylpyridine-4(1H)-thione (Step 1, 1.252 g, 0.01 mol) in 7.5 mL concentrated hydrochloric acid/2 mL water solution in a 50 mL 3-neck round-bottomed flask and cooled in dry ice/acetone bath to −10° C. Chlorine gas was introduced through a sparge tube for about 45 min, dry ice being added as necessary to the acetone bath in order to keep the reaction temperature −10 C.°+/−5° C. Afterwards, nitrogen was sparged through the system for 15 min. Calcium carbonate (1 g) was cautiously added to the reaction mixture, to avoid excessive bubbling. The reaction mixture was then transferred into chloroform, previously cooled to −10° C. More calcium carbonate (7 g) was cautiously added portionwise. After the addition was complete, the organic layer was decanted from the semi-solid gum. The gum was then washed twice with 20 mL portions of cold chloroform. The combined chloroform layer was dried over sodium sulfate and filtered and used immediately without further purification. SC 9 was prepared in two steps as described below:

### Step 1

### 5-Amino-pyridine-2-carboxylic acid methylamide

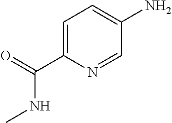

To a suspension of 5-amino-pyridine-2-carboxylic acid (2.0 g, 14.5 mmol) and CDI (2.6 g, 15.9 mmol) in THF (20 mL) was added DMF (10 mL). The reaction mixture became turbid in 10 min. Methyl amine in THF (2M, 21.8 mL) was added and the reaction mixture was allowed to stir at room temperature over night. The reaction mixture was concentrated. The residue was diluted with ethyl acetate, washed with water followed by brine, dried (Na₂SO₄), filtered and concentrated. The white solid obtained was triturated with cold ether to give 5 as a white solid. Yield: 1.2 g (55%). ¹H NMR (301 MHz, DMSO-d₆) δ ppm 8.23-8.34 (m, 1H), 7.89 (d, J=2.8 Hz, 1H), 7.69 (d, J=8.5 Hz, 1H), 6.95 (dd, J=8.5, 2.8 Hz, 1H), 5.90 (s, 2H), 2.75 (d, J=4.7 Hz, 3H). (M+1)/Z=152.

### Step 2

### 6-Methylcarbamoyl-pyridine-3-sulfonyl chloride

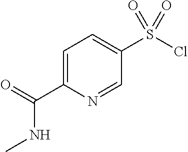

Thionyl chloride (4.1 mL, 56.0 mmol) was added to water (22 mL) at 0° C. over a period of 1 h maintaining the reaction temperature below 5° C. The reaction mixture was allowed to warm to 18° C. over a period of 20 h. To this mixture was added copper (I) chloride (0.17 g, 0.2 mol) and the resulting yellow-green solution was cooled to −5° C. In parallel, 5-amino-pyridine-2-carboxylic acid methylamide 5 (1.2 g, 8.0 mmol) was dissolved in concentrated HCl (12 mL). To this mixture was added dropwise over a period of 1 h a solution of NaNO₂ (1.0 g, 14.0 mmol) in water (6 mL), maintaining the reaction temperature at −5° C. This slurry was then added dropwise over a period of 1 h to the above mixture (thionyl chloride/water mixture), maintaining temperature between −5 and 0° C. (Note: the diazotized mixture should be kept below −5° C. through out the addition). As the addition proceeds, a white solid precipitates. The reaction mixture was stirred for an additional hour below 0° C. The precipitate was collected by filtration, washed with cold water and dried under vacuum to afford the title compound as light yellow solid (0.18 g, Yield: 10%).

SC 10 was prepared by modifying the procedure for SC 1 in WO 2007/023186 as detailed below:

### 6-Carbamoyl-pyridine-3-sulfonyl chloride

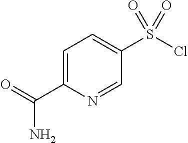

Thionyl chloride (31.4 mL, 0.47 mol) was added to water (182 mL) at 0° C. over a period of 1 h maintaining the reaction temperature below 5° C. The reaction mixture was allowed to warm to 18° C. over a period of 20 h. To this mixture was added copper (1) chloride (0.14 g, 0.001 mol) and the resulting yellow-green solution was cooled to −5° C. In parallel, 5-amino-2-cyano pyridine (10.0 g, 0.08 mol) was dissolved slowing in concentrated HCl (98 mL) and allowed to stir at room temperature over the weekend. To this mixture was added dropwise over a period of 1 h a solution of NaNO₂ (8.2 g, 0.12 mol) in water (50 mL), maintaining the reaction temperature at −5° C. This slurry was then added dropwise over a period of 1 h to the above mixture (thionyl chloride/water mixture), maintaining temperature between −5 and 0° C. (Note: the diazotized mixture should be kept below −5° C. through out the addition). As the addition proceeds, a white solid precipitates. The reaction mixture was stirred for an additional hour below 0° C. The precipitate was collected by filtration, washed with cold water and dried under vacuum to afford the title compound as light yellow solid (5.1 g, Yield: 27%).

TABLE 6

| SC# | Structure | NMR | M/Z |
|---|---|---|---|
| 1 | 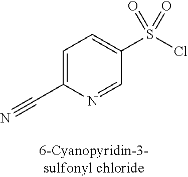 | ¹H NMR (CDCl₃) δ ppm 7.97 (m, 1 H) 8.45-8.52 (m, 1 H) 9.32 (m, 1 H). | — |
| 2 | 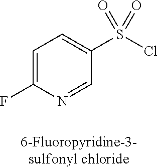 | ¹H NMR (CDCl₃) 7.20 (dd, J = 8.72, 2.91 Hz, 1 H) 8.42 (ddd, J = 8.97, 6.57, 2.65 Hz, 1 H) 8.92 (d, J = 2.53 Hz, 1 H). | — |
| 3 | 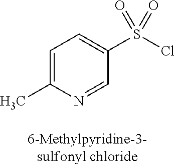 | — | — |
| 4 | 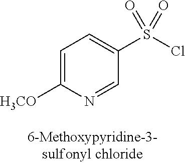 | ¹H NMR (CDCl₃) 4.05 (s, 3 H) 6.90 (m, 1 H) 8.10 (m, 1 H) 8.82 (m, 1 H) | — |
| 5 | 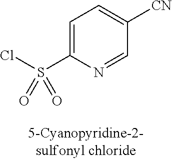 | — | — |
| 6 | 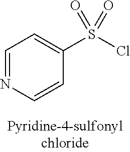 | — | — |
| 7 | 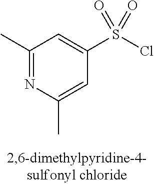 | — | — |
| 8 | 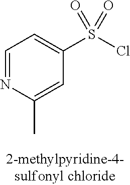 | — | — |
| 9 | 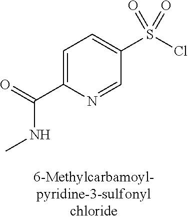 | ¹H NMR (301 MHz, CDCl₃) δ ppm 9.16 (t, J = 1.5 Hz, 1 H), 8.47 (d, J = 1.4 Hz, 2 H), 7.99 (br. s., 1 H), 3.09 (d, J = 5.2 Hz, 3 H). | — |
| 10 | 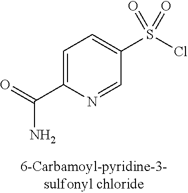 | ¹H NMR (301 MHz, DMSO-d₆) δ ppm 9.20 (s, 1 H), 8.48 (s, 2 H), 7.82 (bs, 1 H), 5.87 (bs, 1 H) | — |

1. A compound of formula (I): 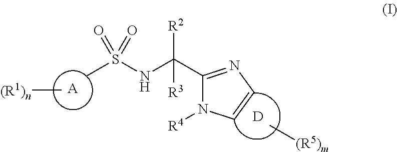
Ring A is carbocyclyl or heterocyclyl; wherein if said heterocyclyl contains an —NH— moiety that nitrogen may be optionally substituted by a group selected from R⁶;
R¹ is independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, hydrazinyl, ureido, N,N-di(C₁₋₃alkyl)ureido, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl; wherein R¹ may be optionally substituted on carbon by one or more R⁷; and wherein if said heterocyclyl contains an —NH— moiety that nitrogen may be optionally substituted by a group selected from R⁸.
n is 0-5; wherein the values of R¹ may be the same or different;
R² is selected from C₁₋₆alkyl, C₂₋₆alkenyl or C₂₋₆alkynyl, carbocyclyl, and heterocyclyl; wherein R² may be optionally substituted on carbon by one or more R⁹; wherein if said heterocyclyl contains an NH moiety that nitrogen may be optionally substituted by a group selected from R¹⁹;
R³ is selected from hydrogen, C₁₋₆alkyl, C₂₋₆alkenyl or C₂₋₆alkynyl, carbocyclyl, heterocyclyl; wherein R³ may be optionally substituted on carbon by one or more R¹¹; wherein if said heterocyclyl contains an NH moiety that nitrogen may be optionally substituted by a group selected from R²⁰;
or, alternatively, R² and R³ may, together with the carbon to which they are attached, form a C₃₋₆carbocyclic ring;
R⁴ is selected from C₁₋₆alkyl or carbocyclyl; wherein R⁴ may be optionally substituted on carbon by one or more R¹⁰;
Ring D is fused to the imidazole of formula (I) and is a 5-7 membered ring; wherein if said ring contains an —NH— moiety that nitrogen may be optionally substituted by a group selected from R¹⁴;
R⁵ is a substituent on carbon and is independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, heterocyclylcarbonyl, N—(C₁₋₆alkyl)sulphamoyl, N,N—(C₁₋₆alkyl)₂sulphamoyl, C₁₋₆alkylsulphonylamino, carbocyclyl or heterocyclyl, or two R⁵ may together with the carbon atoms of ring D to which they are attached form a 5 to 8-membered carbocyclyl or heterocyclyl ring; wherein R⁵ may be optionally substituted on carbon by one or more R¹⁵; and wherein if said heterocyclyl or heterocyclyl ring contains an —NH— moiety that nitrogen may be optionally substituted by a group selected from R¹⁶;
m is 0-5; wherein the values of R⁵ may be the same or different;
R⁷, R⁹, R¹¹ and R¹⁵ are independently selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁷, R⁹, R¹¹ and R¹⁵ may be independently optionally substituted on carbon by one or more R¹⁷; and wherein if said heterocyclyl contains an —NH— moiety that nitrogen may be optionally substituted by a group selected from R¹⁸;
R⁶, R⁸, R¹³, R¹⁴, R¹⁶, R¹⁸, R¹⁹ and R²⁰ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R¹⁰ is selected from halo, nitro, hydroxy, amino, carboxy, mercapto, sulphamoyl, C₁₋₆alkyl, C₂₋₆alkenyl, C₂₋₆alkynyl, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R¹⁰ may be optionally substituted on carbon by one or more R¹²; and wherein if said heterocyclyl contains an —NH— moiety that nitrogen may be optionally substituted by a group selected from R¹³;
R¹² and R¹⁷ are selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof; provided the compound is not 4-methyl-N-[(1-methyl-1H-benzimidazol-2-yl)phenylmethyl]benzenesulfonamide.

2. A compound of formula (I) according to claim 1 having formula (Ia) 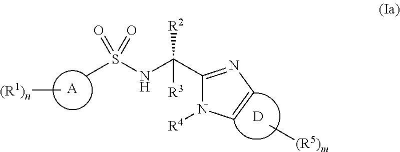
wherein R³ is hydrogen and A, D, R¹, R², R⁴, R⁵, m and n are as defined in claim 1, and pharmaceutically acceptable salts thereof.

3. A compound according to claim 1 wherein Ring A is phenyl or pyridinyl.

4. A compound of formula (I) according to claim 1 selected from
(R)—N-(1-(1-Ethyl-6-(trifluoromethyl)-1H-imidazo[4,5-c]pyridin-2-yl)ethyl)-4-fluorobenzenesulfonamide;
(R)-6-Cyano-N-(1-(1-ethyl-6-(trifluoromethyl)-1H-imidazo[4,5-c]pyridin-2-yl)ethyl)pyridine-3-sulfonamide;
(R)-5-(N-(1-(1-Ethyl-6-(trifluoromethyl)-1H-imidazo[4,5-c]pyridin-2-yl)ethyl)sulfamoyl)picolinamide;
(R)-4-Cyano-N-(1-(1-ethyl-6-methoxy-1H-imidazo[4,5-c]pyridin-2-yl)ethyl)benzenesulfonamide;
(R)-6-Cyano-N-(1-(1-ethyl-6-methoxy-1H-imidazo[4,5-c]pyridin-2-yl)ethyl)pyridine-3-sulfonamide;
(R)-6-Cyano-N-(1-(1-ethyl-6-methoxy-1H-imidazo[4,5-c]pyridin-2-yl)ethyl)pyridine-3-sulfonamide;
(R)-5-(N-(1-(1-Ethyl-6-(trifluoromethyl)-1H-benzo[d]imidazol-2-yl)ethyl)sulfamoyl)-1-methyl-1H-pyrrole-2-carboxamide;
(R)—N-(1-(1-Ethyl-6-(trifluoromethyl)-1H-benzo[d]imidazol-2-yl)ethyl)-2,6-dimethylpyridine-4-sulfonamide;
(R)—N-(1-(1-Ethyl-6-(trifluoromethyl)-1H-benzo[d]imidazol-2-yl)ethyl)-2-methylpyridine-4-sulfonamide;
6-Cyano-N-[(1R)-1-(6-cyclopropyl-1-ethyl-1H-imidazo[4,5-c]pyridin-2-yl)ethyl]pyridine-3-sulfonamide;
5-({[(1R)-1-(6-Cyclopropyl-1-ethyl-1H-imidazo[4,5-c]pyridin-2-yl)ethyl]amino}sulfonyl)pyridine-2-carboxamide;
4-Cyano-N-{(1R)-1-[1-ethyl-6-(trifluoromethyl)-1H-imidazo[4,5-b]pyridin-2-yl]ethyl}benzenesulfonamide;
4-Cyano-N-[1-(6-cyclopropyl-1-ethyl-1H-imidazo[4,5-c]pyridin-2-yl)-ethyl]-benzenesulfonamide; and pharmaceutically acceptable salts thereof.

5. A pharmaceutical composition which comprises a compound of the formula (I), or a pharmaceutically acceptable salt thereof, as claimed in claim 1 in association with a pharmaceutically-acceptable carrier, diluent or excipient.

6. A compound of the formula (I), or a pharmaceutically acceptable salt thereof, as claimed in claim 1 for use as a medicament.

7. (canceled)

8. (canceled)

9. (canceled)

10. A method for producing an Edg-1 antagonistic effect in a warm-blooded animal, such as man, which comprises administering to said animal an effective amount of a compound of formula (I), or a pharmaceutically acceptable salt thereof, as claimed in claim 1.

11. A method for producing an anti-cancer effect in a warm-blooded animal, which comprises administering to said animal an effective amount of a compound of formula (I), or a pharmaceutically acceptable salt thereof, as claimed in claim 1.

12. A method of treating of angiogenesis-related diseases including non-solid tumors, solid tumors and their metastases, non-small cell lung cancer, glioma, hepatocellular (liver) carcinoma, glioblastoma, carcinoma of the thyroid, bile duct, bone, gastric, brain/CNS, head and neck, hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid tumors, in a warm-blooded animal in need of such treatment which comprises administering to said animal an effective amount of a compound of formula (I) or a pharmaceutically acceptable salt thereof, as claimed in claim 1.

13. A pharmaceutical composition which comprises a compound of the formula (I), or a pharmaceutically acceptable salt thereof, as claimed in claim 1, in association with a pharmaceutically-acceptable carrier, diluent or excipient for use in the production of a Edg-1 antagonistic effect in a warm-blooded animal such as man.

14. A pharmaceutical composition which comprises a compound of the formula (I), or a pharmaceutically acceptable salt thereof, as claimed in claim 1, in association with a pharmaceutically-acceptable carrier, diluent or excipient for use in the production of an anti-cancer effect in a warm-blooded animal such as man.

15. Processes for preparing a compound of formula (I) or a pharmaceutically acceptable salt thereof as claimed in claim 1, wherein the variables are, unless otherwise specified, as defined in claim 1, which processes comprise
Process a) reacting of a compound of formula (II): 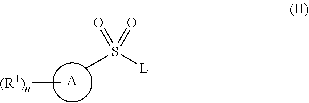
with an amine of formula (III): 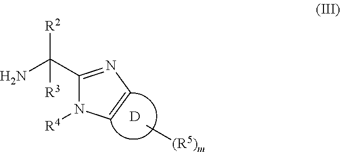
wherein L is a displaceable group,
and thereafter if necessary:
i) converting a compound of the formula (I) into another compound of the formula (I);
ii) removing any protecting groups;
iii) forming a pharmaceutically acceptable salt.
